SEQUENCE LISTING (1276 sequences)

SEQ ID NO: 1 - DNA, 21 bp, Artificial Sequence
  siRNA
aaggttctacttgagcaagaa

SEQ ID NO: 2 - DNA, 21 bp, Artificial Sequence
  siRNA
caccatgagcgacgtggctat

SEQ ID NO: 3 - DNA, 21 bp, Artificial Sequence
  siRNA
aagtgctttcactgtggagga

SEQ ID NO: 4 - DNA, 21 bp, Artificial Sequence
  siRNA
accaaggacatgtttgataaa

SEQ ID NO: 5 - DNA, 21 bp, Artificial Sequence
  siRNA
ccctactgtctttgagaacta

SEQ ID NO: 6 - DNA, 21 bp, Artificial Sequence
  siRNA
ccctactgtctttgagaacta

SEQ ID NO: 7 - DNA, 21 bp, Artificial Sequence
  siRNA
gcgagccatgctagtttgata

SEQ ID NO: 8 - DNA, 21 bp, Artificial Sequence
  siRNA
cgccaagaaagatacgagata

SEQ ID NO: 9 - DNA, 21 bp, Artificial Sequence
  siRNA
tacctcttcccaggacattaa

SEQ ID NO: 10 - DNA, 21 bp, Artificial Sequence
  siRNA
caggtgttgcagcatcattga

SEQ ID NO: 11 - DNA, 21 bp, Artificial Sequence
  siRNA
aacggctgatgtggactgtct

SEQ ID NO: 12 - DNA, 21 bp, Artificial Sequence
  siRNA
aaggtacttcgatgatgaatt

SEQ ID NO: 13 - DNA, 21 bp, Artificial Sequence
  siRNA
ctggtcttcaattaccaagaa

SEQ ID NO: 14 - DNA, 21 bp, Artificial Sequence
  siRNA
ttcgcggatcttcgggaagaa

SEQ ID NO: 15 - DNA, 21 bp, Artificial Sequence
  siRNA
cacgctgtgcgctacctcgaa

SEQ ID NO: 16 - DNA, 21 bp, Artificial Sequence
  siRNA
caacgtgaacgcgcaaatgta

SEQ ID NO: 17 - DNA, 21 bp, Artificial Sequence
  siRNA
gcggcagacattaaaggtaca

SEQ ID NO: 18 - DNA, 21 bp, Artificial Sequence
  siRNA
ctggttgtatcttattagcaa

SEQ ID NO: 19 - DNA, 21 bp, Artificial Sequence
  siRNA
aagcaagtgtacgacgcgcac

SEQ ID NO: 20 - DNA, 21 bp, Artificial Sequence
  siRNA
cagggccgtgcgggaccgcaa

SEQ ID NO: 21 - DNA, 21 bp, Artificial Sequence
  siRNA
ctgatcgtatgcagaaggaaa

SEQ ID NO: 22 - DNA, 21 bp, Artificial Sequence
  siRNA
ctgggcctgtataccggataa

SEQ ID NO: 23 - DNA, 21 bp, Artificial Sequence
  siRNA
ccgggatggcatgagtgtcta

SEQ ID NO: 24 - DNA, 21 bp, Artificial Sequence
  siRNA
agcgccaagtccagaaccata

SEQ ID NO: 25 - DNA, 21 bp, Artificial Sequence
  siRNA
ccagttgtacttcagcaccaa

SEQ ID NO: 26 - DNA, 21 bp, Artificial Sequence
  siRNA
aaggtcaacgacaaagaggtg

SEQ ID NO: 27 - DNA, 21 bp, Artificial Sequence
  siRNA
acggatattgtttcacgatga

SEQ ID NO: 28 - DNA, 21 bp, Artificial Sequence
  siRNA
aagcccttctgcacatctaaa

SEQ ID NO: 29 - DNA, 21 bp, Artificial Sequence
  siRNA
acgctaataagtgacaaataa

SEQ ID NO: 30 - DNA, 21 bp, Artificial Sequence
  siRNA
catgcggaagatgctggctta

SEQ ID NO: 31 - DNA, 21 bp, Artificial Sequence
  siRNA
cccgcaaatcatcaactccaa

SEQ ID NO: 32 - DNA, 21 bp, Artificial Sequence
  siRNA
ccaataaagaccagcaagcaa

SEQ ID NO: 33 - DNA, 21 bp, Artificial Sequence
  siRNA
atgattgacagtagtttatca

SEQ ID NO: 34 - DNA, 21 bp, Artificial Sequence
  siRNA
aaccttgtatctcttctgcaa

SEQ ID NO: 35 - DNA, 21 bp, Artificial Sequence
  siRNA
ctcgacttctccagctggtta

SEQ ID NO: 36 - DNA, 21 bp, Artificial Sequence
  siRNA
ctccatccgttggttcttcaa

SEQ ID NO: 37 - DNA, 21 bp, Artificial Sequence
  siRNA
aacatatagaggccctattgg

SEQ ID NO: 38 - DNA, 21 bp, Artificial Sequence
  siRNA
ccgcagagctgcgtcacgtaa

SEQ ID NO: 39 - DNA, 21 bp, Artificial Sequence
  siRNA
cagctttgacggcgtgtggaa

SEQ ID NO: 40 - DNA, 21 bp, Artificial Sequence
  siRNA
aaggcaaacgtgaccgttttt

SEQ ID NO: 41 - DNA, 21 bp, Artificial Sequence
  siRNA
caccgtcatcaatgccctcaa

SEQ ID NO: 42 - DNA, 21 bp, Artificial Sequence
  siRNA
cacggcctgagcgtccagaaa

SEQ ID NO: 43 - DNA, 21 bp, Artificial Sequence
  siRNA
agggaagaccacgatcctcta

SEQ ID NO: 44 - DNA, 21 bp, Artificial Sequence
  siRNA
taccttatagttactgtgtaa

SEQ ID NO: 45 - DNA, 21 bp, Artificial Sequence
  siRNA
aaggatagattgtgtttcaaa

SEQ ID NO: 46 - DNA, 21 bp, Artificial Sequence
  siRNA
ctggtaagcgactgtcatcaa

SEQ ID NO: 47 - DNA, 21 bp, Artificial Sequence
  siRNA
atccatgagatagctattata

SEQ ID NO: 48 - DNA, 21 bp, Artificial Sequence
  siRNA
ctcggatatgtcgacttctga

SEQ ID NO: 49 - DNA, 21 bp, Artificial Sequence
  siRNA
caggatacaaggcagatccaa

SEQ ID NO: 50 - DNA, 21 bp, Artificial Sequence
  siRNA
ctcagtatcctggatctgaaa

SEQ ID NO: 51 - DNA, 21 bp, Artificial Sequence
  siRNA
aggctcttgatggctatagta

SEQ ID NO: 52 - DNA, 21 bp, Artificial Sequence
  siRNA
ccgacaaactttacaaatcca

SEQ ID NO: 53 - DNA, 21 bp, Artificial Sequence
  siRNA
cacctatatgaccaatcccta

SEQ ID NO: 54 - DNA, 21 bp, Artificial Sequence
  siRNA
cccggactcgctggagaacat

SEQ ID NO: 55 - DNA, 21 bp, Artificial Sequence
  siRNA
tccaagattctagatgattaa

SEQ ID NO: 56 - DNA, 21 bp, Artificial Sequence
  siRNA
caccgaattaatatttatgaa

SEQ ID NO: 57 - DNA, 21 bp, Artificial Sequence
  siRNA
caggaatgatttcctgaaaga

SEQ ID NO: 58 - DNA, 21 bp, Artificial Sequence
  siRNA
agggctgagtttgtattatta

SEQ ID NO: 59 - DNA, 21 bp, Artificial Sequence
  siRNA
ccgtactatcttgtcaagata

SEQ ID NO: 60 - DNA, 21 bp, Artificial Sequence
  siRNA
aacgagctcaatctaggacga

SEQ ID NO: 61 - DNA, 21 bp, Artificial Sequence
  siRNA
cccaagatactcatcagtcaa

SEQ ID NO: 62 - DNA, 21 bp, Artificial Sequence
  siRNA
cacgcttggtcccgaggccaa

SEQ ID NO: 63 - DNA, 21 bp, Artificial Sequence
  siRNA
ggccggaatcttaatattcga

SEQ ID NO: 64 - DNA, 21 bp, Artificial Sequence
  siRNA
cccatttggaattattcctaa

SEQ ID NO: 65 - DNA, 21 bp, Artificial Sequence
  siRNA
caccatggctgcaatccgaaa

SEQ ID NO: 66 - DNA, 21 bp, Artificial Sequence
  siRNA
aacaccagctcctgtgctgcg

SEQ ID NO: 67 - DNA, 21 bp, Artificial Sequence
  siRNA
cagcagcatccctgagaacga

SEQ ID NO: 68 - DNA, 21 bp, Artificial Sequence
  siRNA
atggatcaaagtttgaattaa

SEQ ID NO: 69 - DNA, 21 bp, Artificial Sequence
  siRNA
ctcattgaacatatgcaagaa

SEQ ID NO: 70 - DNA, 21 bp, Artificial Sequence
  siRNA
acgggcaataagactctttaa

SEQ ID NO: 71 - DNA, 21 bp, Artificial Sequence
  siRNA
aaccaagcctttgaaacaatg

SEQ ID NO: 72 - DNA, 21 bp, Artificial Sequence
  siRNA
aacaacttctccgtagcagaa

SEQ ID NO: 73 - DNA, 21 bp, Artificial Sequence
  siRNA
acccaattaagtcctacttta

SEQ ID NO: 74 - DNA, 21 bp, Artificial Sequence
  siRNA
catctttgtggtggacagtaa

SEQ ID NO: 75 - DNA, 21 bp, Artificial Sequence
  siRNA
gagaaggtgaatgtaccctaa

SEQ ID NO: 76 - DNA, 21 bp, Artificial Sequence
  siRNA
ccggccggaggcgctttacta

SEQ ID NO: 77 - DNA, 21 bp, Artificial Sequence
  siRNA
cagctacgccggacaatagaa

SEQ ID NO: 78 - DNA, 21 bp, Artificial Sequence
  siRNA
cggcaacttacacacattgaa

SEQ ID NO: 79 - DNA, 21 bp, Artificial Sequence
  siRNA
caccttcactgtgacgaaata

SEQ ID NO: 80 - DNA, 21 bp, Artificial Sequence
  siRNA
aagaaatagacttgcacctta

SEQ ID NO: 81 - DNA, 21 bp, Artificial Sequence
  siRNA
tcctagcaccatgaagattaa

SEQ ID NO: 82 - DNA, 21 bp, Artificial Sequence
  siRNA
cacctacgtatttaagatgaa

SEQ ID NO: 83 - DNA, 21 bp, Artificial Sequence
  siRNA
ccgactcatcaagggacgaaa

SEQ ID NO: 84 - DNA, 21 bp, Artificial Sequence
  siRNA
aagcatagagttatccttcta

SEQ ID NO: 85 - DNA, 21 bp, Artificial Sequence
  siRNA
acgcgttatagtaactcccat

SEQ ID NO: 86 - DNA, 21 bp, Artificial Sequence
  siRNA
cagcattccgctgaccatcaa

SEQ ID NO: 87 - DNA, 21 bp, Artificial Sequence
  siRNA
aacgaatgtaataaagaccta

SEQ ID NO: 88 - DNA, 21 bp, Artificial Sequence
  siRNA
gaccttgtacagaatgtgtta

SEQ ID NO: 89 - DNA, 21 bp, Artificial Sequence
  siRNA
ccggctcaactcgcatctcaa

SEQ ID NO: 90 - DNA, 21 bp, Artificial Sequence
  siRNA
tgggacagaattggatacata

SEQ ID NO: 91 - DNA, 21 bp, Artificial Sequence
  siRNA
acgcagcatcgtggactacaa

SEQ ID NO: 92 - DNA, 21 bp, Artificial Sequence
  siRNA
caggccatccgcggaactcga

SEQ ID NO: 93 - DNA, 21 bp, Artificial Sequence
  siRNA
atggatttgaggttacctcaa

SEQ ID NO: 94 - DNA, 21 bp, Artificial Sequence
  siRNA
ttggataaccttccttctaaa

SEQ ID NO: 95 - DNA, 21 bp, Artificial Sequence
  siRNA
ccgcgtgaagctggtgaactt

SEQ ID NO: 96 - DNA, 21 bp, Artificial Sequence
  siRNA
caagacgatcatagtcactga

SEQ ID NO: 97 - DNA, 21 bp, Artificial Sequence
  siRNA
ttgcttatatgttaaattgaa

SEQ ID NO: 98 - DNA, 21 bp, Artificial Sequence
  siRNA
caccaattgattgactgagaa

SEQ ID NO: 99 - DNA, 21 bp, Artificial Sequence
  siRNA
ttgcgttcacttgtactgtaa

SEQ ID NO: 100 - DNA, 21 bp, Artificial Sequence
  siRNA
caagatttctttgaccggtat

SEQ ID NO: 101 - DNA, 21 bp, Artificial Sequence
  siRNA
ttggatggctacagtaagaaa

SEQ ID NO: 102 - DNA, 21 bp, Artificial Sequence
  siRNA
cggcaagtccctgtactatta

SEQ ID NO: 103 - DNA, 21 bp, Artificial Sequence
  siRNA
cacgatcctctacaagcttaa

SEQ ID NO: 104 - DNA, 21 bp, Artificial Sequence
  siRNA
caagctagacgtgggaagaaa

SEQ ID NO: 105 - DNA, 21 bp, Artificial Sequence
  siRNA
caggatgatcgtgacaagttt

SEQ ID NO: 106 - DNA, 21 bp, Artificial Sequence
  siRNA
caacatgaaggtggccattaa

SEQ ID NO: 107 - DNA, 21 bp, Artificial Sequence
  siRNA
caccagaagtcctgaaactaa

SEQ ID NO: 108 - DNA, 21 bp, Artificial Sequence
  siRNA
ccgatgagaaacctcgtgtta

SEQ ID NO: 109 - DNA, 21 bp, Artificial Sequence
  siRNA
caggtcgttcttatctagaga

SEQ ID NO: 110 - DNA, 21 bp, Artificial Sequence
  siRNA
cacaattgtcatagtggacat

SEQ ID NO: 111 - DNA, 21 bp, Artificial Sequence
  siRNA
tcggatatccgcaactgtaaa

SEQ ID NO: 112 - DNA, 21 bp, Artificial Sequence
  siRNA
aaggctcttcaaggctatgaa

SEQ ID NO: 113 - DNA, 21 bp, Artificial Sequence
  siRNA
ttccaatttactggatttaaa

SEQ ID NO: 114 - DNA, 21 bp, Artificial Sequence
  siRNA
acaaggcattctctaaagcta

SEQ ID NO: 115 - DNA, 21 bp, Artificial Sequence
  siRNA
ccggtgttctaagatgtgata

SEQ ID NO: 116 - DNA, 21 bp, Artificial Sequence
  siRNA
ctcagcatgttcggcctgaaa

SEQ ID NO: 117 - DNA, 21 bp, Artificial Sequence
  siRNA
acggtgtgaatcacacatcca

SEQ ID NO: 118 - DNA, 21 bp, Artificial Sequence
  siRNA
atcgatcaatggatacctata

SEQ ID NO: 119 - DNA, 21 bp, Artificial Sequence
  siRNA
cccgccgaactctctcagata

SEQ ID NO: 120 - DNA, 21 bp, Artificial Sequence
  siRNA
caggaaggcctgtacaatgaa

SEQ ID NO: 121 - DNA, 21 bp, Artificial Sequence
  siRNA
aagcagaatacattcacgcta

SEQ ID NO: 122 - DNA, 21 bp, Artificial Sequence
  siRNA
tcggcctgaagtgactcgtaa

SEQ ID NO: 123 - DNA, 21 bp, Artificial Sequence
  siRNA
cacggagatgttctgctgcta

SEQ ID NO: 124 - DNA, 21 bp, Artificial Sequence
  siRNA
caggaagatctgtggaccaaa

SEQ ID NO: 125 - DNA, 21 bp, Artificial Sequence
  siRNA
ccaggcgatctgcatacttta

SEQ ID NO: 126 - DNA, 21 bp, Artificial Sequence
  siRNA
ctcgggatgttcacaaccgaa

SEQ ID NO: 127 - DNA, 21 bp, Artificial Sequence
  siRNA
caggatatgagttgcaggtga

SEQ ID NO: 128 - DNA, 21 bp, Artificial Sequence
  siRNA
aaggactccgaatacataata

SEQ ID NO: 129 - DNA, 21 bp, Artificial Sequence
  siRNA
caggtcggagaccatgagaaa

SEQ ID NO: 130 - DNA, 21 bp, Artificial Sequence
  siRNA
gagcgaggagcaattgattaa

SEQ ID NO: 131 - DNA, 21 bp, Artificial Sequence
  siRNA
aactccatctgtgcagcaaac

SEQ ID NO: 132 - DNA, 21 bp, Artificial Sequence
  siRNA
aagcctcttacctgccgtaaa

SEQ ID NO: 133 - DNA, 21 bp, Artificial Sequence
  siRNA
gaccatggagcggtacctaaa

SEQ ID NO: 134 - DNA, 21 bp, Artificial Sequence
  siRNA
aagcccagccacagattgcca

SEQ ID NO: 135 - DNA, 21 bp, Artificial Sequence
  siRNA
aagtacaacttccacggcact

SEQ ID NO: 136 - DNA, 21 bp, Artificial Sequence
  siRNA
ctggtgttgatcaacaaatca

SEQ ID NO: 137 - DNA, 21 bp, Artificial Sequence
  siRNA
caggtcatgcttctcatcgat

SEQ ID NO: 138 - DNA, 21 bp, Artificial Sequence
  siRNA
gtcggaaatggcgatcagcaa

SEQ ID NO: 139 - DNA, 21 bp, Artificial Sequence
  siRNA
cagaataggaacaaggttcta

SEQ ID NO: 140 - DNA, 21 bp, Artificial Sequence
  siRNA
cggcaacaagaccgtgaccta

SEQ ID NO: 141 - DNA, 21 bp, Artificial Sequence
  siRNA
taggtgtgacttgaactagat

SEQ ID NO: 142 - DNA, 21 bp, Artificial Sequence
  siRNA
caaggacataacaccacgaaa

SEQ ID NO: 143 - DNA, 21 bp, Artificial Sequence
  siRNA
aagcagaagattattgatcta

SEQ ID NO: 144 - DNA, 21 bp, Artificial Sequence
  siRNA
aatccgggacaagcctgaaga

SEQ ID NO: 145 - DNA, 21 bp, Artificial Sequence
  siRNA
ctggtcgcttacatcacttta

SEQ ID NO: 146 - DNA, 21 bp, Artificial Sequence
  siRNA
aacagaattatttgacctgaa

SEQ ID NO: 147 - DNA, 21 bp, Artificial Sequence
  siRNA
aacgaggtccagcgatttgaa

SEQ ID NO: 148 - DNA, 21 bp, Artificial Sequence
  siRNA
caaggtggtttcaacaagttt

SEQ ID NO: 149 - DNA, 21 bp, Artificial Sequence
  siRNA
ctggaagttcgagaccaccaa

SEQ ID NO: 150 - DNA, 21 bp, Artificial Sequence
  siRNA
tcggacagacatataggatat

SEQ ID NO: 151 - DNA, 21 bp, Artificial Sequence
  siRNA
ccggcgacgatttgtgcgtaa

SEQ ID NO: 152 - DNA, 21 bp, Artificial Sequence
  siRNA
aagatggacggagcttgttat

SEQ ID NO: 153 - DNA, 21 bp, Artificial Sequence
  siRNA
gacggaagagttgagacctaa

SEQ ID NO: 154 - DNA, 21 bp, Artificial Sequence
  siRNA
tagtttatcattaaccactta

SEQ ID NO: 155 - DNA, 21 bp, Artificial Sequence
  siRNA
tacgctgggaatattctgtat

SEQ ID NO: 156 - DNA, 21 bp, Artificial Sequence
  siRNA
tagagcatgaacatccagtaa

SEQ ID NO: 157 - DNA, 21 bp, Artificial Sequence
  siRNA
cccggttacagctgagaatga

SEQ ID NO: 158 - DNA, 21 bp, Artificial Sequence
  siRNA
ctgaataaacagtttatttaa

SEQ ID NO: 159 - DNA, 21 bp, Artificial Sequence
  siRNA
gcggttgttcctcacccgata

SEQ ID NO: 160 - DNA, 21 bp, Artificial Sequence
  siRNA
acgagacgtgattatgaggga

SEQ ID NO: 161 - DNA, 21 bp, Artificial Sequence
  siRNA
aagggttatctctttcataca

SEQ ID NO: 162 - DNA, 21 bp, Artificial Sequence
  siRNA
ccggacttggtgcgtctaaga

SEQ ID NO: 163 - DNA, 21 bp, Artificial Sequence
  siRNA
ctggtcgtcctcatcttaata

SEQ ID NO: 164 - DNA, 21 bp, Artificial Sequence
  siRNA
ctcagtgatacgtacagccaa

SEQ ID NO: 165 - DNA, 21 bp, Artificial Sequence
  siRNA
aaagattcccacccaattcaa

SEQ ID NO: 166 - DNA, 21 bp, Artificial Sequence
  siRNA
caagaagattgccttggtaaa

SEQ ID NO: 167 - DNA, 21 bp, Artificial Sequence
  siRNA
tcccataattgctttgccaaa

SEQ ID NO: 168 - DNA, 21 bp, Artificial Sequence
  siRNA
gacggcgttctggatctcaaa

SEQ ID NO: 169 - DNA, 21 bp, Artificial Sequence
  siRNA
caggaactggatattctgaaa

SEQ ID NO: 170 - DNA, 21 bp, Artificial Sequence
  siRNA
tgccatattgcacatgtctta

SEQ ID NO: 171 - DNA, 21 bp, Artificial Sequence
  siRNA
catcagatttgaaatatttaa

SEQ ID NO: 172 - DNA, 21 bp, Artificial Sequence
  siRNA
cgggccctgagtaatcgctta

SEQ ID NO: 173 - DNA, 21 bp, Artificial Sequence
  siRNA
aagatggattatcaagtgtca

SEQ ID NO: 174 - DNA, 21 bp, Artificial Sequence
  siRNA
aaggacagccctgtggctata

SEQ ID NO: 175 - DNA, 21 bp, Artificial Sequence
  siRNA
caccctgatgtcgcagcctat

SEQ ID NO: 176 - DNA, 21 bp, Artificial Sequence
  siRNA
aagctagatactggaaaccta

SEQ ID NO: 177 - DNA, 21 bp, Artificial Sequence
  siRNA
ctggttcaacgaggccattga

SEQ ID NO: 178 - DNA, 21 bp, Artificial Sequence
  siRNA
tacaagggacactataaataa

SEQ ID NO: 179 - DNA, 21 bp, Artificial Sequence
  siRNA
cccgtcggtggccaccacgta

SEQ ID NO: 180 - DNA, 21 bp, Artificial Sequence
  siRNA
tcagatgtacatagagatcta

SEQ ID NO: 181 - DNA, 21 bp, Artificial Sequence
  siRNA
cacgaatattatgcccagttt

SEQ ID NO: 182 - DNA, 21 bp, Artificial Sequence
  siRNA
ctaggtattgtctactctgaa

SEQ ID NO: 183 - DNA, 21 bp, Artificial Sequence
  siRNA
cccgagatgttcatggatgaa

SEQ ID NO: 184 - DNA, 21 bp, Artificial Sequence
  siRNA
cacggccaagtttacctccga

SEQ ID NO: 185 - DNA, 21 bp, Artificial Sequence
  siRNA
tcctataacttcagtattgta

SEQ ID NO: 186 - DNA, 21 bp, Artificial Sequence
  siRNA
taagaattgagtaatggtgta

SEQ ID NO: 187 - DNA, 21 bp, Artificial Sequence
  siRNA
gacgagagtggtcaaggttaa

SEQ ID NO: 188 - DNA, 21 bp, Artificial Sequence
  siRNA
tacccagaattctgttctaaa

SEQ ID NO: 189 - DNA, 21 bp, Artificial Sequence
  siRNA
accgagcacattggtgacaga

SEQ ID NO: 190 - DNA, 21 bp, Artificial Sequence
  siRNA
tccactctctacaatccgtaa

SEQ ID NO: 191 - DNA, 21 bp, Artificial Sequence
  siRNA
ctggcgcagatcgatttgaat

SEQ ID NO: 192 - DNA, 21 bp, Artificial Sequence
  siRNA
cagatgaaatcggcaacttta

SEQ ID NO: 193 - DNA, 21 bp, Artificial Sequence
  siRNA
ctggtagaagtcagtcggtat

SEQ ID NO: 194 - DNA, 21 bp, Artificial Sequence
  siRNA
aactgattcccaaaagcgaag

SEQ ID NO: 195 - DNA, 21 bp, Artificial Sequence
  siRNA
tacgcgcctcattaaaccaaa

SEQ ID NO: 196 - DNA, 21 bp, Artificial Sequence
  siRNA
aagggttaagatctatgggaa

SEQ ID NO: 197 - DNA, 21 bp, Artificial Sequence
  siRNA
gaggaatgtctacaaggatta

SEQ ID NO: 198 - DNA, 21 bp, Artificial Sequence
  siRNA
tacggacactattatcactaa

SEQ ID NO: 199 - DNA, 21 bp, Artificial Sequence
  siRNA
caccgagacatttaggtgaaa

SEQ ID NO: 200 - DNA, 21 bp, Artificial Sequence
  siRNA
cagcgactcggtagtccatta

SEQ ID NO: 201 - DNA, 21 bp, Artificial Sequence
  siRNA
caaagctgggttacaagtgta

SEQ ID NO: 202 - DNA, 21 bp, Artificial Sequence
  siRNA
accacgaaacgtgaagttcaa

SEQ ID NO: 203 - DNA, 21 bp, Artificial Sequence
  siRNA
ttccataagcttggtgacaaa

SEQ ID NO: 204 - DNA, 21 bp, Artificial Sequence
  siRNA
caggatgtaccgagcacttta

SEQ ID NO: 205 - DNA, 21 bp, Artificial Sequence
  siRNA
tccattgaagctgaaatggta

SEQ ID NO: 206 - DNA, 21 bp, Artificial Sequence
  siRNA
cagcgcctgcacgatattgaa

SEQ ID NO: 207 - DNA, 21 bp, Artificial Sequence
  siRNA
ccgaggttacacgttacgaaa

SEQ ID NO: 208 - DNA, 21 bp, Artificial Sequence
  siRNA
tacgactttgtcaagaggaaa

SEQ ID NO: 209 - DNA, 21 bp, Artificial Sequence
  siRNA
caaggagaagacccacatcaa

SEQ ID NO: 210 - DNA, 21 bp, Artificial Sequence
  siRNA
cagcgccaagtaaacagggta

SEQ ID NO: 211 - DNA, 21 bp, Artificial Sequence
  siRNA
caggccattcttaattaccta

SEQ ID NO: 212 - DNA, 21 bp, Artificial Sequence
  siRNA
aacttgccttaaacactcacc

SEQ ID NO: 213 - DNA, 21 bp, Artificial Sequence
  siRNA
ctggatcgactacaagttgaa

SEQ ID NO: 214 - DNA, 21 bp, Artificial Sequence
  siRNA
caggtgtttataattaatcct

SEQ ID NO: 215 - DNA, 21 bp, Artificial Sequence
  siRNA
cacggtgagggatgtaaatga

SEQ ID NO: 216 - DNA, 21 bp, Artificial Sequence
  siRNA
aaggttggccttagtagtcaa

SEQ ID NO: 217 - DNA, 21 bp, Artificial Sequence
  siRNA
caggtctatgctgcgctcata

SEQ ID NO: 218 - DNA, 21 bp, Artificial Sequence
  siRNA
ccgggattccgccaatttgga

SEQ ID NO: 219 - DNA, 21 bp, Artificial Sequence
  siRNA
tacgactaatcacctactcaa

SEQ ID NO: 220 - DNA, 21 bp, Artificial Sequence
  siRNA
agggatggacatcttgtcatt

SEQ ID NO: 221 - DNA, 21 bp, Artificial Sequence
  siRNA
ctggccaaatagcaaagacaa

SEQ ID NO: 222 - DNA, 21 bp, Artificial Sequence
  siRNA
cccggcaactctagtatttag

SEQ ID NO: 223 - DNA, 21 bp, Artificial Sequence
  siRNA
agggctgtatcacatcggtta

SEQ ID NO: 224 - DNA, 21 bp, Artificial Sequence
  siRNA
cagagaactgcggttacttaa

SEQ ID NO: 225 - DNA, 21 bp, Artificial Sequence
  siRNA
ctgatcgttcaaagcatgaaa

SEQ ID NO: 226 - DNA, 21 bp, Artificial Sequence
  siRNA
aagggtgtgcattacatttca

SEQ ID NO: 227 - DNA, 21 bp, Artificial Sequence
  siRNA
gtgctggatttgatgagttaa

SEQ ID NO: 228 - DNA, 21 bp, Artificial Sequence
  siRNA
ctggaggtacccattccagaa

SEQ ID NO: 229 - DNA, 21 bp, Artificial Sequence
  siRNA
ataggtattggtgaatttaaa

SEQ ID NO: 230 - DNA, 21 bp, Artificial Sequence
  siRNA
aaagatataagtgctgtataa

SEQ ID NO: 231 - DNA, 21 bp, Artificial Sequence
  siRNA
tacaattgtggttaccttcaa

SEQ ID NO: 232 - DNA, 21 bp, Artificial Sequence
  siRNA
caccctgcggaacttgttcaa

SEQ ID NO: 233 - DNA, 21 bp, Artificial Sequence
  siRNA
aagggacatattcatttggaa

SEQ ID NO: 234 - DNA, 21 bp, Artificial Sequence
  siRNA
aagcatgtctgtgatccacca

SEQ ID NO: 235 - DNA, 21 bp, Artificial Sequence
  siRNA
gcgaattgtggcagagtgtaa

SEQ ID NO: 236 - DNA, 21 bp, Artificial Sequence
  siRNA
tacgtgcgctctgccattcaa

SEQ ID NO: 237 - DNA, 21 bp, Artificial Sequence
  siRNA
cagttgtatgtttgctgatta

SEQ ID NO: 238 - DNA, 21 bp, Artificial Sequence
  siRNA
agggtgttggttaaagttgta

SEQ ID NO: 239 - DNA, 21 bp, Artificial Sequence
  siRNA
caggacaattgaaatttgcta

SEQ ID NO: 240 - DNA, 21 bp, Artificial Sequence
  siRNA
cagactctgacaatcattaaa

SEQ ID NO: 241 - DNA, 21 bp, Artificial Sequence
  siRNA
agggagtcatctcttcctata

SEQ ID NO: 242 - DNA, 21 bp, Artificial Sequence
  siRNA
accagttggatctccaatgaa

SEQ ID NO: 243 - DNA, 21 bp, Artificial Sequence
  siRNA
cccagtgagaaggctaacaaa

SEQ ID NO: 244 - DNA, 21 bp, Artificial Sequence
  siRNA
tagcattaagcaggaacgaat

SEQ ID NO: 245 - DNA, 21 bp, Artificial Sequence
  siRNA
ctggcggttcatggagagcaa

SEQ ID NO: 246 - DNA, 21 bp, Artificial Sequence
  siRNA
caggctcttccggcaagtcaa

SEQ ID NO: 247 - DNA, 21 bp, Artificial Sequence
  siRNA
tacgttgatttcagagaatat

SEQ ID NO: 248 - DNA, 21 bp, Artificial Sequence
  siRNA
aagaagcaggatgctgatcta

SEQ ID NO: 249 - DNA, 21 bp, Artificial Sequence
  siRNA
caggtgagcgacattacacca

SEQ ID NO: 250 - DNA, 21 bp, Artificial Sequence
  siRNA
ccgggcggttgtctacagcaa

SEQ ID NO: 251 - DNA, 21 bp, Artificial Sequence
  siRNA
atgcaacttattgtatctgaa

SEQ ID NO: 252 - DNA, 21 bp, Artificial Sequence
  siRNA
caggctgcgaaggaagtacta

SEQ ID NO: 253 - DNA, 21 bp, Artificial Sequence
  siRNA
tcgggatttggcagcccgtaa

SEQ ID NO: 254 - DNA, 21 bp, Artificial Sequence
  siRNA
ctcaccgtatccagcacatta

SEQ ID NO: 255 - DNA, 21 bp, Artificial Sequence
  siRNA
cagaacaacttagtaggataa

SEQ ID NO: 256 - DNA, 21 bp, Artificial Sequence
  siRNA
caccacacgggttcagttcaa

SEQ ID NO: 257 - DNA, 21 bp, Artificial Sequence
  siRNA
cacgctcttggtcaacaggaa

SEQ ID NO: 258 - DNA, 21 bp, Artificial Sequence
  siRNA
accgagggcgatgaagaagca

SEQ ID NO: 259 - DNA, 21 bp, Artificial Sequence
  siRNA
atgcttagagtggactattaa

SEQ ID NO: 260 - DNA, 21 bp, Artificial Sequence
  siRNA
cagatgatgcccggcaattat

SEQ ID NO: 261 - DNA, 21 bp, Artificial Sequence
  siRNA
gaaggagtagatatcatccta

SEQ ID NO: 262 - DNA, 21 bp, Artificial Sequence
  siRNA
cagatgatcagggatccaata

SEQ ID NO: 263 - DNA, 21 bp, Artificial Sequence
  siRNA
caggaatattaagttctataa

SEQ ID NO: 264 - DNA, 21 bp, Artificial Sequence
  siRNA
tgggtgctttgtgatggataa

SEQ ID NO: 265 - DNA, 21 bp, Artificial Sequence
  siRNA
cagcctgaggctgagtaccaa

SEQ ID NO: 266 - DNA, 21 bp, Artificial Sequence
  siRNA
ttctatgacattgaaacacta

SEQ ID NO: 267 - DNA, 21 bp, Artificial Sequence
  siRNA
tgggttcattattggaattaa

SEQ ID NO: 268 - DNA, 21 bp, Artificial Sequence
  siRNA
tagatgctggattgacattta

SEQ ID NO: 269 - DNA, 21 bp, Artificial Sequence
  siRNA
tacccgggctacagtccgcaa

SEQ ID NO: 270 - DNA, 21 bp, Artificial Sequence
  siRNA
ccgggagtatcaactcaacga

SEQ ID NO: 271 - DNA, 21 bp, Artificial Sequence
  siRNA
cacgttacagttagcagacga

SEQ ID NO: 272 - DNA, 21 bp, Artificial Sequence
  siRNA
tacctttgtcctggatcggaa

SEQ ID NO: 273 - DNA, 21 bp, Artificial Sequence
  siRNA
cagctttagcaaagaactctt

SEQ ID NO: 274 - DNA, 21 bp, Artificial Sequence
  siRNA
cagactcaataccagacacta

SEQ ID NO: 275 - DNA, 21 bp, Artificial Sequence
  siRNA
ccggcctctatgcttgcgtaa

SEQ ID NO: 276 - DNA, 21 bp, Artificial Sequence
  siRNA
cccgagtttagtaacagtgca

SEQ ID NO: 277 - DNA, 21 bp, Artificial Sequence
  siRNA
ttcctggtttccgaaaggcaa

SEQ ID NO: 278 - DNA, 21 bp, Artificial Sequence
  siRNA
aaggtcggagtcaacggattt

SEQ ID NO: 279 - DNA, 21 bp, Artificial Sequence
  siRNA
ctgaccgatggaggtagtata

SEQ ID NO: 280 - DNA, 21 bp, Artificial Sequence
  siRNA
ccgggagaaggtggagactga

SEQ ID NO: 281 - DNA, 21 bp, Artificial Sequence
  siRNA
tcccatgcatttaatatatta

SEQ ID NO: 282 - DNA, 21 bp, Artificial Sequence
  siRNA
tacatcggtagaaacgttgaa

SEQ ID NO: 283 - DNA, 21 bp, Artificial Sequence
  siRNA
gagacttgaattaataagtga

SEQ ID NO: 284 - DNA, 21 bp, Artificial Sequence
  siRNA
caggagaacttaaagcccaaa

SEQ ID NO: 285 - DNA, 21 bp, Artificial Sequence
  siRNA
ccgatgttattagatgttaca

SEQ ID NO: 286 - DNA, 21 bp, Artificial Sequence
  siRNA
gtggaagaagatccagcagaa

SEQ ID NO: 287 - DNA, 21 bp, Artificial Sequence
  siRNA
ctgggagtacctagaacccta

SEQ ID NO: 288 - DNA, 21 bp, Artificial Sequence
  siRNA
tcggtgcttccagccacataa

SEQ ID NO: 289 - DNA, 21 bp, Artificial Sequence
  siRNA
cggccgcgagttcgaccagaa

SEQ ID NO: 290 - DNA, 21 bp, Artificial Sequence
  siRNA
caagaactacatagaaatgaa

SEQ ID NO: 291 - DNA, 21 bp, Artificial Sequence
  siRNA
ttggatagtttcctacgtaaa

SEQ ID NO: 292 - DNA, 21 bp, Artificial Sequence
  siRNA
cacgtttgagtccatgcccaa

SEQ ID NO: 293 - DNA, 21 bp, Artificial Sequence
  siRNA
ttggaactgcatctaacatta

SEQ ID NO: 294 - DNA, 21 bp, Artificial Sequence
  siRNA
ctggaaggaccataaatttaa

SEQ ID NO: 295 - DNA, 21 bp, Artificial Sequence
  siRNA
cagcatatgtgtaaagattta

SEQ ID NO: 296 - DNA, 21 bp, Artificial Sequence
  siRNA
cactacagtattagcaagcaa

SEQ ID NO: 297 - DNA, 21 bp, Artificial Sequence
  siRNA
aagatcaatcctccatgagta

SEQ ID NO: 298 - DNA, 21 bp, Artificial Sequence
  siRNA
ggcgggagacactatattcaa

SEQ ID NO: 299 - DNA, 21 bp, Artificial Sequence
  siRNA
tacgacctggttctagctgaa

SEQ ID NO: 300 - DNA, 21 bp, Artificial Sequence
  siRNA
ctggatctgtctccacctcat

SEQ ID NO: 301 - DNA, 21 bp, Artificial Sequence
  siRNA
ctgctgagagagcaaggcaat

SEQ ID NO: 302 - DNA, 21 bp, Artificial Sequence
  siRNA
cccggcagtatgtgtctgtaa

SEQ ID NO: 303 - DNA, 21 bp, Artificial Sequence
  siRNA
cttcgtcatgttgaactataa

SEQ ID NO: 304 - DNA, 21 bp, Artificial Sequence
  siRNA
gcgccactactacaaactaaa

SEQ ID NO: 305 - DNA, 21 bp, Artificial Sequence
  siRNA
ttgaatgttcgtaatagtaga

SEQ ID NO: 306 - DNA, 21 bp, Artificial Sequence
  siRNA
ccgcctgaccttcggacccta

SEQ ID NO: 307 - DNA, 21 bp, Artificial Sequence
  siRNA
accaattcaagtgaagattat

SEQ ID NO: 308 - DNA, 21 bp, Artificial Sequence
  siRNA
gtggccctttatgactatgaa

SEQ ID NO: 309 - DNA, 21 bp, Artificial Sequence
  siRNA
aagtgttaatccaggcctaaa

SEQ ID NO: 310 - DNA, 21 bp, Artificial Sequence
  siRNA
cagcccaagtccaaatgttta

SEQ ID NO: 311 - DNA, 21 bp, Artificial Sequence
  siRNA
caccaggagcatatcaacata

SEQ ID NO: 312 - DNA, 21 bp, Artificial Sequence
  siRNA
accagtcatgatagtaataga

SEQ ID NO: 313 - DNA, 21 bp, Artificial Sequence
  siRNA
ctacgtgttagtggctcttaa

SEQ ID NO: 314 - DNA, 21 bp, Artificial Sequence
  siRNA
agccgacgggtctttaaacaa

SEQ ID NO: 315 - DNA, 21 bp, Artificial Sequence
  siRNA
cagatagatcctagtacagaa

SEQ ID NO: 316 - DNA, 21 bp, Artificial Sequence
  siRNA
cacgtggaacggcagcactaa

SEQ ID NO: 317 - DNA, 21 bp, Artificial Sequence
  siRNA
caccgtgtgggctgagtttaa

SEQ ID NO: 318 - DNA, 21 bp, Artificial Sequence
  siRNA
aagctctatcgggaaacaaat

SEQ ID NO: 319 - DNA, 21 bp, Artificial Sequence
  siRNA
cagggaatcaagccatgctta

SEQ ID NO: 320 - DNA, 21 bp, Artificial Sequence
  siRNA
aaagtgtgattcgatcgtcaa

SEQ ID NO: 321 - DNA, 21 bp, Artificial Sequence
  siRNA
ttcgagcgtacagcaagtaaa

SEQ ID NO: 322 - DNA, 21 bp, Artificial Sequence
  siRNA
ccagtgcagactcaagactta

SEQ ID NO: 323 - DNA, 21 bp, Artificial Sequence
  siRNA
caccatggtatcatatattaa

SEQ ID NO: 324 - DNA, 21 bp, Artificial Sequence
  siRNA
aaggcagttcaagctgttgaa

SEQ ID NO: 325 - DNA, 21 bp, Artificial Sequence
  siRNA
aaggccaagtttctacaggaa

SEQ ID NO: 326 - DNA, 21 bp, Artificial Sequence
  siRNA
caggttgtaagtgcttgccaa

SEQ ID NO: 327 - DNA, 21 bp, Artificial Sequence
  siRNA
gaccaatattatactaagaaa

SEQ ID NO: 328 - DNA, 21 bp, Artificial Sequence
  siRNA
cccgaccagattcagtgcata

SEQ ID NO: 329 - DNA, 21 bp, Artificial Sequence
  siRNA
ctcgctagtggcctacatcaa

SEQ ID NO: 330 - DNA, 21 bp, Artificial Sequence
  siRNA
atggacactttgggcatcgaa

SEQ ID NO: 331 - DNA, 21 bp, Artificial Sequence
  siRNA
cagagtctccgctatggataa

SEQ ID NO: 332 - DNA, 21 bp, Artificial Sequence
  siRNA
caggtggatgttagaagtcga

SEQ ID NO: 333 - DNA, 21 bp, Artificial Sequence
  siRNA
ctgggtcagtaattacaagta

SEQ ID NO: 334 - DNA, 21 bp, Artificial Sequence
  siRNA
cagtcacataagtataataaa

SEQ ID NO: 335 - DNA, 21 bp, Artificial Sequence
  siRNA
cagagatttacccatcgggta

SEQ ID NO: 336 - DNA, 21 bp, Artificial Sequence
  siRNA
aaccaagtagcctgttatcaa

SEQ ID NO: 337 - DNA, 21 bp, Artificial Sequence
  siRNA
aagggtgcgccgggaacgaaa

SEQ ID NO: 338 - DNA, 21 bp, Artificial Sequence
  siRNA
ccgagccacatcgctcagaca

SEQ ID NO: 339 - DNA, 21 bp, Artificial Sequence
  siRNA
ccgccttatctagtagcccta

SEQ ID NO: 340 - DNA, 21 bp, Artificial Sequence
  siRNA
accatgtttcctctcaataaa

SEQ ID NO: 341 - DNA, 21 bp, Artificial Sequence
  siRNA
caggctaagggtgatgctgaa

SEQ ID NO: 342 - DNA, 21 bp, Artificial Sequence
  siRNA
tagcctataaataaattccaa

SEQ ID NO: 343 - DNA, 21 bp, Artificial Sequence
  siRNA
tccgagtatgatcctaccaga

SEQ ID NO: 344 - DNA, 21 bp, Artificial Sequence
  siRNA
aagctgatcggcatcattgaa

SEQ ID NO: 345 - DNA, 21 bp, Artificial Sequence
  siRNA
accctacgttaccgtgctcaa

SEQ ID NO: 346 - DNA, 21 bp, Artificial Sequence
  siRNA
cacccatggagtagtgaacaa

SEQ ID NO: 347 - DNA, 21 bp, Artificial Sequence
  siRNA
caggacagtcaaggtgatata

SEQ ID NO: 348 - DNA, 21 bp, Artificial Sequence
  siRNA
aaggcagtgcgtggcgggcaa

SEQ ID NO: 349 - DNA, 21 bp, Artificial Sequence
  siRNA
gtccgtttaactcgatagaaa

SEQ ID NO: 350 - DNA, 21 bp, Artificial Sequence
  siRNA
aagtttggaaacgatgtgcag

SEQ ID NO: 351 - DNA, 21 bp, Artificial Sequence
  siRNA
tcggatatgattgtttctcaa

SEQ ID NO: 352 - DNA, 21 bp, Artificial Sequence
  siRNA
aacaaagaaatcttagacgaa

SEQ ID NO: 353 - DNA, 21 bp, Artificial Sequence
  siRNA
agggagtatgtcttaaatgta

SEQ ID NO: 354 - DNA, 21 bp, Artificial Sequence
  siRNA
caagttgctaagtgaacagaa

SEQ ID NO: 355 - DNA, 21 bp, Artificial Sequence
  siRNA
ttagttgaggataccacatta

SEQ ID NO: 356 - DNA, 21 bp, Artificial Sequence
  siRNA
accgcatatggtatccctcaa

SEQ ID NO: 357 - DNA, 21 bp, Artificial Sequence
  siRNA
acaggataattcagacaacaa

SEQ ID NO: 358 - DNA, 21 bp, Artificial Sequence
  siRNA
cagaggtttgaccgaattgca

SEQ ID NO: 359 - DNA, 21 bp, Artificial Sequence
  siRNA
cgggcggatgatgcacgccaa

SEQ ID NO: 360 - DNA, 21 bp, Artificial Sequence
  siRNA
cctgcagaaagtgaagcatta

SEQ ID NO: 361 - DNA, 21 bp, Artificial Sequence
  siRNA
ctagatgacgggaacaccaaa

SEQ ID NO: 362 - DNA, 21 bp, Artificial Sequence
  siRNA
cccgctgacatttccgttgta

SEQ ID NO: 363 - DNA, 21 bp, Artificial Sequence
  siRNA
ctccagtggctctgactacta

SEQ ID NO: 364 - DNA, 21 bp, Artificial Sequence
  siRNA
aggaaataacattgcacttta

SEQ ID NO: 365 - DNA, 21 bp, Artificial Sequence
  siRNA
ttggactgtgatattcgttat

SEQ ID NO: 366 - DNA, 21 bp, Artificial Sequence
  siRNA
aagaactcggataatgataaa

SEQ ID NO: 367 - DNA, 21 bp, Artificial Sequence
  siRNA
tccgggtaccgcgaagggcaa

SEQ ID NO: 368 - DNA, 21 bp, Artificial Sequence
  siRNA
cacggataacatcagcttcat

SEQ ID NO: 369 - DNA, 21 bp, Artificial Sequence
  siRNA
cggctgggtcagcttctacaa

SEQ ID NO: 370 - DNA, 21 bp, Artificial Sequence
  siRNA
ccgcgaggtcaccattaacca

SEQ ID NO: 371 - DNA, 21 bp, Artificial Sequence
  siRNA
cagacctatttcccaaagcaa

SEQ ID NO: 372 - DNA, 21 bp, Artificial Sequence
  siRNA
cacccggaggaaagtctgtta

SEQ ID NO: 373 - DNA, 21 bp, Artificial Sequence
  siRNA
aatcatgactgaccgaggcag

SEQ ID NO: 374 - DNA, 21 bp, Artificial Sequence
  siRNA
aattgtattctccgagtcaga

SEQ ID NO: 375 - DNA, 21 bp, Artificial Sequence
  siRNA
ctggactcagtacgccgttta

SEQ ID NO: 376 - DNA, 21 bp, Artificial Sequence
  siRNA
cacgtgtatccctgagaacaa

SEQ ID NO: 377 - DNA, 21 bp, Artificial Sequence
  siRNA
caagatctacttcgccttcaa

SEQ ID NO: 378 - DNA, 21 bp, Artificial Sequence
  siRNA
ctgccttacgatgacagaaat

SEQ ID NO: 379 - DNA, 21 bp, Artificial Sequence
  siRNA
aaggctaatacaactcttcaa

SEQ ID NO: 380 - DNA, 21 bp, Artificial Sequence
  siRNA
cctgagagctctcctcaccaa

SEQ ID NO: 381 - DNA, 21 bp, Artificial Sequence
  siRNA
ctgggtccggtccataatcat

SEQ ID NO: 382 - DNA, 21 bp, Artificial Sequence
  siRNA
tagcatgtcaagtggagtgaa

SEQ ID NO: 383 - DNA, 21 bp, Artificial Sequence
  siRNA
aatctgatgctgtggaatgct

SEQ ID NO: 384 - DNA, 21 bp, Artificial Sequence
  siRNA
tgcactgtaagacgtatgtaa

SEQ ID NO: 385 - DNA, 21 bp, Artificial Sequence
  siRNA
caagaacgtcatgatgatcca

SEQ ID NO: 386 - DNA, 21 bp, Artificial Sequence
  siRNA
atggaccaacccaagtattaa

SEQ ID NO: 387 - DNA, 21 bp, Artificial Sequence
  siRNA
acaataggccatgttaattaa

SEQ ID NO: 388 - DNA, 21 bp, Artificial Sequence
  siRNA
aagaacgtgacagatgagcag

SEQ ID NO: 389 - DNA, 21 bp, Artificial Sequence
  siRNA
aaggagaatttaataaagata

SEQ ID NO: 390 - DNA, 21 bp, Artificial Sequence
  siRNA
tccttgcctgatgacaataaa

SEQ ID NO: 391 - DNA, 21 bp, Artificial Sequence
  siRNA
ctgcatttatcgttaacctaa

SEQ ID NO: 392 - DNA, 21 bp, Artificial Sequence
  siRNA
tggctgattctggagagtata

SEQ ID NO: 393 - DNA, 21 bp, Artificial Sequence
  siRNA
aggagcgatgacggaatataa

SEQ ID NO: 394 - DNA, 21 bp, Artificial Sequence
  siRNA
cagggtttggtgacaatagaa

SEQ ID NO: 395 - DNA, 21 bp, Artificial Sequence
  siRNA
ctggagaagtacagcgagcaa

SEQ ID NO: 396 - DNA, 21 bp, Artificial Sequence
  siRNA
ctcgcccttgatgccaacaaa

SEQ ID NO: 397 - DNA, 21 bp, Artificial Sequence
  siRNA
aatccttaatggctcagacat

SEQ ID NO: 398 - DNA, 21 bp, Artificial Sequence
  siRNA
cccgacgaccaccatcagcta

SEQ ID NO: 399 - DNA, 21 bp, Artificial Sequence
  siRNA
acgcatgtctctgacacctca

SEQ ID NO: 400 - DNA, 21 bp, Artificial Sequence
  siRNA
ccgccggatagtggatggcaa

SEQ ID NO: 401 - DNA, 21 bp, Artificial Sequence
  siRNA
cagctacatcattctgtacaa

SEQ ID NO: 402 - DNA, 21 bp, Artificial Sequence
  siRNA
caggttagggtgctagagcta

SEQ ID NO: 403 - DNA, 21 bp, Artificial Sequence
  siRNA
ccagatcaatgccatgaccta

SEQ ID NO: 404 - DNA, 21 bp, Artificial Sequence
  siRNA
cacgccttatagagtctccat

SEQ ID NO: 405 - DNA, 21 bp, Artificial Sequence
  siRNA
gtgcacctacttcaagttcta

SEQ ID NO: 406 - DNA, 21 bp, Artificial Sequence
  siRNA
aacctccttagggttcgtcaa

SEQ ID NO: 407 - DNA, 21 bp, Artificial Sequence
  siRNA
cacgtgtggcctgttcttcta

SEQ ID NO: 408 - DNA, 21 bp, Artificial Sequence
  siRNA
cgcggccggcagcatgatgaa

SEQ ID NO: 409 - DNA, 21 bp, Artificial Sequence
  siRNA
ttcactctttgcaattgctaa

SEQ ID NO: 410 - DNA, 21 bp, Artificial Sequence
  siRNA
caccctcaagctgtcgcccta

SEQ ID NO: 411 - DNA, 21 bp, Artificial Sequence
  siRNA
ccgggatagcgagaccactaa

SEQ ID NO: 412 - DNA, 21 bp, Artificial Sequence
  siRNA
tgccgtgatgtggaggaggaa

SEQ ID NO: 413 - DNA, 21 bp, Artificial Sequence
  siRNA
tgggataaggaaacacttcta

SEQ ID NO: 414 - DNA, 21 bp, Artificial Sequence
  siRNA
ccagtcgccaagaatcatgaa

SEQ ID NO: 415 - DNA, 21 bp, Artificial Sequence
  siRNA
ctgccaggtgtactttactta

SEQ ID NO: 416 - DNA, 21 bp, Artificial Sequence
  siRNA
acccaagaacagcttcaataa

SEQ ID NO: 417 - DNA, 21 bp, Artificial Sequence
  siRNA
aacgatgacaaccgacctatt

SEQ ID NO: 418 - DNA, 21 bp, Artificial Sequence
  siRNA
ccgcatctccgaggacaagaa

SEQ ID NO: 419 - DNA, 21 bp, Artificial Sequence
  siRNA
ctctctttaattgctaaccat

SEQ ID NO: 420 - DNA, 21 bp, Artificial Sequence
  siRNA
aggcatcaagttgaccatcaa

SEQ ID NO: 421 - DNA, 21 bp, Artificial Sequence
  siRNA
ccgccgcaaagcaggatgtta

SEQ ID NO: 422 - DNA, 21 bp, Artificial Sequence
  siRNA
cacacgctaactacaaggtca

SEQ ID NO: 423 - DNA, 21 bp, Artificial Sequence
  siRNA
cacaggagagtttgacacctt

SEQ ID NO: 424 - DNA, 21 bp, Artificial Sequence
  siRNA
aacactgtattgtaagtggaa

SEQ ID NO: 425 - DNA, 21 bp, Artificial Sequence
  siRNA
aaagtttagttgtaaacttaa

SEQ ID NO: 426 - DNA, 21 bp, Artificial Sequence
  siRNA
cacgacaattctgacgcccaa

SEQ ID NO: 427 - DNA, 21 bp, Artificial Sequence
  siRNA
ttggacggtggtagacattga

SEQ ID NO: 428 - DNA, 21 bp, Artificial Sequence
  siRNA
accggatgaggttctatttca

SEQ ID NO: 429 - DNA, 21 bp, Artificial Sequence
  siRNA
cgcaggtgggctggacttcta

SEQ ID NO: 430 - DNA, 21 bp, Artificial Sequence
  siRNA
tgccctgaatgatgagatcaa

SEQ ID NO: 431 - DNA, 21 bp, Artificial Sequence
  siRNA
aagaatgtatttcacctgcaa

SEQ ID NO: 432 - DNA, 21 bp, Artificial Sequence
  siRNA
cccgttcttaactttgaacca

SEQ ID NO: 433 - DNA, 21 bp, Artificial Sequence
  siRNA
ctcttaagccatcttggtaaa

SEQ ID NO: 434 - DNA, 21 bp, Artificial Sequence
  siRNA
aagagacttcctgagcagaaa

SEQ ID NO: 435 - DNA, 21 bp, Artificial Sequence
  siRNA
tcgaagaatcgcatcatcata

SEQ ID NO: 436 - DNA, 21 bp, Artificial Sequence
  siRNA
aagcccagcgagcatgtgaaa

SEQ ID NO: 437 - DNA, 21 bp, Artificial Sequence
  siRNA
cagctcggatttcgagatcca

SEQ ID NO: 438 - DNA, 21 bp, Artificial Sequence
  siRNA
agccatcatacgagatcttaa

SEQ ID NO: 439 - DNA, 21 bp, Artificial Sequence
  siRNA
aacgctgacatgtacggtcta

SEQ ID NO: 440 - DNA, 21 bp, Artificial Sequence
  siRNA
caccgcagttacggtcaacca

SEQ ID NO: 441 - DNA, 21 bp, Artificial Sequence
  siRNA
caggaaagatttggtgttgta

SEQ ID NO: 442 - DNA, 21 bp, Artificial Sequence
  siRNA
ctggagttccatgataccaca

SEQ ID NO: 443 - DNA, 21 bp, Artificial Sequence
  siRNA
ttccattgtatgcaaattgaa

SEQ ID NO: 444 - DNA, 21 bp, Artificial Sequence
  siRNA
aaccctgaccattccattatt

SEQ ID NO: 445 - DNA, 21 bp, Artificial Sequence
  siRNA
aaccctttacaaggccagaaa

SEQ ID NO: 446 - DNA, 21 bp, Artificial Sequence
  siRNA
cacattgtacatggtacgaat

SEQ ID NO: 447 - DNA, 21 bp, Artificial Sequence
  siRNA
ccggtatcgtttcgcatggaa

SEQ ID NO: 448 - DNA, 21 bp, Artificial Sequence
  siRNA
cccgagctggagcgcctgata

SEQ ID NO: 449 - DNA, 21 bp, Artificial Sequence
  siRNA
gtggacgaatatgatccaaca

SEQ ID NO: 450 - DNA, 21 bp, Artificial Sequence
  siRNA
caccgtggacaatgccaacat

SEQ ID NO: 451 - DNA, 21 bp, Artificial Sequence
  siRNA
aacactggtcacgtttggaaa

SEQ ID NO: 452 - DNA, 21 bp, Artificial Sequence
  siRNA
tcggctgcaggttccaaacta

SEQ ID NO: 453 - DNA, 21 bp, Artificial Sequence
  siRNA
cacagatgggatcacagtaaa

SEQ ID NO: 454 - DNA, 21 bp, Artificial Sequence
  siRNA
caccaccagatgagaagttaa

SEQ ID NO: 455 - DNA, 21 bp, Artificial Sequence
  siRNA
aaaccgagtttgggatcacat

SEQ ID NO: 456 - DNA, 21 bp, Artificial Sequence
  siRNA
cacaaccgccctcctggacaa

SEQ ID NO: 457 - DNA, 21 bp, Artificial Sequence
  siRNA
cagggtctacgtctacctgca

SEQ ID NO: 458 - DNA, 21 bp, Artificial Sequence
  siRNA
aaacacgcacttagtctctaa

SEQ ID NO: 459 - DNA, 21 bp, Artificial Sequence
  siRNA
aagccgagtcctggtatcaga

SEQ ID NO: 460 - DNA, 21 bp, Artificial Sequence
  siRNA
tcgctccaccttctccaccaa

SEQ ID NO: 461 - DNA, 21 bp, Artificial Sequence
  siRNA
aacgatgcctttgttctgaaa

SEQ ID NO: 462 - DNA, 21 bp, Artificial Sequence
  siRNA
cacaacaatgggtatccttaa

SEQ ID NO: 463 - DNA, 21 bp, Artificial Sequence
  siRNA
aaagacgaagagcaagaataa

SEQ ID NO: 464 - DNA, 21 bp, Artificial Sequence
  siRNA
cacgatggctttgcaggcgat

SEQ ID NO: 465 - DNA, 21 bp, Artificial Sequence
  siRNA
ctggaggaagtgctaaattta

SEQ ID NO: 466 - DNA, 21 bp, Artificial Sequence
  siRNA
cccgggactgttgacagccaa

SEQ ID NO: 467 - DNA, 21 bp, Artificial Sequence
  siRNA
caagctgaacctaatagccat

SEQ ID NO: 468 - DNA, 21 bp, Artificial Sequence
  siRNA
cccgccgttgtcgcccatcga

SEQ ID NO: 469 - DNA, 21 bp, Artificial Sequence
  siRNA
ctggagcttcactgttactaa

SEQ ID NO: 470 - DNA, 21 bp, Artificial Sequence
  siRNA
ccgcggatacaatgtgacgta

SEQ ID NO: 471 - DNA, 21 bp, Artificial Sequence
  siRNA
cggcagggagataccgtgaaa

SEQ ID NO: 472 - DNA, 21 bp, Artificial Sequence
  siRNA
tagaaccttagtataaattta

SEQ ID NO: 473 - DNA, 21 bp, Artificial Sequence
  siRNA
tagggtgtgtgttcaccttca

SEQ ID NO: 474 - DNA, 21 bp, Artificial Sequence
  siRNA
cagttggagctgaactcggaa

SEQ ID NO: 475 - DNA, 21 bp, Artificial Sequence
  siRNA
acagacggcggtggaaccaaa

SEQ ID NO: 476 - DNA, 21 bp, Artificial Sequence
  siRNA
caagttcttcatcgagttcta

SEQ ID NO: 477 - DNA, 21 bp, Artificial Sequence
  siRNA
gtggatgagttccggaataaa

SEQ ID NO: 478 - DNA, 21 bp, Artificial Sequence
  siRNA
aaccatcggcttgatcaggaa

SEQ ID NO: 479 - DNA, 21 bp, Artificial Sequence
  siRNA
tcaggagttctcatctgacaa

SEQ ID NO: 480 - DNA, 21 bp, Artificial Sequence
  siRNA
ctacgggacattcaccatcaa

SEQ ID NO: 481 - DNA, 21 bp, Artificial Sequence
  siRNA
ccgggtgccgattctcagtaa

SEQ ID NO: 482 - DNA, 21 bp, Artificial Sequence
  siRNA
ctggtgctctattgtctacta

SEQ ID NO: 483 - DNA, 21 bp, Artificial Sequence
  siRNA
gaccacgagacgggaacatta

SEQ ID NO: 484 - DNA, 21 bp, Artificial Sequence
  siRNA
cccgacgtctggaggactcaa

SEQ ID NO: 485 - DNA, 21 bp, Artificial Sequence
  siRNA
accgttatgcagaataatcca

SEQ ID NO: 486 - DNA, 21 bp, Artificial Sequence
  siRNA
ctgagatacgtctgtgactta

SEQ ID NO: 487 - DNA, 21 bp, Artificial Sequence
  siRNA
caaggctgccttcaattagaa

SEQ ID NO: 488 - DNA, 21 bp, Artificial Sequence
  siRNA
ttccagtactttgtacaggaa

SEQ ID NO: 489 - DNA, 21 bp, Artificial Sequence
  siRNA
ctccgtgaggctacattaata

SEQ ID NO: 490 - DNA, 21 bp, Artificial Sequence
  siRNA
cagtatttattgttcccacaa

SEQ ID NO: 491 - DNA, 21 bp, Artificial Sequence
  siRNA
caatacaaagctggataataa

SEQ ID NO: 492 - DNA, 21 bp, Artificial Sequence
  siRNA
caggtaatgtatcatgatcca

SEQ ID NO: 493 - DNA, 21 bp, Artificial Sequence
  siRNA
ctccgggtgtttcaactagaa

SEQ ID NO: 494 - DNA, 21 bp, Artificial Sequence
  siRNA
aggtgtggtgtggaacctata

SEQ ID NO: 495 - DNA, 21 bp, Artificial Sequence
  siRNA
cctgaccgtcatagagcagaa

SEQ ID NO: 496 - DNA, 21 bp, Artificial Sequence
  siRNA
ctgggagtggttagccggaat

SEQ ID NO: 497 - DNA, 21 bp, Artificial Sequence
  siRNA
caggtctacagtgatagagca

SEQ ID NO: 498 - DNA, 21 bp, Artificial Sequence
  siRNA
aagcgccacctcagaagataa

SEQ ID NO: 499 - DNA, 21 bp, Artificial Sequence
  siRNA
tcgggaagctaccatttctta

SEQ ID NO: 500 - DNA, 21 bp, Artificial Sequence
  siRNA
ctgcccgatggtcttccgtaa

SEQ ID NO: 501 - DNA, 21 bp, Artificial Sequence
  siRNA
ccaggagcttctggacatcaa

SEQ ID NO: 502 - DNA, 21 bp, Artificial Sequence
  siRNA
atggtgcgagaaggcggtcaa

SEQ ID NO: 503 - DNA, 21 bp, Artificial Sequence
  siRNA
ccacgtgtctgtgcaccacaa

SEQ ID NO: 504 - DNA, 21 bp, Artificial Sequence
  siRNA
caggtctgcgtagatggtgaa

SEQ ID NO: 505 - DNA, 21 bp, Artificial Sequence
  siRNA
gacgccatctatgacagtaaa

SEQ ID NO: 506 - DNA, 21 bp, Artificial Sequence
  siRNA
gggctccttaaggataactaa

SEQ ID NO: 507 - DNA, 21 bp, Artificial Sequence
  siRNA
cacggttccacggtataaata

SEQ ID NO: 508 - DNA, 21 bp, Artificial Sequence
  siRNA
ctgccgagcaactttgatcaa

SEQ ID NO: 509 - DNA, 21 bp, Artificial Sequence
  siRNA
cgccgtgatcgagaaagccaa

SEQ ID NO: 510 - DNA, 21 bp, Artificial Sequence
  siRNA
ctgcacaatatgagcatgcaa

SEQ ID NO: 511 - DNA, 21 bp, Artificial Sequence
  siRNA
tacgaccgcatcgagacgata

SEQ ID NO: 512 - DNA, 21 bp, Artificial Sequence
  siRNA
ctggtgctcggtcgcctacta

SEQ ID NO: 513 - DNA, 21 bp, Artificial Sequence
  siRNA
ctcaagcatcgcatagtttaa

SEQ ID NO: 514 - DNA, 21 bp, Artificial Sequence
  siRNA
cagcagcctctcttacacaaa

SEQ ID NO: 515 - DNA, 21 bp, Artificial Sequence
  siRNA
ccgagcacatagactccttta

SEQ ID NO: 516 - DNA, 21 bp, Artificial Sequence
  siRNA
ctggttggagcgaatctgcta

SEQ ID NO: 517 - DNA, 21 bp, Artificial Sequence
  siRNA
atgcgcatgaacactttgtta

SEQ ID NO: 518 - DNA, 21 bp, Artificial Sequence
  siRNA
cacgcctaacaggacgtatta

SEQ ID NO: 519 - DNA, 21 bp, Artificial Sequence
  siRNA
cacctttactctataactcaa

SEQ ID NO: 520 - DNA, 21 bp, Artificial Sequence
  siRNA
tccgtctttggcaagacatta

SEQ ID NO: 521 - DNA, 21 bp, Artificial Sequence
  siRNA
cctgcagatgcttctaataaa

SEQ ID NO: 522 - DNA, 21 bp, Artificial Sequence
  siRNA
gacggattctaaggactctaa

SEQ ID NO: 523 - DNA, 21 bp, Artificial Sequence
  siRNA
aacgaagcgagccaagggcaa

SEQ ID NO: 524 - DNA, 21 bp, Artificial Sequence
  siRNA
ctcggtgcagccgtatttcta

SEQ ID NO: 525 - DNA, 21 bp, Artificial Sequence
  siRNA
cagcggtgtaccattgacatt

SEQ ID NO: 526 - DNA, 21 bp, Artificial Sequence
  siRNA
cacccacaacctatgatccaa

SEQ ID NO: 527 - DNA, 21 bp, Artificial Sequence
  siRNA
ttgaagagaactgcaactgaa

SEQ ID NO: 528 - DNA, 21 bp, Artificial Sequence
  siRNA
aaggcctatgattatttccaa

SEQ ID NO: 529 - DNA, 21 bp, Artificial Sequence
  siRNA
tagctggttatgaactagtaa

SEQ ID NO: 530 - DNA, 21 bp, Artificial Sequence
  siRNA
caagttcttcttgacagacaa

SEQ ID NO: 531 - DNA, 21 bp, Artificial Sequence
  siRNA
cgggaaatatgggatgtataa

SEQ ID NO: 532 - DNA, 21 bp, Artificial Sequence
  siRNA
gaggtgcaatgagctacgtta

SEQ ID NO: 533 - DNA, 21 bp, Artificial Sequence
  siRNA
caacacccatccagaatgtca

SEQ ID NO: 534 - DNA, 21 bp, Artificial Sequence
  siRNA
caggagcagctccaggcagaa

SEQ ID NO: 535 - DNA, 21 bp, Artificial Sequence
  siRNA
ccagttaaagcaggcaataga

SEQ ID NO: 536 - DNA, 21 bp, Artificial Sequence
  siRNA
tacgacactgtccacctttaa

SEQ ID NO: 537 - DNA, 21 bp, Artificial Sequence
  siRNA
ccggatcatacgaaatcaatt

SEQ ID NO: 538 - DNA, 21 bp, Artificial Sequence
  siRNA
cactgtagacttgcttatcta

SEQ ID NO: 539 - DNA, 21 bp, Artificial Sequence
  siRNA
ttggtacgtgggcaagatcaa

SEQ ID NO: 540 - DNA, 21 bp, Artificial Sequence
  siRNA
gacgacggttgtgaatgataa

SEQ ID NO: 541 - DNA, 21 bp, Artificial Sequence
  siRNA
aagctgctcttagaaagataa

SEQ ID NO: 542 - DNA, 21 bp, Artificial Sequence
  siRNA
ctcccaatagcagttacccaa

SEQ ID NO: 543 - DNA, 21 bp, Artificial Sequence
  siRNA
gacctaaagatgcgtatataa

SEQ ID NO: 544 - DNA, 21 bp, Artificial Sequence
  siRNA
ccggaggagaaacgtcttcga

SEQ ID NO: 545 - DNA, 21 bp, Artificial Sequence
  siRNA
agcagtcgtcaataacctaaa

SEQ ID NO: 546 - DNA, 21 bp, Artificial Sequence
  siRNA
aacctgtttgttggacatact

SEQ ID NO: 547 - DNA, 21 bp, Artificial Sequence
  siRNA
ccgcgcgttcctggtgaagaa

SEQ ID NO: 548 - DNA, 21 bp, Artificial Sequence
  siRNA
caagaaggaattaattattaa

SEQ ID NO: 549 - DNA, 21 bp, Artificial Sequence
  siRNA
ctgagtcagtataagtatata

SEQ ID NO: 550 - DNA, 21 bp, Artificial Sequence
  siRNA
gaccgccagattctcccttaa

SEQ ID NO: 551 - DNA, 21 bp, Artificial Sequence
  siRNA
atgcgttatctgggtctggaa

SEQ ID NO: 552 - DNA, 21 bp, Artificial Sequence
  siRNA
aagccgtctatcagctaacta

SEQ ID NO: 553 - DNA, 21 bp, Artificial Sequence
  siRNA
cacgcatgtcaaattctcata

SEQ ID NO: 554 - DNA, 21 bp, Artificial Sequence
  siRNA
caccgttacaagtatacttat

SEQ ID NO: 555 - DNA, 21 bp, Artificial Sequence
  siRNA
cagaagctctatcaagtgcca

SEQ ID NO: 556 - DNA, 21 bp, Artificial Sequence
  siRNA
cacatcatcgagaacccacaa

SEQ ID NO: 557 - DNA, 21 bp, Artificial Sequence
  siRNA
ccgctatgtctggctggttta

SEQ ID NO: 558 - DNA, 21 bp, Artificial Sequence
  siRNA
cagctttatcaacgagaagaa

SEQ ID NO: 559 - DNA, 21 bp, Artificial Sequence
  siRNA
cccttcgataagattattgaa

SEQ ID NO: 560 - DNA, 21 bp, Artificial Sequence
  siRNA
atggaattaagtgccatgaaa

SEQ ID NO: 561 - DNA, 21 bp, Artificial Sequence
  siRNA
aactggacttccagaagaaca

SEQ ID NO: 562 - DNA, 21 bp, Artificial Sequence
  siRNA
aaggagcaccttgacagactt

SEQ ID NO: 563 - DNA, 21 bp, Artificial Sequence
  siRNA
caggaatactcagatcgggaa

SEQ ID NO: 564 - DNA, 21 bp, Artificial Sequence
  siRNA
tcccggtgacacagacacaaa

SEQ ID NO: 565 - DNA, 21 bp, Artificial Sequence
  siRNA
ctgggtatacttcatgtgaca

SEQ ID NO: 566 - DNA, 21 bp, Artificial Sequence
  siRNA
cagcgcttcagtggttctata

SEQ ID NO: 567 - DNA, 21 bp, Artificial Sequence
  siRNA
ctccaactacgcgtatgtgaa

SEQ ID NO: 568 - DNA, 21 bp, Artificial Sequence
  siRNA
ccgagcaactttgatcaacga

SEQ ID NO: 569 - DNA, 21 bp, Artificial Sequence
  siRNA
ccggtcacgcatgaaggcaaa

SEQ ID NO: 570 - DNA, 21 bp, Artificial Sequence
  siRNA
aaggatggaagaagagacgaa

SEQ ID NO: 571 - DNA, 21 bp, Artificial Sequence
  siRNA
ctggctattgacttccctgaa

SEQ ID NO: 572 - DNA, 21 bp, Artificial Sequence
  siRNA
cagatgaaacaaagcagtgaa

SEQ ID NO: 573 - DNA, 21 bp, Artificial Sequence
  siRNA
caccaatccctgaaagattaa

SEQ ID NO: 574 - DNA, 21 bp, Artificial Sequence
  siRNA
ctggccattgtctatctcatt

SEQ ID NO: 575 - DNA, 21 bp, Artificial Sequence
  siRNA
cacagcggtgagtgctgcaaa

SEQ ID NO: 576 - DNA, 21 bp, Artificial Sequence
  siRNA
cacggataactttatcttgtt

SEQ ID NO: 577 - DNA, 21 bp, Artificial Sequence
  siRNA
cagaatcttctgaaccatcta

SEQ ID NO: 578 - DNA, 21 bp, Artificial Sequence
  siRNA
aagcggttaggctgtgaggaa

SEQ ID NO: 579 - DNA, 21 bp, Artificial Sequence
  siRNA
atcgaagtttgcagagacaaa

SEQ ID NO: 580 - DNA, 21 bp, Artificial Sequence
  siRNA
tccggaggtcacccacgctta

SEQ ID NO: 581 - DNA, 21 bp, Artificial Sequence
  siRNA
acagatctttggagtgcctaa

SEQ ID NO: 582 - DNA, 21 bp, Artificial Sequence
  siRNA
acggattctaaggactctaaa

SEQ ID NO: 583 - DNA, 21 bp, Artificial Sequence
  siRNA
ccgggaatctatactcaatga

SEQ ID NO: 584 - DNA, 21 bp, Artificial Sequence
  siRNA
gatcccggagttggaaaacaa

SEQ ID NO: 585 - DNA, 21 bp, Artificial Sequence
  siRNA
ttagatgataatggacaacta

SEQ ID NO: 586 - DNA, 21 bp, Artificial Sequence
  siRNA
aaagaagcgtttcacaacaaa

SEQ ID NO: 587 - DNA, 21 bp, Artificial Sequence
  siRNA
tccactgattgctgcagctaa

SEQ ID NO: 588 - DNA, 21 bp, Artificial Sequence
  siRNA
atgactgatattacacctcaa

SEQ ID NO: 589 - DNA, 21 bp, Artificial Sequence
  siRNA
cagcattaaaccagaccttat

SEQ ID NO: 590 - DNA, 21 bp, Artificial Sequence
  siRNA
cacgctctctttctggcggaa

SEQ ID NO: 591 - DNA, 21 bp, Artificial Sequence
  siRNA
cccggacgacttgtctttcaa

SEQ ID NO: 592 - DNA, 21 bp, Artificial Sequence
  siRNA
cagcgtttggatagggcacaa

SEQ ID NO: 593 - DNA, 21 bp, Artificial Sequence
  siRNA
cgcgccgtgatgaatatcgaa

SEQ ID NO: 594 - DNA, 21 bp, Artificial Sequence
  siRNA
aaccgggacgaagccatcaaa

SEQ ID NO: 595 - DNA, 21 bp, Artificial Sequence
  siRNA
cagggccttgctgtgacaata

SEQ ID NO: 596 - DNA, 21 bp, Artificial Sequence
  siRNA
ctggatttagaacatggacta

SEQ ID NO: 597 - DNA, 21 bp, Artificial Sequence
  siRNA
ccgcgaccggatcatacgaaa

SEQ ID NO: 598 - DNA, 21 bp, Artificial Sequence
  siRNA
gagggaggagctaaacggtta

SEQ ID NO: 599 - DNA, 21 bp, Artificial Sequence
  siRNA
ccgcgagtatgaccagcttta

SEQ ID NO: 600 - DNA, 21 bp, Artificial Sequence
  siRNA
gggcgggaccctgaaatacta

SEQ ID NO: 601 - DNA, 21 bp, Artificial Sequence
  siRNA
tccgaattgataatctttcaa

SEQ ID NO: 602 - DNA, 21 bp, Artificial Sequence
  siRNA
cacgccagagctggccgagca

SEQ ID NO: 603 - DNA, 21 bp, Artificial Sequence
  siRNA
cagaaggtggtgaaactgaaa

SEQ ID NO: 604 - DNA, 21 bp, Artificial Sequence
  siRNA
cagcagcaagtgcaacatcaa

SEQ ID NO: 605 - DNA, 21 bp, Artificial Sequence
  siRNA
cacaagaagtgtattgataaa

SEQ ID NO: 606 - DNA, 21 bp, Artificial Sequence
  siRNA
atcgtgaacttgtcctcttaa

SEQ ID NO: 607 - DNA, 21 bp, Artificial Sequence
  siRNA
ttggaatactctaagagaata

SEQ ID NO: 608 - DNA, 21 bp, Artificial Sequence
  siRNA
acggacgttggttctgcacta

SEQ ID NO: 609 - DNA, 21 bp, Artificial Sequence
  siRNA
cacgaccatctgggacggaat

SEQ ID NO: 610 - DNA, 21 bp, Artificial Sequence
  siRNA
gacatgcttgctgatcagcta

SEQ ID NO: 611 - DNA, 21 bp, Artificial Sequence
  siRNA
cagcaaggtgctcatcgcaga

SEQ ID NO: 612 - DNA, 21 bp, Artificial Sequence
  siRNA
cccgaccagcattgacaagat

SEQ ID NO: 613 - DNA, 21 bp, Artificial Sequence
  siRNA
ctgaccaatcaagtacactaa

SEQ ID NO: 614 - DNA, 21 bp, Artificial Sequence
  siRNA
cacggaaacacccgtacctta

SEQ ID NO: 615 - DNA, 21 bp, Artificial Sequence
  siRNA
gaccaaatttacgccatgaaa

SEQ ID NO: 616 - DNA, 21 bp, Artificial Sequence
  siRNA
tccgagcacaataaactcaaa

SEQ ID NO: 617 - DNA, 21 bp, Artificial Sequence
  siRNA
ccgcacctacttctacgccaa

SEQ ID NO: 618 - DNA, 21 bp, Artificial Sequence
  siRNA
cacaaaggagttacatcttaa

SEQ ID NO: 619 - DNA, 21 bp, Artificial Sequence
  siRNA
tccgagtatgtctaccattta

SEQ ID NO: 620 - DNA, 21 bp, Artificial Sequence
  siRNA
tgggaaatagaagccagtgaa

SEQ ID NO: 621 - DNA, 21 bp, Artificial Sequence
  siRNA
ccagactaatatattaatata

SEQ ID NO: 622 - DNA, 21 bp, Artificial Sequence
  siRNA
ttcaagatgactaatgtcaaa

SEQ ID NO: 623 - DNA, 21 bp, Artificial Sequence
  siRNA
ctggatcgccatctatgagaa

SEQ ID NO: 624 - DNA, 21 bp, Artificial Sequence
  siRNA
ctacgccatcaagatcttgaa

SEQ ID NO: 625 - DNA, 21 bp, Artificial Sequence
  siRNA
gaccctgttgacagtacttta

SEQ ID NO: 626 - DNA, 21 bp, Artificial Sequence
  siRNA
ctggatcaagtcctgaagaaa

SEQ ID NO: 627 - DNA, 21 bp, Artificial Sequence
  siRNA
aaggcgtatacaggaacaata

SEQ ID NO: 628 - DNA, 21 bp, Artificial Sequence
  siRNA
taggccctgatgagaacgcta

SEQ ID NO: 629 - DNA, 21 bp, Artificial Sequence
  siRNA
tccgactttgatagatttcta

SEQ ID NO: 630 - DNA, 21 bp, Artificial Sequence
  siRNA
cgcggtgcagattcttcttaa

SEQ ID NO: 631 - DNA, 21 bp, Artificial Sequence
  siRNA
ccagtgtataatcagccagta

SEQ ID NO: 632 - DNA, 21 bp, Artificial Sequence
  siRNA
cactattggatgtgattctaa

SEQ ID NO: 633 - DNA, 21 bp, Artificial Sequence
  siRNA
ctggaccgacctagatttgaa

SEQ ID NO: 634 - DNA, 21 bp, Artificial Sequence
  siRNA
gtgccggtacatttacttaaa

SEQ ID NO: 635 - DNA, 21 bp, Artificial Sequence
  siRNA
atcatggtagtcactaacata

SEQ ID NO: 636 - DNA, 21 bp, Artificial Sequence
  siRNA
ccggcctgccatggccatctt

SEQ ID NO: 637 - DNA, 21 bp, Artificial Sequence
  siRNA
aggctctattccaataaacta

SEQ ID NO: 638 - DNA, 21 bp, Artificial Sequence
  siRNA
ccgctcatgactatacgctaa

SEQ ID NO: 639 - DNA, 21 bp, Artificial Sequence
  siRNA
aacccaaactgtatgtcttga

SEQ ID NO: 640 - DNA, 21 bp, Artificial Sequence
  siRNA
tgacgagtttgtagaagacta

SEQ ID NO: 641 - DNA, 21 bp, Artificial Sequence
  siRNA
ccgcccgcgcccttaatttaa

SEQ ID NO: 642 - DNA, 21 bp, Artificial Sequence
  siRNA
ctcaaacgtcagagcaacgta

SEQ ID NO: 643 - DNA, 21 bp, Artificial Sequence
  siRNA
tacaagttgcttaaccaagaa

SEQ ID NO: 644 - DNA, 21 bp, Artificial Sequence
  siRNA
cacgtcaaagtatgatatcta

SEQ ID NO: 645 - DNA, 21 bp, Artificial Sequence
  siRNA
ctccctgacccgtctaatata

SEQ ID NO: 646 - DNA, 21 bp, Artificial Sequence
  siRNA
ccgggccaccgtgaactcaca

SEQ ID NO: 647 - DNA, 21 bp, Artificial Sequence
  siRNA
ccggtcgaatgataaggtgta

SEQ ID NO: 648 - DNA, 21 bp, Artificial Sequence
  siRNA
aagcttaatgaggaaatatca

SEQ ID NO: 649 - DNA, 21 bp, Artificial Sequence
  siRNA
atgcatttcctggagaatata

SEQ ID NO: 650 - DNA, 21 bp, Artificial Sequence
  siRNA
cagggccagaatttagcaaga

SEQ ID NO: 651 - DNA, 21 bp, Artificial Sequence
  siRNA
tacccgctttatcgaaggcaa

SEQ ID NO: 652 - DNA, 21 bp, Artificial Sequence
  siRNA
cagcgtattctgatatagtaa

SEQ ID NO: 653 - DNA, 21 bp, Artificial Sequence
  siRNA
ccggatgaaactgaccgattt

SEQ ID NO: 654 - DNA, 21 bp, Artificial Sequence
  siRNA
aagcggcacattcccatttaa

SEQ ID NO: 655 - DNA, 21 bp, Artificial Sequence
  siRNA
atgatgtgtcttcaatgtcaa

SEQ ID NO: 656 - DNA, 21 bp, Artificial Sequence
  siRNA
caggaagagaccaaagtataa

SEQ ID NO: 657 - DNA, 21 bp, Artificial Sequence
  siRNA
ctccgcgactctgatgagaaa

SEQ ID NO: 658 - DNA, 21 bp, Artificial Sequence
  siRNA
catcgtgtttgcaaaggttaa

SEQ ID NO: 659 - DNA, 21 bp, Artificial Sequence
  siRNA
aactattcagccaatgtgatg

SEQ ID NO: 660 - DNA, 21 bp, Artificial Sequence
  siRNA
cagtataatgtcttagattaa

SEQ ID NO: 661 - DNA, 21 bp, Artificial Sequence
  siRNA
atgggatatttgattacgtaa

SEQ ID NO: 662 - DNA, 21 bp, Artificial Sequence
  siRNA
cggctctattgtgcactcgga

SEQ ID NO: 663 - DNA, 21 bp, Artificial Sequence
  siRNA
caaggacaactcaaacttata

SEQ ID NO: 664 - DNA, 21 bp, Artificial Sequence
  siRNA
aactctaccgtgccacgtttt

SEQ ID NO: 665 - DNA, 21 bp, Artificial Sequence
  siRNA
aaccatagttatttacttgaa

SEQ ID NO: 666 - DNA, 21 bp, Artificial Sequence
  siRNA
aaagtcgatttatgtgtatta

SEQ ID NO: 667 - DNA, 21 bp, Artificial Sequence
  siRNA
cggatgggattcacttaagaa

SEQ ID NO: 668 - DNA, 21 bp, Artificial Sequence
  siRNA
caggaataggcatttgcctaa

SEQ ID NO: 669 - DNA, 21 bp, Artificial Sequence
  siRNA
atcaccgtggatagacttgaa

SEQ ID NO: 670 - DNA, 21 bp, Artificial Sequence
  siRNA
ccgggagattggctctgtgaa

SEQ ID NO: 671 - DNA, 21 bp, Artificial Sequence
  siRNA
tgggcggacggttctgaacaa

SEQ ID NO: 672 - DNA, 21 bp, Artificial Sequence
  siRNA
cagaaacttctccaaagtgat

SEQ ID NO: 673 - DNA, 21 bp, Artificial Sequence
  siRNA
cagcctgtgtagtgtgacaaa

SEQ ID NO: 674 - DNA, 21 bp, Artificial Sequence
  siRNA
cccgaccatggtagtgttcaa

SEQ ID NO: 675 - DNA, 21 bp, Artificial Sequence
  siRNA
cggaagcatgacagcattaaa

SEQ ID NO: 676 - DNA, 21 bp, Artificial Sequence
  siRNA
ccgcatgtgtctgtattcata

SEQ ID NO: 677 - DNA, 21 bp, Artificial Sequence
  siRNA
ctggcatttgtcaggaatata

SEQ ID NO: 678 - DNA, 21 bp, Artificial Sequence
  siRNA
ccgctgtacttggaaattcga

SEQ ID NO: 679 - DNA, 21 bp, Artificial Sequence
  siRNA
tcgggtagaaataaccaccaa

SEQ ID NO: 680 - DNA, 21 bp, Artificial Sequence
  siRNA
cagatcttagtaagctatata

SEQ ID NO: 681 - DNA, 21 bp, Artificial Sequence
  siRNA
ccgccaggtgaagctggtgaa

SEQ ID NO: 682 - DNA, 21 bp, Artificial Sequence
  siRNA
cagtaatgggttacttctgaa

SEQ ID NO: 683 - DNA, 21 bp, Artificial Sequence
  siRNA
aaccagctggatcgccatcta

SEQ ID NO: 684 - DNA, 21 bp, Artificial Sequence
  siRNA
cccggagatcattgcctacca

SEQ ID NO: 685 - DNA, 21 bp, Artificial Sequence
  siRNA
ttcggctaactcgccagttta

SEQ ID NO: 686 - DNA, 21 bp, Artificial Sequence
  siRNA
ctggaagaattcctgaacaaa

SEQ ID NO: 687 - DNA, 21 bp, Artificial Sequence
  siRNA
tcgacttagacttgacctata

SEQ ID NO: 688 - DNA, 21 bp, Artificial Sequence
  siRNA
ccggaacaaatgcgtcccata

SEQ ID NO: 689 - DNA, 21 bp, Artificial Sequence
  siRNA
ccgactgaaactggaaccctt

SEQ ID NO: 690 - DNA, 21 bp, Artificial Sequence
  siRNA
taggaactcactctttagata

SEQ ID NO: 691 - DNA, 21 bp, Artificial Sequence
  siRNA
cagcgccacagcctccattaa

SEQ ID NO: 692 - DNA, 21 bp, Artificial Sequence
  siRNA
tacgaggatgtcgtccaggaa

SEQ ID NO: 693 - DNA, 21 bp, Artificial Sequence
  siRNA
cagctagtgccaaataattga

SEQ ID NO: 694 - DNA, 21 bp, Artificial Sequence
  siRNA
cagattgttctttgttataga

SEQ ID NO: 695 - DNA, 21 bp, Artificial Sequence
  siRNA
aacacttgtcaagaagcgtta

SEQ ID NO: 696 - DNA, 21 bp, Artificial Sequence
  siRNA
cagcatttgcatggaacacat

SEQ ID NO: 697 - DNA, 21 bp, Artificial Sequence
  siRNA
acccataattgaagtgtataa

SEQ ID NO: 698 - DNA, 21 bp, Artificial Sequence
  siRNA
cagaagcacagtaccgattta

SEQ ID NO: 699 - DNA, 21 bp, Artificial Sequence
  siRNA
attcatggagacatccgctaa

SEQ ID NO: 700 - DNA, 21 bp, Artificial Sequence
  siRNA
caaggtgttctttgacctaat

SEQ ID NO: 701 - DNA, 21 bp, Artificial Sequence
  siRNA
cccgcccatggacgatgtata

SEQ ID NO: 702 - DNA, 21 bp, Artificial Sequence
  siRNA
aagcaatagtcaggagcgata

SEQ ID NO: 703 - DNA, 21 bp, Artificial Sequence
  siRNA
cgcagtggaatgagtccttta

SEQ ID NO: 704 - DNA, 21 bp, Artificial Sequence
  siRNA
aaagtatgatatctacgcaaa

SEQ ID NO: 705 - DNA, 21 bp, Artificial Sequence
  siRNA
cccgtctaatatataaatata

SEQ ID NO: 706 - DNA, 21 bp, Artificial Sequence
  siRNA
acggatatcctgcatgtccaa

SEQ ID NO: 707 - DNA, 21 bp, Artificial Sequence
  siRNA
aatcacacaccaaattcgagt

SEQ ID NO: 708 - DNA, 21 bp, Artificial Sequence
  siRNA
ttgcaggttatcagagatcaa

SEQ ID NO: 709 - DNA, 21 bp, Artificial Sequence
  siRNA
cagcacgtgttcccgacataa

SEQ ID NO: 710 - DNA, 21 bp, Artificial Sequence
  siRNA
cccaacgatagaagattccta

SEQ ID NO: 711 - DNA, 21 bp, Artificial Sequence
  siRNA
ccgcagcgtctaaccactaca

SEQ ID NO: 712 - DNA, 21 bp, Artificial Sequence
  siRNA
ctgtatcatggccatagtata

SEQ ID NO: 713 - DNA, 21 bp, Artificial Sequence
  siRNA
caagagctaagtagatgtgta

SEQ ID NO: 714 - DNA, 21 bp, Artificial Sequence
  siRNA
tcgattatttccagtgttcta

SEQ ID NO: 715 - DNA, 21 bp, Artificial Sequence
  siRNA
atgaaatgtgttaatcacaaa

SEQ ID NO: 716 - DNA, 21 bp, Artificial Sequence
  siRNA
aaagatgacagtggcgattgt

SEQ ID NO: 717 - DNA, 21 bp, Artificial Sequence
  siRNA
acgcgtgtgctcctctcctta

SEQ ID NO: 718 - DNA, 21 bp, Artificial Sequence
  siRNA
cagcgctatctagataggtaa

SEQ ID NO: 719 - DNA, 21 bp, Artificial Sequence
  siRNA
caatgtgatggtggacagcaa

SEQ ID NO: 720 - DNA, 21 bp, Artificial Sequence
  siRNA
gacgagtactgtggatgtgaa

SEQ ID NO: 721 - DNA, 21 bp, Artificial Sequence
  siRNA
ccagtgtattccgtcaagtaa

SEQ ID NO: 722 - DNA, 21 bp, Artificial Sequence
  siRNA
cagggttgtgtcgaaattgga

SEQ ID NO: 723 - DNA, 21 bp, Artificial Sequence
  siRNA
cagcttagggataagtgtcta

SEQ ID NO: 724 - DNA, 21 bp, Artificial Sequence
  siRNA
tccaaacattatcactctaaa

SEQ ID NO: 725 - DNA, 21 bp, Artificial Sequence
  siRNA
ttggacgaggagcttatggtt

SEQ ID NO: 726 - DNA, 21 bp, Artificial Sequence
  siRNA
atggatgtattcatgaaagga

SEQ ID NO: 727 - DNA, 21 bp, Artificial Sequence
  siRNA
aagcatttgatccttatgaaa

SEQ ID NO: 728 - DNA, 21 bp, Artificial Sequence
  siRNA
caagatggagttcatcgacaa

SEQ ID NO: 729 - DNA, 21 bp, Artificial Sequence
  siRNA
aggcacgtggaggaactctta

SEQ ID NO: 730 - DNA, 21 bp, Artificial Sequence
  siRNA
cccgcatgtcacttagtctaa

SEQ ID NO: 731 - DNA, 21 bp, Artificial Sequence
  siRNA
caggaaatgttggagaactca

SEQ ID NO: 732 - DNA, 21 bp, Artificial Sequence
  siRNA
cagctatccctctatataata

SEQ ID NO: 733 - DNA, 21 bp, Artificial Sequence
  siRNA
caccttaggattcattataaa

SEQ ID NO: 734 - DNA, 21 bp, Artificial Sequence
  siRNA
ctcggccaaactgcgtctcaa

SEQ ID NO: 735 - DNA, 21 bp, Artificial Sequence
  siRNA
caaggagaattaggatttaaa

SEQ ID NO: 736 - DNA, 21 bp, Artificial Sequence
  siRNA
ctgagccttatttctctggaa

SEQ ID NO: 737 - DNA, 21 bp, Artificial Sequence
  siRNA
caggtcagttggcagactcta

SEQ ID NO: 738 - DNA, 21 bp, Artificial Sequence
  siRNA
ctccgcgctcttcttcggaaa

SEQ ID NO: 739 - DNA, 21 bp, Artificial Sequence
  siRNA
atgggactcagtagatcttga

SEQ ID NO: 740 - DNA, 21 bp, Artificial Sequence
  siRNA
gacgacaagaaggatatcaaa

SEQ ID NO: 741 - DNA, 21 bp, Artificial Sequence
  siRNA
gagcgtgtaattaccttgaaa

SEQ ID NO: 742 - DNA, 21 bp, Artificial Sequence
  siRNA
ccgacgcaagatggcgagtaa

SEQ ID NO: 743 - DNA, 21 bp, Artificial Sequence
  siRNA
accgagaagggttaaactata

SEQ ID NO: 744 - DNA, 21 bp, Artificial Sequence
  siRNA
cggaggtgcatcagcgagcaa

SEQ ID NO: 745 - DNA, 21 bp, Artificial Sequence
  siRNA
ctgaaatttgctggaatgcca

SEQ ID NO: 746 - DNA, 21 bp, Artificial Sequence
  siRNA
tgccattggtgttgagaataa

SEQ ID NO: 747 - DNA, 21 bp, Artificial Sequence
  siRNA
aaggttgaagcagaagaataa

SEQ ID NO: 748 - DNA, 21 bp, Artificial Sequence
  siRNA
ccagatgtctatgatcattta

SEQ ID NO: 749 - DNA, 21 bp, Artificial Sequence
  siRNA
acggataggcaggaacataca

SEQ ID NO: 750 - DNA, 21 bp, Artificial Sequence
  siRNA
gagcggaacctgaatcccaaa

SEQ ID NO: 751 - DNA, 21 bp, Artificial Sequence
  siRNA
tcctgtttcgccggacttgaa

SEQ ID NO: 752 - DNA, 21 bp, Artificial Sequence
  siRNA
taggctggttctcaaccggaa

SEQ ID NO: 753 - DNA, 21 bp, Artificial Sequence
  siRNA
cagcaaagccttgcaatccca

SEQ ID NO: 754 - DNA, 21 bp, Artificial Sequence
  siRNA
ccgctatggcctcctcataaa

SEQ ID NO: 755 - DNA, 21 bp, Artificial Sequence
  siRNA
ttggttcagggctcaactaaa

SEQ ID NO: 756 - DNA, 21 bp, Artificial Sequence
  siRNA
gagggccgctttagaaaggta

SEQ ID NO: 757 - DNA, 21 bp, Artificial Sequence
  siRNA
aacgcttccttgctgacttcc

SEQ ID NO: 758 - DNA, 21 bp, Artificial Sequence
  siRNA
aacgttcaggtggttcaggaa

SEQ ID NO: 759 - DNA, 21 bp, Artificial Sequence
  siRNA
cggcgcctaagagtaaactaa

SEQ ID NO: 760 - DNA, 21 bp, Artificial Sequence
  siRNA
atggttggaactcatcgggaa

SEQ ID NO: 761 - DNA, 21 bp, Artificial Sequence
  siRNA
cagacctaataggttattaca

SEQ ID NO: 762 - DNA, 21 bp, Artificial Sequence
  siRNA
ctggtccctcagtgtgcctaa

SEQ ID NO: 763 - DNA, 21 bp, Artificial Sequence
  siRNA
tgccacgtactccgcactcaa

SEQ ID NO: 764 - DNA, 21 bp, Artificial Sequence
  siRNA
ttgaagatacttggcactatt

SEQ ID NO: 765 - DNA, 21 bp, Artificial Sequence
  siRNA
aaggctctggaaagtccttca

SEQ ID NO: 766 - DNA, 21 bp, Artificial Sequence
  siRNA
aagtgctgccatgttaggtaa

SEQ ID NO: 767 - DNA, 21 bp, Artificial Sequence
  siRNA
tgggagacgcatataatatga

SEQ ID NO: 768 - DNA, 21 bp, Artificial Sequence
  siRNA
caggctggtaatttatataat

SEQ ID NO: 769 - DNA, 21 bp, Artificial Sequence
  siRNA
caccatttgctgtgcgaatta

SEQ ID NO: 770 - DNA, 21 bp, Artificial Sequence
  siRNA
catgatgacatagaagtacta

SEQ ID NO: 771 - DNA, 21 bp, Artificial Sequence
  siRNA
ccgcagtttaatgaaatcttt

SEQ ID NO: 772 - DNA, 21 bp, Artificial Sequence
  siRNA
caggaccatggcacccttaga

SEQ ID NO: 773 - DNA, 21 bp, Artificial Sequence
  siRNA
ccggaggtcctgaagcgtcaa

SEQ ID NO: 774 - DNA, 21 bp, Artificial Sequence
  siRNA
gtgcattgacccgaagctaaa

SEQ ID NO: 775 - DNA, 21 bp, Artificial Sequence
  siRNA
ctccacaaccatcaacaggaa

SEQ ID NO: 776 - DNA, 21 bp, Artificial Sequence
  siRNA
aaggaggtcctaggttataaa

SEQ ID NO: 777 - DNA, 21 bp, Artificial Sequence
  siRNA
ctccatgtgcgtccatattta

SEQ ID NO: 778 - DNA, 21 bp, Artificial Sequence
  siRNA
tcagaggccgttggtacttaa

SEQ ID NO: 779 - DNA, 21 bp, Artificial Sequence
  siRNA
caccttcggcatcctaatatt

SEQ ID NO: 780 - DNA, 21 bp, Artificial Sequence
  siRNA
ctggctgtccttatcatcaca

SEQ ID NO: 781 - DNA, 21 bp, Artificial Sequence
  siRNA
accggcaccttcatcgagaaa

SEQ ID NO: 782 - DNA, 21 bp, Artificial Sequence
  siRNA
cgcgtgtaaattctactgcaa

SEQ ID NO: 783 - DNA, 21 bp, Artificial Sequence
  siRNA
ccacatttaattaacagctga

SEQ ID NO: 784 - DNA, 21 bp, Artificial Sequence
  siRNA
agcgctgagaatggacagcaa

SEQ ID NO: 785 - DNA, 21 bp, Artificial Sequence
  siRNA
agggtgtatagtgttcacaaa

SEQ ID NO: 786 - DNA, 21 bp, Artificial Sequence
  siRNA
aaagagggtgttctctatgta

SEQ ID NO: 787 - DNA, 21 bp, Artificial Sequence
  siRNA
cagcataatatttgctgctaa

SEQ ID NO: 788 - DNA, 21 bp, Artificial Sequence
  siRNA
aaggagcggcctcgccataaa

SEQ ID NO: 789 - DNA, 21 bp, Artificial Sequence
  siRNA
ttcgaagttaggaggactcaa

SEQ ID NO: 790 - DNA, 21 bp, Artificial Sequence
  siRNA
tagagtcatccctgtaatcaa

SEQ ID NO: 791 - DNA, 21 bp, Artificial Sequence
  siRNA
cactgagatctactggataaa

SEQ ID NO: 792 - DNA, 21 bp, Artificial Sequence
  siRNA
aagactccttccagtgatgtt

SEQ ID NO: 793 - DNA, 21 bp, Artificial Sequence
  siRNA
aaggatgaatgtagttctgaa

SEQ ID NO: 794 - DNA, 21 bp, Artificial Sequence
  siRNA
ccgggtcactgctgtatataa

SEQ ID NO: 795 - DNA, 21 bp, Artificial Sequence
  siRNA
cgcctggattacgattataaa

SEQ ID NO: 796 - DNA, 21 bp, Artificial Sequence
  siRNA
cagctgctactttgacatcga

SEQ ID NO: 797 - DNA, 21 bp, Artificial Sequence
  siRNA
atgcctaatattcagtatcaa

SEQ ID NO: 798 - DNA, 21 bp, Artificial Sequence
  siRNA
cagccgcaatctctcggccaa

SEQ ID NO: 799 - DNA, 21 bp, Artificial Sequence
  siRNA
gccaccctaatttgacatcaa

SEQ ID NO: 800 - DNA, 21 bp, Artificial Sequence
  siRNA
agccgaattgaagatcgttta

SEQ ID NO: 801 - DNA, 21 bp, Artificial Sequence
  siRNA
ctgccagttcagttaatcaaa

SEQ ID NO: 802 - DNA, 21 bp, Artificial Sequence
  siRNA
gcggataagaccgtagcttta

SEQ ID NO: 803 - DNA, 21 bp, Artificial Sequence
  siRNA
aaggtaattgctctaacttta

SEQ ID NO: 804 - DNA, 21 bp, Artificial Sequence
  siRNA
aacgcgcttgtccacaaacaa

SEQ ID NO: 805 - DNA, 21 bp, Artificial Sequence
  siRNA
aagagccacctgaccatcagt

SEQ ID NO: 806 - DNA, 21 bp, Artificial Sequence
  siRNA
ccgcaacaataagaaactcct

SEQ ID NO: 807 - DNA, 21 bp, Artificial Sequence
  siRNA
gagcgacgtggtggaagtcta

SEQ ID NO: 808 - DNA, 21 bp, Artificial Sequence
  siRNA
cagaaagagcttgacagtaaa

SEQ ID NO: 809 - DNA, 21 bp, Artificial Sequence
  siRNA
cagcggctctatgtcgacttt

SEQ ID NO: 810 - DNA, 21 bp, Artificial Sequence
  siRNA
ctggatgattgggactttaaa

SEQ ID NO: 811 - DNA, 21 bp, Artificial Sequence
  siRNA
tacgactgtatggaaacgttt

SEQ ID NO: 812 - DNA, 21 bp, Artificial Sequence
  siRNA
ccgctggtggactgtaataat

SEQ ID NO: 813 - DNA, 21 bp, Artificial Sequence
  siRNA
cagaaacaggttgacaactca

SEQ ID NO: 814 - DNA, 21 bp, Artificial Sequence
  siRNA
ctgggaatggacgatagagaa

SEQ ID NO: 815 - DNA, 21 bp, Artificial Sequence
  siRNA
aggcaagtaagtccatatcaa

SEQ ID NO: 816 - DNA, 21 bp, Artificial Sequence
  siRNA
ctgggtctttatgagaagctt

SEQ ID NO: 817 - DNA, 21 bp, Artificial Sequence
  siRNA
ccggctgcgtacgtgaagaaa

SEQ ID NO: 818 - DNA, 21 bp, Artificial Sequence
  siRNA
taggccgattaactaccctaa

SEQ ID NO: 819 - DNA, 21 bp, Artificial Sequence
  siRNA
ccggcgcctaagagtaaacta

SEQ ID NO: 820 - DNA, 21 bp, Artificial Sequence
  siRNA
aaccgtgaatctcctagttat

SEQ ID NO: 821 - DNA, 21 bp, Artificial Sequence
  siRNA
acggacctgtcccgtgattta

SEQ ID NO: 822 - DNA, 21 bp, Artificial Sequence
  siRNA
ctggattcttgcaccagggaa

SEQ ID NO: 823 - DNA, 21 bp, Artificial Sequence
  siRNA
cccaacatcatcactctgaaa

SEQ ID NO: 824 - DNA, 21 bp, Artificial Sequence
  siRNA
ctctcagtataatgcctataa

SEQ ID NO: 825 - DNA, 21 bp, Artificial Sequence
  siRNA
cagtgccttagatacaagaaa

SEQ ID NO: 826 - DNA, 21 bp, Artificial Sequence
  siRNA
ctggatcagcaacttaggaaa

SEQ ID NO: 827 - DNA, 21 bp, Artificial Sequence
  siRNA
ctcgagtacttgttgacatta

SEQ ID NO: 828 - DNA, 21 bp, Artificial Sequence
  siRNA
cagcctctctgcagaattcaa

SEQ ID NO: 829 - DNA, 21 bp, Artificial Sequence
  siRNA
aggattcatagcatcactata

SEQ ID NO: 830 - DNA, 21 bp, Artificial Sequence
  siRNA
cagaggcggatagaacggata

SEQ ID NO: 831 - DNA, 21 bp, Artificial Sequence
  siRNA
ctacctgaaagtaacattaaa

SEQ ID NO: 832 - DNA, 21 bp, Artificial Sequence
  siRNA
cagactgttctgggcacggaa

SEQ ID NO: 833 - DNA, 21 bp, Artificial Sequence
  siRNA
ccgagtgagatcgaagatgga

SEQ ID NO: 834 - DNA, 21 bp, Artificial Sequence
  siRNA
ctgaagaacaacaacagacaa

SEQ ID NO: 835 - DNA, 21 bp, Artificial Sequence
  siRNA
accagagaagtttgccctgaa

SEQ ID NO: 836 - DNA, 21 bp, Artificial Sequence
  siRNA
tgggttgaatccatcactaaa

SEQ ID NO: 837 - DNA, 21 bp, Artificial Sequence
  siRNA
cggcttgtgggtgatgtttga

SEQ ID NO: 838 - DNA, 21 bp, Artificial Sequence
  siRNA
tggaatcaagtccaacagtta

SEQ ID NO: 839 - DNA, 21 bp, Artificial Sequence
  siRNA
tcccagcgcattcctttgcaa

SEQ ID NO: 840 - DNA, 21 bp, Artificial Sequence
  siRNA
aagccggtaaatgcctcaata

SEQ ID NO: 841 - DNA, 21 bp, Artificial Sequence
  siRNA
ctgccttattatgatcttgta

SEQ ID NO: 842 - DNA, 21 bp, Artificial Sequence
  siRNA
cccagacaagaagataaagat

SEQ ID NO: 843 - DNA, 21 bp, Artificial Sequence
  siRNA
tggatttaaggtgggcattaa

SEQ ID NO: 844 - DNA, 21 bp, Artificial Sequence
  siRNA
aagcgagtaaccagctcataa

SEQ ID NO: 845 - DNA, 21 bp, Artificial Sequence
  siRNA
ttggccatgtttatgatttga

SEQ ID NO: 846 - DNA, 21 bp, Artificial Sequence
  siRNA
cacgatcaatgagctgagcaa

SEQ ID NO: 847 - DNA, 21 bp, Artificial Sequence
  siRNA
tacgtatctggaagttatcaa

SEQ ID NO: 848 - DNA, 21 bp, Artificial Sequence
  siRNA
ccgacatttcctggcattgaa

SEQ ID NO: 849 - DNA, 21 bp, Artificial Sequence
  siRNA
cttgtcgaacatcaagtagaa

SEQ ID NO: 850 - DNA, 21 bp, Artificial Sequence
  siRNA
atgattctgtattaatgtaaa

SEQ ID NO: 851 - DNA, 21 bp, Artificial Sequence
  siRNA
ccggcggttcatgaggagtga

SEQ ID NO: 852 - DNA, 21 bp, Artificial Sequence
  siRNA
aaggaaatttgcgtgtggagt

SEQ ID NO: 853 - DNA, 21 bp, Artificial Sequence
  siRNA
ccgcactagtagagaatccat

SEQ ID NO: 854 - DNA, 21 bp, Artificial Sequence
  siRNA
gaggctcctgcttatttataa

SEQ ID NO: 855 - DNA, 21 bp, Artificial Sequence
  siRNA
aaggtgagcagtattgatttg

SEQ ID NO: 856 - DNA, 21 bp, Artificial Sequence
  siRNA
ccggaactctggcgtcaacta

SEQ ID NO: 857 - DNA, 21 bp, Artificial Sequence
  siRNA
tacccagtatctttgcacaaa

SEQ ID NO: 858 - DNA, 21 bp, Artificial Sequence
  siRNA
aactttgatgtgattaaggga

SEQ ID NO: 859 - DNA, 21 bp, Artificial Sequence
  siRNA
caggatgagcaacaacagtaa

SEQ ID NO: 860 - DNA, 21 bp, Artificial Sequence
  siRNA
ctccaagttgatgctcttaaa

SEQ ID NO: 861 - DNA, 21 bp, Artificial Sequence
  siRNA
ccagtatctgataatgaagaa

SEQ ID NO: 862 - DNA, 21 bp, Artificial Sequence
  siRNA
ctggcccaacttcatttccat

SEQ ID NO: 863 - DNA, 21 bp, Artificial Sequence
  siRNA
tagatagtacgaagtcttcaa

SEQ ID NO: 864 - DNA, 21 bp, Artificial Sequence
  siRNA
aaagagtaccgtgagaagata

SEQ ID NO: 865 - DNA, 21 bp, Artificial Sequence
  siRNA
aagttgtggtctgatcagtta

SEQ ID NO: 866 - DNA, 21 bp, Artificial Sequence
  siRNA
gacgggctatgtaccgtccaa

SEQ ID NO: 867 - DNA, 21 bp, Artificial Sequence
  siRNA
cagaatagcctacatttgtat

SEQ ID NO: 868 - DNA, 21 bp, Artificial Sequence
  siRNA
cagaacggtccagtcaccaaa

SEQ ID NO: 869 - DNA, 21 bp, Artificial Sequence
  siRNA
aaccttaatgtaatttactta

SEQ ID NO: 870 - DNA, 21 bp, Artificial Sequence
  siRNA
ctgtggcatatttgtcactaa

SEQ ID NO: 871 - DNA, 21 bp, Artificial Sequence
  siRNA
caccatgaactcgatcacaga

SEQ ID NO: 872 - DNA, 21 bp, Artificial Sequence
  siRNA
cagcgtgatatgtaccgtatt

SEQ ID NO: 873 - DNA, 21 bp, Artificial Sequence
  siRNA
caaggagaggttcctcgtcta

SEQ ID NO: 874 - DNA, 21 bp, Artificial Sequence
  siRNA
accattagcaaatggaaatta

SEQ ID NO: 875 - DNA, 21 bp, Artificial Sequence
  siRNA
cacgctcaggttgaaggtcga

SEQ ID NO: 876 - DNA, 21 bp, Artificial Sequence
  siRNA
ctcagtgtagtggaagtgata

SEQ ID NO: 877 - DNA, 21 bp, Artificial Sequence
  siRNA
cagtgtattgacgcatattta

SEQ ID NO: 878 - DNA, 21 bp, Artificial Sequence
  siRNA
cccatttatatctctcgcata

SEQ ID NO: 879 - DNA, 21 bp, Artificial Sequence
  siRNA
tacctagtgcatgctctgcaa

SEQ ID NO: 880 - DNA, 21 bp, Artificial Sequence
  siRNA
ctggatctcttacgtaattca

SEQ ID NO: 881 - DNA, 21 bp, Artificial Sequence
  siRNA
aacagagacccctgaagatcc

SEQ ID NO: 882 - DNA, 21 bp, Artificial Sequence
  siRNA
tcgcctcagtttgaacattga

SEQ ID NO: 883 - DNA, 21 bp, Artificial Sequence
  siRNA
ctgaaagtctgccacgataaa

SEQ ID NO: 884 - DNA, 21 bp, Artificial Sequence
  siRNA
caatccaagcataattgttaa

SEQ ID NO: 885 - DNA, 21 bp, Artificial Sequence
  siRNA
ccctgccttgataatatgtta

SEQ ID NO: 886 - DNA, 21 bp, Artificial Sequence
  siRNA
atgaattatgataaattgaaa

SEQ ID NO: 887 - DNA, 21 bp, Artificial Sequence
  siRNA
ccgcacttccatattctggaa

SEQ ID NO: 888 - DNA, 21 bp, Artificial Sequence
  siRNA
aaggagacgtcagaacgtggc

SEQ ID NO: 889 - DNA, 21 bp, Artificial Sequence
  siRNA
tcgggagttcctggaccagta

SEQ ID NO: 890 - DNA, 21 bp, Artificial Sequence
  siRNA
ctgagcatcgatggcgagaat

SEQ ID NO: 891 - DNA, 21 bp, Artificial Sequence
  siRNA
tccgaagatgattcacctaga

SEQ ID NO: 892 - DNA, 21 bp, Artificial Sequence
  siRNA
caggaggagactgtgagaatt

SEQ ID NO: 893 - DNA, 21 bp, Artificial Sequence
  siRNA
ttgctgcagcacattataata

SEQ ID NO: 894 - DNA, 21 bp, Artificial Sequence
  siRNA
cacggaagagatgatgcgcga

SEQ ID NO: 895 - DNA, 21 bp, Artificial Sequence
  siRNA
aagggttaccttccagttcaa

SEQ ID NO: 896 - DNA, 21 bp, Artificial Sequence
  siRNA
ttcggaaatgcctcacatata

SEQ ID NO: 897 - DNA, 21 bp, Artificial Sequence
  siRNA
tggcggcgatatagtgaattt

SEQ ID NO: 898 - DNA, 21 bp, Artificial Sequence
  siRNA
taggaggaaacccagaaatga

SEQ ID NO: 899 - DNA, 21 bp, Artificial Sequence
  siRNA
gagtatgacaatgttgtacta

SEQ ID NO: 900 - DNA, 21 bp, Artificial Sequence
  siRNA
ctacgagcagatcaagataaa

SEQ ID NO: 901 - DNA, 21 bp, Artificial Sequence
  siRNA
tgggattgtactataccagta

SEQ ID NO: 902 - DNA, 21 bp, Artificial Sequence
  siRNA
tagatgtaacagagctcacta

SEQ ID NO: 903 - DNA, 21 bp, Artificial Sequence
  siRNA
cagctgatcaccgggaaggaa

SEQ ID NO: 904 - DNA, 21 bp, Artificial Sequence
  siRNA
aagcataactatgagtgttta

SEQ ID NO: 905 - DNA, 21 bp, Artificial Sequence
  siRNA
taccaacagttatctcatgta

SEQ ID NO: 906 - DNA, 21 bp, Artificial Sequence
  siRNA
gcggagatgcacaacatctta

SEQ ID NO: 907 - DNA, 21 bp, Artificial Sequence
  siRNA
atgcgtttggcagttgcatta

SEQ ID NO: 908 - DNA, 21 bp, Artificial Sequence
  siRNA
taccactagtctgacggataa

SEQ ID NO: 909 - DNA, 21 bp, Artificial Sequence
  siRNA
cagaaggagaatccagcacga

SEQ ID NO: 910 - DNA, 21 bp, Artificial Sequence
  siRNA
tccgacctctttatttctata

SEQ ID NO: 911 - DNA, 21 bp, Artificial Sequence
  siRNA
agcccgttgtgcagagttcaa

SEQ ID NO: 912 - DNA, 21 bp, Artificial Sequence
  siRNA
cagagtgcattgtgagggtta

SEQ ID NO: 913 - DNA, 21 bp, Artificial Sequence
  siRNA
cacactccagtttgtaataaa

SEQ ID NO: 914 - DNA, 21 bp, Artificial Sequence
  siRNA
ccagcctacattcacttctaa

SEQ ID NO: 915 - DNA, 21 bp, Artificial Sequence
  siRNA
accaagaatgatcctttcaaa

SEQ ID NO: 916 - DNA, 21 bp, Artificial Sequence
  siRNA
ccgagcggccactctgagtaa

SEQ ID NO: 917 - DNA, 21 bp, Artificial Sequence
  siRNA
taaagtcagtttaggtaataa

SEQ ID NO: 918 - DNA, 21 bp, Artificial Sequence
  siRNA
acggaaatttgaacagttgaa

SEQ ID NO: 919 - DNA, 21 bp, Artificial Sequence
  siRNA
cacgttgattcaccattgtga

SEQ ID NO: 920 - DNA, 21 bp, Artificial Sequence
  siRNA
aagaagagtattaaatatata

SEQ ID NO: 921 - DNA, 21 bp, Artificial Sequence
  siRNA
aaggatccatatcccgaggaa

SEQ ID NO: 922 - DNA, 21 bp, Artificial Sequence
  siRNA
ttgcatatttccttcattcta

SEQ ID NO: 923 - DNA, 21 bp, Artificial Sequence
  siRNA
atgctcagtcatacacgcgaa

SEQ ID NO: 924 - DNA, 21 bp, Artificial Sequence
  siRNA
cagcaatatgttcactatgtt

SEQ ID NO: 925 - DNA, 21 bp, Artificial Sequence
  siRNA
tccaacctatactaacctgaa

SEQ ID NO: 926 - DNA, 21 bp, Artificial Sequence
  siRNA
gacactaatacagatgattaa

SEQ ID NO: 927 - DNA, 21 bp, Artificial Sequence
  siRNA
cacacccagtgatccatccaa

SEQ ID NO: 928 - DNA, 21 bp, Artificial Sequence
  siRNA
ccgagacttatctaaaccaga

SEQ ID NO: 929 - DNA, 21 bp, Artificial Sequence
  siRNA
cagatacgacagggtatccaa

SEQ ID NO: 930 - DNA, 21 bp, Artificial Sequence
  siRNA
ttgcctgtttgtcgactataa

SEQ ID NO: 931 - DNA, 21 bp, Artificial Sequence
  siRNA
cccgggcagccgggatttaaa

SEQ ID NO: 932 - DNA, 21 bp, Artificial Sequence
  siRNA
gagggtgaagatagtaatgaa

SEQ ID NO: 933 - DNA, 21 bp, Artificial Sequence
  siRNA
gtcgaggtcaagcacattaaa

SEQ ID NO: 934 - DNA, 21 bp, Artificial Sequence
  siRNA
aagcatctaagagagaggtta

SEQ ID NO: 935 - DNA, 21 bp, Artificial Sequence
  siRNA
cacgagcagcagattaaggat

SEQ ID NO: 936 - DNA, 21 bp, Artificial Sequence
  siRNA
aagcggatcattggtgggaaa

SEQ ID NO: 937 - DNA, 21 bp, Artificial Sequence
  siRNA
tcgctatagaataatgcatta

SEQ ID NO: 938 - DNA, 21 bp, Artificial Sequence
  siRNA
ttcgagaggtgtatcaatata

SEQ ID NO: 939 - DNA, 21 bp, Artificial Sequence
  siRNA
cacatgcatgtgactcctcaa

SEQ ID NO: 940 - DNA, 21 bp, Artificial Sequence
  siRNA
aagccagtcattcgagatgaa

SEQ ID NO: 941 - DNA, 21 bp, Artificial Sequence
  siRNA
tgggaaagctttggactacta

SEQ ID NO: 942 - DNA, 21 bp, Artificial Sequence
  siRNA
aagttgatgtggatggtacat

SEQ ID NO: 943 - DNA, 21 bp, Artificial Sequence
  siRNA
ttggcgatgtacatcaatgaa

SEQ ID NO: 944 - DNA, 21 bp, Artificial Sequence
  siRNA
caggtttatgttacttctatt

SEQ ID NO: 945 - DNA, 21 bp, Artificial Sequence
  siRNA
cagcctcttgatgtcaatcca

SEQ ID NO: 946 - DNA, 21 bp, Artificial Sequence
  siRNA
cagggctgtagtattcagtaa

SEQ ID NO: 947 - DNA, 21 bp, Artificial Sequence
  siRNA
caggttgaacatggaaataaa

SEQ ID NO: 948 - DNA, 21 bp, Artificial Sequence
  siRNA
tcctatggttgtggtccgaaa

SEQ ID NO: 949 - DNA, 21 bp, Artificial Sequence
  siRNA
cagaagagcctattacatcta

SEQ ID NO: 950 - DNA, 21 bp, Artificial Sequence
  siRNA
aggcaccgagttcatgcaaga

SEQ ID NO: 951 - DNA, 21 bp, Artificial Sequence
  siRNA
ctggttaatagtgatagttga

SEQ ID NO: 952 - DNA, 21 bp, Artificial Sequence
  siRNA
tgggctgctttgtatgttcta

SEQ ID NO: 953 - DNA, 21 bp, Artificial Sequence
  siRNA
cacggcttatgcaagcaaaga

SEQ ID NO: 954 - DNA, 21 bp, Artificial Sequence
  siRNA
ccgcaacgtcctgctggttaa

SEQ ID NO: 955 - DNA, 21 bp, Artificial Sequence
  siRNA
tacactctattcaaacagcaa

SEQ ID NO: 956 - DNA, 21 bp, Artificial Sequence
  siRNA
ctcctctagttcagagacata

SEQ ID NO: 957 - DNA, 21 bp, Artificial Sequence
  siRNA
aggcgacggattggtttagaa

SEQ ID NO: 958 - DNA, 21 bp, Artificial Sequence
  siRNA
cagcttgtgatgcaagtgtga

SEQ ID NO: 959 - DNA, 21 bp, Artificial Sequence
  siRNA
ttgggctgcttggctatgcaa

SEQ ID NO: 960 - DNA, 21 bp, Artificial Sequence
  siRNA
cgccatcatcgatgactacaa

SEQ ID NO: 961 - DNA, 21 bp, Artificial Sequence
  siRNA
cagatgtttcatcatttgaaa

SEQ ID NO: 962 - DNA, 21 bp, Artificial Sequence
  siRNA
aagcagtttgaacgactggaa

SEQ ID NO: 963 - DNA, 21 bp, Artificial Sequence
  siRNA
cccgttgattatgcaaggcaa

SEQ ID NO: 964 - DNA, 21 bp, Artificial Sequence
  siRNA
taggatactgggcgttaccaa

SEQ ID NO: 965 - DNA, 21 bp, Artificial Sequence
  siRNA
cggcagtcagatcctgcataa

SEQ ID NO: 966 - DNA, 21 bp, Artificial Sequence
  siRNA
cagcactgcactgttagctga

SEQ ID NO: 967 - DNA, 21 bp, Artificial Sequence
  siRNA
cgggactttctattaatatca

SEQ ID NO: 968 - DNA, 21 bp, Artificial Sequence
  siRNA
tcgggagaaagtggagtccga

SEQ ID NO: 969 - DNA, 21 bp, Artificial Sequence
  siRNA
tagcgaagaccaagggataaa

SEQ ID NO: 970 - DNA, 21 bp, Artificial Sequence
  siRNA
taggattaaattggtttccta

SEQ ID NO: 971 - DNA, 21 bp, Artificial Sequence
  siRNA
tcccagtgtcctttgaatcga

SEQ ID NO: 972 - DNA, 21 bp, Artificial Sequence
  siRNA
cgccgtcgagtcgccatggaa

SEQ ID NO: 973 - DNA, 21 bp, Artificial Sequence
  siRNA
cacttggctacccatcaacaa

SEQ ID NO: 974 - DNA, 21 bp, Artificial Sequence
  siRNA
gccgtgatttctagcagttga

SEQ ID NO: 975 - DNA, 21 bp, Artificial Sequence
  siRNA
cagggactgaacaaatggaaa

SEQ ID NO: 976 - DNA, 21 bp, Artificial Sequence
  siRNA
cagcttaactgacagacgtta

SEQ ID NO: 977 - DNA, 21 bp, Artificial Sequence
  siRNA
aagagccaatttaacaaacta

SEQ ID NO: 978 - DNA, 21 bp, Artificial Sequence
  siRNA
ctggactaaggatcaccatta

SEQ ID NO: 979 - DNA, 21 bp, Artificial Sequence
  siRNA
tacgtgggtgtactccatcaa

SEQ ID NO: 980 - DNA, 21 bp, Artificial Sequence
  siRNA
cagagatgatccggtcatata

SEQ ID NO: 981 - DNA, 21 bp, Artificial Sequence
  siRNA
cacagttaatggatctgtaaa

SEQ ID NO: 982 - DNA, 21 bp, Artificial Sequence
  siRNA
ccggagctggaagtacaagta

SEQ ID NO: 983 - DNA, 21 bp, Artificial Sequence
  siRNA
aaagtgcgtcatcctaaggaa

SEQ ID NO: 984 - DNA, 21 bp, Artificial Sequence
  siRNA
atcgatgttggttatgagaga

SEQ ID NO: 985 - DNA, 21 bp, Artificial Sequence
  siRNA
ctgaagggagaagctgagcaa

SEQ ID NO: 986 - DNA, 21 bp, Artificial Sequence
  siRNA
cacatccaatgtcttcgcaat

SEQ ID NO: 987 - DNA, 21 bp, Artificial Sequence
  siRNA
caggaaataatttatccacca

SEQ ID NO: 988 - DNA, 21 bp, Artificial Sequence
  siRNA
accgtcatgacgattattaca

SEQ ID NO: 989 - DNA, 21 bp, Artificial Sequence
  siRNA
aacagttagcacggcagctta

SEQ ID NO: 990 - DNA, 21 bp, Artificial Sequence
  siRNA
tcccactaaagcatagttgta

SEQ ID NO: 991 - DNA, 21 bp, Artificial Sequence
  siRNA
cacctcggcggcctttattta

SEQ ID NO: 992 - DNA, 21 bp, Artificial Sequence
  siRNA
gcggacggtcgtgatggataa

SEQ ID NO: 993 - DNA, 21 bp, Artificial Sequence
  siRNA
accagaggtgttagaagataa

SEQ ID NO: 994 - DNA, 21 bp, Artificial Sequence
  siRNA
ccagcctgttatggctaacta

SEQ ID NO: 995 - DNA, 21 bp, Artificial Sequence
  siRNA
aagcatgaaaggactcatgaa

SEQ ID NO: 996 - DNA, 21 bp, Artificial Sequence
  siRNA
accagggatgagaccaactat

SEQ ID NO: 997 - DNA, 21 bp, Artificial Sequence
  siRNA
tggcttcttcccacggagtta

SEQ ID NO: 998 - DNA, 21 bp, Artificial Sequence
  siRNA
ttcgttgactacgcccagaaa

SEQ ID NO: 999 - DNA, 21 bp, Artificial Sequence
  siRNA
aacctgaagtttggagatgaa

SEQ ID NO: 1000 - DNA, 21 bp, Artificial Sequence
  siRNA
tggcgtttccacggtaccaaa

SEQ ID NO: 1001 - DNA, 21 bp, Artificial Sequence
  siRNA
ctcaactcgtttataaataaa

SEQ ID NO: 1002 - DNA, 21 bp, Artificial Sequence
  siRNA
cctgatgaatctactgctaaa

SEQ ID NO: 1003 - DNA, 21 bp, Artificial Sequence
  siRNA
ctagtgttgcttatcaaataa

SEQ ID NO: 1004 - DNA, 21 bp, Artificial Sequence
  siRNA
tggatcgatttgtataccgaa

SEQ ID NO: 1005 - DNA, 21 bp, Artificial Sequence
  siRNA
caggtccattcttctgcacga

SEQ ID NO: 1006 - DNA, 21 bp, Artificial Sequence
  siRNA
aagagggagttcattcaggaa

SEQ ID NO: 1007 - DNA, 21 bp, Artificial Sequence
  siRNA
acccaaggaaatgattataaa

SEQ ID NO: 1008 - DNA, 21 bp, Artificial Sequence
  siRNA
cagttctatttaactgaatta

SEQ ID NO: 1009 - DNA, 21 bp, Artificial Sequence
  siRNA
cccgatcacgccgcaagcgaa

SEQ ID NO: 1010 - DNA, 21 bp, Artificial Sequence
  siRNA
tagcaagtcttgtacaacgaa

SEQ ID NO: 1011 - DNA, 21 bp, Artificial Sequence
  siRNA
cagcctcaagtcagtgcagga

SEQ ID NO: 1012 - DNA, 21 bp, Artificial Sequence
  siRNA
ccgcatcttggtgaaacgtga

SEQ ID NO: 1013 - DNA, 21 bp, Artificial Sequence
  siRNA
cacttcgaagcgaaatattaa

SEQ ID NO: 1014 - DNA, 21 bp, Artificial Sequence
  siRNA
tacgtgcaggtgtctcgtgaa

SEQ ID NO: 1015 - DNA, 21 bp, Artificial Sequence
  siRNA
tacgcgaacgcccacccttaa

SEQ ID NO: 1016 - DNA, 21 bp, Artificial Sequence
  siRNA
cacgactttctcgaacaccta

SEQ ID NO: 1017 - DNA, 21 bp, Artificial Sequence
  siRNA
ccaggacgccttcctcttcaa

SEQ ID NO: 1018 - DNA, 21 bp, Artificial Sequence
  siRNA
tacccgcaccaccacctttaa

SEQ ID NO: 1019 - DNA, 21 bp, Artificial Sequence
  siRNA
ctgcacgttcataaccaacaa

SEQ ID NO: 1020 - DNA, 21 bp, Artificial Sequence
  siRNA
ctcacctatgatgaggtccaa

SEQ ID NO: 1021 - DNA, 21 bp, Artificial Sequence
  siRNA
cagtgctagtccagactacaa

SEQ ID NO: 1022 - DNA, 21 bp, Artificial Sequence
  siRNA
ctggatggaggactatgacta

SEQ ID NO: 1023 - DNA, 21 bp, Artificial Sequence
  siRNA
cagcgttgagagtcaagacaa

SEQ ID NO: 1024 - DNA, 21 bp, Artificial Sequence
  siRNA
ccggcgctatgagaagttcga

SEQ ID NO: 1025 - DNA, 21 bp, Artificial Sequence
  siRNA
acgcaagtcgtggatgagtaa

SEQ ID NO: 1026 - DNA, 21 bp, Artificial Sequence
  siRNA
acgagcgtcagccttactcaa

SEQ ID NO: 1027 - DNA, 21 bp, Artificial Sequence
  siRNA
ttgggcatatgtatctttata

SEQ ID NO: 1028 - DNA, 21 bp, Artificial Sequence
  siRNA
ctgcatgaaggcagtgaccga

SEQ ID NO: 1029 - DNA, 21 bp, Artificial Sequence
  siRNA
ccgactcggtgtactgcagta

SEQ ID NO: 1030 - DNA, 21 bp, Artificial Sequence
  siRNA
cacgtgtagttctttattata

SEQ ID NO: 1031 - DNA, 21 bp, Artificial Sequence
  siRNA
ggcgttaaagttcatatccca

SEQ ID NO: 1032 - DNA, 21 bp, Artificial Sequence
  siRNA
agcaagaaagcccgtctggaa

SEQ ID NO: 1033 - DNA, 21 bp, Artificial Sequence
  siRNA
caccaattctgaagacaacta

SEQ ID NO: 1034 - DNA, 21 bp, Artificial Sequence
  siRNA
caggtgccacaggatggagaa

SEQ ID NO: 1035 - DNA, 21 bp, Artificial Sequence
  siRNA
cagcaggaagtccaattcaaa

SEQ ID NO: 1036 - DNA, 21 bp, Artificial Sequence
  siRNA
cccgccctagtgcgttactta

SEQ ID NO: 1037 - DNA, 21 bp, Artificial Sequence
  siRNA
tgcaacatccttcgagttgaa

SEQ ID NO: 1038 - DNA, 21 bp, Artificial Sequence
  siRNA
atgcaatcgggtggtatcata

SEQ ID NO: 1039 - DNA, 21 bp, Artificial Sequence
  siRNA
ctgactaagagtcccaagaaa

SEQ ID NO: 1040 - DNA, 21 bp, Artificial Sequence
  siRNA
tcgagattacatggatgttaa

SEQ ID NO: 1041 - DNA, 21 bp, Artificial Sequence
  siRNA
ctggtaattagttgatgcaaa

SEQ ID NO: 1042 - DNA, 21 bp, Artificial Sequence
  siRNA
ctgcaggagttcctcagtgaa

SEQ ID NO: 1043 - DNA, 21 bp, Artificial Sequence
  siRNA
cagtaacagtttgcacatgaa

SEQ ID NO: 1044 - DNA, 21 bp, Artificial Sequence
  siRNA
ccggctgaaattaagtgagga

SEQ ID NO: 1045 - DNA, 21 bp, Artificial Sequence
  siRNA
cagggtagtatgagtaaggaa

SEQ ID NO: 1046 - DNA, 21 bp, Artificial Sequence
  siRNA
cgccaaggataaagacgacta

SEQ ID NO: 1047 - DNA, 21 bp, Artificial Sequence
  siRNA
ctggaccaaagtggaaccaaa

SEQ ID NO: 1048 - DNA, 21 bp, Artificial Sequence
  siRNA
tagatgtagtgttgtaataaa

SEQ ID NO: 1049 - DNA, 21 bp, Artificial Sequence
  siRNA
tagtcctttctgtgaagctaa

SEQ ID NO: 1050 - DNA, 21 bp, Artificial Sequence
  siRNA
gagaataaatatcaaatctaa

SEQ ID NO: 1051 - DNA, 21 bp, Artificial Sequence
  siRNA
tcgcctcaccatcaagaagta

SEQ ID NO: 1052 - DNA, 21 bp, Artificial Sequence
  siRNA
ccggacctacaaatcggtcta

SEQ ID NO: 1053 - DNA, 21 bp, Artificial Sequence
  siRNA
cagcaggcacgttaactcgaa

SEQ ID NO: 1054 - DNA, 21 bp, Artificial Sequence
  siRNA
accaagttcggctctaccaaa

SEQ ID NO: 1055 - DNA, 21 bp, Artificial Sequence
  siRNA
ttggatagccgtcagagatta

SEQ ID NO: 1056 - DNA, 21 bp, Artificial Sequence
  siRNA
ttggcacacgctagaagttta

SEQ ID NO: 1057 - DNA, 21 bp, Artificial Sequence
  siRNA
ccgccgtggcgacatcattga

SEQ ID NO: 1058 - DNA, 21 bp, Artificial Sequence
  siRNA
acgatccgttggtgcatgaaa

SEQ ID NO: 1059 - DNA, 21 bp, Artificial Sequence
  siRNA
taggaactttcttgtcacaaa

SEQ ID NO: 1060 - DNA, 21 bp, Artificial Sequence
  siRNA
ccggcctgtgaaacctccaaa

SEQ ID NO: 1061 - DNA, 21 bp, Artificial Sequence
  siRNA
ccgggtgtatcccatgtgcaa

SEQ ID NO: 1062 - DNA, 21 bp, Artificial Sequence
  siRNA
taggcagttctgtgagtcaaa

SEQ ID NO: 1063 - DNA, 21 bp, Artificial Sequence
  siRNA
gagggcgctgatcgagagtta

SEQ ID NO: 1064 - DNA, 21 bp, Artificial Sequence
  siRNA
cagtaccacaccgaaagtaaa

SEQ ID NO: 1065 - DNA, 21 bp, Artificial Sequence
  siRNA
aaccaacatagcatcattaat

SEQ ID NO: 1066 - DNA, 21 bp, Artificial Sequence
  siRNA
tgggcaagggattataattaa

SEQ ID NO: 1067 - DNA, 21 bp, Artificial Sequence
  siRNA
cccagagatggtcatatttaa

SEQ ID NO: 1068 - DNA, 21 bp, Artificial Sequence
  siRNA
aaggattcattgagcagcata

SEQ ID NO: 1069 - DNA, 21 bp, Artificial Sequence
  siRNA
cagaactgcctttgcactaaa

SEQ ID NO: 1070 - DNA, 21 bp, Artificial Sequence
  siRNA
aagcgtcagtgtaaagttctt

SEQ ID NO: 1071 - DNA, 21 bp, Artificial Sequence
  siRNA
cagaaagaactcacgactaca

SEQ ID NO: 1072 - DNA, 21 bp, Artificial Sequence
  siRNA
atgattcgtcgcagaagccta

SEQ ID NO: 1073 - DNA, 21 bp, Artificial Sequence
  siRNA
caccgtcaacgtggcatggaa

SEQ ID NO: 1074 - DNA, 21 bp, Artificial Sequence
  siRNA
cggaacgttccctggaaatta

SEQ ID NO: 1075 - DNA, 21 bp, Artificial Sequence
  siRNA
cccgagcgcgcaggagctcaa

SEQ ID NO: 1076 - DNA, 21 bp, Artificial Sequence
  siRNA
atggactgcgaagaactccta

SEQ ID NO: 1077 - DNA, 21 bp, Artificial Sequence
  siRNA
aaccgttacagccacaaccaa

SEQ ID NO: 1078 - DNA, 21 bp, Artificial Sequence
  siRNA
ctgcgctactttgacgagaaa

SEQ ID NO: 1079 - DNA, 21 bp, Artificial Sequence
  siRNA
taaccttatatttacaataaa

SEQ ID NO: 1080 - DNA, 21 bp, Artificial Sequence
  siRNA
atggccaacactcctcaagaa

SEQ ID NO: 1081 - DNA, 21 bp, Artificial Sequence
  siRNA
cacgtcggctgctgatgtcta

SEQ ID NO: 1082 - DNA, 21 bp, Artificial Sequence
  siRNA
cagactggatatggcgcaaga

SEQ ID NO: 1083 - DNA, 21 bp, Artificial Sequence
  siRNA
tagcttatatatgacggtata

SEQ ID NO: 1084 - DNA, 21 bp, Artificial Sequence
  siRNA
cagctgctccttgatacccga

SEQ ID NO: 1085 - DNA, 21 bp, Artificial Sequence
  siRNA
ccgggtcatctgcagtgacaa

SEQ ID NO: 1086 - DNA, 21 bp, Artificial Sequence
  siRNA
caagatgttatcaattggtta

SEQ ID NO: 1087 - DNA, 21 bp, Artificial Sequence
  siRNA
cagaattacactgaagaagta

SEQ ID NO: 1088 - DNA, 21 bp, Artificial Sequence
  siRNA
ctcgaggttgaaaccacagat

SEQ ID NO: 1089 - DNA, 21 bp, Artificial Sequence
  siRNA
aaggagctatctgtttatgta

SEQ ID NO: 1090 - DNA, 21 bp, Artificial Sequence
  siRNA
ctgacaaagtacgaggtttaa

SEQ ID NO: 1091 - DNA, 21 bp, Artificial Sequence
  siRNA
ttcggatcggtttgtaattaa

SEQ ID NO: 1092 - DNA, 21 bp, Artificial Sequence
  siRNA
aaggatgagtgcgtcatagca

SEQ ID NO: 1093 - DNA, 21 bp, Artificial Sequence
  siRNA
caccttggattctttgttaaa

SEQ ID NO: 1094 - DNA, 21 bp, Artificial Sequence
  siRNA
acactcgcagttaatatcata

SEQ ID NO: 1095 - DNA, 21 bp, Artificial Sequence
  siRNA
ctgctagcgtatattcataaa

SEQ ID NO: 1096 - DNA, 21 bp, Artificial Sequence
  siRNA
taggtatagctgacgagacta

SEQ ID NO: 1097 - DNA, 21 bp, Artificial Sequence
  siRNA
tacctcggatccattcacgaa

SEQ ID NO: 1098 - DNA, 21 bp, Artificial Sequence
  siRNA
ccaaggaatctgtgcgggtaa

SEQ ID NO: 1099 - DNA, 21 bp, Artificial Sequence
  siRNA
caagatatccatggtgaataa

SEQ ID NO: 1100 - DNA, 21 bp, Artificial Sequence
  siRNA
cacgaacagcgtcatctgcaa

SEQ ID NO: 1101 - DNA, 21 bp, Artificial Sequence
  siRNA
cacgagcatcactctgaccat

SEQ ID NO: 1102 - DNA, 21 bp, Artificial Sequence
  siRNA
gccgctgaggatgtcccgaaa

SEQ ID NO: 1103 - DNA, 21 bp, Artificial Sequence
  siRNA
ccaggtttcctcagtcataga

SEQ ID NO: 1104 - DNA, 21 bp, Artificial Sequence
  siRNA
cagagtatgaaccaagggtta

SEQ ID NO: 1105 - DNA, 21 bp, Artificial Sequence
  siRNA
aaggaaacaactcgtactgaa

SEQ ID NO: 1106 - DNA, 21 bp, Artificial Sequence
  siRNA
aagtgtatatgaagttattta

SEQ ID NO: 1107 - DNA, 21 bp, Artificial Sequence
  siRNA
ccgcggacttaaagcaataaa

SEQ ID NO: 1108 - DNA, 21 bp, Artificial Sequence
  siRNA
ctacagctcgttgacaagaaa

SEQ ID NO: 1109 - DNA, 21 bp, Artificial Sequence
  siRNA
aacatcctagaccctgacgaa

SEQ ID NO: 1110 - DNA, 21 bp, Artificial Sequence
  siRNA
aggcttcatcatcgacccaaa

SEQ ID NO: 1111 - DNA, 21 bp, Artificial Sequence
  siRNA
cgggagaagaagcgtctagaa

SEQ ID NO: 1112 - DNA, 21 bp, Artificial Sequence
  siRNA
ccctcggagcacgaatatata

SEQ ID NO: 1113 - DNA, 21 bp, Artificial Sequence
  siRNA
cgcggtcggatagttacacta

SEQ ID NO: 1114 - DNA, 21 bp, Artificial Sequence
  siRNA
gagggtggatttatcttctca

SEQ ID NO: 1115 - DNA, 21 bp, Artificial Sequence
  siRNA
aagggaaacatcagtaagaaa

SEQ ID NO: 1116 - DNA, 21 bp, Artificial Sequence
  siRNA
ctggaccaggattcaactaca

SEQ ID NO: 1117 - DNA, 21 bp, Artificial Sequence
  siRNA
tagccgagttgtgcagcgtaa

SEQ ID NO: 1118 - DNA, 21 bp, Artificial Sequence
  siRNA
aaccctgatgtctgccaacta

SEQ ID NO: 1119 - DNA, 21 bp, Artificial Sequence
  siRNA
cggcgttgatatcggtggtaa

SEQ ID NO: 1120 - DNA, 21 bp, Artificial Sequence
  siRNA
tgccgccgacaaatacaaata

SEQ ID NO: 1121 - DNA, 21 bp, Artificial Sequence
  siRNA
aagaaataaattcaccaacaa

SEQ ID NO: 1122 - DNA, 21 bp, Artificial Sequence
  siRNA
ctggtagatagaagagctaaa

SEQ ID NO: 1123 - DNA, 21 bp, Artificial Sequence
  siRNA
cgggtcgatcttccacattca

SEQ ID NO: 1124 - DNA, 21 bp, Artificial Sequence
  siRNA
ctccaaacagatcatagcgaa

SEQ ID NO: 1125 - DNA, 21 bp, Artificial Sequence
  siRNA
tcaaccgtacatcaaattata

SEQ ID NO: 1126 - DNA, 21 bp, Artificial Sequence
  siRNA
tacgtgggctatactgtatta

SEQ ID NO: 1127 - DNA, 21 bp, Artificial Sequence
  siRNA
aaagagatacctcatatcgta

SEQ ID NO: 1128 - DNA, 21 bp, Artificial Sequence
  siRNA
aagaaatcaagaaacaattaa

SEQ ID NO: 1129 - DNA, 21 bp, Artificial Sequence
  siRNA
tcccaattagtctgtatctat

SEQ ID NO: 1130 - DNA, 21 bp, Artificial Sequence
  siRNA
ctgtaaatgtctattgccgta

SEQ ID NO: 1131 - DNA, 21 bp, Artificial Sequence
  siRNA
ttaaagcattcctttctttaa

SEQ ID NO: 1132 - DNA, 21 bp, Artificial Sequence
  siRNA
ccggaagacgccggagtcatt

SEQ ID NO: 1133 - DNA, 21 bp, Artificial Sequence
  siRNA
aaagtcatatagaaatattaa

SEQ ID NO: 1134 - DNA, 21 bp, Artificial Sequence
  siRNA
aaccaattagttactatcgta

SEQ ID NO: 1135 - DNA, 21 bp, Artificial Sequence
  siRNA
ctgcttgtatgaaactgtgaa

SEQ ID NO: 1136 - DNA, 21 bp, Artificial Sequence
  siRNA
aaggcctatcagccaattaaa

SEQ ID NO: 1137 - DNA, 21 bp, Artificial Sequence
  siRNA
tagactagaggcaagcattta

SEQ ID NO: 1138 - DNA, 21 bp, Artificial Sequence
  siRNA
tggcggtgttccagaagcaga

SEQ ID NO: 1139 - DNA, 21 bp, Artificial Sequence
  siRNA
tcgcatcatcgcagagctaga

SEQ ID NO: 1140 - DNA, 21 bp, Artificial Sequence
  siRNA
aaacatgattcctttaataaa

SEQ ID NO: 1141 - DNA, 21 bp, Artificial Sequence
  siRNA
aagggacagtatacgcacaaa

SEQ ID NO: 1142 - DNA, 21 bp, Artificial Sequence
  siRNA
ctggtacataaagatgagtaa

SEQ ID NO: 1143 - DNA, 21 bp, Artificial Sequence
  siRNA
ctctgagatctcaatgcgaaa

SEQ ID NO: 1144 - DNA, 21 bp, Artificial Sequence
  siRNA
cacgtgcaagccagatgtgaa

SEQ ID NO: 1145 - DNA, 21 bp, Artificial Sequence
  siRNA
ttcgggctgtcatgaacagaa

SEQ ID NO: 1146 - DNA, 21 bp, Artificial Sequence
  siRNA
cccgaagagccaaatataata

SEQ ID NO: 1147 - DNA, 21 bp, Artificial Sequence
  siRNA
cacgaaagagatgatgataat

SEQ ID NO: 1148 - DNA, 21 bp, Artificial Sequence
  siRNA
ctccgctatccgagaggtgta

SEQ ID NO: 1149 - DNA, 21 bp, Artificial Sequence
  siRNA
tagcaccttagggcagcataa

SEQ ID NO: 1150 - DNA, 21 bp, Artificial Sequence
  siRNA
ccaggaaatactggaagtcaa

SEQ ID NO: 1151 - DNA, 21 bp, Artificial Sequence
  siRNA
acggaaccatttcgcctttaa

SEQ ID NO: 1152 - DNA, 21 bp, Artificial Sequence
  siRNA
ccgcgtgtgcctcaagcacta

SEQ ID NO: 1153 - DNA, 21 bp, Artificial Sequence
  siRNA
caccttcaagtcaattcataa

SEQ ID NO: 1154 - DNA, 21 bp, Artificial Sequence
  siRNA
cggatttgcttgttccagtaa

SEQ ID NO: 1155 - DNA, 21 bp, Artificial Sequence
  siRNA
cctgtaagatctatcgttcaa

SEQ ID NO: 1156 - DNA, 21 bp, Artificial Sequence
  siRNA
cagcttaagaaaggtacagaa

SEQ ID NO: 1157 - DNA, 21 bp, Artificial Sequence
  siRNA
caccgcctaaatttcacgaca

SEQ ID NO: 1158 - DNA, 21 bp, Artificial Sequence
  siRNA
caggctaagggtggccagaga

SEQ ID NO: 1159 - DNA, 21 bp, Artificial Sequence
  siRNA
ttgcataatacttgtcttaaa

SEQ ID NO: 1160 - DNA, 21 bp, Artificial Sequence
  siRNA
ctgaatgtactgatttagaaa

SEQ ID NO: 1161 - DNA, 21 bp, Artificial Sequence
  siRNA
ctagttaaggtttaaatgcta

SEQ ID NO: 1162 - DNA, 21 bp, Artificial Sequence
  siRNA
cagccttgacttatccgaatt

SEQ ID NO: 1163 - DNA, 21 bp, Artificial Sequence
  siRNA
atcgtcgaggtgaagagcaaa

SEQ ID NO: 1164 - DNA, 21 bp, Artificial Sequence
  siRNA
cagaagtttcatcttaggata

SEQ ID NO: 1165 - DNA, 21 bp, Artificial Sequence
  siRNA
cagcaagagtcctaacatcta

SEQ ID NO: 1166 - DNA, 21 bp, Artificial Sequence
  siRNA
caggactaaatggaagcgaca

SEQ ID NO: 1167 - DNA, 21 bp, Artificial Sequence
  siRNA
caggccgaaacttcccaacaa

SEQ ID NO: 1168 - DNA, 21 bp, Artificial Sequence
  siRNA
ccagcggttgtcctcccttaa

SEQ ID NO: 1169 - DNA, 21 bp, Artificial Sequence
  siRNA
cacattcagcttataatggaa

SEQ ID NO: 1170 - DNA, 21 bp, Artificial Sequence
  siRNA
aagcaggaaaccagcaacaaa

SEQ ID NO: 1171 - DNA, 21 bp, Artificial Sequence
  siRNA
ctctgtcattcgtgaacttaa

SEQ ID NO: 1172 - DNA, 21 bp, Artificial Sequence
  siRNA
cacgagagccttctaatgcca

SEQ ID NO: 1173 - DNA, 21 bp, Artificial Sequence
  siRNA
agggattggaaccctgatcta

SEQ ID NO: 1174 - DNA, 21 bp, Artificial Sequence
  siRNA
ccgcaaggtctcggtgcagta

SEQ ID NO: 1175 - DNA, 21 bp, Artificial Sequence
  siRNA
atggatctatacaacaaataa

SEQ ID NO: 1176 - DNA, 21 bp, Artificial Sequence
  siRNA
ctgatactatctcaaagacga

SEQ ID NO: 1177 - DNA, 21 bp, Artificial Sequence
  siRNA
cagcaagaagatgaaccttca

SEQ ID NO: 1178 - DNA, 21 bp, Artificial Sequence
  siRNA
aagatgcaagaaggagtgaaa

SEQ ID NO: 1179 - DNA, 21 bp, Artificial Sequence
  siRNA
ttggtgcagttaagaattaaa

SEQ ID NO: 1180 - DNA, 21 bp, Artificial Sequence
  siRNA
atgggtagcctcaccatgaaa

SEQ ID NO: 1181 - DNA, 21 bp, Artificial Sequence
  siRNA
cgggatgtggatgatagcaaa

SEQ ID NO: 1182 - DNA, 21 bp, Artificial Sequence
  siRNA
caggtggatgtagagatcaaa

SEQ ID NO: 1183 - DNA, 21 bp, Artificial Sequence
  siRNA
tgcagtgacgctgtcattaaa

SEQ ID NO: 1184 - DNA, 21 bp, Artificial Sequence
  siRNA
cagcaaacacctttaaggcaa

SEQ ID NO: 1185 - DNA, 21 bp, Artificial Sequence
  siRNA
tggcatattgaccctatataa

SEQ ID NO: 1186 - DNA, 21 bp, Artificial Sequence
  siRNA
tacgagaatcagtctaacaga

SEQ ID NO: 1187 - DNA, 21 bp, Artificial Sequence
  siRNA
aagcactagaatatccagcta

SEQ ID NO: 1188 - DNA, 21 bp, Artificial Sequence
  siRNA
cacaagaatggaacaaatcta

SEQ ID NO: 1189 - DNA, 21 bp, Artificial Sequence
  siRNA
tccattgacaacaaggcttta

SEQ ID NO: 1190 - DNA, 21 bp, Artificial Sequence
  siRNA
attgccgtaaattgttaataa

SEQ ID NO: 1191 - DNA, 21 bp, Artificial Sequence
  siRNA
atgccaggtctagcaaacata

SEQ ID NO: 1192 - DNA, 21 bp, Artificial Sequence
  siRNA
cccgacgagttctctgacttt

SEQ ID NO: 1193 - DNA, 21 bp, Artificial Sequence
  siRNA
aagcacatagttattgctgaa

SEQ ID NO: 1194 - DNA, 21 bp, Artificial Sequence
  siRNA
tagactgtattaataaacata

SEQ ID NO: 1195 - DNA, 21 bp, Artificial Sequence
  siRNA
aacagagagccttaatcttta

SEQ ID NO: 1196 - DNA, 21 bp, Artificial Sequence
  siRNA
caggtttaatttgatggtcta

SEQ ID NO: 1197 - DNA, 21 bp, Artificial Sequence
  siRNA
cggattcctatttatgcccta

SEQ ID NO: 1198 - DNA, 21 bp, Artificial Sequence
  siRNA
caggaagtgggtatcaaattg

SEQ ID NO: 1199 - DNA, 21 bp, Artificial Sequence
  siRNA
caggccactaaaccatctaaa

SEQ ID NO: 1200 - DNA, 21 bp, Artificial Sequence
  siRNA
cagcaactcatttatatatat

SEQ ID NO: 1201 - DNA, 21 bp, Artificial Sequence
  siRNA
cccaaagatcatgatattcca

SEQ ID NO: 1202 - DNA, 21 bp, Artificial Sequence
  siRNA
gagcaagaacctggtgcgcaa

SEQ ID NO: 1203 - DNA, 21 bp, Artificial Sequence
  siRNA
taggacatgctccaaagaaga

SEQ ID NO: 1204 - DNA, 21 bp, Artificial Sequence
  siRNA
ccgcaccaagctgcagaacaa

SEQ ID NO: 1205 - DNA, 21 bp, Artificial Sequence
  siRNA
gagatggtctatcctaaccaa

SEQ ID NO: 1206 - DNA, 21 bp, Artificial Sequence
  siRNA
ttcaacaaagctacagagtta

SEQ ID NO: 1207 - DNA, 21 bp, Artificial Sequence
  siRNA
cacgatgggaggacaagttca

SEQ ID NO: 1208 - DNA, 21 bp, Artificial Sequence
  siRNA
ctggatgacagttgggtgata

SEQ ID NO: 1209 - DNA, 21 bp, Artificial Sequence
  siRNA
cagggccaatcaatttcacca

SEQ ID NO: 1210 - DNA, 21 bp, Artificial Sequence
  siRNA
tccacacatcataaagcttta

SEQ ID NO: 1211 - DNA, 21 bp, Artificial Sequence
  siRNA
caggctgaatggcgaaggcat

SEQ ID NO: 1212 - DNA, 21 bp, Artificial Sequence
  siRNA
ttcaacaaagctacagagtta

SEQ ID NO: 1213 - DNA, 21 bp, Artificial Sequence
  siRNA
ccaagtctgagtggttggata

SEQ ID NO: 1214 - DNA, 21 bp, Artificial Sequence
  siRNA
caggatactgtttccttacta

SEQ ID NO: 1215 - DNA, 21 bp, Artificial Sequence
  siRNA
aacgctcttcaagctcacaaa

SEQ ID NO: 1216 - DNA, 21 bp, Artificial Sequence
  siRNA
ctggatgcgataagacatcga

SEQ ID NO: 1217 - DNA, 21 bp, Artificial Sequence
  siRNA
caaggagaaactatacaggaa

SEQ ID NO: 1218 - DNA, 21 bp, Artificial Sequence
  siRNA
ctccctgttcggagattgata

SEQ ID NO: 1219 - DNA, 21 bp, Artificial Sequence
  siRNA
tggagtgttctgcacttacaa

SEQ ID NO: 1220 - DNA, 21 bp, Artificial Sequence
  siRNA
cccggccttcaacgagttcta

SEQ ID NO: 1221 - DNA, 21 bp, Artificial Sequence
  siRNA
aactaccgagattatttgata

SEQ ID NO: 1222 - DNA, 21 bp, Artificial Sequence
  siRNA
tagttaaattagaatggtgta

SEQ ID NO: 1223 - DNA, 21 bp, Artificial Sequence
  siRNA
aaagttgtagctaaatattta

SEQ ID NO: 1224 - DNA, 21 bp, Artificial Sequence
  siRNA
atgaagctccaggaactttaa

SEQ ID NO: 1225 - DNA, 21 bp, Artificial Sequence
  siRNA
ctagagtatgcagctggtaaa

SEQ ID NO: 1226 - DNA, 21 bp, Artificial Sequence
  siRNA
cccggcagggttgtttcttaa

SEQ ID NO: 1227 - DNA, 21 bp, Artificial Sequence
  siRNA
ttcgctctgaaatggaacaaa

SEQ ID NO: 1228 - DNA, 21 bp, Artificial Sequence
  siRNA
ctgcatggactgtatactcga

SEQ ID NO: 1229 - DNA, 21 bp, Artificial Sequence
  siRNA
ttggattactattaagtggtt

SEQ ID NO: 1230 - DNA, 21 bp, Artificial Sequence
  siRNA
ccgcctgttgttaccggtatt

SEQ ID NO: 1231 - DNA, 21 bp, Artificial Sequence
  siRNA
ccaagtctgagtggttggata

SEQ ID NO: 1232 - DNA, 21 bp, Artificial Sequence
  siRNA
cagagtatgaaccaagggtta

SEQ ID NO: 1233 - DNA, 21 bp, Artificial Sequence
  siRNA
ccaaatgtcgatggaacatca

SEQ ID NO: 1234 - DNA, 21 bp, Artificial Sequence
  siRNA
ccagaagattcatatctccaa

SEQ ID NO: 1235 - DNA, 21 bp, Artificial Sequence
  siRNA
aaggaaatccattacttcaaa

SEQ ID NO: 1236 - DNA, 21 bp, Artificial Sequence
  siRNA
caagatgaaagaaggcataaa

SEQ ID NO: 1237 - DNA, 21 bp, Artificial Sequence
  siRNA
catggaattattggttataaa

SEQ ID NO: 1238 - DNA, 21 bp, Artificial Sequence
  siRNA
cacagaagacgaagagactat

SEQ ID NO: 1239 - DNA, 21 bp, Artificial Sequence
  siRNA
ctgctaggcaaccaaatttaa

SEQ ID NO: 1240 - DNA, 21 bp, Artificial Sequence
  siRNA
cacctccatgacctccgtcaa

SEQ ID NO: 1241 - DNA, 21 bp, Artificial Sequence
  siRNA
atgaagcagcttcatatctaa

SEQ ID NO: 1242 - DNA, 21 bp, Artificial Sequence
  siRNA
tcccttcagtgtgccactgaa

SEQ ID NO: 1243 - DNA, 21 bp, Artificial Sequence
  siRNA
cagagaagcgcctattaacaa

SEQ ID NO: 1244 - DNA, 21 bp, Artificial Sequence
  siRNA
gaccgaatgtctaggatttaa

SEQ ID NO: 1245 - DNA, 21 bp, Artificial Sequence
  siRNA
acgaactggtataatgattta

SEQ ID NO: 1246 - DNA, 21 bp, Artificial Sequence
  siRNA
aggagtagaaggctcaaacaa

SEQ ID NO: 1247 - DNA, 21 bp, Artificial Sequence
  siRNA
actgaaagaactttagctaaa

SEQ ID NO: 1248 - DNA, 21 bp, Artificial Sequence
  siRNA
tacctcggacgtctacggaaa

SEQ ID NO: 1249 - DNA, 21 bp, Artificial Sequence
  siRNA
cagccctctcatctcctggaa

SEQ ID NO: 1250 - DNA, 21 bp, Artificial Sequence
  siRNA
gaccgaatgtctaggatttaa

SEQ ID NO: 1251 - DNA, 21 bp, Artificial Sequence
  siRNA
caggtcctggcatattgtcca

SEQ ID NO: 1252 - DNA, 21 bp, Artificial Sequence
  siRNA
caacgtgtcaagctacttaaa

SEQ ID NO: 1253 - DNA, 21 bp, Artificial Sequence
  siRNA
atggctatgatttaccagaaa

SEQ ID NO: 1254 - DNA, 21 bp, Artificial Sequence
  siRNA
aagcaccttctttaacaataa

SEQ ID NO: 1255 - DNA, 21 bp, Artificial Sequence
  siRNA
aagaggcaaatttgcagcaaa

SEQ ID NO: 1256 - DNA, 21 bp, Artificial Sequence
  siRNA
tccgaccaccagctcaatcaa

SEQ ID NO: 1257 - DNA, 21 bp, Artificial Sequence
  siRNA
ggcgatggtgctgttagtaaa

SEQ ID NO: 1258 - DNA, 21 bp, Artificial Sequence
  siRNA
cccgatgggtttcctaaattt

SEQ ID NO: 1259 - DNA, 21 bp, Artificial Sequence
  siRNA
ctgctgaaatgtaaacaagaa

SEQ ID NO: 1260 - DNA, 21 bp, Artificial Sequence
  siRNA
aagagtcactccctaaaccta

SEQ ID NO: 1261 - DNA, 21 bp, Artificial Sequence
  siRNA
tacactacaattgttaataaa

SEQ ID NO: 1262 - DNA, 21 bp, Artificial Sequence
  siRNA
ttggagaatatttctactaca

SEQ ID NO: 1263 - DNA, 21 bp, Artificial Sequence
  siRNA
cgcccagattcaggcgtgtaa

SEQ ID NO: 1264 - DNA, 21 bp, Artificial Sequence
  siRNA
aaaggtaaataagctccctaa

SEQ ID NO: 1265 - DNA, 21 bp, Artificial Sequence
  siRNA
caggagtgtatatgtttcata

SEQ ID NO: 1266 - DNA, 21 bp, Artificial Sequence
  siRNA
ttgacaagaaatataacccta

SEQ ID NO: 1267 - DNA, 21 bp, Artificial Sequence
  siRNA
ctgctatataagaaagaggta

SEQ ID NO: 1268 - DNA, 21 bp, Artificial Sequence
  siRNA
caccaataaattctactaact

SEQ ID NO: 1269 - DNA, 21 bp, Artificial Sequence
  siRNA
caggtcctggcatattgtcca

SEQ ID NO: 1270 - DNA, 21 bp, Artificial Sequence
  siRNA
cagaagtttcatcttaggata

SEQ ID NO: 1271 - DNA, 21 bp, Artificial Sequence
  siRNA
cagcaacaaatggtacaggaa

SEQ ID NO: 1272 - DNA, 21 bp, Artificial Sequence
  siRNA
ccgtatttgtgccaacaaata

SEQ ID NO: 1273 - DNA, 21 bp, Artificial Sequence
  siRNA
caggcttaccgtggacgccta

SEQ ID NO: 1274 - DNA, 21 bp, Artificial Sequence
  siRNA
ttcttcaacattgccatcaat

SEQ ID NO: 1275 - DNA, 21 bp, Artificial Sequence
  siRNA
atcctatgcaatatatctaaa

SEQ ID NO: 1276 - DNA, 21 bp, Artificial Sequence
  siRNA
ttgaactacagtacagaaaga

(19) United States
(12) Patent Application Publication
(10) Pub. No.: US 2010/0239656 A1
(43) Pub. Date: Sep. 23, 2010

(54) EGFR/NEDD9/TGF-BETA INTERACTOME AND METHODS OF USE THEREOF FOR THE IDENTIFICATION OF AGENTS HAVING EFFICACY IN THE TREATMENT OF HYPERPROLIFERATIVE DISORDERS

(76) Inventors: Igor Astsaturov, Philadelphia, PA (US); Erica A. Golemis, Oreland, PA (US); Ilya G. Serebriiskii, Rockledge, PA (US); Louis M. Weiner, Washington, DC (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/777,112

(22) Filed: May 10, 2010

Related U.S. Application Data
(63) Continuation-in-part of application No. PCT/US08/83067, filed on Nov. 10, 2008.
(60) Provisional application No. 60/986,964, filed on Nov. 9, 2007.

(51) Int. Cl. A61K 39/395; A61K 31/517; A61K 31/713; A61K 9/127; A61K 31/277; A61K 31/5377; A61K 31/4545; A61K 31/4045; A61P 35/00; G01N 33/574; C12Q 1/68

(52) U.S. Cl. 424/450; 424/145.1; 514/266.4; 514/44 A; 514/523; 514/234.5; 514/266.24; 514/318; 514/414; 435/7.23; 435/6

(57) ABSTRACT

Compositions and methods for the treatment and diagnosis of cancer are disclosed.

This application claims priority to PCT/US08/83067 filed Nov. 10, 2008 which in turn claims priority under 35 U.S.C. §119(e) to U.S. Provisional Application 60/986,964 filed Nov. 9, 2007, the entire contents of each being incorporated by reference herein as though set forth in full.

### FIELD OF THE INVENTION

This invention relates to the fields of system biology, pharmacology and drug discovery. More specifically, the invention provides an EGFR/NEDD9/TGF-β interactome that facilitates the identification of agents for the treatment of proliferative disorders, particularly metastatic cancer. Anti-cancer agents having efficacy when used alone and in combination identified using the methods described herein are also provided.

### BACKGROUND OF THE INVENTION

Several publications and patent documents are cited throughout the specification in order to describe the state of the art to which this invention pertains. Each of these citations is incorporated herein by reference as though set forth in full.

Cancer is a leading cause of death in the United States. Treatments for metastatic cancer are generally limited, and include radiation, chemotherapy with non-specific cytotoxic agents, and therapy with drugs targeted at specific proteins that have been identified as marking cancer cells, and actively contributing to the aggressiveness of cancer growth. Taking metastatic colorectal cancer as an example, among the relatively limited drugs available for treatment of this disease, the DNA damaging agent irenotecan (a pro-drug for camptothecin), and antibodies (cetuximab, panitumumab) and small molecules (erlotinib, gefitinib) targeting the receptor tyrosine kinase (RTK) EGFR, an upstream regulator of the Ras pathway, have shown some efficacy (1-3). It is likely that improvement of therapies directed against EGFR and its family members (e.g., ERBB2/HER2/NEU, ERBB3/HER3) will be beneficial for treatment of numerous metastatic cancers, including those of breast, lung, and pancreas, as these proteins are often abnormally abundant or active in these tumors (e.g. 4,5), and EGFR-family targeting agents such as erlotinib and cetuximab have recently been approved for use in combination therapies in these cancers (1).

While combination of DNA damaging agents such as irenotecan and EGFR-targeting antibodies in the clinic, in some cases, produces substantial therapeutic benefit, in other cases, patients fail to respond. It is extremely likely that the failed response arises from secondary mutations in cancer cells that confer resistance to DNA damage, or relieve dependence of cells on EGFR: for example, mutations in K-Ras are becoming appreciated as a resistance factor for EGFR-directed therapies (6). In other cases, the sources of resistance or sensitivity are obscure (7).

### SUMMARY OF THE INVENTION

In accordance with the present invention, a method for identifying compounds, particularly siRNA molecules which modulate sensitivity to EGFR/MEK-1 targeting agents is provided.

An exemplary method entails providing an EGFR/NEDD9/TGF-β interactome, comprising genes which are involved in cellular proliferation and EGFR/MEK-1 signalling; synthesizing at least one compound (e.g., an siRNA molecule) which targets EGFR/NEDD9/TGF-β interactome genes; contacting a cancer cell with at least one EGFR-MEK-1 targeting agent in the presence and absence of at least one compound from above; and determining cell viability in the presence of said agent alone and in the presence and absence of said at least one compound, compounds which increase or decrease sensitivity modulating cell sensitivity to said agent. Compounds for use in the invention include, without limitation, siRNA, phosphatase inhibitors, kinase inhibitors, inhibitory antibodies, and cholesterol synthesis inhibitors. The method may further include examining the cells for the presence of at least one parameter selected from the group consisting of morphological alterations, altered migratory properties, altered levels of apoptosis, altered angiogenic properties, and altered chromosomal or DNA integrity.

In a preferred embodiment, the EGFR-MEK-1 targeting agent is selected from the group consisting of cetuximab, panitumumab, erlotinib, lapatinib, gefitinib, and U0126. Sequences for the siRNA molecules disclosed herein are provided in Table 3. Sensitizing siRNAs are provided in Table 2.

In yet another aspect of the invention, a pharmaceutical composition comprising an effective amount of at least one EGFR-MEK-1 targeting agent listed above and at least one sensitizing siRNA provided in Table 2, in a pharmaceutically acceptable carrier is provided. Methods of administering the same to patients in need thereof for the treatment of malignancy are also disclosed.

In one embodiment, the cancer cell to be screened is obtained from a cancer cell line. In a particularly preferred embodiment, the cancer cell is isolated from a patient.

Also provided herein is a plurality of biomarkers associated with chemoresistance. Exemplary markers are provided in Table 2.

In yet another aspect, a method for determining whether a patient will respond to EGFR/MEK-1 targeting therapy is provided. An exemplary method comprises assessing a cancer cell from said patient for expression levels of at least one of the biomarkers listed in Table 2.

### BRIEF DESCRIPTION OF THE DRAWINGS

The file of this application contains at least one drawing executed in color. Copies of this patent with color drawings will be provided by the Patent and Trademark Office upon request and payment of the necessary fee.

FIG. 1. Selection of siRNAs for targeted library.

FIG. 2. EGFR/HER2 and connected pro-survival signaling pathways. While EGFR/HER2 directly signal through Ras to ERK and PI3K, cross signaling by the TGFβ/integrin-regulated proteins, Src, FAK, and NEDD9 contribute to activation of pro-survival endpoints, Akt and NF-κB. Drugs inhibiting EGFR/HER2 and Ras/Mek1 cascade are indicated.

FIG. 3. Representative dataset indicating convergence of EGFR/HER2-centered and NEDD9/SRC/TGFβ-centered protein interaction networks. Filled circles represent proteins, lines direct physical interactions. Proteins directly binding EGFR, HER2, or their proximal effectors are indicated in green to left; those directly binding NEDD9 and proximal partners are indicated in red, to right. This is one component of the meta-analysis employed to select components of the 638 siRNA library (see FIG. 1).

FIG. 4A. Primary screens, exploration of efficacy for validated hits. FIG. 4B. Knockdown of 39% (247/638) of genes in the library reduced the viability of DMSO-treated A431 cells by at least 15%, with 7% (45/638) reducing viability more than 30%. 214 primary hits (34% of the library), including 95 strong hits (15%, SI<0.7) sensitized to one or both EGFR-targeting agents. In contrast, 84 primary hits (13%), including 30 strong hits (5%, SI<0.7) were obtained that sensitized to the cytotoxic agent CPT11.

FIG. 5. Distribution of hits in relation to effect of siRNA on cells treated with vehicle.

FIG. 6. Muliplexing of Cell Titer. Blue staining for viability (top) with ImageXpress for visualization of cells stained with Hoechst and calcein vital dyes.

FIG. 7. Examples on ImageXpress cellular profiles. Green line represents trace of profile seen with siRNA negative control, as histogram of values for average nuclear staining with Hoechst. Red lines show generally increased (left) or periodically increased (right) staining, which correlates with DNA condensation, e.g., increased frequency of mitotic or apoptotic cells.

FIG. 8. Sensitization profile of screening hit list. 208 hits, 156 sensitized to one drug, 39 sensitized to two drugs, and 13 to three drugs.

FIG. 9. FIG. 9A: Cytoscape mapping of the profile of interactions among the screening hits. FIG. 9B: color code of map.

FIG. 10. A network view of connected EGFR interactome components.

FIG. 11. Design and screening of a targeted library. A. Distribution of hits as a factor of overall viability reduction with the siRNA. siRNAs in library are listed in order of intrinsic impact on viability of A431 cells treated with DMSO (gray line). Blue triangles, sensitization index (SI) for primary hits with erlotinib; red triangles, SI for validated hits with erlotinib; green squares, SI for primary hits with CPT11. B. Degree of overlap between primary hits obtained for erlotinib, panitumumab, and CPT11. C. Network of validated (red circles) hits sensitizing to EGFR-targeting agents, in the context of the full library. Lines (edges) represent connections based on direct protein-protein interactions or genetic interactions in Drosophila.

FIG. 12. Network properties of hits. A. Hits by source of input in library. MA, microarray indicates transcriptionally responsive to EGFR; DG, Drosophila genetics; PPI1, direct protein interactions with seeds; PM, pathway maps; PPI2, direct protein interactions with a protein within the PPI1 group, or found in a complex with seed proteins; 3S, any 3 sources combined. B. Topological analysis of erlotinib hit network identified in A431 cells. Data shown represents difference between properties of the set of 61 validated hits and the average for 20 randomly generated sets of 61 genes from the library. Measures including degree, topological coefficient, stress, and neighborhood connectivity (see Methods) in each case show highly significant enrichment for hits validated with erlotinib, with the error bars for the random set data reflecting a 99% confidence interval. C. Enriched GO classifications identified among hits. Enrichment is highly significant for proteins annotated as involved in phosphate metabolism and signal transduction (as shown by *, p<0.01). D. Percentage of hits versus library proteins having a recognized ortholog in S. cerevisiae, C. elegans, or D. melanogaster. X axis, the number of species (among listed) having a recognized ortholog.

FIG. 13. Sensitization profile of hits. A. Left, SI values for erlotinib and CPT11, calculated as (test siRNA)/(GL2) for cells grown in drug, divided by (test siRNA/GL2) for cells grown in DMSO: dim green, SI≦0.85; bright green, SI≦0.7; dim red, SI≧1.15; bright red, SI≧1.3. Right, relative ranking of the efficacy of hits to sensitize cells to drugs indicated: black, most sensitizing; white, least sensitizing. For rank order analysis, cluster analysis was performed to identify genes with similar profiles of sensitization (dendrogram, Y axis), and also to cluster cell lines by similarity of response (dendrogram, X axis): these clustered patterns are used to organize the display of genes selected by threshold. Two cell lines, MIA-Paca2 and LoVo, yielded no reproducible hits sensitizing to erlotinib; BCAR1 and TBL1Y were not tested in LoVo B. Left, siRNA-induced viability reduction below 0.85 (dim green), or 0.7 (bright green) that of control siRNA-treated cells, calculated based on the formula (test siRNA)/(GL2 control siRNA) for cells grown in DMSO. Right, relative ranking of hit efficacy in reducing cell line viability. C. Apoptosis was determined by annexin-V labeling and normalized to negative control siRNA. Composite results from two independent experiments are shown as odds ratio columns; black, DMSO treated; white, erlotinib-treated. Asterisks indicate statistically significant (FDR≦0.05) drug-gene interaction in two independent experiments.

FIG. 14. Functional classification of hits. A. Network of validated hits sensitizing to EGFR-targeting agents. Blue lines represent connections based on direct protein-protein interactions or genetic interactions in Drosophila; yellow lines represent connections generated by pathway maps and text mining. Green shadowing on boxes indicates genes that are in the top quartile by rank order of those strongly sensitizing at least 1 (lightest green) to at least 5 (darkest green) cancer cell lines to erlotinib. B. Analysis of ERK (top) and AKT (bottom) status in cell lysates from A431 cells following siRNA transfection, under basal medium conditions and following EGF-stimulation. Average results of three independent Western blots are shown as ratios of amounts of phosphorylated and total proteins. Results were normalized to corresponding ratios in GL2 control; error bars are standard deviations. Asterisks indicate statistically significant difference with negative control; t-test p<0.05. C. Viability curves for erlotinib and Stattic used as single agents, or combined at 1:1 molar ratio in A431 (left) and HCT116 (right) cells, or for erlotinib and Ro-318220 used as single agents, or combined at 1:5 molar ratio in A431 (left) and HCT116 (right) cells. D. Motility was measured by wound healing assay in A431 cells cells treated with 0.5 μM erlotinib alone or in combination with 0.5 μM Stattic (top) or 0.25 μM Ro-318220, and assessed over 18 hours. NS, not significant. *, FDR=3.57*10⁻⁵.

FIG. 15. A. Representative Western blots showing status of EGFR pathway members in A431 cells transfected with siRNAs to the indicated genes. B. Chou Talalay Fa-CI plots obtained for viability of A431 and HCT116 cells treated with protein kinase C inhibitor (Ro, Ro-318220) or STAT3 inhibitor (Stattic) combined with erlotinib at different molar ratios. C. Viability curves for erlotinib and enzastaurin used as single agents, or combined at 5:1 (left) or 1:1 (right) molar ratios in A431 cells.

FIG. 16. Phosphoprotein changes with signaling inhibitors. Western blot quantitative data of the phosphorylated proteins in A431 lysates obtained under serum starvation (basal) or EGF stimulation. The results represent averages of 3 independently conducted experiments; bars indicate SEM. Data shown calculated from 3 independent biological replicates except for IκB, which was derived from 2 replicates.

FIG. 17. Synergy between inhibitors of Aurora-A and of EGFR. A. Kinases directly associated with the BCAR1-NEDD9-SH2D3C cluster. Kinases with no clinical small molecule inhibitor available are indicated in pink; kinases with small molecule inhibitors are indicated in blue, or in green if inhibitor has previously been tested for synergy with EGFR-targeting agents. B. Inhibitors of EGFR and inhibitors of Aurora kinase A synergize to reduce viability of cancer cells in vitro. Summary results of drug interactions calculated as Chou-Talalay coefficient of interaction (CI). The results are based on Cell Titer blue viability determinations; similar results were obtained with BrdU (not shown). C. Combination of PHA-680632 and erlotinib treatment increases apoptosis in HCT116 cells at 72 hours; *, t-test p=0.001. D. Dose-dependent inhibition of HCT116 cell viability by combination of PHA-680632 and erlotinib. E. Cell motility was measured by wound healing assay in cells treated with drugs at concentrations indicated, and assessed over 18 hours. *FDR is <10⁻⁵ for (1), (2). F. Left, relative soft agar colony formation of cells grown for 2-3 weeks in drugs at concentrations indicated. FDR is equal to (1) 0.0003, (2) 0.0006, (3) 0.0003, and (4) 0.004. Right, results from typical experiment. G. Tumor xenografts implanted in SCID mice were treated with drugs as indicated beginning at day 0. P-values are (1) 0.005, (2) 0.008. H, Quantitation of 3 independent Western analyses of protein lysates of cells treated with erlotinib and PHA-680632 for 3 hrs followed by EGF stimulation. Error bars represent standard error of the mean (SEM) of three independent experiments; **, t-test p=0.013.

FIG. 18. PHA-680632 and erlotinib combine to reduce AKT phosphorylation. Representative Western blot showing activity of erlotinib and PHA680632 either alone or in combination on serum starved HCT116 cells treated with the indicated inhibitors for 3 hours and stimulated with EGF.

FIG. 19. Dual inhibition of Aurora-A and EGFR suppresses activation of multiple signaling nodes. Ranked fold increase in phosphorylation signal of 46 proteins in A431 cells stimulated with EGF (A) or grown in 1% serum media (B) and treated with indicated drugs. Inset, graphs illustrate magnified scale of indicated phosphoproteins. Proteins showed in red have consistent decrease of >2-fold in signal intensity in independent biological replicates, as indicated.

FIG. 20. Expression profile of hits in cell lines. Not all siRNAs were active in all cell lines. The phenomenon of cell line-specific activity could reflect the presence or absence of expression of a targeted mRNA in specific cell lines, or might instead reflect the presence of the mRNA in multiple cell lines, but a requirement for the gene product only in a subset of cell lines. Establishing this point has important implications, as often a criterion for targeting a given protein therapeutically has been the observable high levels of expression of that protein within a tumor. We performed quantitative RT-PCR of the set of validated hits in each of the reference cell lines, and compared relative expression level with intrinsic siRNA sensitivity. Over the entire set of genes, there was no significant correlation between the relative abundance of a targeted mRNA in a cell line and the efficacy of the corresponding siRNA in influencing either sensitization to the drugs used, or cell viability. Shown, relative expression of genes targeted by siRNAs measured for each cell lines using quantitative RT-PCR. ΔCt value is in each case calibrated to A431; e.g., strongest blue requires 12 additional amplification cycles to detect mRNA, indicating it is 2¹²-fold less abundant than in A431 cells. Genes with significant correlation (*) or anti-correlation (**) with proliferation ability are indicated; Pearson correlation values are respectively, AKT2, 0.91; RASA3, 0.84; STAT3, 0.81; VAV3, 0.80; GRB7, −0.89; PRKCE, −0.87; DUSP4, −0.81.

FIG. 21. Expression profile of hits in Oncomine. To identify genes among the hit set that have been reported as up- or down-regulated in cancerous vs. normal tissues (with a p-value equal or less than 0.01), the Oncomine database was searched across all tissue types. Tissues with fewer than two independent analyses available were excluded from analysis. For each gene, the total number of cancer vs. normal samples analyses reporting it to be up- or downregulated was normalized to the total number of analyses for the corresponding tissue type. Data were visualized using MeV: Red, up-regulation; blue, down-regulation. Intense hues represent a value of 1, meaning all of the available cancer vs. normal analyses found comparable up- or down-regulation; less intense hues indicate expression change was found in some but not all independent studies. Yellow indicates the genes found to be both up- and down-regulated in the same tissue type by different data sets. White boxes indicate no significant change in expression was identified in any study.

### DETAILED DESCRIPTION OF THE INVENTION

In accordance with the present invention, compositions and methods are provided to better improve the treatment of cancer. Using small interfering RNA (siRNA) technology to identify proteins that contribute to resistance to clinically important therapeutic agents, we have identified suitable candidates which inhibit the action of identified resistance proteins thereby enhancing therapy. Such proteins also provide novel biomarkers to better select individual patients for specific treatment regimens.

An effective strategy has been devised to enhance the potency of EGFR-targeting agents. First, systems biology studies in model organisms have begun to establish that synthetic lethal relationships commonly involve genes that are involved in redundant, parallel pathways, or are vertically linked in the same pathway (8). It is instructive that in a seminal genome-wide screen to identify sensitizers to the microtubule-targeting agent paclitaxel, many hits could be clustered into coherent groups of genes associated with the proteasome or mitotic spindle (9), which a priori had been linked to paclitaxel activity based on existing pathway knowledge. Further, in the design of combination therapies in the clinic, the selection strategy for drugs to combine frequently involves common principles: that is, identifying two drugs that 1) inhibit the same target, 2) inhibit functionally linked and/or semi-redundant targets, or 3) inhibit vertically linked targets (10). Together, these observations suggested that generation of a mid-throughput siRNA library (−500-800 genes) that is large enough to fully represent genes functionally linked to the EGFR-Ras-MAPK signaling axis, would greatly increase the useful “hit” rate for genes that chemosensitize EGFR-targeted therapies.

This strategy has produced numerous gains, outlined below in detail: 1) we have been able to conduct reiterative screens to test strategies of hits selection and validation; 2) we have been able to test drive our experimental system to identify systematic errors and biases and apply statistical and bioinformatics tools to compensate for these biases; 3) the EGFR-centered library allowed us to validate the siRNA screening as successful strategy to identify interesting chemosensitizing hits; 4) we have identified siRNA molecules that sensitize chemoresistance cancer cells to EGFR based therapies.

### DEFINITIONS

As used herein, the phrase “EGFR/MEK1 targeting agent refers to small molecules, antibodies, or RNA agents targeting EGFR, EGFR-related family members, or immediate effectors in the EGFR cascade including but not limited to Ras, Raf, and MEK1.

The phrase “EGFR/NEDD9/TGF-β interactome” refers to proteins linked by close physical or functional association with EGFR, or with the proteins NEDD9, TGF-beta, or their binding partners.

A “small nucleic acid inhibitor” refers to any sequence based nucleic acid molecule which, when introduced into a cell expressing the target nucleic acid, is capable of modulating expression of that target. While siRNA molecules are exemplified herein, antisense, miRNA, shRNA and the like may be utilized in the methods of the invention.

As used herein, the phrase “effective amount” of a compound or pharmaceutical composition refers to an amount sufficient to modulate tumor growth or metastasis in an animal, especially a human, including without limitation decreasing tumor growth or size or preventing formation of tumor growth in an animal lacking any tumor formation prior to administration, i.e., prophylactic administration.

Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers, for example to a diluent, adjuvant, excipient, auxilliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin.

A pharmaceutical composition of the present invention can be administered by any suitable route, for example, by injection, by oral, pulmonary, nasal or other forms of administration. In general, pharmaceutical compositions contemplated to be within the scope of the invention, comprise, inter alia, pharmaceutically acceptable diluents, preservatives, solubilizers, emulsifiers, adjuvants and/or carriers. Such compositions can include diluents of various buffer content (e.g., Tris HCl, acetate, phosphate), pH and ionic strength; additives such as detergents and solubilizing agents (e.g., Tween 80, Polysorbate 80), anti oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435 1712 which are herein incorporated by reference. A pharmaceutical composition of the present invention can be prepared, for example, in liquid form, or can be in dried powder, such as lyophilized form. Particular methods of administering such compositions are described infra.

In yet another embodiment, a pharmaceutical composition of the present invention can be delivered in a controlled release system, such as using an intravenous infusion, an implantable osmotic pump, a transdermal patch, liposomes, or other modes of administration. In a particular embodiment, a pump may be used [see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)]. In another embodiment, polymeric materials can be used [see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)]. In yet another embodiment, a controlled release system can be placed in proximity of the target tissues of the animal, thus requiring only a fraction of the systemic dose [see, e.g., Goodson, in Medical Applications of Controlled Release, supra, vol. 2, pp. 115 138 (1984)]. In particular, a controlled release device can be introduced into an animal in proximity of the site of inappropriate immune activation or a tumor. Other controlled release systems are discussed in the review by Langer [Science 249:1527 1533 (1990)].

As used herein the term “biomarker” refers to a characteristic that is objectively measured and evaluated as an indicator of normal biologic processes, pathogenic processes, or pharmacologic responses to a therapeutic intervention.

As used herein, the terms “modulate”, “modulating” or “modulation” refer to changing the rate at which a particular process occurs, inhibiting a particular process, reversing a particular process, and/or preventing the initiation of a particular process. Accordingly, if the particular process is tumor growth or metastasis, the term “modulation” includes, without limitation, decreasing the rate at which tumor growth and/or metastasis occurs; inhibiting tumor growth and/or metastasis; reversing tumor growth and/or metastasis (including tumor shrinkage and/or eradication) and/or preventing tumor growth and/or metastasis.

As used herein, the terms “tumor”, “tumor growth” or “tumor tissue” can be used interchangeably, and refer to an abnormal growth of tissue resulting from uncontrolled progressive multiplication of cells and serving no physiological function. A solid tumor can be malignant, e.g. tending to metastasize and being life threatening, or benign. Examples of solid tumors that can be treated or prevented according to a method of the present invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastic cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Moreover, tumors comprising dysproliferative changes (such as metaplasias and dysplasias) can be treated or prevented with a pharmaceutical composition or method of the present invention in epithelial tissues such as those in the cervix, esophagus, and lung. Thus, the present invention provides for treatment of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68 to 79). Hyperplasia is a form of controlled cell proliferation involving an increase in cell number in a tissue or organ, without significant alteration in structure or function. For example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

Other examples of tumors that are benign and can be treated or prevented in accordance with a method of the present invention include arteriovenous (AV) malformations, particularly in intracranial sites and myoleomas.

### Methods for Modulating Tumor Growth or Metastasis

As explained above, the present invention is directed towards methods for modulating tumor growth and metastasis comprising, inter alia, the administration of a EGFR/Mek-1 targeting agent and at least one sensitizing siRNA molecule. The agents of the invention can be administered separately (e.g, formulated and administered separately), or in combination as a pharmaceutical composition of the present invention.

The present invention provides for both prophylactic and therapeutic methods of treating a subject at risk of (or susceptible to) a disorder or having a disorder associated with aberrant or unwanted target gene expression or activity. In one embodiment, the subject is administered a lipid/therapeutic agent complex, for example, a liposome comprising an siRNA for suppressing the expression of an the undesired gene product. It is understood that “treatment” or “treating” as used herein, is defined as the application or administration of a therapeutic agent (e.g., an RNAi agent or vector or transgene encoding same, a polypeptide, e.g., an antibody or fragment thereof, or small molecule) to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In another aspect, the invention provides a method for preventing in a subject, a disease or condition associated with an aberrant or unwanted target gene expression or activity, by administering to the subject a lipid/therapeutic agent complex of the invention (e.g., an siRNA agent or vector or transgene encoding same, a polypeptide, e.g., an antibody or fragment thereof, or small molecule). Subjects at risk for a disease which is caused, or contributed to, by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression. Depending on the type of target gene aberrancy, for example, a target gene, target gene agonist or target gene antagonist agent can be used for treating the subject. The appropriate agent can be determined based on screening assays described herein.

In yet another aspect, the invention pertains to methods of modulating target gene expression, protein expression or activity for therapeutic purposes. Accordingly, in an exemplary embodiment, the modulatory method of the invention involves contacting a cell capable of expressing target gene with a lipid/therapeutic agent complex (e.g., an siRNA agent or vector or transgene encoding same) that is specific for the target gene or protein (e.g., is specific for the mRNA encoded by said gene or specifying the amino acid sequence of said protein) such that expression or one or more of the activities of target protein is modulated. These modulatory methods can be performed in vitro (e.g., by culturing the cell with the agent), in vivo (e.g., by administering the agent to a subject), or ex vivo. As such, the present invention provides methods of treating an individual afflicted with a disease or disorder characterized by aberrant or unwanted expression or activity of a target gene polypeptide or nucleic acid molecule. Inhibition of target gene activity is desirable in situations in which target gene is abnormally unregulated and/or in which decreased target gene activity is likely to have a beneficial effect.

When employing the methods or compositions of the present invention, other agents used in the modulation of tumor growth or metastasis in a clinical setting, such as antiemetics, can also be administered as desired.

Materials and methods are provided herein to facilitate the practice of the present invention.

Cell lines, Compounds, Antibodies. The A431 cervical adenocarcinoma (K-Ras wt, p53 mutant (Kwok et al. (1994) Cancer Res. 54:2834), HCT116 and LoVo (K-Ras mutant, p53 wt) colorectal carcinoma and the PANC-1 (K-Ras mutant, p53 mutant) and MIA PaCa-2 (K-Ras mutant, p53 and p16 mutant) pancreatic adenocarcinoma (Brunner et al., (2005) Cancer Res. 65:8433) cell lines were obtained from the ATCC (USA). The DLD-1 (K-Ras mutant, p53 mutant) and DKS-8 (with the activated K-Ras allele disrupted [derived from DLD-1], p53 mutant; Sarthy et al. (2007) Mol. Cancer Ther. 6:269) were a kind gift of Dr. Robert J. Coffey (Vanderbilt University, Tenn.). SCC61 cells (K-Ras wt, p53 mutant), derived from squamous cell carcinomas of the head and neck, were kindly provided by Dr. Tanguy Y. Seiwert (University of Chicago). All cell lines were maintained in DMEM supplemented with 10% v/v fetal bovine serum (FBS) and L-glutamine without antibiotics. Cetuximab, panitumumab and erlotinib were purchased from the Fox Chase Cancer Center pharmacy; CPT11 and C1368 from Sigma-Aldrich (USA); Stattic and Ro-318220 from EMD Chemicals (Gibbstown, N.J., USA). PHA-680632 was obtained from Nerviano Medical Sciences (Nerviano, Italy), as a kind gift of Dr. Jurgen Moll. Enzastaurin was generously provided by the Elli Lilly Company (Indianapolis, Ind.). All antibodies used in Western blot experiments were purchased from Cell Signaling (Danvers, Mass., USA), except anti-p53 mouse monoclonal antibody was from Calbiochem (EMD Biosciences, USA).

### EGFR Network Construction.

### Methods for Library Construction.

Four sources of information were used, including 1) published EGFR pathway maps, 2) human protein-protein interaction (PPI) data, gleaned from various databases, 3) human orthologs of PPIs and genetic interactions modeled from Drosophila, and 4) microarray data obtained at brief intervals after treatment of cells with stimulators or inhibitors of EGFR/ErbB2. Following initial assembly of a larger gene list, genes were parsed into high confidence (“core”, denoted as “1” after the corresponding letter code) versus lower confidence sets (denoted as “2”), based on the confidence criteria outlined for each section below. For each category of information, all “core” components were included in the final library, as were genes noted as lower confidence but included in at least two categories of search criteria (e.g., second order protein-protein interaction and microarray linkage). Finally, for the assembled set of EGFR interactors, multiple paralogous genes were identified in humans using the KEGG Sequence Similarity DataBase (SSDB) resource, see the world wide web at.genome.jp/kegg/ssdb/. 77 paralogs of the best-characterized and biologically significant genes were added to the set. All data storage, handling and analysis were done primarily in Cytoscape (on the world wide web at cytoscape.org).

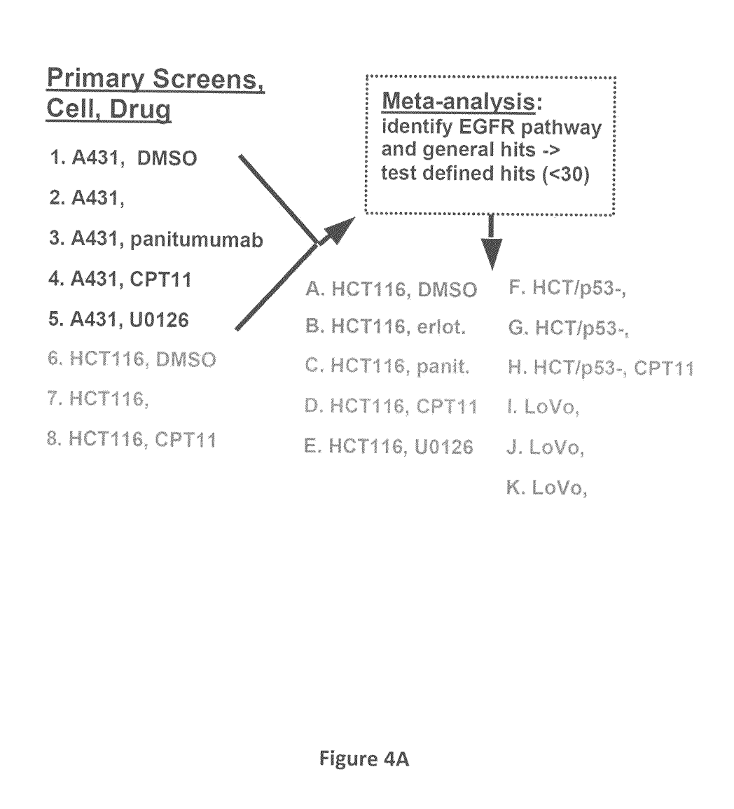

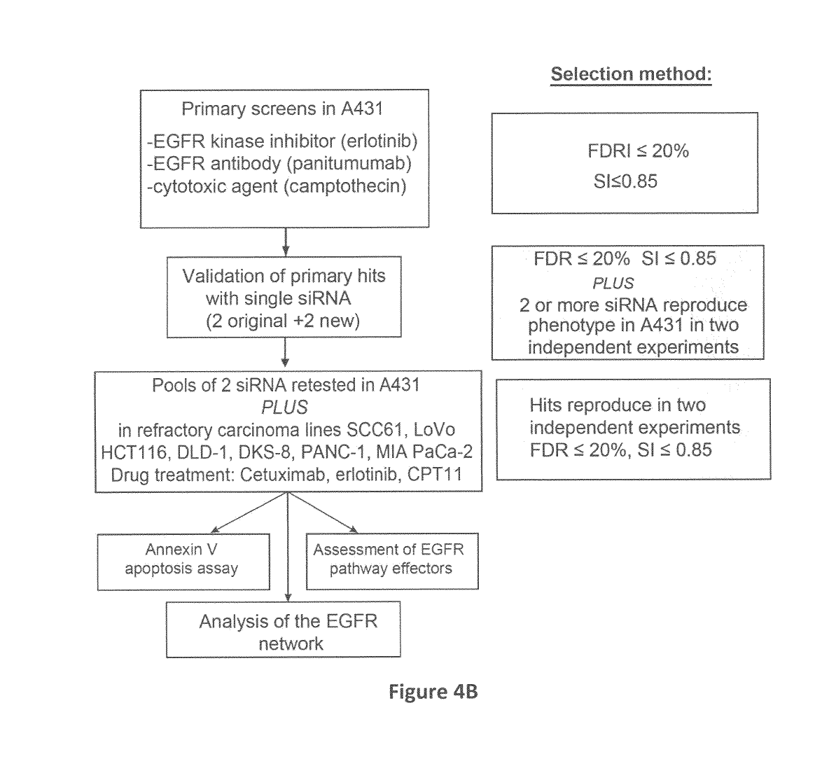

1) Pathway map sources. Protein names for were extracted from the following pathway maps focused on EGFR: STKE (on the world wide web at stke.sciencemag.org/cgi/cm/stkecm%3BCMP_{—}14987); Biocarta (on the world wide web at biocarta.com/pathfiles/PathwayProteinList.asp?showPFID=102); the Systems Biology model repository (on the world wide web at systems biology.org/001/005.html); NetPath (on the world wide web at netpath.org/pathways?path_id=NetPath_{—}4); and from Protein Lounge (on the world wide web at proteinlounge.com/poppathways1.asp?id=EGF+Pathway). Protein names were individually inspected and, where necessary, converted to the corresponding official (NCBI/EMBL) symbols. Proteins mentioned on at least two EGFR-centered pathways were designated as “pathway core”; we note, significant divergence was seen among different interpretations of the “EGFR pathway” by the 5 sources.
2) PPIs. EGFR/ERBB1 and ErbB2 were used as seeds for PPI searches. Curated information regarding human PPIs of these seeds was collected from the following databases: Biomolecular Object Network Databank (BOND) (on the world wide web at bond.unleashedinformatics.com/); General Repository for Interaction Datasets (on the web at thebiogrid.org/); EMBL_EBI IntAct on the web at ebi.ac.uk/intact/site/index.jsf); The Human Protein Reference Database (on the web at hprd.org/); Kyoto Encyclopedia of Genes and Genomes (KEGG) (on the web at genome.jp/about_genomenet/service.html); and Prolinks Database 2.0 (on the web at mysq15.mbi.ucla.edu/cgi-bin/functionator/pronav). Data for first rank (direct) interactors were collected both by export from the corresponding database and subsequent import into Cytoscape, and by directly querying those databases using the BioNetBuilder plugin (on the web at err.bio.nyu.edu/cytoscape/bionetbuilder/), and then cross-comparing retrieved results. Data for the second order interactions were obtained by using EGFR and ERBB2 first rank interactors as seeds for an additional round of query, and only through the BioNetBuilder plugin. Finally, an orthogonal set of second rank interactors was obtained by analysis of protein complexes with more than 2 subunits, which included EGFR. Information for complexes was obtained from BOND and IntAct, and manually compared to the lists in the corresponding publications. We also used the SHC1 and SHC3 adaptors, which bridge between EGFR and downstream signaling effectors, and the CAS (EFS, BCAR1, and NEDD9) scaffolding proteins, which connect EGFR to the SRC and TGF-β core signaling cascades (O'Neill et al., (2000) Trends Cell Biol. 10:111; Defilippi et al. (2006) Trends Cell Biol. 16:257), as seeds for first order PPI searches. Second order PPIs EGFR and ErbB2 were ranked higher (i.e., P1) if they were also first order interactors of SHC or CAS proteins.
3) To extract a set of EGFR-centered interactions potentially conserved between humans and D. melanogaster, information assembled by the Michigan Proteomics Consortium in the Drosophila Interactions Database (DIP) (on the web at proteome.wayne.edu/PIMdb.htm) was used. Briefly, this database integrates genetic and or protein interaction data from a variety of non-mammalian species (yeast, worms, flies). Of 105 EGFR interactors (almost exclusively from Drosophila genetic interactions), 65 had 1-2 conserved human orthologs (117 genes).
4) Microarray data were obtained from The Gene Expression Omnibus (GEO, release date Dec. 15, 2006). In the selected dataset (GSE6521; raw data available at on the web at ncbi.nlm.nih.gov/geo/query/acc.cgi?acc=GSE6521), MCF-7 human breast cancer cells were incubated with the growth hormone heregulin (HRG), or AG1478 (an EGFR kinase inhibitor), or both growth hormone and AG1478. Controls were set as growth hormone/inhibitor non-treated cells. A total of 348 genes with a >1.5 fold change (+ or −) upon AG1478 treatment was identified. In this group, the core set includes 89 genes which showed a >2-fold change in expression level upon AG1478 treatment, or which were inducible by HRG (>1.5 fold) and repressible by AG1478 (>1.5 fold).
High throughput siRNA screening methods. The custom siRNA library targeting 638 human genes was designed and synthesized with two siRNA duplexes for each gene target (Qiagen, Valencia, Calif.). Transfection conditions were established for the A431 cervical adenocarcinoma (K-Ras wt, p53 mutant) cell line (data not shown) using PLK1 & GL2 siRNA controls to achieve Z′ values of 0.5 or greater. IC values using erlotinib, panitumumab, and camptothecin (CPT11) were established (data not shown). Details of establishment of Z′ factor for transfections, and statistical consideration for selection of preliminary positive candidates graphically outlined in FIG. 4B, and based on standard approaches described in detail in Whitehurst et al., (2007) Nature 446:815). For each gene targeted, two independent siRNA duplexes were combined and arrayed in 96-well plates with a layout intended to seed in regular positive siRNA (targeting PLK1) and negative control siRNA (targeting insect luciferase GL2, Thermo Fisher Scientific, USA) amongst test siRNAs. A reverse transfection protocol was used where siRNA at final concentration of 50 nM was mixed with Dharmafect 1 transfection reagent according to the manufacturer's instructions (Thermo Fisher, USA). Cells (3500 per well, resuspended in 1% FBS/DMEM) were added directly to wells using an automated liquid dispenser. At 24 hr following transfection, two replica plates were treated with drugs at previously established IC30 or 0.02% DMSO diluted in culture media. The viability at 96 hr post-transfection was assessed using Alamar blue (CellTiter Blue Viability Assay, Promega, USA). Dose-responses for each drug and cell line were retested in parallel with each screen.

For screening, A431 cells were transfected with siRNA followed by exposure to vehicle (0.02% DMSO), or drug used at inhibitory concentrations of 30% (IC₃₀). Viability was determined for each target gene and normalized to the averaged GL2 viability on each plate. Sensitization index (SI) was calculated for each individual well on a 96-well plate as SI=(V_{drug}/GL2_{drug})/(V_{DMSO}/GL2_{DMSO}), where V was viability in wells transfected with targeting duplexes and GL2 was the averaged viability of 4 wells with non-targeting negative control siRNA on the same plate. All calculations were automated using cellHTS package within open source Bioconductor Package (on the web at bioconductor.org) (Gentleman et al. (2004) Genome Biol. 5:R80). The effect of drug treatment on viability was measured based on the normalized viabilities in the drug treated and vehicle wells using Limma (Smyth, G. K. (2004) Statistical Applications in genetics and molecular biology 3, Article 3). Limma borrows strength across genes based on an empirical Bayes approach and identifies statistically significant changes in viability by combining information from a set of gene-specific tests. Hits were identified based on statistical significance as well as biological significance. Statistical significance was determined by p-value controlled for the false discovery rate (FDR) using the Benjamini-Hochberg step-up method (Benjamini et al. (1995) J. Royal Stat Soc B 57:289) to account for multiple testing. Hits showing an FDR of less than 20% were considered statistically significant. Biological significance was arbitrarily defined as an increase or decrease in SI greater than 15%. Hits identified as statistically and biologically significant were further validated.

Primary sensitizing hits obtained with erlotinib and/or cetuximab were further tested with erlotinib and DMSO in the A431 cell line by using 4 siRNA duplexes individually (the two originally used in the screen, plus two new non-overlapping RNA oligoribonucleotides), to confirm the sensitization phenotype at 10 nM and 50 nM concentrations. Hits were considered as validated by this method if at least 2 out of 4 siRNA reproduced the sensitization phenotype with SI≦0.85, FDR≦20% for each individual siRNA sequence in at least two independent experiments. For a number of hits, we additionally confirmed sensitizing siRNAs reduced mRNA levels for the targeted genes, using qRT-PCR; and used Western analysis to confirm protein knockdown (data not shown).

Cell line specificity of hit activity. Of the confirmed set of 61 siRNA targets identified for erlotinib in A431 cells, 45 were further tested for sensitization to erlotinib, cetuximab and camptothecin in A431 versus refractory adenocarcinoma cell lines for which optimal transfection conditions and drug sensitivity had been established. In this analysis, for each target, the two most active siRNA duplexes identified during the validation stage were pooled in a 96-well format, cells were reverse-transfected and drug-treated under conditions similar to those described above for the initial A431 screen. SI and statistical significance were calculated as in the validation experiments. All experiments were performed at least three times independently.

In subsequent data analysis, two approaches were used. For the relative ranking approach, for each experiment, SI values for each siRNA pool were ranked from the strongest (assigned a value of 0) to the weakest (assigned as 1). For all experiments performed with a given cell:drug combination (e.g., A431:erlotinib, or HCT116:CPT11) averages were determined based on at least three experimental runs. The averaged data were imported and clustered in MultiExperiment Viewer (MeV_{—}4_{—}3) software (Saeed et al. (2003) Biotechniques 34:374), and dendrograms were created using HCL Support Trees (using Euclidian Distance as a metric, and bootstrapping with 100 iterations). For the absolute threshold approach, specific SI thresholds were applied for each data point, considering only data with an FDR≦20% in each independent experiment. Data were visualized in MultiExperiment Viewer using color assignments to indicate SI cutoffs obtained in at least two independent experiments, as described in figure legends. The resulting output of both analytic strategies was processed in standard graphic software to improve visualization of data.

Quantitative RT-PCR. For evaluation of expression of validated target genes, each of the cell lines was grown to 70% confluency in full DMEM media, then total RNA was extracted using RNeasy Minikit (Qiagen, Valencia, Calif.). To confirm mRNA depletion by siRNA, 48 hrs after transfection of A431 cells grown in 96-well plates, total RNA was extracted at using a Cell-to-Ct kit (Applied Biosystems, Foster City, Calif.). Subsequent quantitative RT-PCR reactions were performed using TaqMan probes and primers designed by the manufacturer, using an ABI PRISM 7700 detection system (Applied Biosystems, Foster City, Calif.). The results were analyzed using the comparative Ct method to establish relative expression curves.

To assess whether gene expression levels correlated with the ability of gene-targeted siRNAs to inhibit intrinsic cell growth, we used a Pearson correlation of the mean values of gene expression relative to that obtained in A431 cells measured by RT-PCR, against the mean growth observed in DMSO-treated cells in all experiments. To test significance, we performed 100 permutations of the cell line labels in the RT-PCR measurements and generated Pearson correlation values. Significance was defined as a false discovery rate (FDR) of 5%, setting Pearson correlation greater than 0.745 or less than −0.71 for positive correlated or negative-correlated, respectively. Positive correlation indicates that higher expression (lower number of RT-PCR cycles) is correlated with greater growth inhibition, while negative correlation indicates higher expression is correlated with lower inhibition.

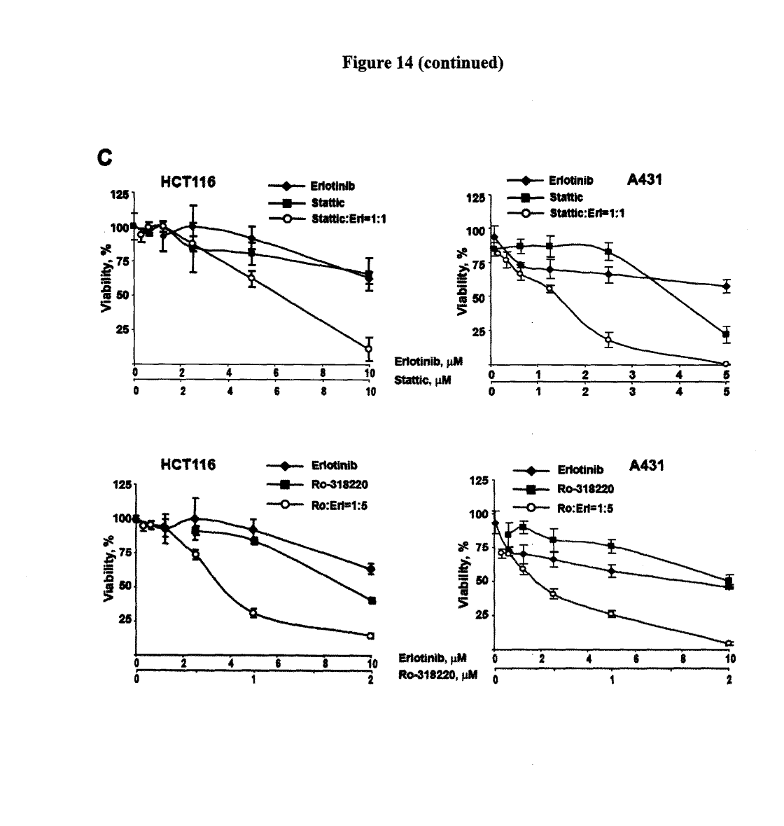

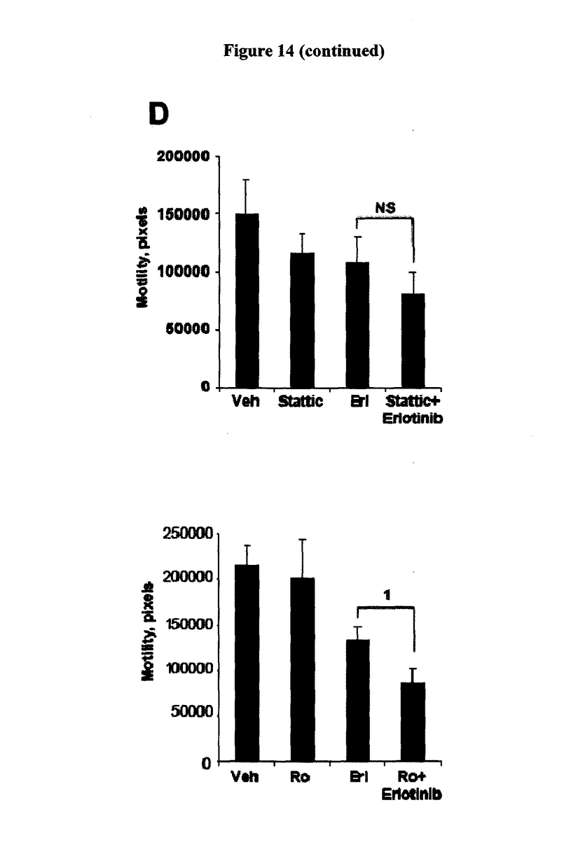

Network analysis with hits. For all genes in the library, the String search engine (Jensen et al., (2009) Nucleic Acids Res. 37:D412) was used in subsequent analysis to augment information on PPIs in human cells, PPIs between homologous genes in model organisms, database/pathway links, and textmining (coappearance of gene names in PubMed). Data regarding experimentally proven interactions in human and model organisms were merged. Topological properties of the library network were assessed using NetworkAnalyzer plugin for Cytoscape (Assenov et al., (2008) Bioinformatics 24: 282), based on STRING-expanded defined interactions among genes in the library (based only on experimental data). In this analysis, for each node, degree (reflecting the number of edges linked to it), stress (reflecting how frequently it lies in the shortest paths connecting other nodes), and neighborhood connectivity (the average number of neighbors for each direct interactor of the node) were separately assessed. The topological coefficient was calculated to provide an estimate for the trend of the nodes in the network to have shared neighbors. To provide additional context in some analyses (FIG. 14) STRING-extracted information from pathway databases and textmining data were merged, and displayed using Cytoscape as indicated in figure legends.
Apoptosis assays. Apoptosis was measured using the Annexin V assay (Guava Technologies, Hayward, Calif.). Annexin V-positive A431 cells were counted using Guava flow cytometry 72 hours post transfection, 48 hours after treatment. Statistical significance versus control GL2 siRNA was determined by logistic regression models to identify genes increasing apoptosis with erlotinib relative to vehicle.
Pathway analysis. To measure the effect of siRNAs on the activity of EGFR effectors, cells were transfected with siRNA, and the culture media replaced with glutamine-supplemented serum-free DMEM at 24 hrs post-transfection. After overnight incubation, cells were treated with DMSO, erlotinib or PHA-680632 for 2 hrs, then either left untreated, or stimulated with EGF at 15 ng/ml for 15 minutes. Cell extracts were prepared using M-PER™ mammalian protein extraction buffer (Thermo Scientific, Rockford, Ill.) supplemented with the Halt™ phosphatase inhibitor cocktail (Thermo Scientific, Rockford, Ill.) and the Complete Mini™ protease inhibitor cocktail (Roche Diagnostics Gmbh, Manheim, Germany). Extracts were centrifuged at 15,000 g for 10 min at 4° C. Western signal detection was performed using antibodies to indicated proteins with LiCor technology (Lincoln, Nebr., USA) or standard X-ray film.

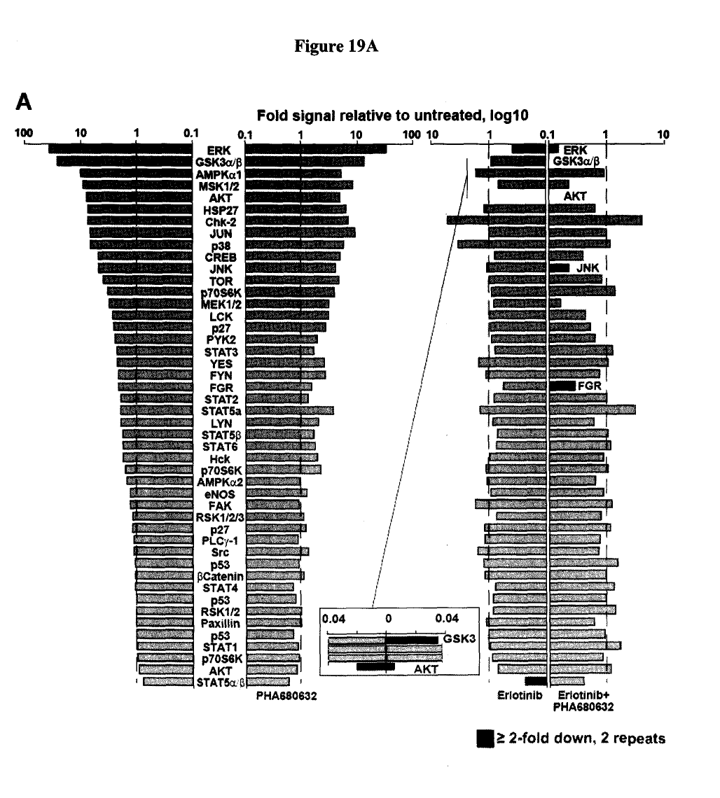

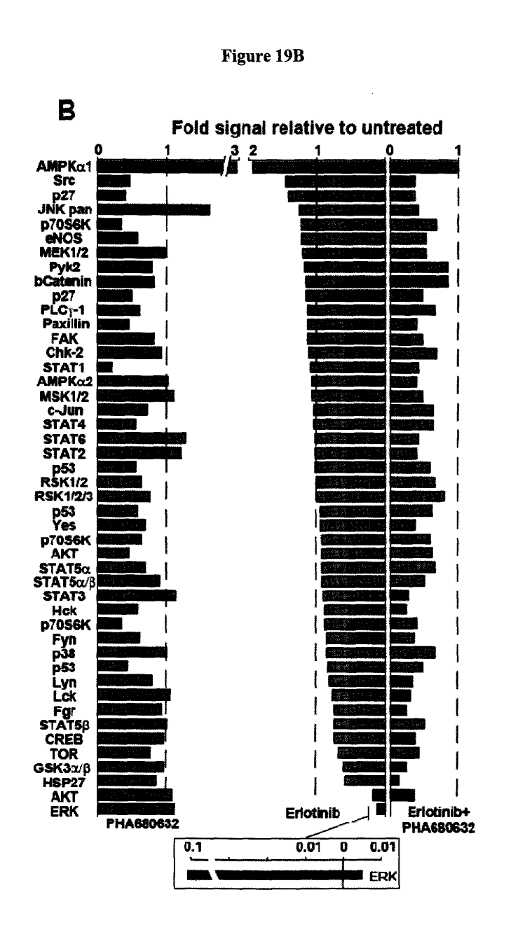

For phosphoproteomic analysis, we used the Proteome Profiler™ array (R&D Systems, Minneapolis, Minn., USA) according to the manufacturer's protocol. In brief, A431 cells were grown for 24 hours in DMEM supplemented with L-glutamine and 1% FBS to 70% confluency. Cells then were either serum starved overnight or remained in the same media. Serum starved and cells incubated in 1% serum were either left untreated or incubated with IC₃₀ concentrations of inhibitors for 3 hours. For FIG. 19A, we used erlotinib at 0.5 μM and PHA-680632 at μM alone or in combination; for FIG. 19B, erlotinib at 1 μM and PHA680632 at 0.5 μM individually, and erlotinib at 0.5 μM plus PHA at 0.1 μM in combination. Serum-starved cells were subsequently stimulated with recombinant EGF (Sigma-Aldrich, USA) at 15 ng/mL for 15 minutes before lysis. For a subset of phospho-proteins, phosphorylation status was confirmed by Western blot. Quantification was done using ImageJ software.

Drug synergy testing. The coefficient of interaction (CI) between pharmacological inhibitors was established by the Chou-Talalay method (Chou et al., (1984) Adv Enzyme Regul 22:27). The software package CalcuSyn (BioSoft, UK) was used to automate calculations. Briefly, for each drug tested, an IC₅₀ curve was established in each cell line, and used to select combination doses of drugs for subsequent synergy tests. 3500 cells were plated per well in 96-well plates. After 24 hours, cells were treated with serial dilutions of individual inhibitors, or combinations of two inhibitors maintained at a constant molar ratio. After 72 hours incubation, cell viability was measured using either CellTiter Blue (Promega, USA) or a WSTI assay (Roche Applied Science, Indianapolis, Ind.). The CI values for each dose and corresponding cytotoxicity were expressed as the fraction affected (Fa) and were calculated using CalcuSyn computer software and presented as Fa-CI plots. CI values<1 indicate synergy, and <0.5 strong synergy, between the two agents in producing cytotoxic effect.
Anchorage-independent growth and cell motility. Soft agar assays were done essentially as described (10). Cells were seeded at 2000 cells per well and grown for 2-3 weeks. Colonies were stained with thiazolyl blue tetrazolium bromide, and scored using a Nikon SMZ1500 microscope coupled with Cool Snap charge coupled device camera (Roper Scientific, Inc., Tucson, Ariz.) with Image Pro-Plus software (Media Cybernetics, Silver Spring, Md.). Survival curves were based on at least two concentration points, with values determined in at least two separate experiments, with each assay done in duplicate. Drug interactions were calculated as above using CalcuSyn software (Biosoft, Ferguson, Mo.) to establish the combination index (CI). For motility assays, movement of A431 cells grown in 1% FCS into a scratched area of the monolayer was monitored with a phase contrast 10× objective using an inverted microscope (Nikon TE2000). Images were obtained every 20 min for 18 hours. Areas of migration were estimated using MetaMorph software. For both studies, analysis of variance was used to determine the treatment effect for each comparison. The logarithm of normalized ratios (to vehicle control) was used in the analysis. Multiple hypothesis testing was accounted for by the false discovery rate method of Benjamini & Hochberg (supra).
Tumor Formation in vivo. Male CB.17/scid mice aged 6-8 weeks were obtained from the Fox Chase Cancer Center breeding colony. All experiments were carried out according to protocols approved by the institutional animal use committee. Mice were injected with 3×10⁶ A431 cells subcutaneously into the flanks. Palpable tumors appeared in all animals in 10-14 days, and were measured 3 times a week in two dimension and volume calculated by modified ellipsoidal formula as Length×Width²×0.52. Mice were randomized and treatments commenced when tumor volume exceeded 65 mm³. Erlotinib at doses 10-20 mg/kg was given by oral gavage as in 10% DMSO/saline. Enzastaurin was suspended in 5% dextrose in water and dosed at 75 mg/kg by gavage twice daily. PHA-680632 was freshly dissolved in acidified 5% dextrose in water and administered intraperitoneally twice daily at 15 mg/kg dose. The generalized estimating equations approach (with an autoregressive correlation structure) was used to model tumor growth. A linear time-effect was included in the model for the logarithm of tumor volume and interacted with the treatments in each comparison.

The following examples are provided to illustrate certain embodiments of the invention. They are not intended to limit the invention in any way.

### Example I

### Identification of Sensitizing siRNA Molecules

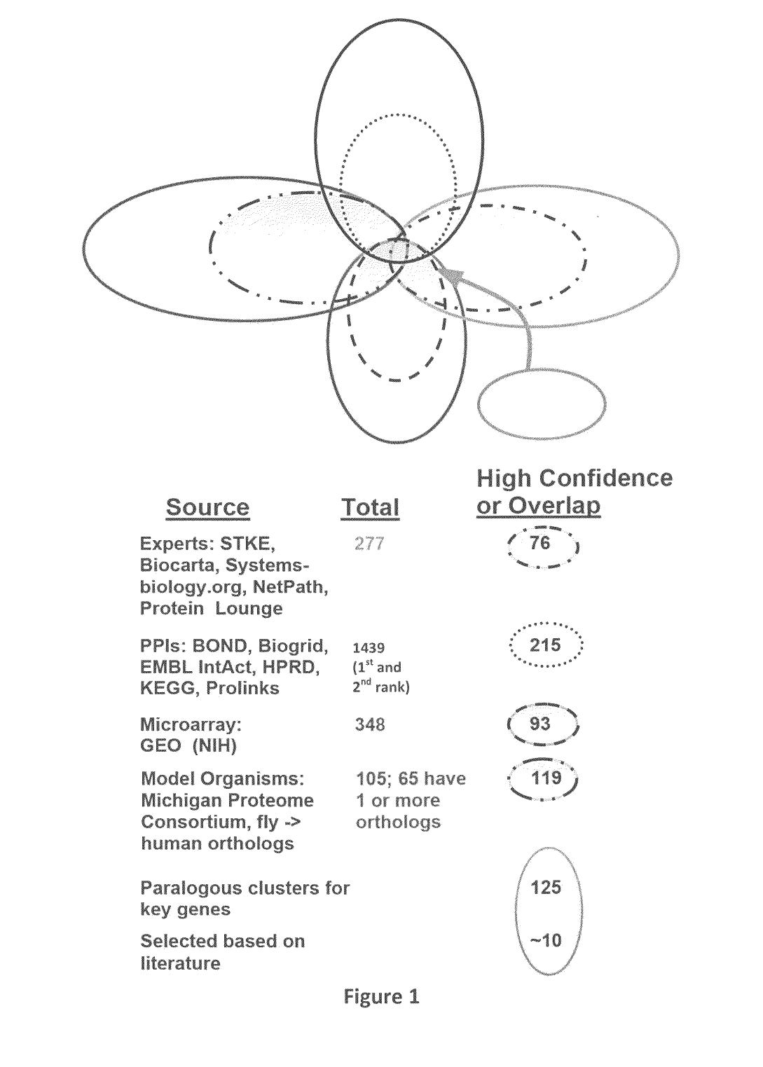

To generate an EGFR interactome library, we extensively mined public access databases containing information about protein-protein interactions and mRNA expression profiles generated in humans. These databases included among others NetPath, BioGrid, DIP, BIND, KEGG, HPRD, CellCircuits, and NCBI GEO, as well as five different “expert systems” focused on pathway analysis (NetPath, Protein Lounge, Molecular Systems Biology, Biocarta, Science's STKE). This approach identified a set of genes for which the encoded proteins either directly bound EGFR, EGFR-family members such as ERBB2 and ERBB3, and their immediate downstream effectors, or were purified in complexes including these proteins; a set of genes transcriptionally upregulated by EGFR family stimulation and downregulated by EGFR pathway inhibition; and a set of genes otherwise involved in EGFR signaling based on published literature. We also incorporated data generated from genetic interactions reported in Drosophila, C. elegans, and other organisms for strongly conserved evolutionary orthologs of genes in this pathway (11, 12). Using resources in the systems analysis programs Cytoscape and Osprey, we then identified a core high value set of genes that fell into at least two of these linkage categories, and based on other weighting functions. FIG. 1 schematically represents the source for each of the genes in the final custom library.

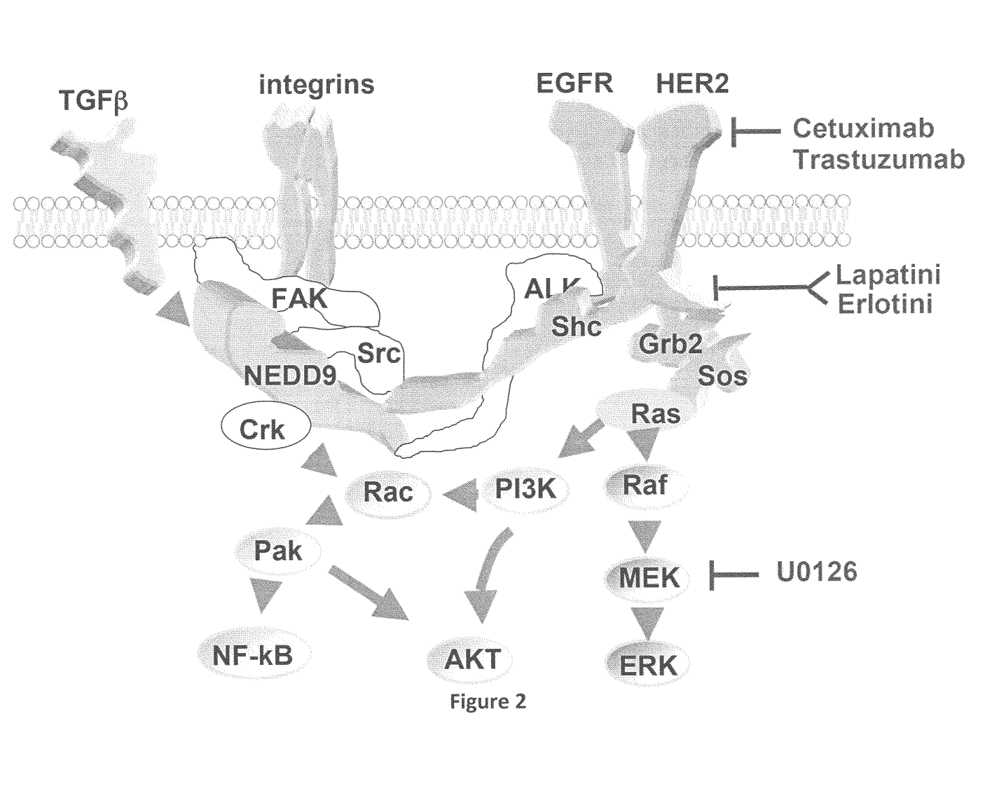

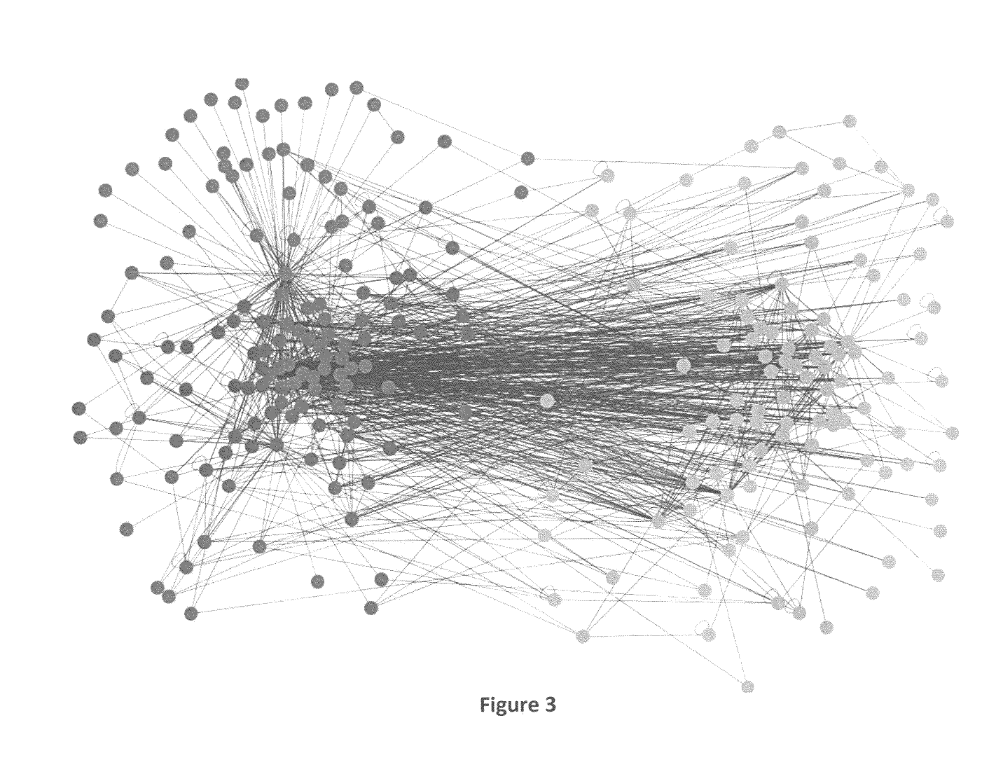

Drug resistance and propensity to metastasis commonly both characterize the most aggressive tumors. A growing number of proteins that function in pro-metastatic signaling pathways related to cellular invasiveness are also known to function in promoting apoptosis resistance (via modulation of integrin- and cadherin-dependent signaling cascades). To enrich our candidate siRNA set, we then performed a similar analysis for genes physically and functionally linked to TGFβ, Src and NEDD9. It is becoming well established that cancer metastasis requires parallel inputs from EGFR superfamily members, and the Src and TGFβ signaling pathways (13, 14). NEDD9 (also known as HEF1) has long been studied in the Golemis laboratory (15, 16), and elevated expression of NEDD9 has recently been shown to be a critical driver of melanoma metastasis, and linked to metastasis in lung cancers (17, 18); moreover, NEDD9 physically interacts with multiple components of the EGFR/Ras, TGFβ, and Src signaling pathways (19, 20). FIGS. 2 and 3 show an example of the degree of functional overlap between EGFR family and TGF-β/NEDD9 signaling networks.

From these combined data mining efforts, we selected a set of 638 target genes (see Table 1). These genes were ordered as a custom library from Qiagen. Two independent siRNAs for each gene were pooled and arrayed in wells. For the screening experiment, aliquots of the library were transferred to the inner 60 wells of 11 assay plates with a layout intended to seed in negative control siRNA (siControl targeting insect luciferase transcript, Dharmacon), positive control cytotoxic siRNA (targeting Polo-like kinase1), and cytotoxic drug (0.7 mM campthotecin). The siRNA was mixed with the transfection reagent (Dharmafect 1, Dharmacon) diluted 1:100 in Hank's balanced saline in a total volume of 22 ml to produce 25 nM final concentration of each siRNA oligo. The mixture was incubated for 45 minutes. A reverse transfection protocol was used (9) where cells resuspended in DMEM/1% FBS/Glutamine were added at 4000 per well to the assay plates using a Thermo Multidrop bulk reagent dispenser. After overnight incubation, 10 ml per well of the drug was added. For each screening experiment, the effective inhibitory concentration (IC) of the screen drug was predetermined by rigorous IC50 testing, and screens were consistently performed aiming for IC30-40 range. Viability was measured in the Perkin Elmer Envision plate reader using fluorescent metabolite of Alamar blue (CellTiterBlue™, Promega).

### Screening Conditions: Initial Selection and Optimization.

Our initial screens were performed in A431 cells, for two compelling reasons. First, we wanted to maximize our chances of obtaining any hits that would sensitize to EGFR-targeting antibodies. A431 cells are well known to over-express the EGFR receptor, and to be exquisitely sensitive to inhibition of EGFR-dependent signaling. Second, as outlined below, we have had the opportunity to add value to our screening strategy based on addition of a high throughput microscope-based detection system: based on their regular growth properties, A431 cells have proven excellent for this purpose.

As a second conservative approach, initial EGFR-pathway sensitization screens were performed with panitumumab, and also with the small molecule erlotinib. This was done because of literature suggesting that a significant component of the anti-tumor effect of EGFR-targeting antibodies arises from cell-mediated immunity, which would not apply in cultured cell lines. This restriction should not apply with erlotinib. Because of the relatively low cost of screening, this project also serves as an opportunity to probe the overall “resistance structure” of the EGFR signaling pathway, by examining the overlapping pattern of “hits” arising from drugs targeting different points along the pathway. Hence, we have now screened A431 cells for siRNA sensitizers to erlotinib, panitumumab, and U0126 (targets MEK1), in each case normalizing against the base line inhibitory profile of each siRNA in cells treated with a DMSO (vehicle) control. We have also screened the A431 cell line with CPT11 (an active camptothecin analog of irenotecan). We anticipated that comparison of the CPT11 hit pattern with the EGFR-Ras-MEK1 inhibitor pattern would segment siRNAs into 3 classes: those that sensitize to CPT11 and EGFR-Ras-MEK1 inhibitors, targeting general apoptosis-resistance genes; and those specific either to CPT11 or the EGFR-Ras-MEK1 pathway. Knowledge of each these classes has specific value for specific therapeutic/biomarker applications.

### Library Screening: Additional Cell Line Models, Strategy.

A growing consensus among clinicians is that a major source of resistance to EGFR-targeted therapies is the presence of an activating K-Ras mutation (present in up to 70% of resistant tumors) or an activating B-Raf mutation (present in up to 10% of resistant tumors). The colon cancer cell lines HCT116 harbors an activating K-Ras mutation, conferring relative resistance to the EGFR antagonists in vitro. The HCT116 colorectal carcinoma cell line offers advantage of comparison between p53null and p53-positive (intact apoptotic checkpoint) isogenic variants (9) which will be important to determine the p53-dependancy of the synthetic lethal phenotype and the mechanism of apoptosis induction. Conditions for these cell lines have been established for siRNA-based screening with high siRNA transfection efficiency (>75% depletion of two housekeeping genes GADPH, PLK1); and consistency in Alamar Blue viability yielding robust Z′-scores with control drugs (see Results below). Additional cell lines with activating mutations in B-Raf will also be worked up for hit validation.

We are using these additional cell lines in two ways. For HCT116, we will repeat interactome library screens essentially as performed in the A431 cell line, using DMSO (vehicle), erlotinib, and CPT11. We will determine whether the overall landscape of hits is similar, or distinct. We anticipate that CPT11 may yield a similar profile in the two lines, while erlotinib may yield a different, extremely restricted, hit map. Hits arising from this screen that also were detected in the A431 line would obviously be of particular interest. Second, for selected, validated hits in the A431 cell line that are of specific interest based on the validation steps described below, we will selectively analyze these hits in HCT116 positive and negative for p53, and in LoVo cells. A summary of our complete set of screens is shown in FIG. 4.

### Statistics for Preliminary Selection of Positives.

Positive candidates from the first 5 screens were selected based on standard approaches described in detail in (9) and Swanton et al. (9, 21). Specifically, the Alamar blue fluorescence intensity in the assay wells (R) read after 2 hours of incubation was normalized to the mean siControl (C) on each plate (R/C). The effect of drug treatment on viability was measured based on the normalized viabilities in the drug treated and vehicle wells using Limma (22), Limma borrows strength across genes based on an empirical Bayes approach and identifies statistically significant changes in viability by combining information from a set of gene-specific tests. We utilized the Limma implementation in the Open Source R/Bioconductor Package (available on the world wide web at bioconductor.org) (23), Hits were identified based on statistical significance as well as biological significance. Statistical significance was measured by p-values controlled for the false discovery rate (FDR) using the Benjamini-Hochberg step-up method (24) to account for multiple testing. Hits showing an FDR of less than the desired cut-off were considered statistically significant. The choice of the cut-off itself was flexible. Biological significance was measured by a change of at least 20% in viability relative to vehicle treated cells. Hits identified from each of the above filters were combined and a list of common hits showing greater statistical and biological significance (lower FDR and at least 20% change in viability) were identified.

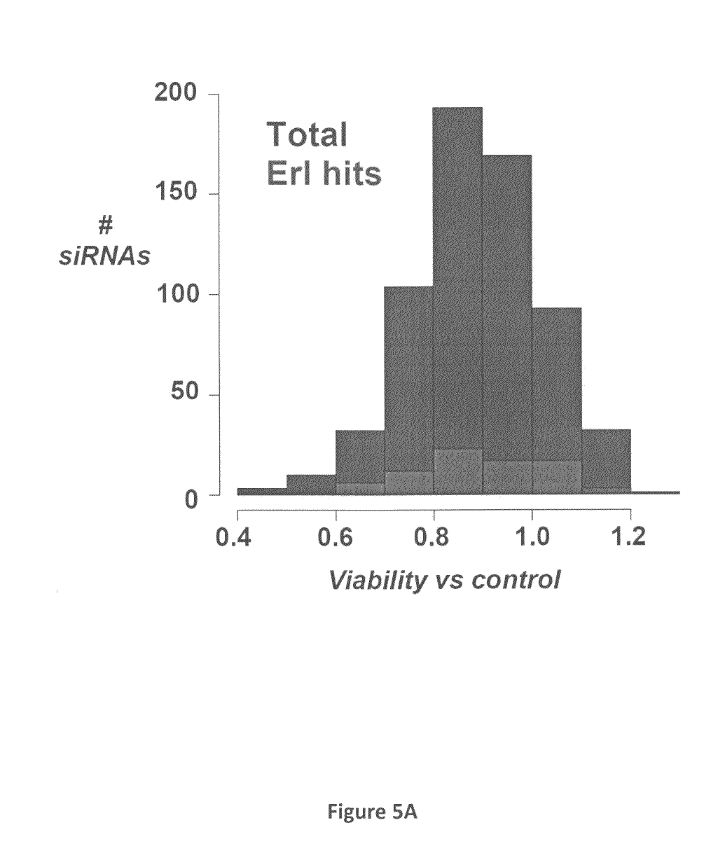

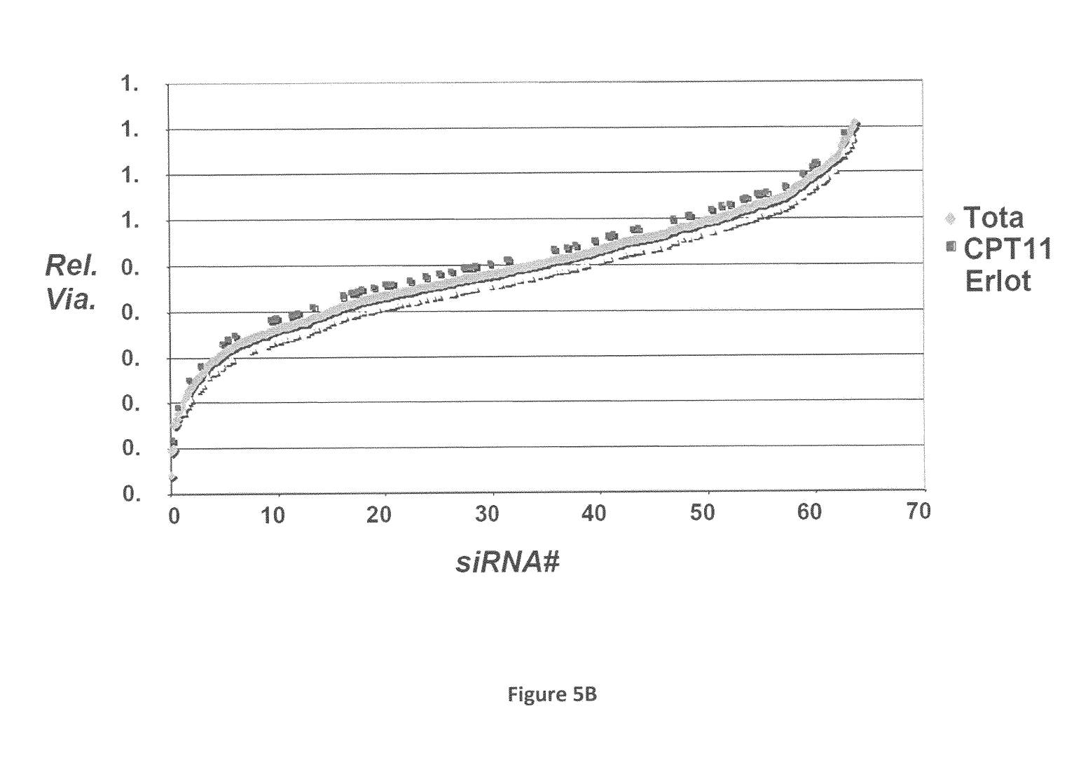

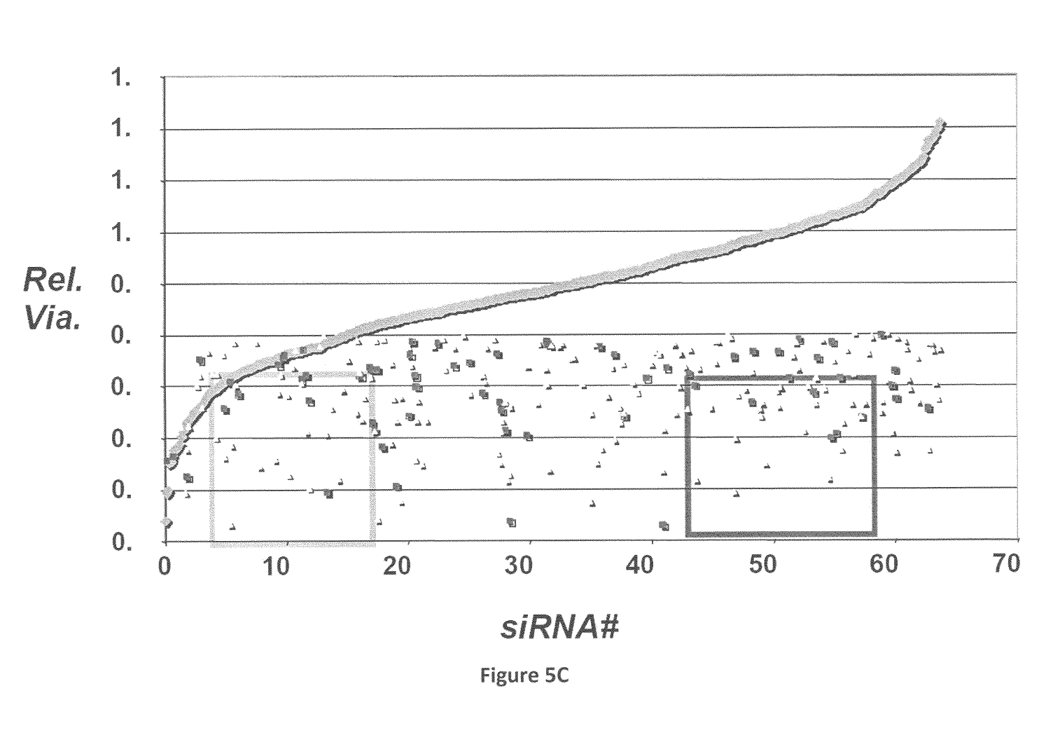

An initial question working with a custom library is whether a custom panel of siRNAs pre-selected to be relevant to EGFR signaling might contain many siRNAs that strongly inhibit cell growth even in the absence of drug treatment. FIG. 5 plots the baseline degree of growth inhibition for each of the siRNAs used in A431 cells treated with DMSO vehicle. From 638 genes, only 145 reduced cell growth more than 20%, 44 more than 30%, and 13 more than 40% (FIG. 5A). This profile contrasted favorably with results observed in the high throughput screening facility with other custom siRNA libraries such as a “cell cycle” library, in which a high percentage of the siRNAs significantly inhibited cell growth, and suggested that inhibition of the EGFR interactome siRNA targets did not induce broad cell cycle arrest or cell death absent drug treatment.

An important fundamental question in relationship of the likelihood of identifying drug sensitizing siRNAs that are useful in predicting a useful clinical strategy is, do the sensitizing siRNAs selectively emerge from the part of the library that intrinsically inhibits cell growth? Or in other words, does sensitization simply reflect the idea that an unhealthy cell is more likely to succumb to drug treatment, or are there specific siRNA-drug interactions? We compared the distribution of siRNAs selected as hits for erlotinib (FIGS. 5A, 5B) or CPT11 (FIG. 5B) versus the ability of the siRNA to reduce cell viability in cells treated only with DMSO (blue). FIG. 5A shows that sensitizing hits were found in bins containing siRNAs intrinsically inducing a 0.6-1.2 relative viability versus control-treated cells. FIG. 5B, which graphically represents all 638 siRNAs aligned from greatest inhibitory effect (left) to least (right) based on behavior with DMSO (blue line): CPT11 (red) and erlotinib (gold) hits are evenly distributed along the gradient. Importantly, FIG. 5C redraws FIG. 5B to indicate the degree of sensitization obtained with each siRNA. Strong hits (indicating those selectively reducing viability to 0.7 or less in the presence of drug versus DMSO) were found among siRNAs that had intrinsic activity in reducing cell growth (e.g., green box) and among those that had no such activity (purple box). siRNAs in the purple box particularly interesting for therapeutic development, as they possess no intrinsic toxicity, and exhibit specific dependence on drug treatment for efficacy.

### Microscopy-Based Analysis:

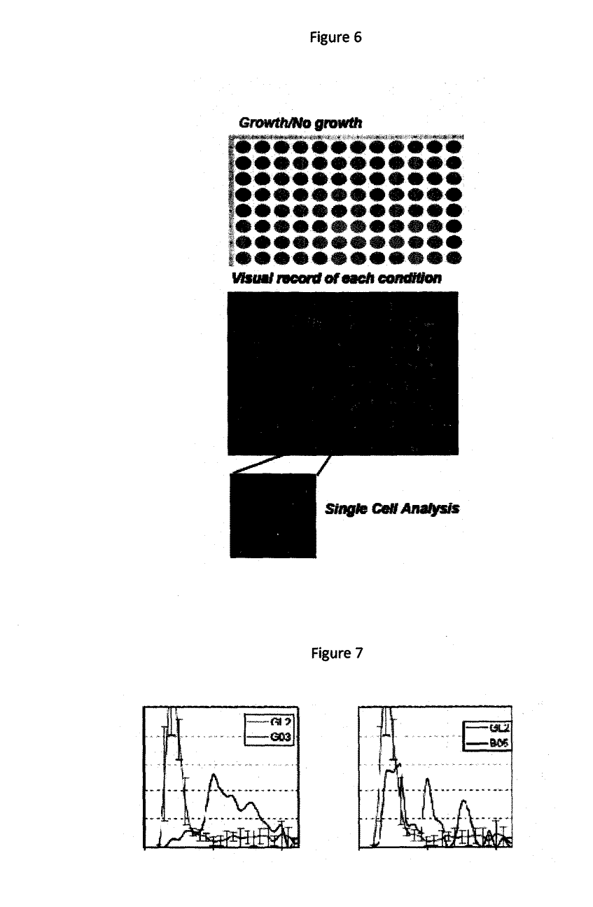

In addition to possessing high throughput robotics supporting a platereader, we also employed a Molecular Devices ImageXpress automated microscope. The ImageXpress captures 1-2 fields, representing 100-200 cells, for each individual well of a 96 well microtiter plate (see FIG. 6); time required to obtain readout from 22 plates in a single screen is typically 2-3 hours. The Acuity software used in association with this system is a sophisticated capture/analysis program for the acquired images; in addition, the system exports specified information classes in Excel format, for application of additional analytic approaches. This capacity allows us to record changes in cell spreading and morphology, or staining with specific indicator dyes and antibodies, as additional primary parameters indicating physiological response to an siRNA/drug combination. We have used calcein labeling to paint metabolically active, viable cells, and Hoechst to stain the DNA of all cells. It is clear that a subset of the siRNAs are inducing striking differences in calcein/Hoechst staining suggestive of specific biological responses to drug treatment (e.g., cytokinetic failure; see FIG. 7), yielding a potential hit list that is non-equivalent to the platereader-based hit list. Further analysis of this data is discussed below.

### Initial Hits.

Table 2 summarizes the set of hits obtained to date based on screening the library with erlotinib, panitumumab, U0126, and CPT11, so far extracted solely from plate reader data. In each case the SI (sensitization index) value reflects reduction of Alamar blue signal in relation to the same siRNA used in combination with DMSO/vehicle. Taking as initial threshold for hit selection reduction of signal 20% below vehicle, 145 hits (representing a hit rate of 23% of the total library) were obtained with erlotinib, 19 (3%) with panitumumab, 55 (9%) with CPT11, and 7 (1%) with U0126.

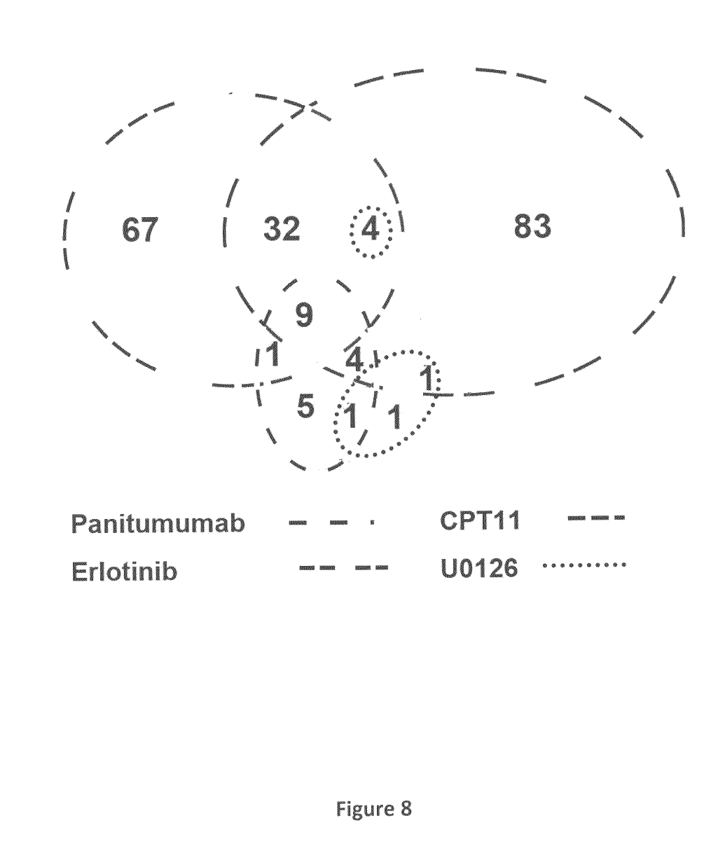

Color-coding indicates a subset of hits that are identified with more than one drug treatment (Table 1; also see Venn Diagram, FIG. 8). In particular, the three classes of hits predicted—common for CPT11 and EGFR-pathway targeting, or specific to each class—were obtained. Hits highlighted in pink were found in common with erlotinib, panitumumab, and CPT11, and are taken to represent general survival factors. Hits highlighted in green were specific to CPT11, while hits highlighted in purple were specific for erlotinib and/or panitumumab. While U0126 yielded many fewer hits overall, all of the hits identified were found to overlap either with the general survival factor group, or the erlotinib/panitumumab group.

### Analysis and Conclusions.

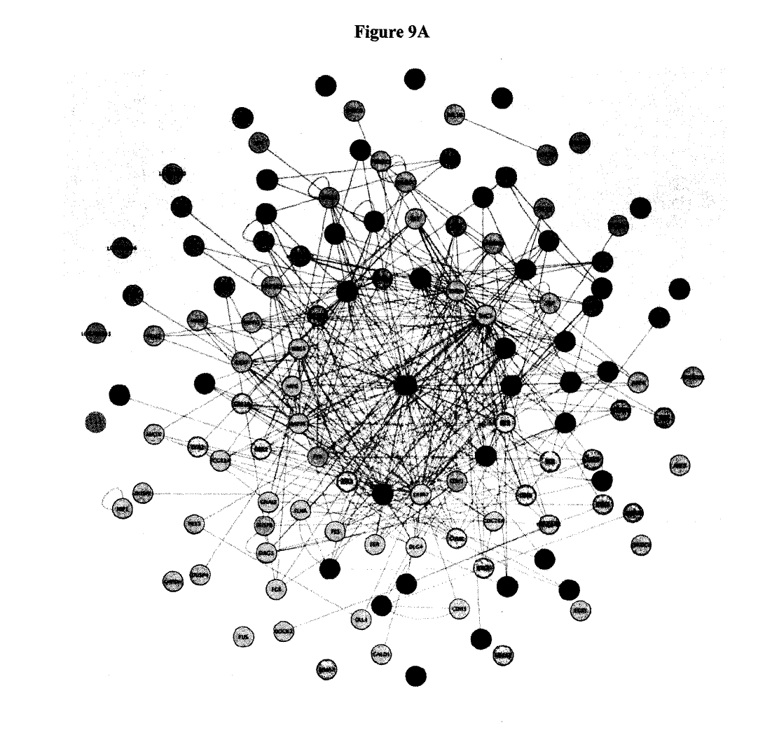

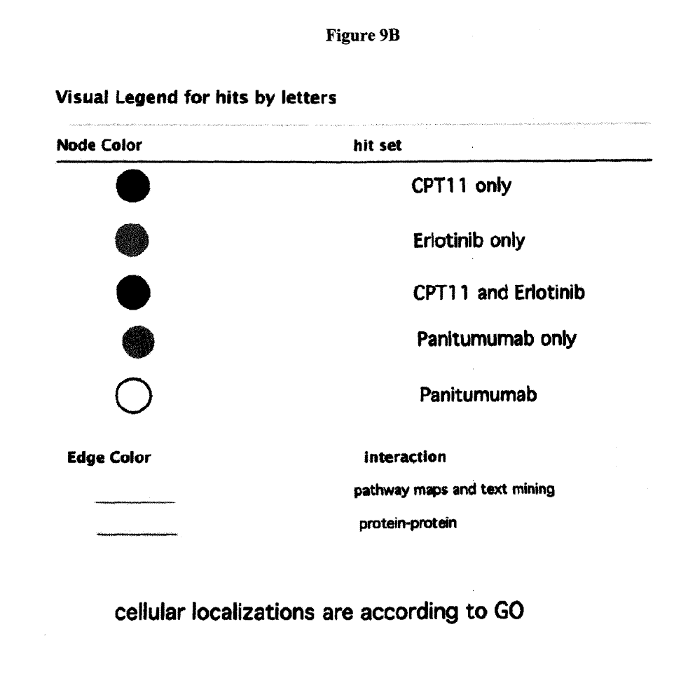

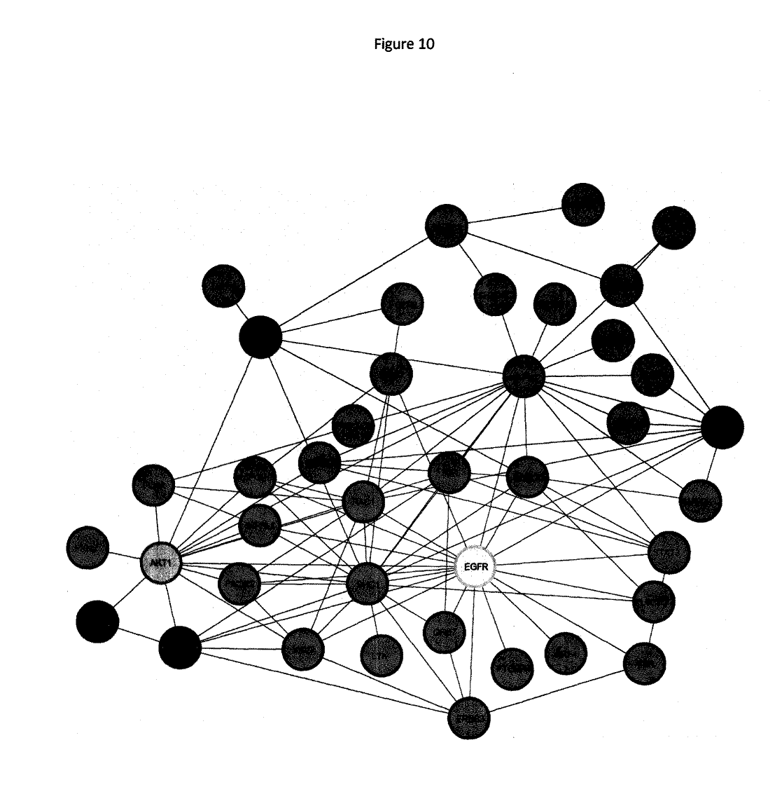

We will also perform additional validation experiments described below. However, preliminary consideration of the hit profile obtained to date is suggestive in a number of ways that indicate the clinical relevance of this data. First, erlotinib and panitumumab yielded a large number of overlapping hits. This seems highly unlikely to be a random occurrence. Second, we have used the Cytoscape resource to map the profile of interactions among the screening hits (FIG. 9). A number of the hits that have emerged physically interact with each other, or are known to act in common signaling cascades. As one provocative example, ALK, BCAR1, BCAR3, and CXCL 12 are common components of an integrin-Iinked general survival pathway (25-27); siRNAs for these genes were identified as strong hits for both erlotinib and CPT11. Third, some siRNAs emerging as strong hits have been previously implicated as highly relevant to EGFR-dependent cell signaling, based mechanistic analyses performed in cell culture, and in some cases demonstrations of resistance factors in tumors. Examples of these include ERBB3 (28,29), PLCG2, and specific protein kinase C family members (30).

We have also identified a set of siRNAs that induce resistance to erlotinib, panitumumab, and CPT11. These include siRNAs that have little or no effect on the viability of cells treated solely with vehicle. However, these siRNAs reduce the ability of panitumumab and/or other agents to decrease cell viability. It appears such genes may target suicide pathways specifically triggered by drug treatment, and as such, these genes may also be valuable for exploitation to improve therapy. Further study of these genes will also inform our understanding of drug resistance mechanisms.

### Example 2

### Screening Additional Cell Lines for Altered Sensitivity

There is an emerging consensus that a significant degree of the resistance to EGFR-targeting agents is due to activating mutations in K-Ras or B-Raf. Because of the central role of Ras in modulating apoptosis, these mutations may similarly confer resistance to agents such as irenotecan. Following screening and hit validation in the A431 cell line we will perform two classes of screen in colorectal cancer cell lines with mutated K-Ras (HCT116, LoVo) or B-Raf (e.g, WiDr, TCO. First, we will use the same regimen of comparing DMSO, erlotinib, and CPT11 screens as described above for A431 cells, but instead use HCT116 cells to identify a validated sensitization network. Second, we will take all validated hits from the A431 and HCT116 screens, and test them for sensitization in the LoVo and WiDr cell lines.

A relatively small subset of A431-predicted sensitizing siRNAs will be active in conjunction with erlotinib in the resistant cell lines. Among these, siRNAs identified as broadly sensitizing (to erlotinib as well as CPT11), are likely to maintain activity. A higher percentage of the siRNAs identified as sensitizing solely to CPT11 would maintain activity. It also appears that siRNAs targeting genes “upstream” of KRas would be less likely to function in K-Ras mutated lines than siRNAs targeting genes “downstream”, or in independent signaling pathways.

Sensitizing siRNAs will be identified for erlotinib and/or CPT11, but they will be very different from those identified with A431 cells. This outcome would initially suggest that some mRNAs for sensitizers well expressed in A431 cells are not present in HCT116 and other model cell lines, and vice versa. This could be immediately tested with qRT-PCR; if so, sensitization strategies will be tailored based on cell lineage. Regardless of outcome, the results of these experiments will facilitate identification of the factors controlling resistance as a factor of drug resistance network function.

### Example 3

### Identification of the Network Structure of the Validated Hits

The sensitization network construction shown in FIG. 8 will be refined and expanded. We will create a “live”, interactive resource, with each hit portrayed as a clickable “node” linked back to full information for the gene, and each interaction among hits as a clickable “edge” linked to full information describing the validation of the interaction. We will systematically compare the networks in the sensitive versus resistant cell line. We will compare the properties of the networks conferring resistance to EGFR-targeting agents, CPT11, or both. We will compare the networks of genes sensitizing to erlotinib, panitumumab, and U0126, to gain insight into the epistatic interactions regulating cell survival following inhibition at different stages of the EGFR-Ras-Raf-MEK1 signaling pathway.

### Example 4

### Identification of “Complementation Groups” that can be Targeted in Parallel to Achieve Super-Sensitization

Complementation groups define sets of genes whose protein products work in a single pathway or multi-protein complex, providing a single chain of input into a biological endpoint. In synthetic lethal analysis, targeting two members of the same complementation group will not enhance the endpoint phenotype, but targeting two members of different complementation groups, which provide parallel input into the biological endpoint, will enhance the final phenotype. The network mapping analysis described above has the potential to identify important “sensitization complementation groups”, which we define as small clusters of proteins known to physically interact with each other, or act in proximity on a sub-pathway: the BCAR1-BCAR3-CXCL 12-ALK cluster would define one such group. After segmentation of the hit network into proposed complementation groups, we would determine the consequences for sensitization of simultaneously targeting two siRNAs within the same group (e.g., BCAR1 and BCAR3) versus different groups (e.g., BCAR1 and BCL3). We will thus identify synergistic interactions that can greatly sensitize cells to the effect of treatment with EGFR-targeting agents, CPT11, or potentially both.

### Example 5

### Extrapolation from siRNA-Based Sensitization to Drug-Based Sensitization

The set of genes included in the EGFR interactome includes many plasma membrane associated receptors and kinases that had already drawn clinical interest, and for which small molecule and/or antibody inhibitory agents already exist. Some of these inhibitory agents have already passed through Phase I/II trials, and are being effectively used in the clinic. First, we will test whether combination of the siRNA-matched drug with erlotinib (and panitumumab, or CPT11, as appropriate) produces a notable synergistic effect using Alamar blue assay to detect reduced viability in A431 and HCT116 cells. We will determine whether the erlotinib-drug combination has a similar mode of action (see Example 6) as the erlotinib-siRNA combination. We will use the A431 and/or HCT116 cell lines to establish xenografts in nude mice, and erlotinib-drug combination in vivo synergy. This analysis provides the means to rapidly identify valuable synergies that would be immediately translatable to the clinic.

### Example 6

### Elucidation of the Mode of Action of Sensitizer siRNAs

We will first assess if siRNAs directly affect the expression, activation, or localization of the EGFR receptor itself. We will use antibodies to EGFR and phospho(active) EGFR in Western analysis of cell lysates treated with each siRNA, to look for siRNA-dependent loss of signal. We will use antibody to EGFR and the early endosomal marker EEA 1 in immunofluorescence experiments to determine whether siRNAs induce increased internalization of EGFR from the cell surface. As part of microscope-based analysis, we will also determine whether siRNAs specifically alter the morphology (attachment; cytoskeletal integrity) of drug-treated cells. Reciprocally, we will also determine whether EGFR signaling regulates the genes targeted by the sensitizing siRNAs. We will use qRT-PCR to determine whether treatment of quiescent cells with EGFR stimulates expression of the sensitizing genes, and whether treatment of actively growing cells with panitumumab or erlotinib influences expression of these genes. We will also use FACS and/or Guava analysis to measure whether specific siRNAs confer cell cycle arrest and/or apoptosis.

### Example 7

### Microscope-Based Analysis of Cells Following Identification of Hits Detected by Reduced Alamar Blue Signal

A complete database of images of calcein- and Hoechst-stained cells from experiments performed in exact parallel with the Alamar blue values (FIGS. 5 and 6) is being assembled. As noted above, even cursory analysis has indicated that some of the siRNAs are producing unusual patterns suggestive of cytokinetic blocks, early stages of apoptosis, unusual cell morphology, etc, and that some of these hits are nonequivalent to those identified by searching for loss of Alamar blue signal. We will extend this analysis in several ways. First, we will run a series of controls (drug treatments, siRNA treatment) known to induce the specific biological endpoints (cytokinetic block, etc) that we believe are suggested by the data, and compare profiles. We will analyze overall cell population properties using the Acuity software associated with the high throughput microscope, and also use customized analysis developed together with the FCCC Biostatistical facility to quantitate the appearance of sub-groups within the larger profiles.

The congruence of specific endpoint phenotypes (cytokinetic block; reduction in cell spreading; etc) with the overall Alamar blue hit list can be established. We will also validate the hits by a similar strategy to that used above for Alamar blue hits, i.e., regenerating the phenotypes with independent siRNAs, and confirming that degree of mRNA depletion of target correlates with degree of induction of the phenotype. This analysis is extremely likely to result in the addition of new siRNAs to the hit list, ie., siRNAs that induce phenotypes that are ultimately likely to result in cell death, but which have sufficiently delayed action that moribund cells did not score as positive using Alamar blue-based cutoff values.

Next, we will repeat the network construction analysis described above (Examples 3 and 4). This will be done in two ways: by analyzing the expanded network (Alamar blue hits+microscope hits), and by analyzing networks associated with specific phenotypic endpoints. This can extend definition of sensitization clusters/complementation groups, and may bring more druggable (or already drugged) targets into the groups. It will also illuminate the mechanism by which sensitization is induced. As a hypothetical example, one cluster of closely interacting proteins includes some with strong Alamar blue sensitization to erlotinib, and several members that induce loss of cell area only in cells treated with erlotinib. This might imply the entire group is functionally antagonizing the integrin-dependent cell adhesion machinery, and that antibodies generally antagonizing this process might be of interest for exploring experimental synergies with erlotinib.

### Example 8

### Treatment of Additional Cancer Types in the Clinic with EGFR-Targeting Agents and/or Irenotecan

Besides colorectal cancer, lung cancers, head and neck cancers, and a number of other types of cancer respond to EGFR-targeting agents and/or irenotecan. However, because of differences in cell lineage, these cells will not express an identical complement of proteins as colorectal tumors, implying that their cell signaling/cell survival networks will be non-equivalent. Hence, a subset of the siRNAs that sensitize colorectal tumor cells to EGFR-targeting agents in colorectal cells may not be active in other tumor types, while additional sensitizing siRNAs may be detected in screens of these tumors. We will focus on lung cancer cell lines as a first counter-model to compare with A431 and HCT116 data, and essentially parallel the three Validation Steps outlined above. For the collection of validated hits from A431 and HCT116 screening, we will first use qRT-PCR to determine if the mRNAs are expressed in two erlotinib-sensitive and two K-Ras-mutated, erlotinib resistant lung cancer cell lines. We will then determine what percent of the expressed siRNAs are sensitizing in the lung cancer cell lines. If a significant percentage of A431/HCT116 hits maintain function in lung cancer cell lines, such hits are relevant for improved chemotherapy, particularly if they include siRNAs depleting proteins with existing clinical agents (as discussed in Example 5). If only a very limited number of hits are found to be functional, we will instead repeat the primary screens outlined above, to define the sensitization network of lung cancer tumors. We note this last experiment is of interest in its own right as an inquiry into the basic properties of alternative network construction.

### Example 9

### Identification of Additional Drugs which Show Promising Experimental Synergies with EGFR-Targeting Agents

Although preclinical and clinical analyses are empirically establishing useful synergies between erlotinib or EGFR-targeting antibodies and drugs targeting the mTOR pathway, such as rapamycin or temsirolimus (Costa, 2007; Jimeno, 2007; Buck, 2006, IGF Morgillo, 2006), and other high-value targets, it is currently largely unknown as to which EGFR-dependent signaling pathways contribute to the sensitization. Using the EGFR interactome library, we can rapidly establish this point, using a strategy similar to that outlined for primary screening above to look for a network of sensitizers to selected drugs in the A431 and HCT116 colorectal cancer cell lines. This screen should identify a number of the hits already identified as common sensitizers to erlotinib, panitumumab, and CPT11, but may also identify a sub-network (sensitization complementation group) of hits that is specific to inhibition by the new test drug.

### Example 10

### Preferred Combinations for the Treatment of Different Cancer Types

The table below provides 16 genes and combinations of agents which should have efficacy for the treatment of the indicated cancer types. Anti-EGFr drugs (cetuximab, an anti-EGFR antibody; erlotinib, lapatinib or any tyrosine kinase inhibitor specific for the EGFR kinase) can be combined with either existing or potentially available inhibitors of the 16 targets presented. In general, cetuximab is used in colorectal, lung, head and neck (HNSCC). Erlotinib is used in lung, pancreas. Potentially, cetuximab or any other anti-EGFR antibody can be approved for lung cancer (NSCLC), and cancer of the pancreas. Lapatinib is preferred for combination treatment of breast, and ovarian cancers. The gene target activity may be inhibited using small interfering molecules, agents already known to inhibit their function, (e.g., commercially available and clinically safe phosphatase and kinase inhibitors or the small siRNAs interfering molecules described herein.

TABLE A

| | | | HNSCC | | | | | Other cancers |
|---|---|---|---|---|---|---|---|---|
| | | Colorectal | cetuximab | | Pancreas | | | cetuximab, |
| | | cancer | or | Lung | erlotinib or | Glioma | Breast | erlotinib, |
| Gene Target | Class of inhibitor | #cetuximab | erlotinib | erlotinib | cetuximab | erlotinib | lapatinib | lapatinib |
| ANXA6 | Interfering small | 1* | 1 | 1 | | 1 | 1 | |
| | molecule | | | | | | | |
| ARF4; ARF5 | Ribosyltransferase | 1 | 1 | 1 | 1 | 1 | 1 | |
| | inhibitor | | | | | | | |
| ASCL2 | Transcription factor | 1 | 1 | 1 | 1 | 1 | 1 | Important in |
| | inhibitor | | | | | | | ovarian cancer |
| CD59 | Antibody to CD59 | 0 | 1 | 2 | 2 | 0 | 1 | |
| DIXDC1 | Interferring small | 1 | 1 | 1 | 1 | 1 | 1 | |
| | molecule | | | | | | | |
| DUSP4 | Phosphatase inhibitor | 1 | 1 | 1 | 1 | 1 | 1 | |
| DUSP6 | Phosphatase inhibitor | 2 | 1 | 1 | 1 | 1 | 1 | |
| DUSP7 | Phosphatase inhibitor | 1 | 1 | 1 | 1 | 1 | 1 | |
| FER | Kinase inhibitor | 2 | 1 | 1 | 1 | 1 | 1 | |
| MATK | Kinase inhibitor | 4 | 4 | 4 | 4 | 2 | 1 | |
| NEDD9 | Interfering small | 1 | 1 | 1 | 1 | 1 | 1 | |
| | molecule | | | | | | | |
| PRIAP19/SLP1 | Interfering small | 0 | 0 | 1 | 1 | 1 | 1 | Important in |
| | molecule | | | | | | | ovarian and |
| | | | | | | | | endometrial |
| | | | | | | | | cancer |
| PRKACB | Kinase inhibitor | 2 | 3 | 2 | 2 | 4 | 1 | |
| RAPGEF1 | Small molecule | 2 | 1 | 1 | 2 | 1 | 1 | |
| | inhibitor | | | | | | | |
| SC4MOL | Cholesterol synthesis | 1 | 1 | 1 | 1 | 1 | 1 | |
| | inhibitor | | | | | | | |
| SH2DC3 | Interfering small | 1 | 1 | 1 | 1 | 1 | 1 | |
| | molecule | | | | | | | |

*numbers represent expression levels
#anti-EGFR agents (cetuximab, erlotinib, and/or lapatinib) to be used in combinations with agents downregulating gene targets listed in column 1

### Example 11

### Generation of Biomarkers that Predict Patient Response to Treatment

One of the most important uses of the information generated from this study will be as a guide to select patients likely to respond to EGFR-centered treatments. Patients with tumors expressing high levels of the mRNAs and proteins targeted by sensitization hits would prove to be resistant to therapy, while patients with low levels of these mRNAs and proteins might be excellent candidates for treatment with the synergistic combination described herein. Additionally, it will be more informative and better exploit the sensitization network that we have identified if we screen tumors for the expression of sets of sensitizing hits, rather than “cherrypicking” a small number of individual hits. Already, hits of clinical relevance have been identified. We will use these genes to generate a screening chip; alternatively, we can generate a Taqman primer set for qRT-PCR. We will then employ a set of at least 10 pre-treatment colorectal tumors from patients who responded to EGFR-targeted therapy, and a matching set of non-responder tumors, and we will systematically compare the expression of the sensitization panel.

### Example 12

As described above, we have developed a protein network centered on the highly validated target EGFR, and used siRNA screening to comparatively probe this network for proteins that regulate the effectiveness of both EGFR-targeted and chemotherapeutic agents. This approach identified sub-networks of proteins influencing resistance, with hits enriched among first order protein interactors of the network seeds. Extrapolation from the network structure led to the identification of synergy between EGFR antagonists and drugs targeting PRKC, STAT3, and AURKA, suggesting a direct path to clinical exploitation of study results. Such a focused approach has significant potential to enhance the future coherent design of combination therapies.

A robust network paradigm has critical implications for targeted cancer therapies, predicting that in cells treated with therapies inhibiting an oncogenic node, rescue signaling can be provided by modifying signaling output from any of a number of distinct proteins that are components of a web of interactions centered around the target of inhibition. This concept is reinforced by studies in model organisms demonstrating that quantitatively significant signal-modulating relationships commonly involve proteins that have closely linked functions. In this example, we describe additional regulators of resistance to EGFR-targeted therapies, which can be used to clinical advantage to overcome therapeutic resistance.

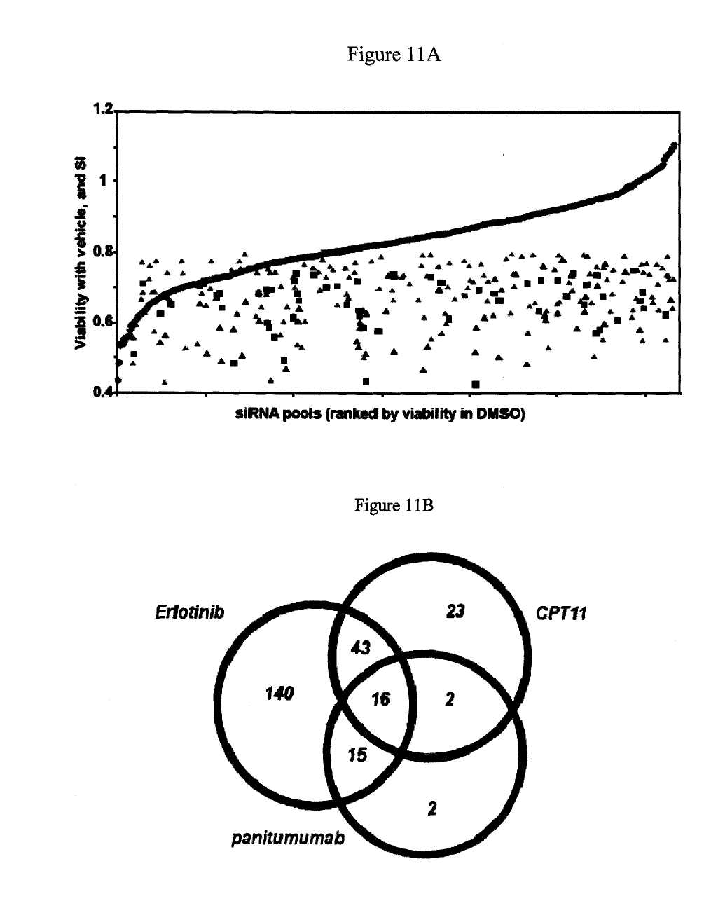

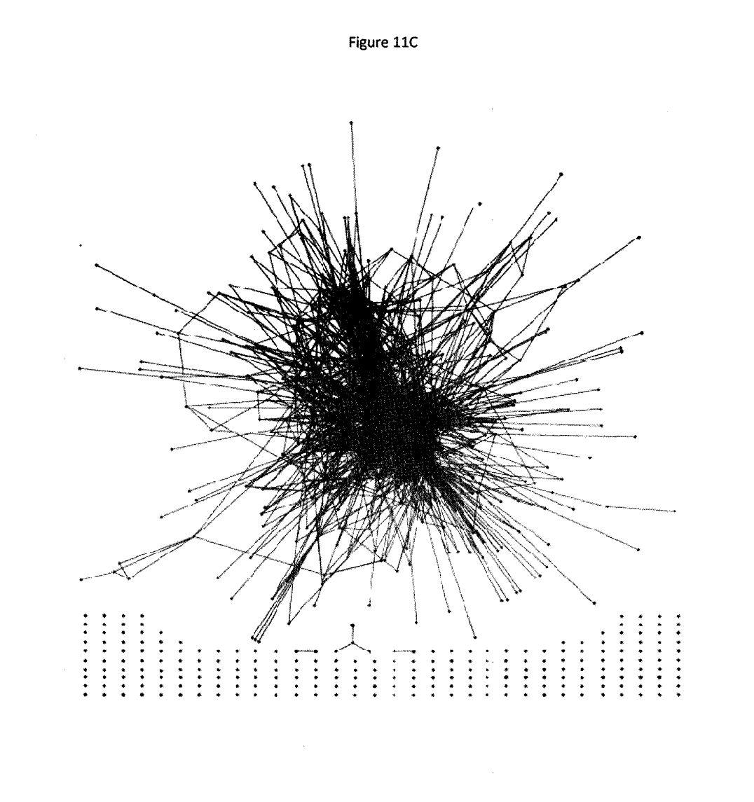

As described in Example 1, the A431 cervical adenocarcinoma cell line is highly dependent on EGFR pathway signaling. This cell line was reiteratively screened with the targeted siRNA library used in combination with DMSO (vehicle), or with EGFR-targeting small molecule and antibody inhibitors, or the non-specific cytotoxic agent camptothecin (CPT11) applied at IC₂₅-IC₃₅ concentrations. Primary hits were defined as siRNAs reducing negative control-normalized viability by at least 15% in the presence of a drug compared to DMSO (defined as the Sensitization Index (SI<0.85), with a false discovery rate (FDR)<20%. The distribution of primary hits was independent of the tendency of a siRNA to affect cell viability in the absence of drug treatment (FIG. 11A). The vast majority of hits obtained with an EGFR-targeting antibody were included within the larger set of EGFR-targeting small molecule hits (FIG. 11B). Subsequent validations confirmed a set of 61 genes (Table B) sensitizing to EGFR-targeting agents in which 2 or more of 4 independent siRNAs recapitulated sensitization to erlotinib. The majority of sensitizing hits (48/61) encoded proteins connected in a physically interacting network (FIG. 11C). The remaining 13 encoded proteins are not known to interact physically with EGFR or its direct partners, but instead are linked to EGFR based on transcriptional response to pathway manipulation.

TABLE B
Validated screen hits.

| Symbol | ID | Gene Description | Origin | | Clinical agent |
|---|---|---|---|---|---|
| ABL1 | 25 | v-abl Abelson murine leukemia viral | PPI1 | | Imatinib, dasatinib |
| | | oncogene homolog 1 | | | |
| AKT2 | 208 | v-akt murine thymoma viral oncogene | PG | | Perifosine, triciribine, |
| | | homolog 2 | | | GSK690693 |
| ANXA6 | 309 | annexin A6 | C, PPI2 | | |
| APP | 351 | amyloid beta (A4) precursor protein | PPI1 | | |
| | | (peptidase nexin-II, Alzheimer disease) | | | |
| ARF4 | 378 | ADP-ribosylation factor 4 | PM, PPI1 | | |
| ARF5 | 381 | ADP-ribosylation factor 5 | PG | | |
| ASCL2 | 430 | achaete-scute complex homolog 2 | PPI2, DG | | |
| | | (Drosophila) | | | |
| BCAR1 | 9564 | breast cancer anti-estrogen resistance 1 | PM, C, | | |
| | | | PPI1 | | |
| CALM1 | 801 | calmodulin 1 (phosphorylase kinase, | C PPI1 | | phenothiazines |
| | | delta) | | | |
| CBLC | 23624 | Cas-Br-M (murine) ecotropic retroviral | PM PPI1 | | |
| | | transforming sequence c | | | |
| CCND1 | 595 | cyclin D1 | PM, PPI2 | | |
| CD59 | 966 | CD59 molecule, complement regulatory | C, PPI2 | | Roche, Preclinical |
| | | protein | | | |
| CDH3 | 1001 | cadherin 3, type 1, P-cadherin (placental) | PG | | |
| CXCL12 | 6387 | chemokine (C—X—C motif) ligand 12 | MA | | |
| | | (stromal cell-derived factor 1) | | | |
| DCN | 1634 | decorin | PPI1 | | |
| DDR2 | 4921 | discoidin domain receptor family, member 2 | PPI1 | | |
| DIXDC1 | 85458 | DIX domain containing 1 | MA | | |
| DLG4 | 1742 | discs, large homolog 4 (Drosophila) | PPI1 | | |
| DUSP4 | 1846 | dual specificity phosphatase 4 | MA | | |
| DUSP6 | 1848 | dual specificity phosphatase 6 | DG | | |
| DUSP7 | 1849 | dual specificity phosphatase 7 | DG | | |
| EPHA5 | 2044 | EPH receptor A5 | PG | | |
| ERBB3 | 2065 | v-erb-b2 erythroblastic leukemia viral | PM, PPI1, | | antibodies, e.g. MM-121 |
| | | oncogene homolog 3 (avian) | DG | | |
| FER | 2241 | fer (fps/fes related) tyrosine kinase | PPI1 | | |
| | | (phosphoprotein NCP94) | | | |
| FGFR2 | 2263 | fibroblast growth factor receptor 2 | DG | | e.g. brivanib, masitinib, |
| | | (bacteria-expressed kinase, keratinocyte | | | TKI258, PHA-739358 |
| FLNA | 2316 | filamin PG, alpha (actin binding protein | C, PPI2 | | |
| | | 280) | | | |
| GRB7 | 2886 | growth factor receptor-bound protein 7 | PM, PPI1 | | |
| HSPA9 | 3313 | heat shock 70 kDa protein 9 (mortalin) | C, PPI2 | | |
| INPPL1 | 3636 | inositol polyphosphate phosphatase-like 1 | PM, PPI1 | | |
| KLF10 | 7071 | Kruppel-like factor 10 | MA | | |
| LOC284393 | 284393 | similar to ribosomal protein L10 | PG | | |
| LOC63920 | 63920 | transposon-derived Buster3 transposase- | MA | | |
| | | like | | | |
| LTK | 4058 | leukocyte tyrosine kinase | PPI1 | | |
| MAP3K1 | 4214 | mitogen-activated protein kinase kinase | PM, PPI2 | | |
| | | kinase 1 | | | |
| MAPK1 | 5594 | mitogen-activated protein kinase 1 | PM, PPI1 | | |
| MATK | 4145 | megakaryocyte-associated tyrosine | PM, PPI1 | | |
| | | kinase | | | |
| NEDD9 | 4739 | neural precursor cell expressed, | PPI1 | | |
| | | developmentally down-regulated 9 | | | |
| PIK3R1 | 5295 | phosphoinositide-3-kinase, regulatory | PM, PPI1 | | |
| | | subunit 1 (p85 alpha) | | | e.g. PX-866, BGT226, |
| PIK3R2 | 5296 | phosphoinositide-3-kinase, regulatory | PM, PPI1 | {close oversize bracket} | GDC-0941, XL765 |
| | | subunit 2 (p85 beta) | | | |
| PIN1 | 5300 | protein (peptidylprolyl cis/trans | PM | | |
| | | isomerase) NIMA-interacting 1 | | | |
| PKN2 | 5586 | protein kinase N2 | PM, PPI2 | | |
| PLSCR1 | 5359 | phospholipid scramblase 1 | PM, PPI1, | | |
| | | | MA | | |
| PPIA | 5478 | peptidylprolyl isomerase PG (cyclophilin | C, PPI2 | | |
| | | PG) | | | |
| PRKACB | 5567 | protein kinase, cAMP-dependent, | PG | | |
| | | catalytic, beta | | | |
| PRKCD | 5580 | protein kinase C, delta | PM, PPI1 | | ruboxistaurin, |
| PRKCE | 5581 | protein kinase C, epsilon | PM, PPI2 | {close oversize bracket} | enzastaurin, |
| PRKCZ | 5590 | protein kinase C, zeta | PM, PPI2 | | tamoxifen |
| RAC1 | 5879 | ras-related C3 botulinum toxin substrate 1 | PM, PPI2 | | |
| | | (rho family, small GTP binding protein | | | |
| RACGAP1 | 29127 | Rac GTPase activating protein 1 | C, PPI2 | | |
| RAPGEF1 | 2889 | Rap guanine nucleotide exchange factor | PPI1 | | |
| | | (GEF) 1 | | | |
| RASA3 | 22821 | RAS p21 protein activator 3 | DG | | |
| RET | 5979 | ret proto-oncogene | PPI1, M2 | | valdetanib |
| RPS6KA5 | 9252 | ribosomal protein S6 kinase, 90 kDa, | PM | | ruboxistaurin |
| | | polypeptide 5 | | | |
| SC4MOL | 6307 | sterol-C4-methyl oxidase-like | MA | | |
| SH2D3C | 10044 | SH2 domain containing 3C | PM, PPI1 | | |
| SHC1 | 6464 | SHC (Src homology 2 domain containing) | PM, C, PPI1, | | |
| | | transforming protein 1 | DG | | |
| SMAD2 | 4087 | SMAD family member 2 | PM, PPI1 | | peptide 144 targets |
| | | | | | TGFβ1RIII |
| SOS2 | 6655 | son of sevenless homolog 2 (Drosophila) | PM, PPI1, | | |
| | | | DG | | |
| STAT3 | 6774 | signal transducer and activator of | PM, C, | | STAT 3 decoy oligo |
| | | transcription 3 (acute-phase response | PPI1 | | |
| TBL1Y | 90665 | transducin (beta)-like 1Y-linked | DG | | |
| VAV3 | 10451 | vav 3 oncogene | PM, C, | | |
| | | | PPI1 | | |

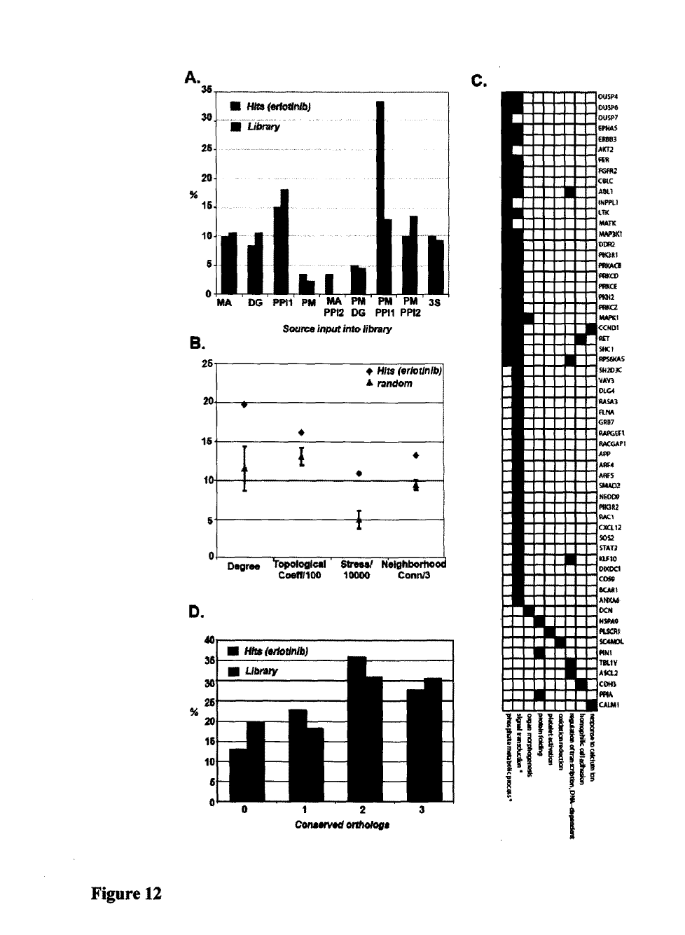

In analyzing the erlotinib-sensitizing hits in comparison to the overall properties of the 638-gene library, there was a highly significant enrichment for genes that were first order PPIs of the seeds, and were also nominated by pathway maps (FIG. 12A). The erlotinib-sensitizing hits were also significantly more likely to have topology parameters distinct from the overall network, suggesting a higher degree of connectivity for these nodes (FIG. 12B). Based on their GO function, erlotinib-sensitizing hits were enriched for proteins classified as involved in phosphate metabolism (kinases or phosphatases) and signal transduction (FIG. 12C) with p value cut-off of 0.01. Validated hits were equally likely to occur among siRNAs that independently reduced cell viability, or had little effect on cell growth in the absence of drug treatment. A weak trend was observed for hits to be evolutionarily conserved, as reflected by the presence one or more defined orthologs in lower eukaryotes than genes in the overall library (FIG. 12D).

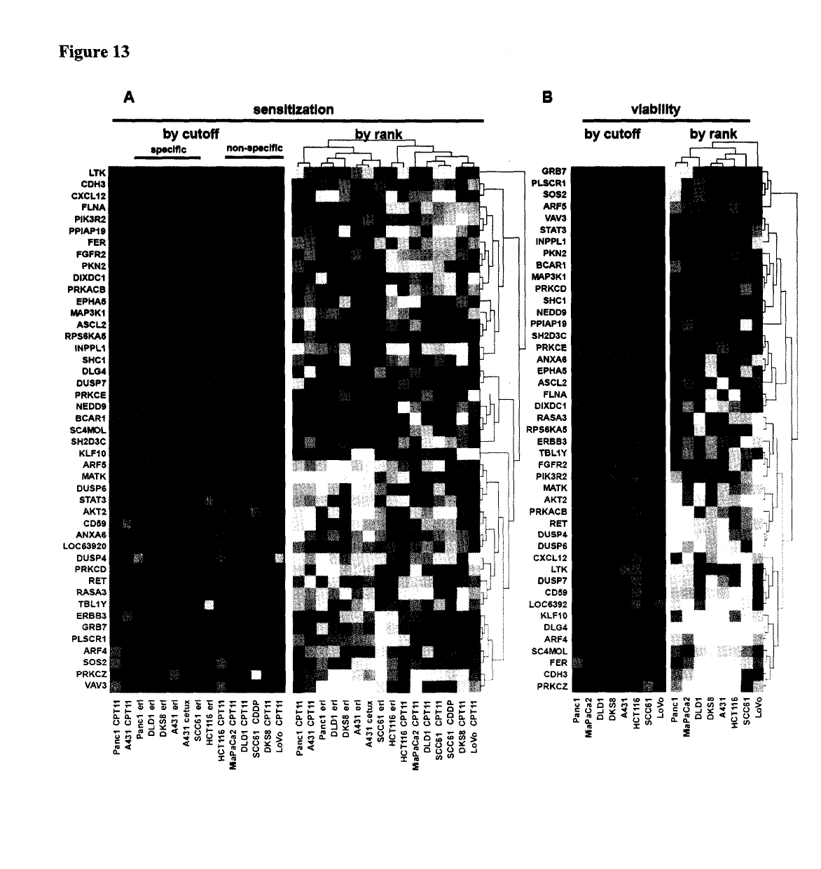

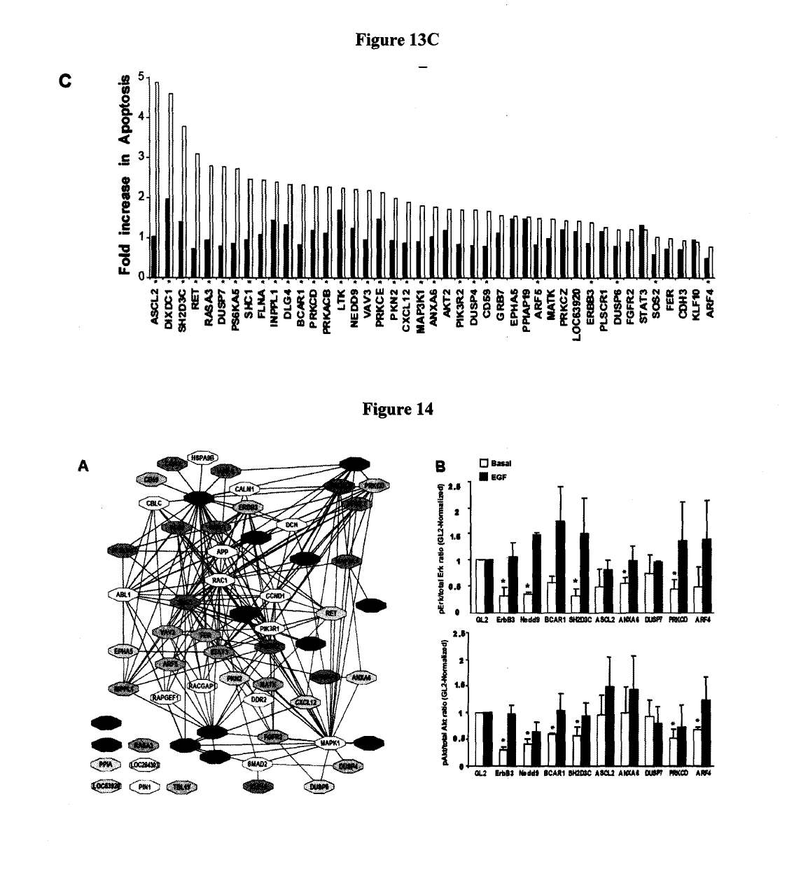

In additional experiments, we comparatively profiled the efficacy of the hit panel as sensitizers of erlotinib, cetuximab, and CPT11 across a set of cell lines, including A431, the colorectal adenocarcinoma cell lines HCT116, DLD-1, DKS-8, and LoVo, the head and neck squamous cell carcinoma cell line SCC61, and the pancreatic adenocarcinoma cell lines PANC-1 and MIA PaCa-2 (FIG. 13A). In this analysis, cell lines with intrinsic drug resistance mutations (for example, in K-Ras and/or p53) had more noise in sensitization responses, with the result that highly resistant lines (DLD1, DKS-8, LoVo, MIA PaCa-2) yielded far fewer sensitizing hits than A431 by rigorous statistical criteria. To compensate for this difference, we analyzed the data in two ways: first, by conventional threshold analysis (FIG. 13A, left), and second, by assessing the rank order of sensitization phenotype, using relaxed statistical criteria (FIG. 13A, right; see Materials and Methods). The ranking order method mitigates the stochastic “noise” common in resistant tumors, and which we observed in the erlotinib-refractory cell lines.

No gene target sensitized to erlotinib in all tested cell lines. Considering only statistically significant thresholds (FIG. 13A, left), depletion of genes initially identified and validated in A431 most consistently sensitized this cell line to erlotinib, with many in this group also sensitizing A431 cells to cetuximab. Depletion of a further small subset of these genes also consistently sensitized cells to erlotinib in at least two additional cell lines with known resistance mutations. These included SH2D3C, DUSP7, and SC4MOL. Other siRNAs (included RPS6KA5, FLNA, PRKCE, PRKACB, SC4MOL, and ASCL2) sensitized to erlotinib and/or CPT11, in 3-5 cell lines, suggesting a broader action in resistance, but less specificity for EGFR-targeting agents; this overlap may reflect the important role of some EGFR effectors in supporting general survival signaling.

Considering instead sensitization rank (FIG. 13A, right), although all genes detected based on thresholds were again detected as highly sensitizing, a broader pattern of activity was detected for some hits. For example, PRKCE is consistently one of the most sensitizing targets in 11//15 conditions assessed, although it only scored as significantly sensitizing in 6. The broader set of genes now detected as particularly sensitizing to erlotinib and cetuximab based on rank order activity in the majority of the cell lines tested included BCAR1, DUSP7, DLG4, SC4MOL, SH2D3C, and NEDD9, beyond those noted above.

As the in vivo effects of inhibiting a selected target will reflect the cumulative sum of intrinsic effect on viability and sensitizing activity, we also established the baseline intrinsic activity of the validated siRNAs in reducing cell growth in DMSO-treated cells (FIG. 13B). Down-modulation of certain genes intrinsically affected cell growth very significantly in multiple cell lines in the presence of vehicle alone. Other effective sensitizers, including DUSP7 and DLG4, exhibited only a minimal effect on viability in the absence of drug treatment. Based on the composite of intrinsic and sensitizing effects, a significant number of the hits (including PRKCE, INPPL1, SH2D3C, SHC1, STAT3, FLNA, and NEDD9) very strongly reduced the growth of multiple tumor cell lines treated with EGFR-targeting agents. 18 of the hits selectively enhanced apoptosis by 2-fold or more in erlotinib-treated versus DMSO treated A431 cells (FIG. 13C), suggesting these genes as desirable targets for cancer therapy.

These findings supported the idea that a cogently designed network focused around a core cancer target such as EGFR would provide a rich source of genes that modulate resistance to EGFR pathway-targeted agents. In general, a greater effect was seen on the core viability of cell lines containing wt versus mutant Ras, although the stronger hits were typically active in both; in contrast, it was impossible to establish a meaningful correlation between sensitization profile and Ras mutational status, suggesting that sensitizing activity occurred downstream or independently from core Ras signaling outputs. We investigated the relative interactions of the stronger hits within the overall topology of the EGFR signaling network (FIG. 14A). The majority of hits could be placed in a connected sub-network based on direct physical interactions. Notably, the analysis identified 2 separate members of the protein kinase C family as sensitizing in multiple cell lines (PRKCD, and PRKCE), with some of these proteins also directly connecting to the strong sensitizers DLG4 and PRKACB. A second cluster included SH2D3C, BCAR1, and NEDD9, which sensitized preferentially to erlotinib and cetuximab, and were all connected by direct physical interactions. Many of these most sensitizing hits were directly connected to MAPK1, PIK3R, STAT3, SHC1, and EGFR itself, supporting the idea that these proteins modulated core outputs of the central EGFR signaling pathway.

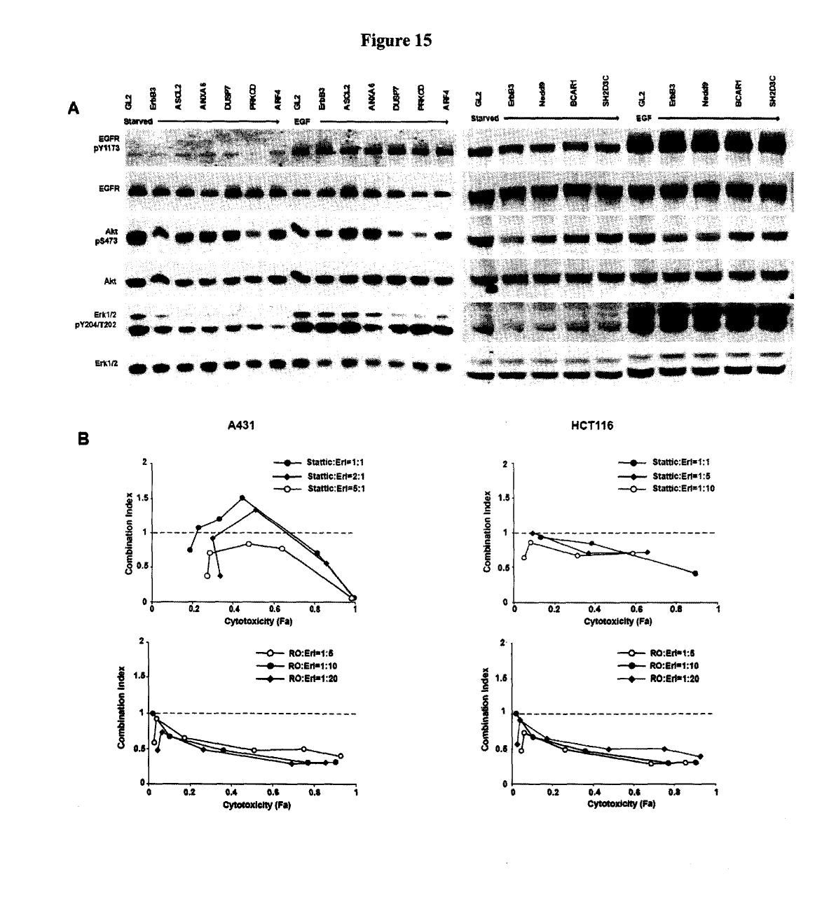

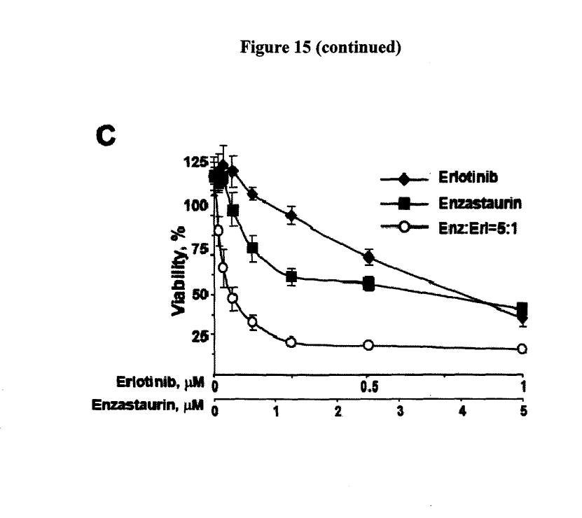

We directly tested the ability of a number of hits to directly modulate both basal and EGF-stimulated activation of the core pathway effectors MAPK1 and AKT (FIGS. 14B and 15A). The inhibition of expression of ERBB3, ANXA6, PRKCD, NEDD9, BCAR1 and SH2D3C in each case reduces basal activation of MAPK1 and AKT, implying collateral input to the canonical EGFR-MAPK-AKT pathway from these target genes. By contrast, a small number of the hits, including TBL1Y, PIN1, SC4MOL, and ASCL2, were not connected by direct protein-protein interactions to the core network, suggesting either a different mode of action, or previously undetected connections. Indeed, upon direct testing, ASCL2 affects neither MAPK1 nor AKT activation, although it potently sensitizes erlotinib-treated cells to apoptosis (FIG. 13C). Interestingly, ASCL2 is a target of Wnt signaling that is up-regulated in a subset of colon carcinomas and has very recently been shown to control the expansion of epithelial stem cells, suggesting a possible mode of activity.

A major goal of this work was to gain insights that could be rapidly translated to the clinic. Although the clinical use of RNAi is a topic of intense current research, small molecules and monoclonal antibodies remain the most broadly applicable therapy platforms. Further, given that most drugs target catalytic enzymes, whereas siRNAs typically reduce protein levels by no more than 80-90%, we hypothesized that combining small molecule inhibitors of siRNA-predicted catalytic hits with erlotinib might enhance sensitization phenotypes over those detected in initial screens. For some sensitizing hits, targeted small molecules exist, including Stattic (a small molecule inhibitor of STAT3 activation and dimerization, enzastaurin and Ro-318220 (both targeting the PRKC family, with members well-represented among the hits.

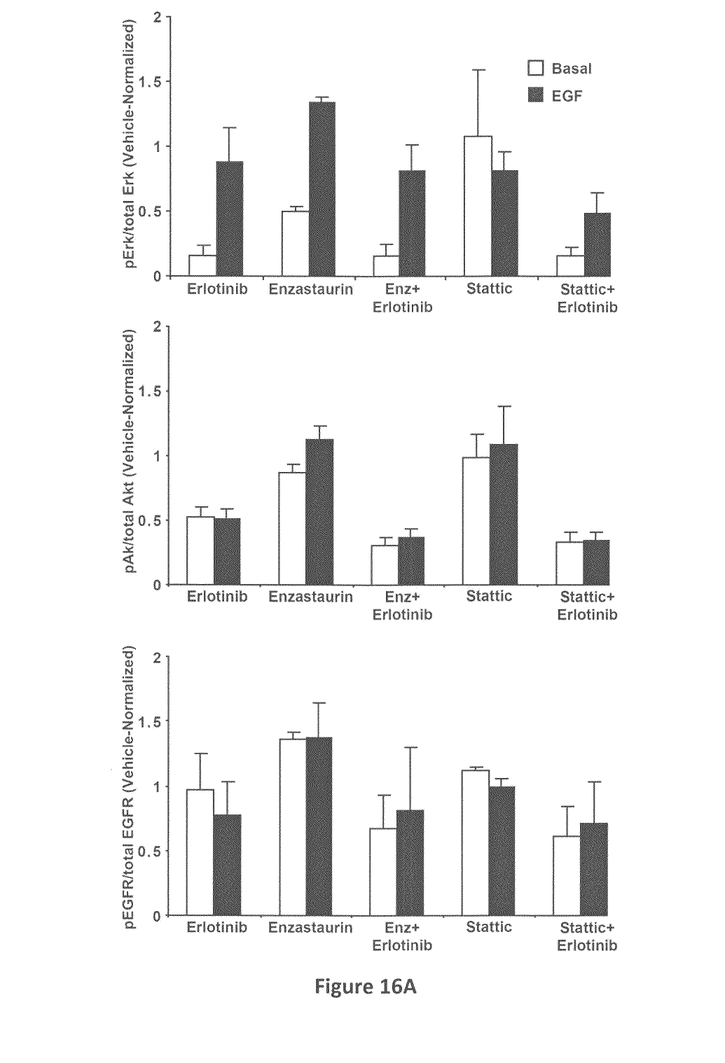

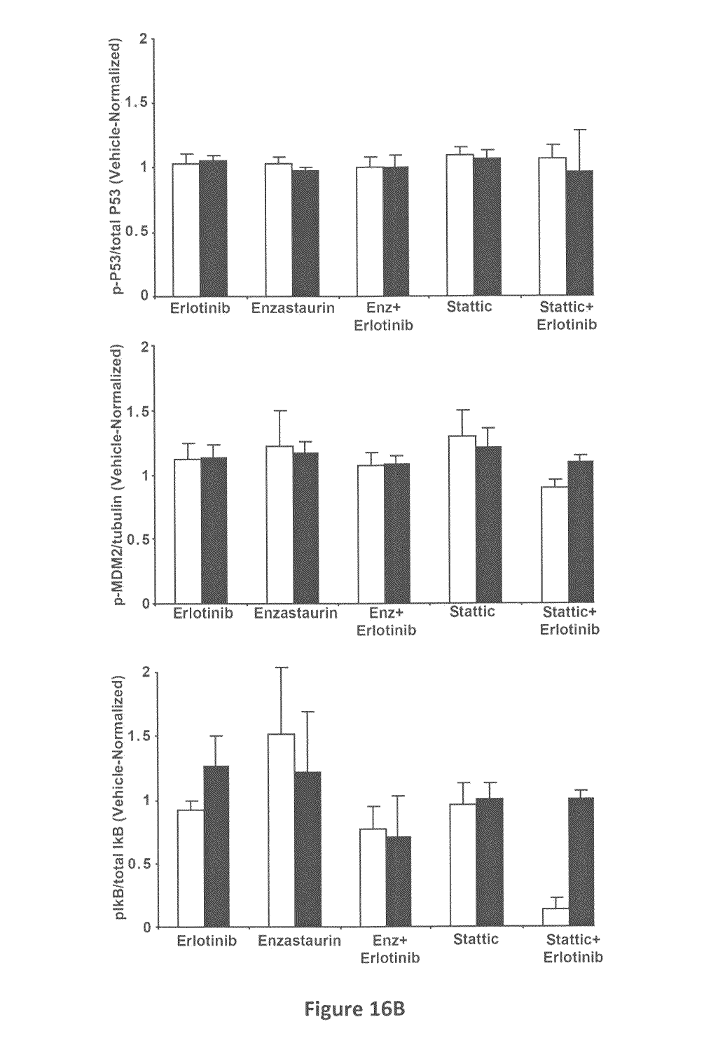

Stattic synergized with erlotinib in inhibiting the growth of both A431 and HCT116 cells (FIGS. 14C and 15B) in keeping with reported dependency of EGFR-driven autocrine growth on STAT3 activation in cancer, but showed no statistically significant synergy in reducing cell motility (FIG. 14D, top). Both Ro-318220 and enzastaurin synergized very significantly with erlotinib in A431 and HCT116 cells (FIGS. 14C and 15C), in experiments employing drugs combined at multiple ratios (1:5, 1:10, 1:20). Combined application of EGFR and Ro-318220 also significantly reduced tumor cell motility (FIG. 14D, bottom). We analyzed the effect of drug combinations on the activation state of a series of benchmark signaling proteins relevant to proliferation and apoptosis, including Akt, Erk, NF-κB, IκB, MDM2, and p53 (FIG. 16). None of these proteins experienced specific activity changes as a consequence of combined application of drugs, with the exception of Akt, which had significantly reduced phosphorylation on S⁴⁷³ in cells treated with erlotinib in combination with either stattic or enzastaurin. Akt-S⁴⁷³—phosphorylation has been described as dependent on integrated signaling via PKC, EGFR, and mTOR. This result suggests a potential pathway by which the enzastaurin-erlotinib combination might reduce cell viability.

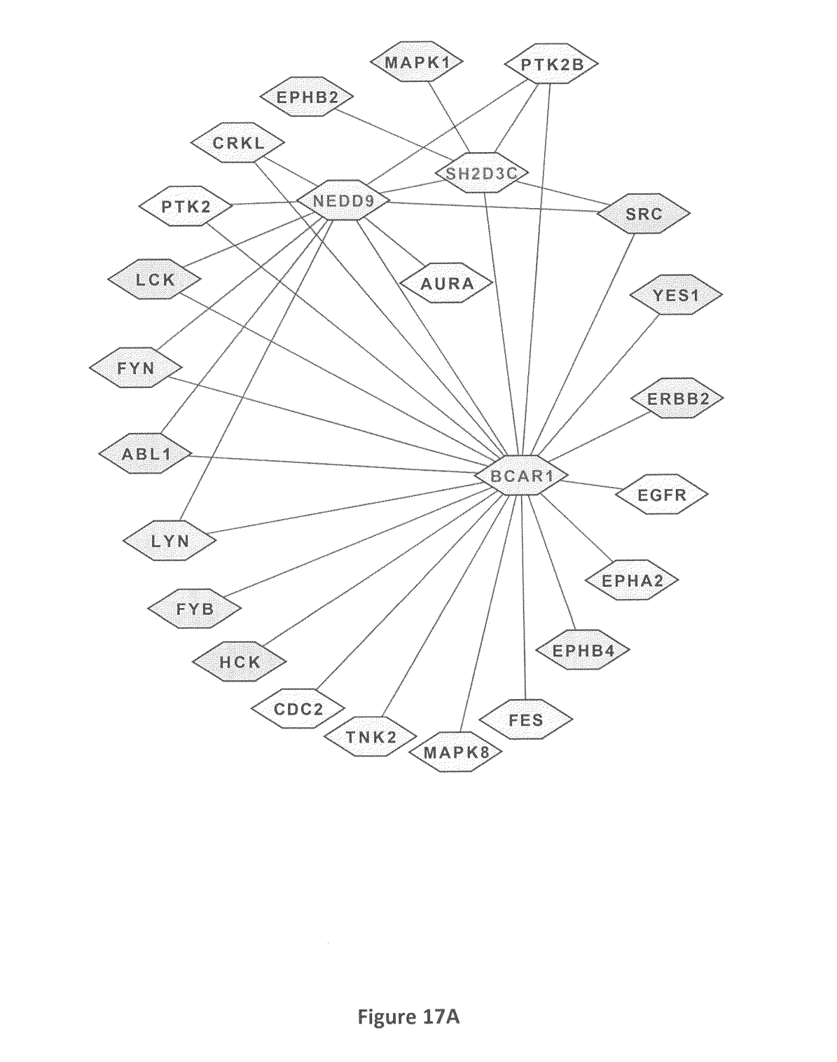

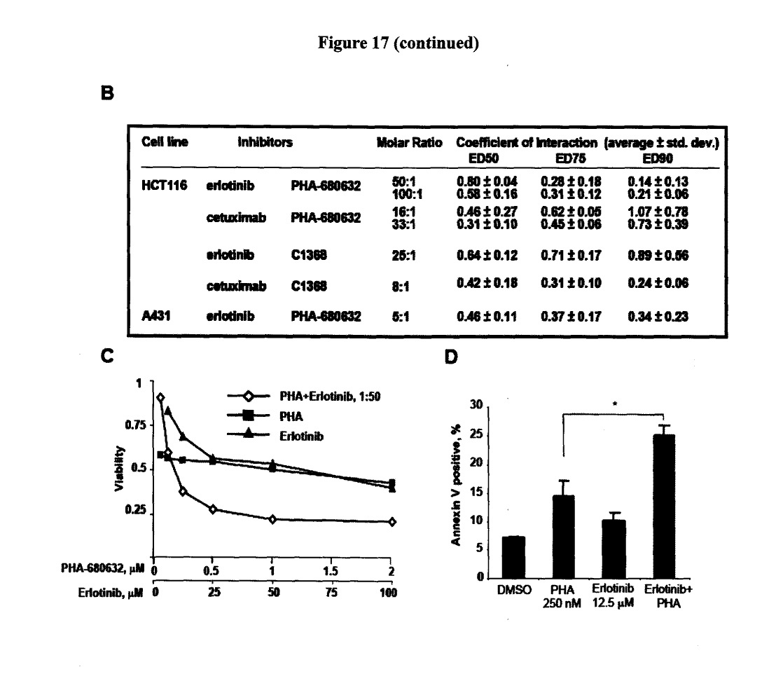

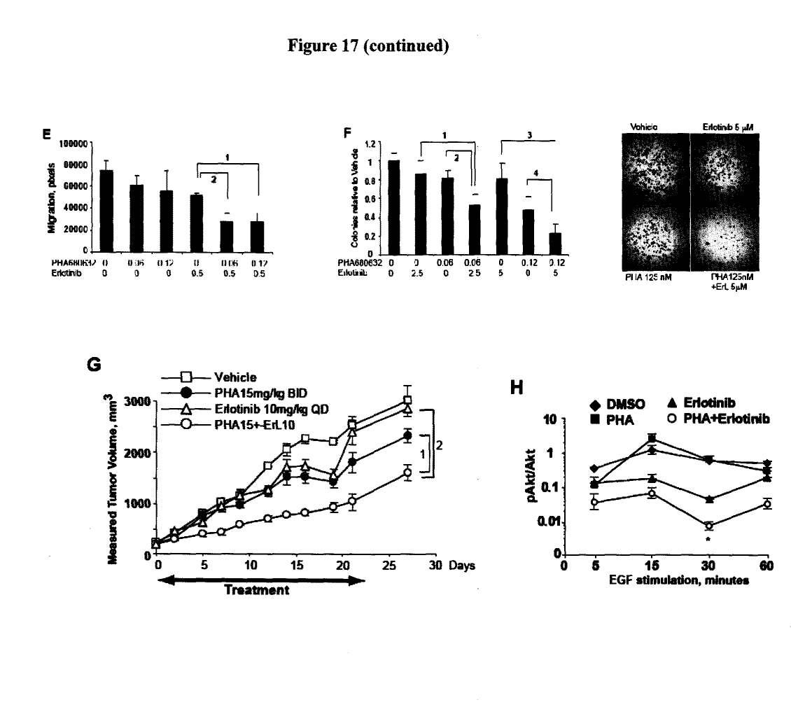

The proteins of the consistently sensitizing BCAR1-SH3D2C-NEDD9 cluster have been linked previously to cell survival control in the context of integrin-mediated signaling cascades, suggesting this cluster is of particular interest for therapeutic exploitation. However, these proteins are not catalytic, and have not been targeted by existing small molecule agents. Given the results suggesting the enrichment of sensitizing genes among proteins closely linked to core hits, we hypothesized that small molecules targeting kinases closely linked to this cluster by physical interactions might similarly provide a rich source of synergizing agents for combination with erlotinib. FIG. 17A shows the direct interaction neighborhood around BCAR1, SH3D3C, and NEDD9, which includes 16 kinases. Drugs that are in pre-clinical or clinical development, or approved agents, target 10 of these kinases (either uniquely, or as one member of a protein family), and some of these drugs have indeed been combined productively with EGFR-directed therapeutics (e.g. dasatinib, targeting Src family kinases. Among these, the NEDD9-interacting kinase AURKA (Aurora-A, STK6) was notable because it positively and directly regulates the important EGFR effector Ral, and has been reported to indirectly upregulate AKT. Moreover, well-tolerated drugs targeting Aurora-A are currently undergoing clinical evaluation.

The small molecule Aurora-A inhibitors PHA-680632 (Soncini et al. (2006) Clin. Cancer Res. 12:4080) synergized strongly with erlotinib in both A431 and HCT116 cells (FIG. 17B). PHA-680632 also synergized with the EGFR-inhibiting antibody cetuximab (FIG. 17B), while erlotinib also synergized with another Aurora-A inhibitor, C1368 (Tari et al., (2007) Bioorg. Med. Chem. Lett. 17:688) Combination of Aurora-A and EGFR-targeting agents did not merely produce cytostasis, but also cell death, increasing the frequency of apoptosis nearly two-fold (FIGS. 17C, 17D). In addition, combination of these drugs significantly reduced cell motility (FIG. 17E), colony growth in soft agar (FIG. 17F), and the growth of tumor xenografts implanted in SCID mice (FIG. 17G).

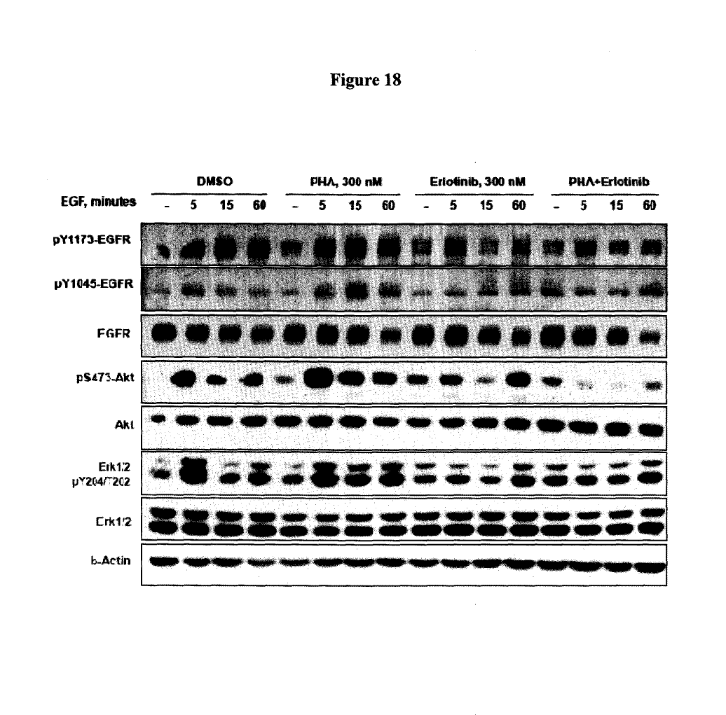

We explored the signaling changes underlying the synergy. Treatment of cells with PHA-680632 alone did not inhibit EGFR expression, autophosphorylation, and activation, and had little effect on ERK1/2 or AKT phosphorylation in response to transient EGF stimulation (FIGS. 17H and 16A). However, in combination with erlotinib treatment, PHA-680632 very significantly reduced S⁴⁷³-AKT phosphorylation below levels seen in cells treated with either agent alone, compatible with the reduced survival of cells treated with the drug combination, while not significantly influencing other EGFR-dependent signaling benchmarks (FIGS. 17H, 18).

To explore signaling consequences of co-inhibition of Aurora-A and EGFR in greater depth, we next undertook a more comprehensive phospho-proteomic analysis of 46 signaling proteins linked to cell proliferation and survival responses following cell treatment with erlotinib, PHA-680632, or both. Analysis of two independently performed screens (FIG. 19A) established that erlotinib blocked EGF-induced activation of multiple signaling pathways (reducing Akt and ERK below background levels), and PHA-680632 had little effect when used as single agent. In contrast, the combination of drugs led to specific inhibition of a subset of proteins, including the greater inhibition of ERK and Akt detected by candidate analysis, but also inhibition of GSK3β (a known functional partner of Aurora-A), JNK, and the Src family kinase FGR. We performed similar experiments to analyze signaling changes under the steady state growth conditions used to assess synergistic killing of cells (i.e., when the activation state of pathways was not strictly dependent on EGF) (FIG. 19B). Strikingly, this analysis re-identified the same targets for the drug combination as those seen with EGF-dependent signaling (FIG. 19A), but in addition showed significant reduction in activity of STAT3, and a group of Src kinases, including FGR, Hck, Lyn, Src, and Lck. These last hits in particular are intriguing, as the BCAR1-NEDD9-SH2D3C proteins that led us to consider AurA are direct activators and substrates of these same Src family kinases (FIG. 17A). One possibility is that use of AurA inhibitors weakened this resistance cluster in the network.

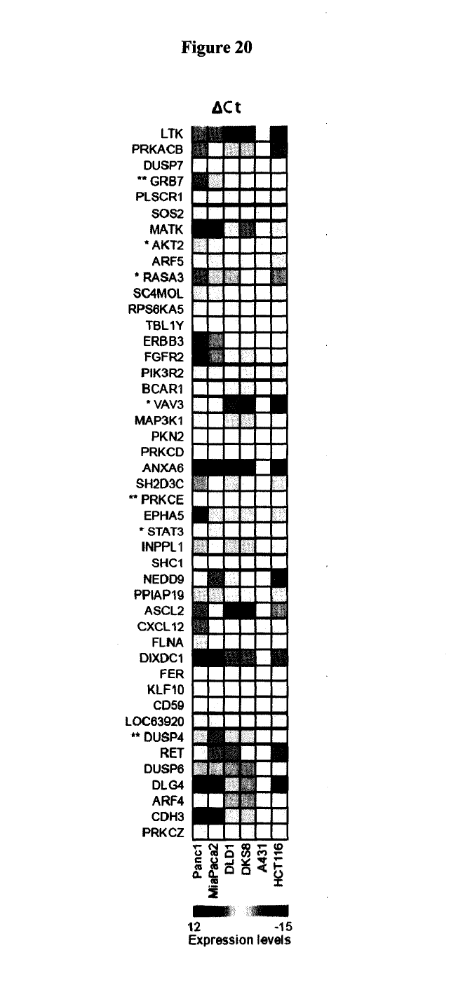

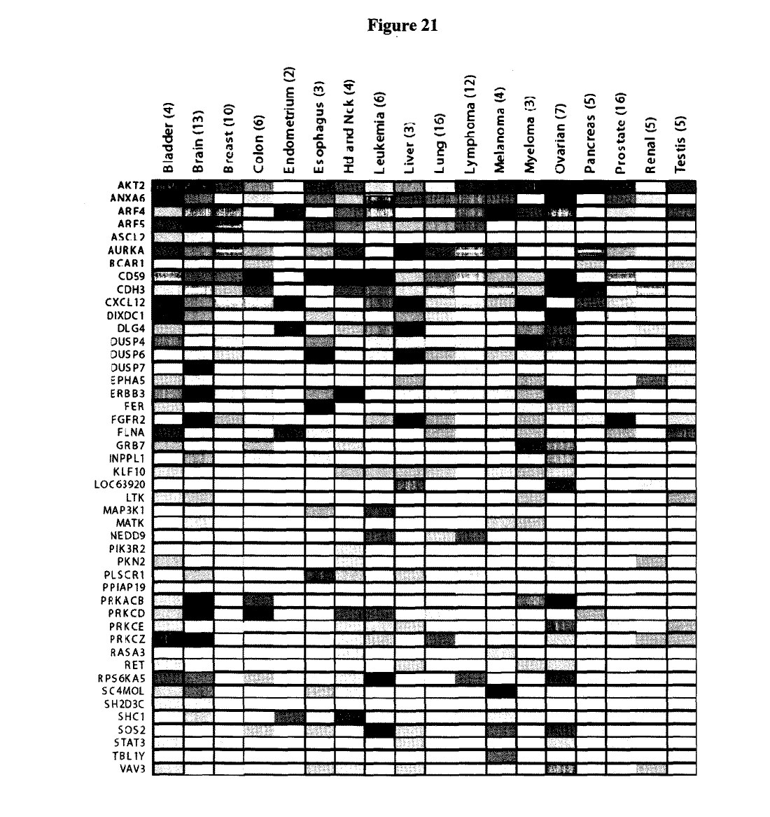

As described in Example 11, another potential use of this data set is for the nomination of new biomarkers for selecting patient responsiveness. However, extensive analysis of the expression of siRNA targets in cell lines used for functional analysis (FIG. 20) showed no statistically significant correlation between expression level and role in modulating resistance, while analysis of Oncomine profiles (FIG. 21) did not reveal specific trends of altered expression in tumors. Large sequencing projects, including among others the Cancer Gene Census, have noted mutations with some frequency for RET, FLNA, FGFR2, SMAD2, PIK3R1, ABL1, CCND1, and AKT2. See the world wide web at sanger.ac.uk/CGP/. However, most of the genes we identified are not common targets for mutations. These observations have potentially important translational implications, as much effort has gone into analyzing gene expression or mutational status to predict drug resistance. This cumulative lack of a clear pattern of expression or mutation likely reflects the complexity of cancer-associated signaling networks. For many solid tumors, no unique oncogenic driver has been yet identified, but instead, tumor cells undergo multiple, sequential process-oriented oncogenic alterations that together reprogram multiple yet discrete aspects of tumor functionality. In such a scenario, fitness of a cancer cell is determined by the robustness of its signaling network as a whole. The new resistance-mediating genes we have identified should undergo scrutiny as alternative EGFR modulators, joining with proteins such as K-Ras, B-Raf, c-MET, IGF-1, and others.

A major goal of systems-level bioinformatics analyses is to nominate critical nodes to target in combination to enhance therapy in the clinic, with clear successes beginning to emerge from this information-driven strategy (Pritchard et al., (2009) Mol Cancer Ther. 8:2183). Separately, screening of siRNA libraries has emerged as a powerful approach to identify genes that can kill cancer cells, or sensitize them to cytotoxic agents. To date, such screening has typically employed either full genome screens, or screens of small libraries targeting limited groups of proteins, such as the kinome/phosphatome. Interestingly, a genome-wide screen to identify sensitizers to the microtubule-targeting agent paclitaxel identified a number of hits that clustered into coherent groups of genes associated with the proteasome or mitotic spindle (Whitehurst et al., (2007) Nature 446:815), which a priori had been linked to paclitaxel activity based on existing pathway knowledge.

In the current study, we employed bioinformatics design and direct screening, and found that many proteins influencing cellular resistance to EGFR-targeting agents clustered in connection-dense, highly interactive portions of the EGFR signaling network, thus supporting our core hypothesis that these characteristics would enrich for synthetic lethal interactions. These sensitizing protein clusters were useful for predicting the efficacy of combining protein-targeted drugs with EGFR-pathway signaling inhibitors, suggesting the potential of this approach for speeding the translation of results to the clinic. We believe this targeted approach has several advantages in comparison to a full genome screen. Beyond the obvious factors of convenience, speed, and cost, all hits arising from a targeted screen already have at least some defined functional relationships to the signaling pathway being probed, accelerating validation and mechanism testing. Further, the limited size of the library being probed allowed the use of more relaxed statistical criteria in nominating hits for validation than would be necessary in a full genome screen, and allowed us to repeat the primary screen multiple times: given the intrinsic noise in siRNA screening, these are important advantages. Finally, our observation that the single greatest source of enrichment for hits (FIG. 12C) is among the proteins with both direct physical interactions and literature-based pathway connections to the library seeds provides guidance for future library optimization.

We have defined the network structure for EGFR-pathway specific and general drug resistance in several cell lines. Accordingly, the present invention provides a unique resource: a deeply probed, heavily annotated library with a linked live database that provides a “Rosetta Stone” for drug resistance studies. This work can be rapidly translated into improved therapy for cancer patients, as the information is used to design new Phase I trials. Indeed, new combinatorial approaches for the eradication of cancer cells is discloses as are efficacious agents for effecting the same.

### REFERENCES

1. Rocha-Lima C M, Soares H P, Raez L E, Singal R. EGFR targeting of solid tumors. Cancer Control 2007; 14:295-304.
2. Ciardiello F, De Vita F. Epidermal growth factor receptor (EGFR) inhibitors in cancer therapy. Prog Drug Res 2005; 63:93-114.
3. Raymond E, Faivre S, Armand J P. Epidermal growth factor receptor tyrosine kinase as a target for anticancer therapy. Drugs 2000; 60:15-23; discussion 41-2.
4. Buchholz T A, Tu X, Ang K K, Esteva F J, Kuerer H M, Pusztai L, Cristofanilli M, Singletary S E, Hortobagyi G N, Sahin A A. Epidermal growth factor receptor expression correlates with poor survival in patients who have breast carcinoma treated with doxorubicin-based neoadjuvant chemotherapy. Cancer 2005; 104:676-81.
5. Chan S K, Hill M E, Gullick W J. The role of the epidermal growth factor receptor in breast cancer. J Mammary Gland Biol Neoplasia 2006; 11:3-11.
6. Cappuzzo F, Toschi L, Finocchiaro G, Ligorio C, Santoro A. Surrogate predictive biomarkers for response to anti-EGFR agents: state of the art and challenges. Int J Biol Markers 2007; 22:S10-23.
7. Jimeno A, Hidalgo M. Pharmacogenomics of epidermal growth factor receptor (EGFR) tyrosine kinase inhibitors. Biochim Biophys Acta 2006; 1766:217-29.
8. Eddy S R. Genetics. Total information awareness for worm genetics. Science 2006; 311:1381-2.
9. Whitehurst A W, Bodemann B O, Cardenas J, Ferguson D, Girard L, Peyton M, Minna J D, Michnoff C, Hao W, Roth M G, Xie X J, White M A. Synthetic lethal screen identification of chemosensitizer loci in cancer cells. Nature 2007; 446:815-9.
10. Skobeleva N, Menon S, Weber L, Golemis E A, Khazak V. In vitro and in vivo synergy of MCP compounds with mitogen-activated protein kinase pathway- and microtubule-targeting inhibitors. Mol Cancer Ther 2007; 6:898-906.
11. Mishra G R, Suresh M, Kumaran K, Kannabiran N, Suresh S, Bala P, Shivakumar K, Anuradha N, Reddy R, Raghavan T M, Menon S, Hanumanthu G, Gupta M, Upendran S, Gupta S, Mahesh M, Jacob B, Mathew P, Chatterjee P, Arun K S, Sharma S, Chandrika K N, Deshpande N, Palvankar K, Raghavnath R, Krishnakanth R, Karathia H, Rekha B, Nayak R, Vishnupriya G, Kumar H G, Nagini M, Kumar G S, Jose R, Deepthi P, Mohan S S, Gandhi T K, Harsha H C, Deshpande K S, Sarker M, Prasad T S, Pandey A. Human protein reference database—2006 update. Nucleic Acids Res 2006; 34:D411-4.
12. Mak H C, Daly M, Gruebel B, Ideker T. CellCircuits: a database of protein network models. Nucleic Acids Res 2007; 35:D538-45.
13. Dimri M, Naramura M, Duan L, Chen J, Ortega-Cava C, Chen G, Goswami R, Fernandes N, Gao Q, Dimri G P, Band V, Band H. Modeling breast cancer-associated c-Src and EGFR overexpression in human MECs: c-Src and EGFR cooperatively promote aberrant three-dimensional acinar structure and invasive behavior. Cancer Res 2007; 67:4164-72.
14. Seton-Rogers S E, Brugge J S. ErbB2 and TGF-beta: a cooperative role in mammary tumor progression? Cell Cycle 2004; 3:597-600.
15. O'Neill G M, Fashena S J, Golemis E A. Integrin signaling: a new Cas(t) of characters enters the stage. Trends Cell Biol 2000; 10:111-9.
16. Singh M K, Cowell L, Seo S, O'Neill G M, Golemis E A. Molecular basis for HEF1/NEDD9/Cas-L action as a multifunctional co-ordinator of invasion, apoptosis, and cell cycle. Cell Biochem Biophys 2007 in press.
17. Ji H, Ramsey M R, Hayes D N, Fan C, McNamara K, Kozlowski P, Torrice C, Wu M C, Shimamura T, Perera S A, Liang M C, Cai D, Naumov G N, Bao L, Contreras C M, Li D, Chen L, Krishnamurthy J, Koivunen J, Chirieac L R, Padera R F, Bronson R T, Lindeman N I, Christiani D C, Lin X, Shapiro G I, Janne P A, Johnson B E, Meyerson M, Kwiatkowski D J, Castrillon D H, Bardeesy N, Sharpless N E, Wong K K. LKB1 modulates lung cancer differentiation and metastasis. Nature 2007; 448:807-10.
18. Kim M, Gans J D, Nogueira C, Wang A, Paik J H, Feng B, Brennan C, Hahn W C, Cordon-Cardo C, Wagner S N, Flotte T J, Duncan L M, Granter S R, Chin L. Comparative oncogenomics identifies NEDD9 as a melanoma metastasis gene. Cell 2006; 125:1269-81.
19. O'Neill G M, Seo S, Serebriiskii I G, Lessin S R, Golemis E A. A new central scaffold for metastasis: parsing HEF1/Cas-L/NEDD9. Cancer Res 2007; 67:8975-9.
20. Singh M, Cowell L, Seo S, O'Neill G, Golemis E. Molecular basis for HEF1/NEDD9/Cas-L action as a multifunctional co-ordinator of invasion, apoptosis and cell cycle. Cell Biochem Biophys 2007; 48:54-72.
21. Swanton C, Marani M, Pardo O, Warne P H, Kelly G, Sahai E, Elustondo F, Chang J, Temple J, Ahmed A A, Brenton J D, Downward J, Nicke B. Regulators of mitotic arrest and ceramide metabolism are determinants of sensitivity to Paclitaxel and other chemotherapeutic drugs. Cancer Cell 2007; 11:498-512.
22. Smyth G K. Linear models and empirical bayes methods for assessing differential expression in microarray experiments. Statistical applications in genetics and molecular biology 2004; 3:Article 3.
23. Gentleman R C, Carey V J, Bates D M, Bolstad B, Dettling M, Dudoit S, Ellis B, Gautier L, Ge Y, Gentry J, Hornik K, Hothorn T, Huber W, Iacus S, Irizarry R, Leisch F, Li C, Maechler M, Rossini A J, Sawitzki G, Smith C, Smyth G, Tierney L, Yang J Y, Zhang J. Bioconductor: open software development for computational biology and bioinformatics. Genome Biol 2004; 5:R80.
24. Benjamini Y, Hochhberg Y. Controlling the False Discovery Rate: A practical and powerful approach to multiple testing. J Royal Stat Soc B 1995; 57:289-300.
25. Defilippi P, Di Stefano P, Cabodi S. p130Cas: a versatile scaffold in signaling networks. Trends Cell Biol 2006; 16:257-63.
26. Cai D, Iyer A, Felekkis K N, Near R I, Luo Z, Chernoff J, Albanese C, Pestell R G, Lerner A. AND-34/BCAR3, a GDP exchange factor whose overexpression confers antiestrogen resistance, activates Rac, PAK1, and the cyclin D1 promoter. Cancer Res 2003; 63:6802-8.
27. Ambrogio C, Voena C, Manazza A D, Riera L, Piva R, Barberis L, Costa C, Tarone G, Defilippi P, Hirsch E, Boeri Erba E, Mohammed S, Jensen O N, Palestro G, Inghirami G, Chiarle R. p130cas mediates the transforming properties of the anaplastic lymphoma kinase. Blood 2005.
28. Engelman J A, Zejnullahu K, Mitsudomi T, Song Y, Hyland C, Park J O, Lindeman N, Gale C M, Zhao X, Christensen J, Kosaka T, Holmes A J, Rogers A M, Cappuzzo F, Mok T, Lee C, Johnson B E, Cantley L C, Janne P A. MET amplification leads to gefitinib resistance in lung cancer by activating ERBB3 signaling. Science 2007; 316:1039-43.
29. Frolov A, Schuller K, Tzeng C W, Cannon E E, Ku B C, Howard J H, Vickers S M, Heslin M J, Buchsbaum D J, Arnoletti J P. ErbB3 Expression and Dimerization with EGFR Influence Pancreatic Cancer Cell Sensitivity to Erlotinib. Cancer Biol Ther 2007; 6.
30. Valkova C, Maerz S, Imhof D, Liebmann C. Protein kinase Cepsilon may act as EGFinducible scaffold protein for phospholipase Cgamma1. Cell Signal 2007; 19:1830-43.

TABLE 1

| | Trancriptional | | | | | | | |
|---|---|---|---|---|---|---|---|---|
| Gene | change (AG/HRG | | | | | Fly | | Local |
| name | fold) | AG | Pathways | PPI | Complex | homolog | Paralog | expert |
| ABHD2 | −2 | −2 | | | | | | |
| ABI1 | | | core | round 2 | | | | |
| ABL1 | | | | round 1 | | | | |
| ACTC | | | | round 2 | complex | | | |
| ACTN4 | 1.5 | 2 | | | complex | | | |
| ACTR3 | | | | round 2 | complex | | | |
| AEBP2 | | | | | | 1 | | |
| AKT1 | | | core | round 2 | | | | |
| AKT2 | | | | | | | paralog | |
| AKT3 | | | | | | | paralog | |
| ALK | | | | round 1 | | | | |
| AMH | | | | round 1 | | | | |
| ANKRD11 | | 1.5 | | round 2 | | | | |
| ANXA2 | | | | round 2 | complex | | | |
| ANXA6 | | | | round 2 | complex | | | |
| AP2A1 | | | five | round 1 | complex | | | |
| AP2A2 | | | | round 1 | | | | |
| APP | | | | round 1 | | | | |
| ARAF | | | core | round 2 | | 3 | | |
| AREG | | | | round 1 | | | | |
| ARF1 | | 1.5 | | round 2 | | | | |
| ARF3 | | | | | | | paralog | |
| ARF4 | | | five | round 1 | | | | |
| ARF5 | | | | | | | paralog | |
| ARG1 | | | | | | | paralog | |
| ARG2 | | | | | | 1 | | |
| ARRDC3 | | −2 | | | | | | |
| ASCL1 | | | | | | 2 | | |
| ASCL2 | | | | round 2 | | 1 | | |
| ATP1B3 | | 2 | | | | | | |
| AVIL | 1.5 | 2 | | round 2 | | | | |
| AXL | | | | round 1 | | | | |
| AZIN1 | | 2 | | | | | | |
| B4GALT1 | 1.5 | 2 | | | | | | |
| BARHL1 | | | | | | 2 | | |
| BARHL2 | | | | | | 1 | | |
| BCAR1 | | | five | round 1 | complex | | | |
| BCAR3 | | | | round 1 | complex | | | |
| BCL10 | | 2 | | | | | | |
| BCL3 | | | | round 1 | | | | |
| BCR | | | | round 1 | | | | |
| BIRC4 | −2 | −2 | | | | | | |
| BLK | | | | | | | paralog | |
| BMP2 | | | | | | 2 | | |
| BMP4 | | | | | | 1 | | |
| BMPR1A | | | | | | 2 | | |
| BMPR1B | | | | round 2 | | 1 | | |
| BRAF | | | five | | | 2 | | |
| BRD4 | | 2 | | | | | | |
| BTC | | | | round 1 | | | | |
| BTRC | | 1.5 | | round 2 | | | | |
| C14orf120 | 1.5 | 2 | | | | | | |
| C20orf119 | | | | | | | paralog | |
| CALCOCO2 | | | | round 1 | | | | |
| CALD1 | | | | round 1 | | | | |
| CALM1 | | | | round 1 | complex | | | |
| CALM2 | | | | round 2 | complex | | | |
| CALM3 | | | | round 2 | complex | | | |
| CAMK2G | | | five | round 2 | | | | |
| CAMLG | | | | round 1 | | | | |
| CASP1 | | | | round 1 | | | | |
| CAV1 | | | core | round 1 | complex | | | |
| CAV2 | | | core | round 2 | complex | | | |
| CAV3 | | | | round 1 | | | | |
| CBL | | | core | round 1 | complex | | | |
| CBLB | | | five | round 1 | complex | | | |
| CBLC | | | five | round 1 | | | | |
| CCND1 | | | five | round 2 | | | | |
| CCND2 | | | | | | | paralog | |
| CCND3 | | | | | | | paralog | |
| CCNE1 | | | | round 2 | | 2 | | |
| CCNE2 | | | five | | | 1 | | |
| CCR1 | | | | | | | paralog | |
| CCR2 | | | | | | | paralog | |
| CCR5 | | | | round 2 | complex | | | |
| CD22 | | | | round 1 | | | | |
| CD247 | | | | round 1 | | | | |
| CD2AP | | | | round 1 | | | | |
| CD3E | | | | round 1 | | | | |
| CD44 | | | | round 1 | | | | |
| CD59 | | | | round 2 | complex | | | |
| CD82 | | | | round 1 | | | | |
| CDC25A | | 1.5 | | round 1 | | | | |
| CDC42 | | −1.5 | core | round 2 | | | | |
| CDCP1 | | | | round 2 | complex | | | |
| CDH1 | | | five | round 1 | | | | |
| CDH3 | | | | | | | paralog | |
| CDH5 | | | | round 1 | | | | |
| CDK2 | | | five | round 2 | | | | |
| CEACAM1 | | | five | round 1 | | | | |
| CEBPA | | | five | round 2 | | | | |
| CEBPB | | | five | round 1 | | | | |
| CEBPZ | −2 | | | | | | | |
| CHAT | | | | round 1 | | | | |
| CHKB | 1.5 | 2 | | | | | | |
| CHL1 | | | | | | 2 | | |
| CHUK | | | five | round 2 | | | | |
| COPS5 | | | | | | 1 | | |
| CPSF6 | | 1.5 | | round 2 | | | | |
| CREB1 | | | core | round 2 | | | | |
| CRK | | | five | round 1 | | | | |
| CRKL | | | five | round 1 | | | | |
| CSF2RB | | | | round 1 | | | | |
| CSF3R | | | | round 1 | | | | |
| CSK | | | core | round 1 | | | | |
| CSNK2A1 | | 1.5 | five | round 2 | | | | |
| CSPG4 | | | | round 1 | | | | |
| CTGF | | 2 | | | | | | |
| CTNNA1 | | −2 | | round 2 | complex | | | |
| CTNNB1 | | 2 | five | round 1 | | 1 | | |
| CTNND1 | | | five | round 2 | complex | | | |
| CTTN | | 2 | | round 2 | complex | | | |
| CUTL1 | | 1.5 | | round 2 | | | | |
| CXCL12 | −2 | | | | | | | |
| CXorf33 | −2 | −2 | | | | | | |
| CXorf56 | −2 | −2 | | | | | | |
| CYR61 | | 2 | | | | | | |
| DAB2 | 1.5 | | | round 2 | | | | |
| DAG1 | | | five | round 1 | | | | |
| DCDC2 | | −2 | | | | | | |
| DCN | | | | round 1 | | | | |
| DDEF1 | | | five | round 2 | | | | |
| DDHD1 | 1.5 | 2 | | | | | | |
| DDR1 | | | | round 1 | | | | |
| DDR2 | | | | round 1 | | | | |
| DDX50 | 1.5 | 2 | | | | | | |
| DEGS1 | | | | round 1 | | | | |
| DIDO1 | 1.5 | 2 | | | | | | |
| DIXDC1 | −2 | −2 | | | | | | |
| DLG4 | | | | round 1 | | | | |
| DLG5 | | 1.5 | | round 2 | | | | |
| DLL1 | | | | | | 2 | | |
| DLL4 | | | | | | 1 | | |
| DNAJA3 | | | | round 2 | complex | | | |
| DNM1 | | | five | round 2 | | | | |
| DOCK1 | | | | round 1 | | | | |
| DOCK2 | | | | | | | paralog | |
| DOCK5 | | | | | | | paralog | |
| DOK2 | | | five | round 2 | | | | |
| DUSP1 | | 2 | five | | | | | |
| DUSP4 | | 2 | | | | | | |
| DUSP6 | | | | | | 3 | | |
| DUSP7 | | | | | | 2 | | |
| DUSP9 | | | | | | 1 | | |
| DYNLL1 | | | | round 2 | complex | | | |
| DYNLL2 | | | | | | | paralog | |
| E2F5 | | | | | | 1 | | |
| EBF | | | | | | 2 | | |
| EBF3 | | | | | | 1 | | |
| EDN1 | | 2 | | | | | | |
| EEF1A1 | | | five | round 2 | complex | | | |
| EEF1A2 | | | | | | | paralog | |
| EFS | | | | round 1 | | | | |
| EGF | | | core | round 1 | | | | |
| EGFR | | | core | round 1 | complex | 4 | | |
| EGR1 | | 2 | | round 2 | | | | |
| EGR2 | | 2 | | | | | | |
| EGR3 | | 2 | | | | | | |
| EIF3S9 | 1.5 | 2 | | | | | | |
| EIF4A1 | | 2 | | | complex | | | |
| EIF4A2 | | | | | | | paralog | |
| EIF4A3 | | | | | | | paralog | |
| ELF3 | 2 | 2 | five | round 2 | | | | |
| ELK1 | | | core | round 2 | | | | |
| EPB41 | | | | | | 4 | | |
| EPB41L1 | | | | | | 3 | | |
| EPB41L2 | | | | | | 2 | | |
| EPB41L3 | | | | | | 1 | | |
| EPGN | | | | | | | | expert |
| EPHA2 | | | | round 1 | complex | | | |
| EPHA3 | | | | | | | paralog | |
| EPHA5 | | | | | | | paralog | |
| EPHA7 | | | | | | | paralog | |
| EPN1 | | | five | round 2 | | | | |
| EPOR | | | | round 1 | | | | |
| EPPK1 | | | five | round 1 | complex | | | |
| EPS15 | | | five | round 1 | | | | |
| EPS15L1 | | 1.5 | five | | | | | |
| EPS8 | | | core | round 1 | | | | |
| ERBB2 | | | core | round 1 | | 3 | | |
| ERBB2IP | | | | round 1 | | | | |
| ERBB3 | | | core | round 1 | | 2 | | |
| ERBB4 | | | | | | | | |
| ERBB4 | | | five | round 2 | | 1 | | |
| EREG | | | | round 1 | | | | |
| ERRFI1 | | | five | round 1 | | | | |
| ESR1 | | | | round 1 | | | | |
| ETF1 | | | | | | 1 | | |
| ETV6 | | | | | | 2 | | |
| ETV7 | | | | | | 1 | | |
| EXOC4 | 1.5 | 1.5 | | round 2 | | | | |
| EYA1 | | | | | | 4 | | |
| EYA2 | | | | round 2 | | 3 | | |
| EYA3 | | | | | | 2 | | |
| EYA4 | | | | | | 1 | | |
| FBRS | | 2 | | | | | | |
| FCGR3A | | | | round 1 | | | | |
| FDFT1 | 1.5 | 2 | | | | | | |
| FER | | | | round 1 | | | | |
| FES | | | | round 1 | | | | |
| FGFR1 | | | | round 1 | | 4 | | |
| FGFR2 | | | | | | 3 | | |
| FGFR3 | | | | round 2 | | 2 | | |
| FGFR4 | | | | | | 1 | | |
| FGR | | | | | | | paralog | |
| FIGF | | | | | | 2 | | |
| FLJ11903 | | 2 | | | | | | |
| FLJ20280 | −2 | −2 | | | | | | |
| FLNA | | | | round 2 | complex | | | |
| FLT1 | | | | round 1 | | 3 | | |
| FLT3 | | | | round 1 | | | | |
| FLT4 | | | | round 1 | | 2 | | |
| FMN2 | | | | | | 1 | | |
| FOS | | 2 | core | | | 1 | | |
| FOSB | | 2 | | | | | | |
| FOXO1A | | | core | round 2 | | | | |
| FRK | | | | | | 1 | | |
| FSCN1 | | | | round 2 | complex | | | |
| FUS | 1.5 | 2 | | | | | | |
| FYN | | | | round 1 | | | | |
| FZR1 | | | | round 1 | | | | |
| GAB1 | | | core | round 1 | | | | |
| GAB2 | | | five | round 2 | | | | |
| GAPDH | | | | round 2 | complex | | | |
| GFRA1 | −2 | | | | | | | |
| GHR | | | | round 1 | | | | |
| GIT1 | | | five | round 2 | | | | |
| GIT2 | | | | | | | paralog | |
| GJA1 | | | five | round 2 | | | | |
| GLI2 | | | | | | 2 | | |
| GLI3 | | | | | | 1 | | |
| GNA12 | | 1.5 | five | | | | | |
| GNAI1 | | | five | round 2 | | | | |
| GNAI2 | | | | round 1 | | | | |
| GNAI3 | | | | | | | paralog | |
| GNAO1 | | | | | | | paralog | |
| GNB2L1 | | | five | round 2 | | | | |
| GRAP | | | | | | | paralog | |
| GRAP2 | | | | round 1 | | | | |
| GRB10 | | | | round 1 | | | | |
| GRB14 | | | five | round 1 | | | | |
| GRB2 | | | core | round 1 | complex | | | |
| GRB7 | | | core | round 1 | | | | |
| GSK3b | | | | | | | | expert |
| GSN | | | | round 1 | complex | | | |
| HBEGF | | | five | round 1 | | | | |
| hCG_1757335 | | | | | | | paralog | |
| HCK | | | | round 1 | | | | |
| HD | | | five | round 1 | | | | |
| HDAC1 | | | five | round 2 | | | | |
| HDAC6 | | | | | | | | expert |
| HES1 | | 2 | | | | | | |
| HGF | | | | | | | | expert |
| HIP1 | | | five | round 2 | | | | |
| HLA-A | | | | round 1 | | | | |
| HNRPA1 | | 1.5 | | | complex | | | |
| HNRPK | | | | round 2 | complex | | | |
| HRAS | | | core | round 2 | | 2 | | |
| HSP90AA1 | | | | round 1 | | | | |
| HSP90B1 | | | | round 1 | | | | |
| HSPA5 | | | | round 2 | complex | | | |
| HSPA8 | | | | round 2 | complex | | | |
| HSPA9B | | | | round 2 | complex | | | |
| HSPD1 | | | | round 2 | complex | | | |
| ID1 | | 2 | | | | | | |
| ID2 | | | | round 1 | | | | |
| IER2 | | 2 | | | | | | |
| IGF1R | 1.5 | 1.5 | | round 1 | | | | |
| IKBKB | | | five | round 2 | | | | |
| IKBKG | | | five | round 2 | | | | |
| IL2 | | | | round 1 | | | | |
| IL2RB | | | | round 1 | | | | |
| IL2RG | | | | round 1 | | | | |
| IL4R | | | | round 1 | | | | |
| IL6ST | | | | round 1 | | | | |
| ILF3 | | | | round 2 | complex | | | |
| INPP5D | | | | round 1 | | | | |
| INPPL1 | | | core | round 1 | | | | |
| INSR | | | | round 1 | | | | |
| INSRR | | | | | | | paralog | |
| IQGAP1 | | | | round 2 | complex | | | |
| IRS1 | | | | round 1 | | | | |
| IRS2 | | | | round 1 | | | | |
| ITCH | | | five | round 1 | | | | |
| ITGA5 | | | | round 1 | | | | |
| ITGB3 | | | | round 1 | | | | |
| ITGB4 | | | | round 1 | | | | |
| JAK1 | | 1.5 | core | round 2 | | | | |
| JAK2 | | | core | round 1 | | | | |
| JUN | | 1.5 | core | round 2 | | 3 | | |
| JUNB | | 2 | | | | 2 | | |
| JUND | | | five | | | 1 | | |
| JUP | | | | round 1 | | | | |
| KDR | | | | round 1 | | 1 | | |
| KLF10 | | 2 | | | | | | |
| KLF6 | 1.5 | 2 | | | | | | |
| KRAS | | | core | | | | | |
| KRT17 | | | five | round 1 | complex | | | |
| KRT18 | | | five | round 1 | complex | | | |
| KRT5 | | | | round 2 | complex | | | |
| KRT6A | | | | round 2 | complex | | | |
| KRT6B | | | | | | | paralog | |
| KRT6C | | | | | | | paralog | |
| KRT6E | | | | | | | paralog | |
| KRT7 | | | five | round 1 | complex | | | |
| KRT8 | | | five | round 1 | complex | | | |
| KSR2 | | | | | | 1 | | |
| L1CAM | | | | round 2 | | 1 | | |
| LCK | | | | round 1 | | | | |
| LCP2 | | | | round 1 | | | | |
| LGALS3 | −2 | −2 | | | | | | |
| LIN7A | | | | round 2 | | 3 | | |
| LIN7B | | | | round 2 | | 2 | | |
| LIN7C | | | | | | 1 | | |
| LMNA | | | | round 2 | complex | | | |
| LOC284393 | | | | | | | paralog | |
| LOC387927 | | | | round 1 | | | | |
| LOC389342 | | | | | | | paralog | |
| LOC390006 | | | | | | | paralog | |
| LOC63920 | | −2 | | | | | | |
| LOC642045 | | | | | | | paralog | |
| LOC642954 | | | | | | | paralog | |
| LOC643751 | | | | | | | paralog | |
| LOC643997 | | | | | | | paralog | |
| LOC645691 | | | | | | | paralog | |
| LOC648695 | | | | | | | paralog | |
| LOC650332 | | | | | | | paralog | |
| LOC728198 | | | | | | | paralog | |
| LOC731173 | | | | | | | paralog | |
| LOC731292 | | | | | | | paralog | |
| LOC731751 | | | | | | | paralog | |
| LRBA | | | | | | 2 | | |
| LRP1 | | | | round 1 | | | | |
| LRP11 | −2 | −2 | | | | | | |
| LRRC4B | | | | | | 1 | | |
| LTK | | | | round 1 | | | | |
| LYN | | | | round 1 | complex | | | |
| MAD1L1 | 2 | 2 | | | | | | |
| MAP2K1 | | | core | round 2 | | 2 | | |
| MAP2K2 | | | core | | | 1 | | |
| MAP2K3 | | | core | | | | | |
| MAP2K4 | | | core | | | | | |
| MAP2K7 | | | core | | | | | |
| MAP3K1 | | | core | round 2 | | | | |
| MAP3K11 | | | five | round 2 | | | | |
| MAP3K14 | | | five | round 1 | | | | |
| MAP3K3 | | | five | round 2 | | | | |
| MAP3K4 | | | core | | | | | |
| MAP3K5 | | 1.5 | | round 2 | | | | |
| MAP4K1 | | | | round 1 | | | | |
| MAPK1 | | | core | round 1 | | | | |
| MAPK10 | | | | | | | paralog | |
| MAPK14 | | | core | round 2 | | | | |
| MAPK3 | | 2 | | | | | | |
| MAPK3 | | | core | | | | | |
| MAPK8 | | | core | round 2 | | | | |
| MAPK9 | | | | | | | paralog | |
| MAPKAPK2 | | | | round 1 | | | | |
| MATK | | | five | round 1 | | | | |
| MCF2 | | | five | round 2 | | | | |
| MDM4 | −2 | | | | | | | |
| MET | | | | round 1 | | | | |
| MGC14376 | | 2 | | | | | | |
| MICAL1 | | | | round 1 | | | | |
| MME | | | | round 1 | | | | |
| MRCL3 | 1.5 | 2 | | | complex | | | |
| MRLC2 | | | | | | | paralog | |
| MST1R | | | | round 1 | | | | |
| MUC1 | | | five | round 1 | | | | |
| MUC5B | | | | | | 1 | | |
| MYC | | | core | round 2 | | | | |
| MYH9 | | 1.5 | | | complex | | | |
| MYL9 | | | | | | | paralog | |
| NBEA | | | | | | 1 | | |
| NCK1 | | | core | round 1 | | | | |
| NCK2 | | | core | round 1 | | | | |
| NDUFA13 | | | five | round 2 | | | | |
| NEDD9 | | | | round 1 | | | | |
| NFKB1 | | 1.5 | five | round 2 | | | | |
| NGFR | | | | round 1 | | | | |
| NOTCH2 | | | | | | 1 | | |
| NPHP1 | | | | round 1 | | | | |
| NPTN | | 2 | | | | | | |
| NR4A1 | 1.5 | 2 | | | | | | |
| NR4A2 | | 2 | | | | | | |
| NRAS | | | core | | | 1 | | |
| NRG1 | | | | round 1 | | | | |
| NRG2 | | | | | | | | expert |
| NTRK1 | | | | round 1 | | | | |
| NTRK2 | | | | round 1 | | | | |
| NTRK3 | | | | round 1 | | | | |
| OVOL1 | | | | | | 1 | | |
| PABPC1 | | 1.5 | | | complex | | | |
| PABPC3 | | | | | | | paralog | |
| PABPC4 | | | | | | | paralog | |
| PAFAH1B1 | | | | | | 1 | | |
| PAG1 | | | | round 1 | | | | |
| PAK1 | | | core | round 1 | | | | |
| PAK2 | | | | | | | paralog | |
| PAK3 | | | | | | | paralog | |
| PAR6 | | | | | | | | expert |
| PARD3 | | | | | | | | expert |
| PCYT1A | | | | | | 2 | | |
| PCYT1B | | | | | | 1 | | |
| PDGFRB | | | | round 1 | | | | |
| PDLIM7 | | | | round 2 | complex | | | |
| PDPK1 | | | core | round 2 | | | | |
| PDZK1 | 2 | 1.5 | | round 2 | | | | |
| PEBP1 | | | five | round 2 | | | | |
| PICK1 | | | | round 1 | | | | |
| PIK3C2B | | | five | round 1 | | | | |
| PIK3CA | | | core | round 2 | | | | |
| PIK3CB | | | five | round 2 | | | | |
| PIK3CD | | | five | round 2 | | | | |
| PIK3CG | | | five | round 2 | | | | |
| PIK3R1 | | | core | round 1 | | | | |
| PIK3R2 | | | five | round 1 | | | | |
| PIK3R3 | | | five | round 2 | | | | |
| Pin1 | | | | | | | | expert |
| PIP5K1A | | | five | round 2 | | | | |
| PIP5K1B | | | five | round 2 | | | | |
| PIP5K2A | | | five | round 2 | | | | |
| PIP5K2B | | | five | round 2 | | | | |
| PITPNA | | | five | round 1 | | 2 | | |
| PITPNB | | | | | | 1 | | |
| PKD1 | | | | round 1 | | | | |
| PKN2 | | | five | round 2 | | | | |
| PLCB1 | | | five | round 2 | | | | |
| PLCG1 | | | core | round 1 | | 2 | | |
| PLCG2 | | | | round 1 | | 1 | | |
| PLD1 | | | core | round 2 | | | | |
| PLD1 | | | | | | | paralog | |
| PLD2 | | | five | round 1 | | | | |
| PLEC1 | | | five | round 1 | complex | | | |
| PLSCR1 | | 1.5 | five | round 1 | | | | |
| POU3F1 | | | | | | 4 | | |
| POU3F2 | | | | | | 3 | | |
| POU3F3 | | | | | | 2 | | |
| POU3F4 | | | | | | 1 | | |
| PPIA | | | | round 2 | complex | | | |
| PPP1CB | | | five | round 2 | complex | | | |
| PPP1R10 | 2 | 2 | | | | | | |
| PPP2R5A | | | | round 1 | | | | |
| PPP4R2 | | 2 | | | | | | |
| PRKACA | | | | round 1 | | | | |
| PRKACB | | | | | | | paralog | |
| PRKACG | | | | | | | paralog | |
| PRKAR1A | | −1.5 | five | round 1 | | | | |
| PRKCA | | | core | round 1 | | | | |
| PRKCB1 | | | core | | | | | |
| PRKCD | | | five | round 1 | | | | |
| PRKCE | | | five | round 2 | | | | |
| PRKCG | | | core | | | | | |
| PRKCI | | | core | | | | | |
| PRKCQ | | | five | round 2 | | | | |
| PRKCZ | | | core | round 2 | | | | |
| PRKD1 | | | core | round 2 | | | | |
| PRKDC | | | | round 2 | complex | | | |
| PRKX | | | | | | | paralog | |
| PRMT5 | | | | round 2 | complex | | | |
| PRODH | | | | | | 1 | | |
| PRSS12 | | | | | | 1 | | |
| PSME4 | | 2 | | | | | | |
| PTEN | | | five | round 2 | | | | |
| PTGER4 | −2 | | five | | | | | |
| PTK2 | | | five | round 1 | complex | | | |
| PTK2B | | | core | round 1 | complex | | | |
| PTK6 | | | five | round 1 | | | | |
| PTPN1 | | | | round 1 | | | | |
| PTPN11 | | | core | round 1 | complex | | | |
| PTPN12 | | | five | round 1 | | | | |
| PTPN2 | | | | | | | paralog | |
| PTPN6 | | | five | round 2 | | | | |
| PTPRF | | | | round 1 | | | | |
| PTPRH | | | | round 1 | | | | |
| PTPRJ | | | | round 1 | | | | |
| PXN | | | core | round 1 | complex | | | |
| RAB22A | | 2 | | | | | | |
| RAB5A | | | core | | | | | |
| RAC1 | | | core | round 2 | | | | |
| RAC2 | | | | | | | paralog | |
| RAC3 | | | | | | | paralog | |
| RACGAP1 | | | | round 2 | complex | | | |
| RAF1 | | 1.5 | core | round 2 | | 1 | | |
| RALA | | | | | | | paralog | |
| RALB | | | five | round 2 | | | | |
| RAP1A | | 1.5 | | round 2 | | | | |
| RAP1B | | | | | | | paralog | |
| RAP2A | | | | | | | paralog | |
| RAPGEF1 | | | | round 1 | | | | |
| RARA | 1.5 | 2 | | round 2 | | | | |
| RARB | | | | | | | paralog | |
| RARG | | | | | | | paralog | |
| RASA1 | | | core | round 1 | | 1 | | |
| RASA2 | | | | | | 2 | | |
| RASA3 | | | | | | 1 | | |
| RASD1 | | 2 | | round 2 | | | | |
| RB1 | | | five | round 2 | | | | |
| RBBP4 | | | | | | | paralog | |
| RBBP7 | | | five | | complex | | | |
| REPS1 | | | five | round 2 | | | | |
| RET | | 1.5 | | round 1 | | | | |
| RGS16 | | | five | round 1 | | | | |
| RGS4 | | | five | round 2 | | | | |
| RHO | | | | | | 1 | | |
| RHOA | | | core | round 2 | | 2 | | |
| RHOB | | | | | | | paralog | |
| RHOC | | | five | | | 1 | | |
| RHOG | | | five | round 2 | | | | |
| RICS | | | | round 1 | | | | |
| RIPK1 | | | five | round 1 | | | | |
| RLF | | 2 | | | | | | |
| ROS1 | | | | round 2 | | 1 | | |
| RP11- | | | | | | | paralog | |
| 78J21.1 | | | | | | | | |
| RPL10 | | 2 | | | complex | | | |
| RPL10L | | | | | | | paralog | |
| RPL23 | | 1.5 | | | complex | | | |
| RPS6KA1 | | | core | | | | | |
| RPS6KA2 | | | core | | | | | |
| RPS6KA3 | | | core | | | | | |
| RPS6KA5 | | | core | | | | | |
| RRAS | | | five | round 2 | | | | |
| RRAS2 | | | five | round 2 | | | | |
| RREB1 | 1.5 | 1.5 | | | | 1 | | |
| RUNX1 | 1.5 | 1.5 | | round 2 | | | | |
| RUVBL2 | | | | round 2 | complex | | | |
| RYR1 | | | five | round 2 | | | | |
| SAFB2 | | | | round 2 | complex | | | |
| SC4MOL | 2 | 2 | | | | | | |
| SERPINA3 | | | | round 1 | | | | |
| SFN | | | | round 2 | complex | | | |
| SH2D3A | | | | round 1 | | | | |
| SH2D3C | | | five | round 1 | | | | |
| SH3GL3 | | | five | round 2 | | | | |
| SH3KBP1 | | | five | round 2 | | | | |
| SHC1 | | | core | round 1 | complex | 3 | | |
| SHC2 | | | | | | 2 | | |
| SHC3 | | | five | round 1 | | 1 | | |
| SHCBP1 | | | | round 1 | | | | |
| SKIL | | 1.5 | | round 2 | | | | |
| SLC35A3 | | −2 | | | | | | |
| SLC39A6 | | −2 | | | | | | |
| SLPI | 1.5 | | | round 2 | | | | |
| SMAD1 | | | | round 1 | | | | |
| SMAD2 | | | five | round 1 | | | | |
| SMAD3 | | | five | round 1 | | | | |
| SMAD9 | | | | | | | paralog | |
| SMARCB1 | | | | round 2 | | 1 | | |
| SNCA | | | five | round 2 | | | | |
| SNF1LK | | 2 | | | | | | |
| SNIP | | | | round 1 | | | | |
| SNRPD2 | | | five | round 1 | complex | | | |
| SNX1 | | | | round 1 | | | | |
| SNX2 | | | | round 1 | | | | |
| SNX4 | | | | round 1 | | | | |
| SNX6 | | | | round 1 | | | | |
| SOCS1 | | | five | round 1 | | | | |
| SOCS3 | 1.5 | | five | round 1 | | | | |
| SORBS3 | | | | round 1 | | | | |
| SOS1 | 1.5 | | core | round 1 | complex | 2 | | |
| SOS2 | | | five | round 1 | | 1 | | |
| SP1 | | | five | round 1 | | | | |
| SPECC1 | | 1.5 | | | complex | | | |
| SPEN | | | | | | 1 | | |
| SPG7 | | 1.5 | | round 2 | | | | |
| SPIRE1 | | | | | | 1 | | |
| SPRY1 | | | | round 2 | | 4 | | |
| SPRY2 | | | core | round 2 | | 3 | | |
| SPRY3 | | | | | | 2 | | |
| SPRY4 | | | | | | 1 | | |
| SPTAN1 | | | | round 2 | complex | | | |
| SQLE | | 2 | | | | | | |
| SRC | | | core | round 1 | | | | |
| SRF | | | five | round 2 | | 1 | | |
| SSR3 | −2 | −2 | | | | | | |
| STAT1 | | | core | round 1 | | | | |
| STAT2 | | | five | round 2 | | | | |
| STAT3 | | | core | round 1 | complex | | | |
| STAT4 | | | | | | | paralog | |
| STAT5A | | | core | round 1 | | 3 | | |
| STAT5B | | | five | round 1 | | 2 | | |
| STAT6 | | | | round 2 | | 1 | | |
| STK3 | | | | | | | paralog | |
| STK4 | | 2 | | | | | | |
| STUB1 | | | | round 1 | | | | |
| TAF9B | | −2 | | | | | | |
| TBL1X | | | | | | | paralog | |
| TBL1XR1 | | | | | | 2 | | |
| TBL1Y | | | | | | 1 | | |
| TCF12 | | | | | | 3 | | |
| TCF3 | | | | round 1 | | 2 | | |
| TCF4 | | | | round 2 | | 1 | | |
| TFDP2 | −2 | | | | | 1 | | |
| TGFA | | | | round 1 | | | | |
| TGFBR1 | | 1.5 | | round 2 | | | | |
| TJP1 | | | | round 1 | | | | |
| TLE3 | | | | | | 1 | | |
| TLN1 | | 1.5 | | round 2 | | | | |
| TMEM1 | | 2 | | | | | | |
| TMPO | | −2 | | | | | | |
| TNC | | | | round 1 | | | | |
| TNK2 | | | five | round 1 | | | | |
| TOB1 | | | | round 1 | | | | |
| TP53 | | | five | round 2 | | | | |
| TPM4 | | 2 | | | | | | |
| TPR | | | | round 1 | | | | |
| TRAF4 | | 1.5 | | round 2 | | | | |
| TRAPPC6A | | | | round 1 | | | | |
| TRIM24 | | | | round 2 | complex | | | |
| TRIP12 | 1.5 | 2 | | | | | | |
| TRIP6 | | | | round 1 | | | | |
| TRPM7 | | 1.5 | | round 2 | | | | |
| TUBA1B | | | | round 2 | complex | | | |
| TUBA2 | | | | | | | paralog | |
| TUBA3 | | | | | | | paralog | |
| TUBA6 | | | | | | | paralog | |
| TXNIP | | −2 | | | | | | |
| UBE1L2 | | 2 | | | | | | |
| UBE2L3 | | | five | round 2 | | | | |
| UQCRFS1 | | | | | | 2 | | |
| VAV1 | | | core | round 1 | | | | |
| VAV2 | | | core | round 1 | complex | | | |
| VAV3 | | | core | round 1 | complex | | | |
| VEGFC | | | | | | 1 | | |
| WASL | −2 | | core | round 2 | | | | |
| WDR1 | | 2 | | | complex | | | |
| WWP1 | | | | | | | paralog | |
| WWP2 | | | | | | | paralog | |
| XRCC5 | | 2 | | round 2 | | | | |
| XRCC6 | | | | round 1 | complex | | | |
| YES1 | | | | round 2 | complex | | | |
| YWHAB | | | five | round 2 | | | | |
| YWHAE | | | | round 2 | complex | | | |
| YWHAQ | | | five | round 2 | complex | | | |
| YWHAZ | | | | round 1 | complex | | | |
| ZAP70 | | | | round 1 | | | | |
| ZNF259 | | | | round 1 | | | | |
| ZNF69 | −2 | | | | | | | |
| ZYX | | | | round 1 | | | | |

TABLE 2

| | Erlotinib | | Panitumumab | | CPT11 | | U0126 | |
|---|---|---|---|---|---|---|---|---|
| | Gene | Ratio | Gene | Ratio | Gene | Ratio | Gene | Ratio |
| 1 | ALK | 0.57 | ALK | 0.77 | ALK | 0.73 | | |
| 2 | ANXA6 | 0.47 | ANXA6 | 0.78 | ANXA6 | 0.82 | ANXA6 | 0.64 |
| 3 | ASCL2 | 0.52 | | | ASCL2 | 0.82 | | |
| 4 | BCL3 | 0.64 | | | BCL3 | 0.64 | | |
| 5 | CAV2 | 0.56 | | | CAV2 | 0.72 | | |
| 6 | CD22 | 0.70 | | | CD22 | 0.79 | | |
| 7 | CD59 | 0.71 | | | CD59 | 0.82 | | |
| 8 | CDC42 | 0.72 | | | CDC42 | 0.73 | | |
| 9 | CTNNA1 | 0.72 | | | CTNNA1 | 0.69 | | |
| 10 | DCN | 0.71 | | | DCN | 0.71 | | |
| 11 | EPB41L1 | 0.44 | | | EPB41L1 | 0.73 | | |
| 12 | EPHA5 | 0.67 | EPHA5 | 0.59 | EPHA5 | 0.61 | | |
| 13 | ERBB3 | 0.61 | | | ERBB3 | 0.49 | ERBB3 | 0.76 |
| 14 | FGFR2 | 0.73 | | | FGFR2 | 0.72 | | |
| 15 | RAPGEF1 | 0.59 | RAPGEF1 | 0.69 | RAPGEF1 | 0.84 | | |
| 16 | PLCG2 | 0.57 | | | PLCG2 | 0.65 | PLCG2 | 0.68 |
| 17 | PLSCR1 | 0.61 | PLSCR1 | 0.71 | PLSCR1 | 0.72 | | |
| 18 | PRKACB | 0.53 | PRKACB | 0.63 | PRKACB | 0.43 | | |
| 19 | PRKCE | 0.52 | PRKCE | 0.66 | PRKCE | 0.70 | | |
| 20 | SC4MOL | 0.61 | | | SC4MOL | 0.65 | | |
| 21 | CXCL12 | 0.61 | | | CXCL12 | 0.70 | CXCL12* | 0.83 |
| 22 | SLP1 | 0.60 | | | SLP1 | 0.84 | | |
| 23 | SOS2 | 0.69 | | | SOS2 | 0.74 | | |
| 24 | STAT3 | 0.56 | | | STAT3 | 0.66 | STAT3* | 0.75 |
| 25 | TLN1 | 0.72 | | | TLN1 | 0.80 | | |
| 26 | PIP5K1B | 0.72 | | | PIP5K1B | 0.75 | | |
| 27 | BCAR3 | 0.58 | | | BCAR3 | 0.52 | | |
| 28 | TRIP12 | 0.63 | | | TRIP12 | 0.50 | | |
| 29 | BCAR1 | 0.67 | | | BCAR1 | 0.80 | | |
| 30 | TOB1 | 0.38 | | | TOB1 | 0.75 | | |
| 31 | VAV3 | 0.66 | | | VAV3 | 0.76 | | |
| 32 | SPECC1 | 0.68 | SPECC1 | 0.73 | SPECC1 | 0.75 | SPECC1 | 0.76 |
| 33 | ARF4 | 0.79 | | | | | | |
| 34 | ARF5 | 0.82 | | | | | | |
| 35 | RHOA | 0.63 | RHOA | 0.67 | | | | |
| 36 | CALD1 | 0.76 | | | | | | |
| 37 | CDC25A | 0.78 | | | | | | |
| 38 | CDH3 | 0.78 | | | | | | |
| 39 | DAG1 | 0.74 | | | | | | |
| 40 | DLG4 | 0.73 | | | | | | |
| 41 | DOCK2 | 0.74 | | | | | | |
| 42 | DUSP4 | 0.71 | | | | | | |
| 43 | DUSP7 | 0.83 | | | | | | |
| 44 | EGR3 | 0.73 | | | | | | |
| 45 | ERBB2 | 0.80 | | | | | | |
| 46 | FCGR3A | 0.52 | | | | | | |
| 47 | FER | 0.63 | | | | | | |
| 48 | FES | 0.66 | | | | | | |
| 49 | FGR | 0.70 | | | | | | |
| 50 | FLNA | 0.48 | | | | | | |
| 51 | FUS | 0.81 | | | | | | |
| 52 | GAPDH | 0.68 | | | | | | |
| 53 | GNAI2 | 0.65 | | | | | GNAI2 | 0.58 |
| 54 | GRB7 | 0.67 | | | | | | |
| 55 | GRB14 | 0.54 | | | | | | |
| 56 | NRG1 | 0.70 | | | | | | |
| 57 | HIP1 | 0.59 | | | | | | |
| 58 | HES1 | 0.66 | | | | | | |
| 59 | INPPL1 | 0.57 | | | | | | |
| 60 | LTK | 0.55 | | | | | LTK | 0.81 |
| 61 | MATK | 0.70 | | | | | | |
| 62 | MAP3K1 | 0.66 | | | | | | |
| 63 | MYC | 0.78 | | | | | | |
| 64 | POU3F2 | 0.65 | POU3F2 | 0.69 | | | | |
| 65 | PKN2 | 0.66 | | | | | | |
| 66 | MAP2K1 | 0.34 | | | | | | |
| 67 | RET | 0.75 | | | | | | |
| 68 | RPL10 | 0.55 | | | | | | |
| 69 | RPS6KA3 | 0.69 | | | | | | |
| 70 | SHC1 | 0.63 | | | | | | |
| 71 | SRF | 0.81 | | | | | | |
| 72 | KLF10 | 0.69 | | | | | | |
| 73 | TMEM1 | 0.77 | | | | | | |
| 74 | RPS6KA5 | 0.62 | | | | | | |
| 75 | RPL23 | 0.69 | | | | | | |
| 76 | NRG2 | 0.69 | | | | | | |
| 77 | SH2D3C | 0.61 | | | | | | |
| 78 | ANKRD11 | 0.74 | | | | | | |
| 79 | DLL4 | 0.75 | | | | | | |
| 80 | PARD3 | 0.72 | | | | | | |
| 81 | LOC63920 | 0.62 | | | | | | |
| 82 | DIXDC1 | 0.81 | | | | | | |
| 83 | TBL1Y | 0.66 | | | | | | |
| 84 | | | | | ACTN4 | 0.83 | | |
| 85 | | | | | BCR | 0.78 | | |
| 86 | | | | | CEACAM1 | 0.61 | | |
| 87 | | | | | BLK | 0.69 | | |
| 88 | | | | | CAMLG | 0.76 | | |
| 89 | | | | | CAV3 | 0.78 | | |
| 90 | | | | | CBLB | 0.60 | | |
| 91 | | | | | CHUK | 0.74 | | |
| 92 | | | | | CCR5 | 0.60 | | |
| 93 | | | | | CTGF | 0.83 | | |
| 94 | | | | | DUSP1 | 0.58 | | |
| 95 | | | | | EPHA7 | 0.73 | | |
| 96 | | | | | ESR1 | 0.76 | | |
| 97 | | | | | EYA2 | 0.81 | | |
| 98 | | | | | GRB10 | 0.76 | | |
| 99 | | | | | HSPA5 | 0.80 | | |
| 100 | | | | | TNC | 0.72 | | |
| 101 | | | | | SMAD9 | 0.71 | | |
| 102 | | | | | PIK3R2 | 0.44 | | |
| 103 | | | | | PLEC1 | 0.67 | | |
| 104 | | | | | PRKACG | 0.74 | | |
| 105 | | | | | PRKCB1 | 0.71 | | |
| 106 | | | | | RHO | 0.82 | | |
| 107 | | | | | RRAS | 0.82 | | |
| 108 | | | | | TGFBR1 | 0.77 | | |
| 109 | | | | | TPR | 0.82 | | |
| 110 | | | | | UBE2L3 | 0.76 | | |
| 111 | | | | | MAPKAPK2 | 0.81 | | |
| 112 | | | | | TRAF4 | 0.67 | | |
| 113 | | | | | EIF4A3 | 0.56 | | |
| 114 | | | | | GAB2 | 0.83 | | |
| 115 | | | | | SORBS3 | 0.56 | | |
| 116 | | | | | FZR1 | 0.68 | | |
| 117 | | | | | FMN2 | 0.64 | | |
| 118 | | | | | REPS1 | 0.70 | | |
| 119 | | | | | | | LYN | 0.82 |
| 120 | | | LOC390006 | 0.76 | | | LOC390006 | 0.66 |
| 121 | | | LOC284393 | 0.75 | | | | |

Notes.
False discovery rate set at 5%, except for 2 U0126 hits marked *, where 7.5% was used (statisticians say up to 20% is ok).

TABLE 3

| | | SEQ ID | Entrez | NCBI gene | |
|---|---|---|---|---|---|
| Plate Id | Plate Name | NO: | Gene Id | symbol | Gene Description |
| 900029-1-A | single siRNA, 0.9 nmol | 1 | 12 | SERPINA3 | serpin peptidase inhibitor, clade [symbol] |
| 900029-1-A | single siRNA, 0.9 nmol | 2 | 207 | AKT1 | v-akt murine thymoma viral onco [symbol] |
| 900029-1-A | single siRNA, 0.9 nmol | 3 | 331 | BIRC4 | baculoviral IAP repeat-containing [symbol] |
| 900029-1-A | single siRNA, 0.9 nmol | 4 | 378 | ARF4 | ADP-ribosylation factor 4 |
| 900029-1-A | single siRNA, 0.9 nmol | 5 | 389 | RHOC | ras homolog gene family, membe [symbol] |
| 900029-1-A | single siRNA, 0.9 nmol | 6 | 595 | CCND1 | cyclin D1 |
| 900029-1-A | single siRNA, 0.9 nmol | 7 | 652 | BMP4 | bone morphogenetic protein 4 |
| 900029-1-A | single siRNA, 0.9 nmol | 8 | 800 | CALD1 | caldesmon 1 |
| 900029-1-A | single siRNA, 0.9 nmol | 9 | 834 | CASP1 | caspase 1, apoptosis-related cyst [symbol] |
| 900029-1-A | single siRNA, 0.9 nmol | 10 | 868 | CBLB | Cas-Br-M (murine) ecotropic retrc [symbol] |
| 900029-1-A | single siRNA, 0.9 nmol | 11 | 25 | ABL1 | v-abl Abelson murine leukemia vi [symbol] |
| 900029-1-A | single siRNA, 0.9 nmol | 12 | 208 | AKT2 | v-akt murine thymoma viral oncog [symbol] |
| 900029-1-A | single siRNA, 0.9 nmol | 13 | 351 | APP | amyloid beta (A4) precursor prote [symbol] |
| 900029-1-A | single siRNA, 0.9 nmol | 14 | 381 | ARF5 | ADP-ribosylation factor 5 |
| 900029-1-A | single siRNA, 0.9 nmol | 15 | 391 | RHOG | ras homolog gene family, membe [symbol] |
| 900029-1-A | single siRNA, 0.9 nmol | 16 | 602 | BCL3 | B-cell CLL/lymphoma 3 |
| 900029-1-A | single siRNA, 0.9 nmol | 17 | 657 | BMPR1A | bone morphogenetic protein rece [symbol] |
| 900029-1-A | single siRNA, 0.9 nmol | 18 | 801 | CALM1 | calmodulin 1 (phosphorylase kina [symbol] |
| 900029-1-A | single siRNA, 0.9 nmol | 19 | 857 | CAV1 | caveolin 1, caveolae protein, 22k [symbol] |
| 900029-1-A | single siRNA, 0.9 nmol | 20 | 894 | CCND2 | cyclin D2 |
| 900029-1-A | single siRNA, 0.9 nmol | 21 | 70 | ACTC1 | actin, alpha, cardiac muscle 1 |
| 900029-1-A | single siRNA, 0.9 nmol | 22 | 238 | ALK | anaplastic lymphoma kinase (Ki-1 [symbol] |
| 900029-1-A | single siRNA, 0.9 nmol | 23 | 369 | ARAF | v-raf murine sarcoma 3611 viral c [symbol] |
| 900029-1-A | single siRNA, 0.9 nmol | 24 | 383 | ARG1 | arginase, liver |
| 900029-1-A | single siRNA, 0.9 nmol | 25 | 429 | ASCL1 | achaete-scute complex homolog [symbol] |
| 900029-1-A | single siRNA, 0.9 nmol | 26 | 613 | BCR | breakpoint cluster region |
| 900029-1-A | single siRNA, 0.9 nmol | 27 | 658 | BMPR1B | bone morphogenetic protein rece [symbol] |
| 900029-1-A | single siRNA, 0.9 nmol | 28 | 805 | CALM2 | calmodulin 2 (phosphorylase kina [symbol] |
| 900029-1-A | single siRNA, 0.9 nmol | 29 | 858 | CAV2 | caveolin 2 |
| 900029-1-A | single siRNA, 0.9 nmol | 30 | 896 | CCND3 | cyclin D3 |
| 900029-1-A | single siRNA, 0.9 nmol | 31 | 81 | ACTN4 | actinin, alpha 4 |
| 900029-1-A | single siRNA, 0.9 nmol | 32 | 268 | AMH | anti-Mullerian hormone |
| 900029-1-A | single siRNA, 0.9 nmol | 33 | 374 | AREG | amphiregulin (schwannoma-deriv [symbol] |
| 900029-1-A | single siRNA, 0.9 nmol | 34 | 384 | ARG2 | arginase, type II |
| 900029-1-A | single siRNA, 0.9 nmol | 35 | 430 | ASCL2 | achaete-scute complex homolog [symbol] |
| 900029-1-A | single siRNA, 0.9 nmol | 36 | 634 | CEACAM1 | carcinoembryonic antigen-related [symbol] |
| 900029-1-A | single siRNA, 0.9 nmol | 37 | 673 | BRAF | v-raf murine sarcoma viral oncog [symbol] |
| 900029-1-A | single siRNA, 0.9 nmol | 38 | 808 | CALM3 | calmodulin 3 (phosphorylase kina [symbol] |
| 900029-1-A | single siRNA, 0.9 nmol | 39 | 859 | CAV3 | caveolin 3 |
| 900029-1-A | single siRNA, 0.9 nmol | 40 | 898 | CCNE1 | cyclin E1 |
| 900029-1-A | single siRNA, 0.9 nmol | 41 | 160 | AP2A1 | adaptor-related protein complex 2 [symbol] |
| 900029-1-A | single siRNA, 0.9 nmol | 42 | 302 | ANXA2 | annexin A2 |
| 900029-1-A | single siRNA, 0.9 nmol | 43 | 375 | ARF1 | ADP-ribosylation factor 1 |
| 900029-1-A | single siRNA, 0.9 nmol | 44 | 387 | RHOA | ras homolog gene family, membe [symbol] |
| 900029-1-A | single siRNA, 0.9 nmol | 45 | 483 | ATP1B3 | ATPase, Na+/K+ transporting, be [symbol] |
| 900029-1-A | single siRNA, 0.9 nmol | 46 | 640 | BLK | B lymphoid tyrosine kinase |
| 900029-1-A | single siRNA, 0.9 nmol | 47 | 685 | BTC | betacellulin [symbol] |
| 900029-1-A | single siRNA, 0.9 nmol | 48 | 818 | CAMK2G | calcium/calmodulin-dependent pr [symbol] |
| 900029-1-A | single siRNA, 0.9 nmol | 49 | 861 | RUNX1 | runt-related transcription factor 1 |
| 900029-1-A | single siRNA, 0.9 nmol | 50 | 916 | CD3E | CD3e molecule, epsilon (CD3-TC [symbol] |
| 900029-1-A | single siRNA, 0.9 nmol | 51 | 161 | AP2A2 | adaptor-related protein complex 2 [symbol] |
| 900029-1-A | single siRNA, 0.9 nmol | 52 | 309 | ANXA6 | annexin A6 |
| 900029-1-A | single siRNA, 0.9 nmol | 53 | 377 | ARF3 | ADP-ribosylation factor 3 |
| 900029-1-A | single siRNA, 0.9 nmol | 54 | 388 | RHOB | ras homolog gene family, membe [symbol] |
| 900029-1-A | single siRNA, 0.9 nmol | 55 | 558 | AXL | AXL receptor tyrosine kinase |
| 900029-1-A | single siRNA, 0.9 nmol | 56 | 650 | BMP2 | bone morphogenetic protein 2 [symbol] |
| 900029-1-A | single siRNA, 0.9 nmol | 57 | 780 | DDR1 | discoidin domain receptor family, [symbol] |
| 900029-1-A | single siRNA, 0.9 nmol | 58 | 819 | CAMLG | calcium modulating ligand |
| 900029-1-A | single siRNA, 0.9 nmol | 59 | 867 | CBL | Cas-Br-M (murine) ecotropic retrc [symbol] |
| 900029-1-A | single siRNA, 0.9 nmol | 60 | 919 | CD247 | CD247 molecule [symbol] |
| 900029-1-B | single siRNA, 0.9 nmol | 61 | 12 | SERPINA3 | serpin peptidase inhibitor, clade [symbol] |
| 900029-1-B | single siRNA, 0.9 nmol | 62 | 207 | AKT1 | v-akt murine thymoma viral onco [symbol] |
| 900029-1-B | single siRNA, 0.9 nmol | 63 | 331 | BIRC4 | baculoviral IAP repeat-containing [symbol] |
| 900029-1-B | single siRNA, 0.9 nmol | 64 | 378 | ARF4 | ADP-ribosylation factor 4 |
| 900029-1-B | single siRNA, 0.9 nmol | 65 | 389 | RHOC | ras homolog gene family, membe [symbol] |
| 900029-1-B | single siRNA, 0.9 nmol | 66 | 595 | CCND1 | cyclin D1 |
| 900029-1-B | single siRNA, 0.9 nmol | 67 | 652 | BMP4 | bone morphogenetic protein 4 |
| 900029-1-B | single siRNA, 0.9 nmol | 68 | 800 | CALD1 | caldesmon 1 [symbol] |
| 900029-1-B | single siRNA, 0.9 nmol | 69 | 834 | CASP1 | caspase 1, apoptosis-related cyst [symbol] |
| 900029-1-B | single siRNA, 0.9 nmol | 70 | 868 | CBLB | Cas-Br-M (murine) ecotropic retrc [symbol] |
| 900029-1-B | single siRNA, 0.9 nmol | 71 | 25 | ABL1 | v-abl Abelson murine leukemia vi [symbol] |
| 900029-1-B | single siRNA, 0.9 nmol | 72 | 208 | AKT2 | v-akt murine thymoma viral onco [symbol] |
| 900029-1-B | single siRNA, 0.9 nmol | 73 | 351 | APP | amyloid beta (A4) precursor prote [symbol] |
| 900029-1-B | single siRNA, 0.9 nmol | 74 | 381 | ARF5 | ADP-ribosylation factor 5 |
| 900029-1-B | single siRNA, 0.9 nmol | 75 | 391 | RHOG | ras homolog gene family, membe [symbol] |
| 900029-1-B | single siRNA, 0.9 nmol | 76 | 602 | BCL3 | B-cell CLL/lymphoma 3 |
| 900029-1-B | single siRNA, 0.9 nmol | 77 | 657 | BMPR1A | bone morphogenetic protein rece [symbol] |
| 900029-1-B | single siRNA, 0.9 nmol | 78 | 801 | CALM1 | calmodulin 1 (phosphorylase kina [symbol] |
| 900029-1-B | single siRNA, 0.9 nmol | 79 | 857 | CAV1 | caveolin 1, caveolae protein, 22k [symbol] |
| 900029-1-B | single siRNA, 0.9 nmol | 80 | 894 | CCND2 | cyclin D2 |
| 900029-1-B | single siRNA, 0.9 nmol | 81 | 70 | ACTC1 | actin, alpha, cardiac muscle 1 |
| 900029-1-B | single siRNA, 0.9 nmol | 82 | 238 | ALK | anaplastic lymphoma kinase (Ki-1 [symbol] |
| 900029-1-B | single siRNA, 0.9 nmol | 83 | 369 | ARAF | v-raf murine sarcoma 3611 viral c [symbol] |
| 900029-1-B | single siRNA, 0.9 nmol | 84 | 383 | ARG1 | arginase, liver |
| 900029-1-B | single siRNA, 0.9 nmol | 85 | 429 | ASCL1 | achaete-scute complex homolog [symbol] |
| 900029-1-B | single siRNA, 0.9 nmol | 86 | 613 | BCR | breakpoint cluster region |
| 900029-1-B | single siRNA, 0.9 nmol | 87 | 658 | BMPR1B | bone morphogenetic protein rece [symbol] |
| 900029-1-B | single siRNA, 0.9 nmol | 88 | 805 | CALM2 | calmodulin 2 (phosphorylase kina [symbol] |
| 900029-1-B | single siRNA, 0.9 nmol | 89 | 858 | CAV2 | caveolin 2 |
| 900029-1-B | single siRNA, 0.9 nmol | 90 | 896 | CCND3 | cyclin D3 |
| 900029-1-B | single siRNA, 0.9 nmol | 91 | 81 | ACTN4 | actinin, alpha 4 |
| 900029-1-B | single siRNA, 0.9 nmol | 92 | 268 | AMH | anti-Mullerian hormone |
| 900029-1-B | single siRNA, 0.9 nmol | 93 | 374 | AREG | amphiregulin (schwannoma-deriv [symbol] |
| 900029-1-B | single siRNA, 0.9 nmol | 94 | 384 | ARG2 | arginase, type II |
| 900029-1-B | single siRNA, 0.9 nmol | 95 | 430 | ASCL2 | achaete-scute complex homolog [symbol] |
| 900029-1-B | single siRNA, 0.9 nmol | 96 | 634 | CEACAM1 | carcinoembryonic antigen-related [symbol] |
| 900029-1-B | single siRNA, 0.9 nmol | 97 | 673 | BRAF | v-raf murine sarcoma viral oncog [symbol] |
| 900029-1-B | single siRNA, 0.9 nmol | 98 | 808 | CALM3 | calmodulin 3 (phosphorylase kina [symbol] |
| 900029-1-B | single siRNA, 0.9 nmol | 99 | 859 | CAV3 | caveolin 3 |
| 900029-1-B | single siRNA, 0.9 nmol | 100 | 898 | CCNE1 | cyclin E1 |
| 900029-1-B | single siRNA, 0.9 nmol | 101 | 160 | AP2A1 | adaptor-related protein complex 2 |
| 900029-1-B | single siRNA, 0.9 nmol | 102 | 302 | ANXA2 | annexin A2 |
| 900029-1-B | single siRNA, 0.9 nmol | 103 | 375 | ARF1 | ADP-ribosylation factor 1 |
| 900029-1-B | single siRNA, 0.9 nmol | 104 | 387 | RHOA | ras homolog gene family, membe [symbol] |
| 900029-1-B | single siRNA, 0.9 nmol | 105 | 483 | ATP1B3 | ATPase, Na+/K+ transporting, be [symbol] |
| 900029-1-B | single siRNA, 0.9 nmol | 106 | 640 | BLK | B lymphoid tyrosine kinase [symbol] |
| 900029-1-B | single siRNA, 0.9 nmol | 107 | 685 | BTC | betacellulin |
| 900029-1-B | single siRNA, 0.9 nmol | 108 | 818 | CAMK2G | calcium/calmodulin-dependent pr [symbol] |
| 900029-1-B | single siRNA, 0.9 nmol | 109 | 861 | RUNX1 | runt-related transcription factor 1 |
| 900029-1-B | single siRNA, 0.9 nmol | 110 | 916 | CD3E | CD3e molecule, epsilon (CD3-TC [symbol] |
| 900029-1-B | single siRNA, 0.9 nmol | 111 | 161 | AP2A2 | adaptor-related protein complex 2 [symbol] |
| 900029-1-B | single siRNA, 0.9 nmol | 112 | 309 | ANXA6 | annexin A6 |
| 900029-1-B | single siRNA, 0.9 nmol | 113 | 377 | ARF3 | ADP-ribosylation factor 3 |
| 900029-1-B | single siRNA, 0.9 nmol | 114 | 388 | RHOB | ras homolog gene family, membe [symbol] |
| 900029-1-B | single siRNA, 0.9 nmol | 115 | 558 | AXL | AXL receptor tyrosine kinase |
| 900029-1-B | single siRNA, 0.9 nmol | 116 | 650 | BMP2 | bone morphogenetic protein 2 |
| 900029-1-B | single siRNA, 0.9 nmol | 117 | 780 | DDR1 | discoidin domain receptor family, [symbol] |
| 900029-1-B | single siRNA, 0.9 nmol | 118 | 819 | CAMLG | calcium modulating ligand |
| 900029-1-B | single siRNA, 0.9 nmol | 119 | 867 | CBL | Cas-Br-M (murine) ecotropic retr [symbol] |
| 900029-1-B | single siRNA, 0.9 nmol | 120 | 919 | CD247 | CD247 molecule |
| 900029-2-A | single siRNA, 0.9 nmol | 121 | 933 | CD22 | CD22 molecule |
| 900029-2-A | single siRNA, 0.9 nmol | 122 | 999 | CDH1 | cadherin 1, type 1, E-cadherin (er [symbol] |
| 900029-2-A | single siRNA, 0.9 nmol | 123 | 1103 | CHAT | choline acetyltransferase |
| 900029-2-A | single siRNA, 0.9 nmol | 124 | 1316 | KLF6 | Kruppel-like factor 6 |
| 900029-2-A | single siRNA, 0.9 nmol | 125 | 1441 | CSF3R | colony stimulating factor 3 recept [symbol] |
| 900029-2-A | single siRNA, 0.9 nmol | 126 | 1499 | CTNNB1 | catenin (cadherin-associated prot [symbol] |
| 900029-2-A | single siRNA, 0.9 nmol | 127 | 1742 | DLG4 | discs, large homolog 4 (Drosophil [symbol] |
| 900029-2-A | single siRNA, 0.9 nmol | 128 | 1846 | DUSP4 | dual specificity phosphatase 4 |
| 900029-2-A | single siRNA, 0.9 nmol | 129 | 1906 | EDN1 | endothelin 1 |
| 900029-2-A | single siRNA, 0.9 nmol | 130 | 1959 | EGR2 | early growth response 2 (Krox-20 [symbol] |
| 900029-2-A | single siRNA, 0.9 nmol | 131 | 960 | CD44 | CD44 molecule (Indian blood gro [symbol] |
| 900029-2-A | single siRNA, 0.9 nmol | 132 | 1001 | CDH3 | cadherin 3, type 1, P-cadherin (p [symbol] |
| 900029-2-A | single siRNA, 0.9 nmol | 133 | 1120 | CHKB | choline kinase beta |
| 900029-2-A | single siRNA, 0.9 nmol | 134 | 1385 | CREB1 | cAMP responsive element bindin [symbol] |
| 900029-2-A | single siRNA, 0.9 nmol | 135 | 1445 | CSK | c-src tyrosine kinase |
| 900029-2-A | single siRNA, 0.9 nmol | 136 | 1500 | CTNND1 | catenin (cadherin-associated prot [symbol] |
| 900029-2-A | single siRNA, 0.9 nmol | 137 | 1759 | DNM1 | dynamin 1 |
| 900029-2-A | single siRNA, 0.9 nmol | 138 | 1848 | DUSP6 | dual specificity phosphatase 6 |
| 900029-2-A | single siRNA, 0.9 nmol | 139 | 1915 | EEF1A1 | eukaryotic translation elongation [symbol] |
| 900029-2-A | single siRNA, 0.9 nmol | 140 | 1960 | EGR3 | early growth response 3 |
| 900029-2-A | single siRNA, 0.9 nmol | 141 | 966 | CD59 | CD59 molecule, complement reg [symbol] |
| 900029-2-A | single siRNA, 0.9 nmol | 142 | 1003 | CDH5 | cadherin 5, type 2, VE-cadherin ( [symbol] |
| 900029-2-A | single siRNA, 0.9 nmol | 143 | 1147 | CHUK | conserved helix-loop-helix ubiquit [symbol] |
| 900029-2-A | single siRNA, 0.9 nmol | 144 | 1398 | CRK | v-crk sarcoma virus CT10 oncog [symbol] |
| 900029-2-A | single siRNA, 0.9 nmol | 145 | 1457 | CSNK2A1 | casein kinase 2, alpha 1 polypept [symbol] |
| 900029-2-A | single siRNA, 0.9 nmol | 146 | 1523 | CUTL1 | cut-like 1, CCAAT displacement [symbol] |
| 900029-2-A | single siRNA, 0.9 nmol | 147 | 1793 | DOCK1 | dedicator of cytokinesis 1 |
| 900029-2-A | single siRNA, 0.9 nmol | 148 | 1849 | DUSP7 | dual specificity phosphatase 7 |
| 900029-2-A | single siRNA, 0.9 nmol | 149 | 1917 | EEF1A2 | eukaryotic translation elongation [symbol] |
| 900029-2-A | single siRNA, 0.9 nmol | 150 | 1969 | EPHA2 | EPH receptor A2 |
| 900029-2-A | single siRNA, 0.9 nmol | 151 | 987 | LRBA | LPS-responsive vesicle trafficking [symbol] |
| 900029-2-A | single siRNA, 0.9 nmol | 152 | 1017 | CDK2 | cyclin-dependent kinase 2 |
| 900029-2-A | single siRNA, 0.9 nmol | 153 | 1230 | CCR1 | chemokine (C-C motif) receptor 1 |
| 900029-2-A | single siRNA, 0.9 nmol | 154 | 1399 | CRKL | v-crk sarcoma virus CT10 oncoge [symbol] |
| 900029-2-A | single siRNA, 0.9 nmol | 155 | 1464 | CSPG4 | chondroitin sulfate proteoglycan 4 |
| 900029-2-A | single siRNA, 0.9 nmol | 156 | 1601 | DAB2 | disabled homolog 2, mitogen-res [symbol] |
| 900029-2-A | single siRNA, 0.9 nmol | 157 | 1794 | DOCK2 | dedicator of cytokinesis 2 |
| 900029-2-A | single siRNA, 0.9 nmol | 158 | 1852 | DUSP9 | dual specificity phosphatase 9 |
| 900029-2-A | single siRNA, 0.9 nmol | 159 | 1950 | EGF | epidermal growth factor (beta-uro [symbol] |
| 900029-2-A | single siRNA, 0.9 nmol | 160 | 1973 | EIF4A1 | eukaryotic translation initiation fa [symbol] |
| 900029-2-A | single siRNA, 0.9 nmol | 161 | 993 | CDC25A | cell division cycle 25 homolog A ( [symbol] |
| 900029-2-A | single siRNA, 0.9 nmol | 162 | 1050 | CEBPA | CCAAT/enhancer binding protein [symbol] |
| 900029-2-A | single siRNA, 0.9 nmol | 163 | 1231 | CCR2 | chemokine (C-C motif) receptor 2 |
| 900029-2-A | single siRNA, 0.9 nmol | 164 | 1432 | MAPK14 | mitogen-activated protein kinase [symbol] |
| 900029-2-A | single siRNA, 0.9 nmol | 165 | 1490 | CTGF | connective tissue growth factor |
| 900029-2-A | single siRNA, 0.9 nmol | 166 | 1605 | DAG1 | dystroglycan 1 (dystrophin-associ [symbol] |
| 900029-2-A | single siRNA, 0.9 nmol | 167 | 1839 | HBEGF | heparin-binding EGF-like growth 1 [symbol] |
| 900029-2-A | single siRNA, 0.9 nmol | 168 | 1875 | E2F5 | E2F transcription factor 5, p130-b [symbol] |
| 900029-2-A | single siRNA, 0.9 nmol | 169 | 1956 | EGFR | epidermal growth factor receptor [symbol] |
| 900029-2-A | single siRNA, 0.9 nmol | 170 | 1974 | EIF4A2 | eukaryotic translation initiation fa [symbol] |
| 900029-2-A | single siRNA, 0.9 nmol | 171 | 998 | CDC42 | cell division cycle 42 (GTP bindin [symbol] |
| 900029-2-A | single siRNA, 0.9 nmol | 172 | 1051 | CEBPB | CCAAT/enhancer binding protein [symbol] |
| 900029-2-A | single siRNA, 0.9 nmol | 173 | 1234 | CCR5 | chemokine (C-C motif) receptor 5 [symbol] |
| 900029-2-A | single siRNA, 0.9 nmol | 174 | 1439 | CSF2RB | colony stimulating factor 2 recept [symbol] |
| 900029-2-A | single siRNA, 0.9 nmol | 175 | 1495 | CTNNA1 | catenin (cadherin-associated prot [symbol] |
| 900029-2-A | single siRNA, 0.9 nmol | 176 | 1634 | DCN | decorin |
| 900029-2-A | single siRNA, 0.9 nmol | 177 | 1843 | DUSP1 | dual specificity phosphatase 1 |
| 900029-2-A | single siRNA, 0.9 nmol | 178 | 1879 | EBF1 | early B-cell factor 1 |
| 900029-2-A | single siRNA, 0.9 nmol | 179 | 1958 | EGR1 | early growth response 1 |
| 900029-2-A | single siRNA, 0.9 nmol | 180 | 1999 | ELF3 | E74-like factor 3 (ets domain tran [symbol] |
| 900029-2-B | single siRNA, 0.9 nmol | 181 | 933 | CD22 | CD22 molecule |
| 900029-2-B | single siRNA, 0.9 nmol | 182 | 999 | CDH1 | cadherin 1, type 1, E-cadherin (e [symbol] |
| 900029-2-B | single siRNA, 0.9 nmol | 183 | 1103 | CHAT | choline acetyltransferase |
| 900029-2-B | single siRNA, 0.9 nmol | 184 | 1316 | KLF6 | Kruppel-like factor 6 |
| 900029-2-B | single siRNA, 0.9 nmol | 185 | 1441 | CSF3R | colony stimulating factor 3 recept [symbol] |
| 900029-2-B | single siRNA, 0.9 nmol | 186 | 1499 | CTNNB1 | catenin (cadherin-associated prot [symbol] |
| 900029-2-B | single siRNA, 0.9 nmol | 187 | 1742 | DLG4 | discs, large homolog 4 (Drosophil [symbol] |
| 900029-2-B | single siRNA, 0.9 nmol | 188 | 1846 | DUSP4 | dual specificity phosphatase 4 |
| 900029-2-B | single siRNA, 0.9 nmol | 189 | 1906 | EDN1 | endothelin 1 |
| 900029-2-B | single siRNA, 0.9 nmol | 190 | 1959 | EGR2 | early growth response 2 (Krox-20 [symbol] |
| 900029-2-B | single siRNA, 0.9 nmol | 191 | 960 | CD44 | CD44 molecule (Indian blood gro [symbol] |
| 900029-2-B | single siRNA, 0.9 nmol | 192 | 1001 | CDH3 | cadherin 3, type 1, P-cadherin (p [symbol] |
| 900029-2-B | single siRNA, 0.9 nmol | 193 | 1120 | CHKB | choline kinase beta |
| 900029-2-B | single siRNA, 0.9 nmol | 194 | 1385 | CREB1 | cAMP responsive element bindin [symbol] |
| 900029-2-B | single siRNA, 0.9 nmol | 195 | 1445 | CSK | c-src tyrosine kinase |
| 900029-2-B | single siRNA, 0.9 nmol | 196 | 1500 | CTNND1 | catenin (cadherin-associated prot [symbol] |
| 900029-2-B | single siRNA, 0.9 nmol | 197 | 1759 | DNM1 | dynamin 1 |
| 900029-2-B | single siRNA, 0.9 nmol | 198 | 1848 | DUSP6 | dual specificity phosphatase 6 |
| 900029-2-B | single siRNA, 0.9 nmol | 199 | 1915 | EEF1A1 | eukaryotic translation elongation [symbol] |
| 900029-2-B | single siRNA, 0.9 nmol | 200 | 1960 | EGR3 | early growth response 3 |
| 900029-2-B | single siRNA, 0.9 nmol | 201 | 966 | CD59 | CD59 molecule, complement reg [symbol] |
| 900029-2-B | single siRNA, 0.9 nmol | 202 | 1003 | CDH5 | cadherin 5, type 2, VE-cadherin ( [symbol] |
| 900029-2-B | single siRNA, 0.9 nmol | 203 | 1147 | CHUK | conserved helix-loop-helix ubiquit [symbol] |
| 900029-2-B | single siRNA, 0.9 nmol | 204 | 1398 | CRK | v-crk sarcoma virus CT10 oncog [symbol] |
| 900029-2-B | single siRNA, 0.9 nmol | 205 | 1457 | CSNK2A1 | casein kinase 2, alpha 1 polypept [symbol] |
| 900029-2-B | single siRNA, 0.9 nmol | 206 | 1523 | CUTL1 | cut-like 1, CCAAT displacement [symbol] |
| 900029-2-B | single siRNA, 0.9 nmol | 207 | 1793 | DOCK1 | dedicator of cytokinesis 1 |
| 900029-2-B | single siRNA, 0.9 nmol | 208 | 1849 | DUSP7 | dual specificity phosphatase 7 |
| 900029-2-B | single siRNA, 0.9 nmol | 209 | 1917 | EEF1A2 | eukaryotic translation elongation [symbol] |
| 900029-2-B | single siRNA, 0.9 nmol | 210 | 1969 | EPHA2 | EPH receptor A2 |
| 900029-2-B | single siRNA, 0.9 nmol | 211 | 987 | LRBA | LPS-responsive vesicle trafficking [symbol] |
| 900029-2-B | single siRNA, 0.9 nmol | 212 | 1017 | CDK2 | cyclin-dependent kinase 2 |
| 900029-2-B | single siRNA, 0.9 nmol | 213 | 1230 | CCR1 | chemokine (C-C motif) receptor 1 |
| 900029-2-B | single siRNA, 0.9 nmol | 214 | 1399 | CRKL | v-crk sarcoma virus CT10 oncog [symbol] |
| 900029-2-B | single siRNA, 0.9 nmol | 215 | 1464 | CSPG4 | chondroitin sulfate proteoglycan 4 [symbol] |
| 900029-2-B | single siRNA, 0.9 nmol | 216 | 1601 | DAB2 | disabled homolog 2, mitogen-res [symbol] |
| 900029-2-B | single siRNA, 0.9 nmol | 217 | 1794 | DOCK2 | dedicator of cytokinesis 2 |
| 900029-2-B | single siRNA, 0.9 nmol | 218 | 1852 | DUSP9 | dual specificity phosphatase 9 |
| 900029-2-B | single siRNA, 0.9 nmol | 219 | 1950 | EGF | epidermal growth factor (beta-uro [symbol] |
| 900029-2-B | single siRNA, 0.9 nmol | 220 | 1973 | EIF4A1 | eukaryotic translation initiation fa [symbol] |
| 900029-2-B | single siRNA, 0.9 nmol | 221 | 993 | CDC25A | cell division cycle 25 homolog A ( [symbol] |
| 900029-2-B | single siRNA, 0.9 nmol | 222 | 1050 | CEBPA | CCAAT/enhancer binding protein [symbol] |
| 900029-2-B | single siRNA, 0.9 nmol | 223 | 1231 | CCR2 | chemokine (C-C motif) receptor 2 [symbol] |
| 900029-2-B | single siRNA, 0.9 nmol | 224 | 1432 | MAPK14 | mitogen-activated protein kinase [symbol] |
| 900029-2-B | single siRNA, 0.9 nmol | 225 | 1490 | CTGF | connective tissue growth factor |
| 900029-2-B | single siRNA, 0.9 nmol | 226 | 1605 | DAG1 | dystroglycan 1 (dystrophin-associ [symbol] |
| 900029-2-B | single siRNA, 0.9 nmol | 227 | 1839 | HBEGF | heparin-binding EGF-like growth [symbol] |
| 900029-2-B | single siRNA, 0.9 nmol | 228 | 1875 | E2F5 | E2F transcription factor 5, p130-b [symbol] |
| 900029-2-B | single siRNA, 0.9 nmol | 229 | 1956 | EGFR | epidermal growth factor receptor [symbol] |
| 900029-2-B | single siRNA, 0.9 nmol | 230 | 1974 | EIF4A2 | eukaryotic translation initiation fa [symbol] |
| 900029-2-B | single siRNA, 0.9 nmol | 231 | 998 | CDC42 | cell division cycle 42 (GTP bindin [symbol] |
| 900029-2-B | single siRNA, 0.9 nmol | 232 | 1051 | CEBPB | CCAAT/enhancer binding protein [symbol] |
| 900029-2-B | single siRNA, 0.9 nmol | 233 | 1234 | CCR5 | chemokine (C-C motif) receptor 5 |
| 900029-2-B | single siRNA, 0.9 nmol | 234 | 1439 | CSF2RB | colony stimulating factor 2 recept [symbol] |
| 900029-2-B | single siRNA, 0.9 nmol | 235 | 1495 | CTNNA1 | catenin (cadherin-associated prot [symbol] |
| 900029-2-B | single siRNA, 0.9 nmol | 236 | 1634 | DCN | decorin |
| 900029-2-B | single siRNA, 0.9 nmol | 237 | 1843 | DUSP1 | dual specificity phosphatase 1 |
| 900029-2-B | single siRNA, 0.9 nmol | 238 | 1879 | EBF1 | early B-cell factor 1 |
| 900029-2-B | single siRNA, 0.9 nmol | 239 | 1958 | EGR1 | early growth response 1 |
| 900029-2-B | single siRNA, 0.9 nmol | 240 | 1999 | ELF3 | E74-like factor 3 (ets domain tran [symbol] |
| 900029-3-A | single siRNA, 0.9 nmol | 241 | 2002 | ELK1 | ELK1, member of ETS oncogene [symbol] |
| 900029-3-A | single siRNA, 0.9 nmol | 242 | 2044 | EPHA5 | EPH receptor A5 |
| 900029-3-A | single siRNA, 0.9 nmol | 243 | 2065 | ERBB3 | v-erb-b2 erythroblastic leukemia [symbol] |
| 900029-3-A | single siRNA, 0.9 nmol | 244 | 2120 | ETV6 | ets variant gene 6 (TEL oncogen [symbol] |
| 900029-3-A | single siRNA, 0.9 nmol | 245 | 2222 | FDFT1 | farnesyl-diphosphate farnesyltran [symbol] |
| 900029-3-A | single siRNA, 0.9 nmol | 246 | 2264 | FGFR4 | fibroblast growth factor receptor 4 [symbol] |
| 900029-3-A | single siRNA, 0.9 nmol | 247 | 2322 | FLT3 | fms-related tyrosine kinase 3 |
| 900029-3-A | single siRNA, 0.9 nmol | 248 | 2534 | FYN | FYN oncogene related to SRC, F [symbol] |
| 900029-3-A | single siRNA, 0.9 nmol | 249 | 2690 | GHR | growth hormone receptor |
| 900029-3-A | single siRNA, 0.9 nmol | 250 | 2771 | GNAI2 | guanine nucleotide binding protei [symbol] |
| 900029-3-A | single siRNA, 0.9 nmol | 251 | 2017 | CTTN | cortactin |
| 900029-3-A | single siRNA, 0.9 nmol | 252 | 2045 | EPHA7 | EPH receptor A7 |
| 900029-3-A | single siRNA, 0.9 nmol | 253 | 2066 | ERBB4 | v-erb-a erythroblastic leukemia vi [symbol] |
| 900029-3-A | single siRNA, 0.9 nmol | 254 | 2138 | EYA1 | eyes absent homolog 1 (Drosoph [symbol] |
| 900029-3-A | single siRNA, 0.9 nmol | 255 | 2241 | FER | fer (fps/fes related) tyrosine kinas [symbol] |
| 900029-3-A | single siRNA, 0.9 nmol | 256 | 2268 | FGR | Gardner-Rasheed feline sarcoma [symbol] |
| 900029-3-A | single siRNA, 0.9 nmol | 257 | 2324 | FLT4 | fms-related tyrosine kinase 4 |
| 900029-3-A | single siRNA, 0.9 nmol | 258 | 2547 | XRCC6 | X-ray repair complementing defe [symbol] |
| 900029-3-A | single siRNA, 0.9 nmol | 259 | 2697 | GJA1 | gap junction protein, alpha 1, 43k [symbol] |
| 900029-3-A | single siRNA, 0.9 nmol | 260 | 2773 | GNAI3 | guanine nucleotide binding protei [symbol] |
| 900029-3-A | single siRNA, 0.9 nmol | 261 | 2035 | EPB41 | erythrocyte membrane protein ba [symbol] |
| 900029-3-A | single siRNA, 0.9 nmol | 262 | 2057 | EPOR | erythropoietin receptor |
| 900029-3-A | single siRNA, 0.9 nmol | 263 | 2069 | EREG | epiregulin |
| 900029-3-A | single siRNA, 0.9 nmol | 264 | 2139 | EYA2 | eyes absent homolog 2 (Drosoph [symbol] |
| 900029-3-A | single siRNA, 0.9 nmol | 265 | 2242 | FES | feline sarcoma oncogene |
| 900029-3-A | single siRNA, 0.9 nmol | 266 | 2277 | FIGF | c-fos induced growth factor (vasc [symbol] |
| 900029-3-A | single siRNA, 0.9 nmol | 267 | 2353 | FOS | v-fos FBJ murine osteosarcoma [symbol] |
| 900029-3-A | single siRNA, 0.9 nmol | 268 | 2549 | GAB1 | GRB2-associated binding protein [symbol] |
| 900029-3-A | single siRNA, 0.9 nmol | 269 | 2736 | GLI2 | GLI-Kruppel family member GLI2 [symbol] |
| 900029-3-A | single siRNA, 0.9 nmol | 270 | 2775 | GNAO1 | guanine nucleotide binding protei [symbol] |
| 900029-3-A | single siRNA, 0.9 nmol | 271 | 2036 | EPB41L1 | erythrocyte membrane protein ba [symbol] |
| 900029-3-A | single siRNA, 0.9 nmol | 272 | 2059 | EPS8 | epidermal growth factor receptor [symbol] |
| 900029-3-A | single siRNA, 0.9 nmol | 273 | 2070 | EYA4 | eyes absent homolog 4 (Drosoph [symbol] |
| 900029-3-A | single siRNA, 0.9 nmol | 274 | 2140 | EYA3 | eyes absent homolog 3 (Drosoph [symbol] |
| 900029-3-A | single siRNA, 0.9 nmol | 275 | 2260 | FGFR1 | fibroblast growth factor receptor 1 [symbol] |
| 900029-3-A | single siRNA, 0.9 nmol | 276 | 2308 | FOXO1 | forkhead box O1 |
| 900029-3-A | single siRNA, 0.9 nmol | 277 | 2354 | FOSB | FBJ murine osteosarcoma viral o [symbol] |
| 900029-3-A | single siRNA, 0.9 nmol | 278 | 2597 | GAPDH | glyceraldehyde-3-phosphate deh [symbol] |
| 900029-3-A | single siRNA, 0.9 nmol | 279 | 2737 | GLI3 | GLI-Kruppel family member GLI3 |
| 900029-3-A | single siRNA, 0.9 nmol | 280 | 2810 | SFN | stratifin |
| 900029-3-A | single siRNA, 0.9 nmol | 281 | 2037 | EPB41L2 | erythrocyte membrane protein ba [symbol] |
| 900029-3-A | single siRNA, 0.9 nmol | 282 | 2060 | EPS15 | epidermal growth factor receptor [symbol] |
| 900029-3-A | single siRNA, 0.9 nmol | 283 | 2099 | ESR1 | estrogen receptor 1 |
| 900029-3-A | single siRNA, 0.9 nmol | 284 | 2185 | PTK2B | PTK2B protein tyrosine kinase 2 [symbol] |
| 900029-3-A | single siRNA, 0.9 nmol | 285 | 2261 | FGFR3 | fibroblast growth factor receptor 3 [symbol] |
| 900029-3-A | single siRNA, 0.9 nmol | 286 | 2316 | FLNA | filamin A, alpha (actin binding pro [symbol] |
| 900029-3-A | single siRNA, 0.9 nmol | 287 | 2444 | FRK | fyn-related kinase |
| 900029-3-A | single siRNA, 0.9 nmol | 288 | 2674 | GFRA1 | GDNF family receptor alpha 1 |
| 900029-3-A | single siRNA, 0.9 nmol | 289 | 2768 | GNA12 | guanine nucleotide binding protei [symbol] |
| 900029-3-A | single siRNA, 0.9 nmol | 290 | 2885 | GRB2 | growth factor receptor-bound prot [symbol] |
| 900029-3-A | single siRNA, 0.9 nmol | 291 | 2042 | EPHA3 | EPH receptor A3 |
| 900029-3-A | single siRNA, 0.9 nmol | 292 | 2064 | ERBB2 | v-erb-b2 erythroblastic leukemia [symbol] |
| 900029-3-A | single siRNA, 0.9 nmol | 293 | 2107 | ETF1 | eukaryotic translation termination [symbol] |
| 900029-3-A | single siRNA, 0.9 nmol | 294 | 2214 | FCGR3A | Fc fragment of IgG, low affinity III [symbol] |
| 900029-3-A | single siRNA, 0.9 nmol | 295 | 2263 | FGFR2 | fibroblast growth factor receptor 2 |
| 900029-3-A | single siRNA, 0.9 nmol | 296 | 2321 | FLT1 | fms-related tyrosine kinase 1 (va [symbol] |
| 900029-3-A | single siRNA, 0.9 nmol | 297 | 2521 | FUS | fusion (involved in t(12;16) in mal [symbol] |
| 900029-3-A | single siRNA, 0.9 nmol | 298 | 2683 | B4GALT1 | UDP-Gal:betaGlcNAc beta 1,4-g [symbol] |
| 900029-3-A | single siRNA, 0.9 nmol | 299 | 2770 | GNAI1 | guanine nucleotide binding protei [symbol] |
| 900029-3-A | single siRNA, 0.9 nmol | 300 | 2886 | GRB7 | growth factor receptor-bound prot [symbol] |
| 900029-3-B | single siRNA, 0.9 nmol | 301 | 2002 | ELK1 | ELK1, member of ETS oncogene [symbol] |
| 900029-3-B | single siRNA, 0.9 nmol | 302 | 2044 | EPHA5 | EPH receptor A5 |
| 900029-3-B | single siRNA, 0.9 nmol | 303 | 2065 | ERBB3 | v-erb-b2 erythroblastic leukemia [symbol] |
| 900029-3-B | single siRNA, 0.9 nmol | 304 | 2120 | ETV6 | ets variant gene 6 (TEL oncogen [symbol] |
| 900029-3-B | single siRNA, 0.9 nmol | 305 | 2222 | FDFT1 | farnesyl-diphosphate farnesyltran [symbol] |
| 900029-3-B | single siRNA, 0.9 nmol | 306 | 2264 | FGFR4 | fibroblast growth factor receptor 4 [symbol] |
| 900029-3-B | single siRNA, 0.9 nmol | 307 | 2322 | FLT3 | fms-related tyrosine kinase 3 |
| 900029-3-B | single siRNA, 0.9 nmol | 308 | 2534 | FYN | FYN oncogene related to SRC, F [symbol] |
| 900029-3-B | single siRNA, 0.9 nmol | 309 | 2690 | GHR | growth hormone receptor |
| 900029-3-B | single siRNA, 0.9 nmol | 310 | 2771 | GNAI2 | guanine nucleotide binding protei [symbol] |
| 900029-3-B | single siRNA, 0.9 nmol | 311 | 2017 | CTTN | cortactin |
| 900029-3-B | single siRNA, 0.9 nmol | 312 | 2045 | EPHA7 | EPH receptor A7 |
| 900029-3-B | single siRNA, 0.9 nmol | 313 | 2066 | ERBB4 | v-erb-a erythroblastic leukemia vi [symbol] |
| 900029-3-B | single siRNA, 0.9 nmol | 314 | 2138 | EYA1 | eyes absent homolog 1 (Drosoph [symbol] |
| 900029-3-B | single siRNA, 0.9 nmol | 315 | 2241 | FER | fer (fps/fes related) tyrosine kinas [symbol] |
| 900029-3-B | single siRNA, 0.9 nmol | 316 | 2268 | FGR | Gardner-Rasheed feline sarcoma [symbol] |
| 900029-3-B | single siRNA, 0.9 nmol | 317 | 2324 | FLT4 | fms-related tyrosine kinase 4 |
| 900029-3-B | single siRNA, 0.9 nmol | 318 | 2547 | XRCC6 | X-ray repair complementing defe [symbol] |
| 900029-3-B | single siRNA, 0.9 nmol | 319 | 2697 | GJA1 | gap junction protein, alpha 1, 43k [symbol] |
| 900029-3-B | single siRNA, 0.9 nmol | 320 | 2773 | GNAI3 | guanine nucleotide binding protei [symbol] |
| 900029-3-B | single siRNA, 0.9 nmol | 321 | 2035 | EPB41 | erythrocyte membrane protein ba [symbol] |
| 900029-3-B | single siRNA, 0.9 nmol | 322 | 2057 | EPOR | erythropoietin receptor |
| 900029-3-B | single siRNA, 0.9 nmol | 323 | 2069 | EREG | epiregulin |
| 900029-3-B | single siRNA, 0.9 nmol | 324 | 2139 | EYA2 | eyes absent homolog 2 (Drosoph [symbol] |
| 900029-3-B | single siRNA, 0.9 nmol | 325 | 2242 | FES | feline sarcoma oncogene |
| 900029-3-B | single siRNA, 0.9 nmol | 326 | 2277 | FIGF | c-fos induced growth factor (vasc [symbol] |
| 900029-3-B | single siRNA, 0.9 nmol | 327 | 2353 | FOS | v-fos FBJ murine osteosarcoma [symbol] |
| 900029-3-B | single siRNA, 0.9 nmol | 328 | 2549 | GAB1 | GRB2-associated binding protein [symbol] |
| 900029-3-B | single siRNA, 0.9 nmol | 329 | 2736 | GLI2 | GLI-Kruppel family member GLI2 [symbol] |
| 900029-3-B | single siRNA, 0.9 nmol | 330 | 2775 | GNAO1 | guanine nucleotide binding protei [symbol] |
| 900029-3-B | single siRNA, 0.9 nmol | 331 | 2036 | EPB41L1 | erythrocyte membrane protein ba [symbol] |
| 900029-3-B | single siRNA, 0.9 nmol | 332 | 2059 | EPS8 | epidermal growth factor receptor [symbol] |
| 900029-3-B | single siRNA, 0.9 nmol | 333 | 2070 | EYA4 | eyes absent homolog 4 (Drosoph [symbol] |
| 900029-3-B | single siRNA, 0.9 nmol | 334 | 2140 | EYA3 | eyes absent homolog 3 (Drosoph [symbol] |
| 900029-3-B | single siRNA, 0.9 nmol | 335 | 2260 | FGFR1 | fibroblast growth factor receptor 1 |
| 900029-3-B | single siRNA, 0.9 nmol | 336 | 2308 | FOXO1 | forkhead box O1 |
| 900029-3-B | single siRNA, 0.9 nmol | 337 | 2354 | FOSB | FBJ murine osteosarcoma viral o [symbol] |
| 900029-3-B | single siRNA, 0.9 nmol | 338 | 2597 | GAPDH | glyceraldehyde-3-phosphate deh [symbol] |
| 900029-3-B | single siRNA, 0.9 nmol | 339 | 2737 | GLI3 | GLI-Kruppel family member GLI3 [symbol] |
| 900029-3-B | single siRNA, 0.9 nmol | 340 | 2810 | SFN | stratifin |
| 900029-3-B | single siRNA, 0.9 nmol | 341 | 2037 | EPB41L2 | erythrocyte membrane protein ba [symbol] |
| 900029-3-B | single siRNA, 0.9 nmol | 342 | 2060 | EPS15 | epidermal growth factor receptor [symbol] |
| 900029-3-B | single siRNA, 0.9 nmol | 343 | 2099 | ESR1 | estrogen receptor 1 |
| 900029-3-B | single siRNA, 0.9 nmol | 344 | 2185 | PTK2B | PTK2B protein tyrosine kinase 2 [symbol] |
| 900029-3-B | single siRNA, 0.9 nmol | 345 | 2261 | FGFR3 | fibroblast growth factor receptor 3 [symbol] |
| 900029-3-B | single siRNA, 0.9 nmol | 346 | 2316 | FLNA | filamin A, alpha (actin binding pro [symbol] |
| 900029-3-B | single siRNA, 0.9 nmol | 347 | 2444 | FRK | fyn-related kinase |
| 900029-3-B | single siRNA, 0.9 nmol | 348 | 2674 | GFRA1 | GDNF family receptor alpha 1 |
| 900029-3-B | single siRNA, 0.9 nmol | 349 | 2768 | GNA12 | guanine nucleotide binding protei [symbol] |
| 900029-3-B | single siRNA, 0.9 nmol | 350 | 2885 | GRB2 | growth factor receptor-bound prot [symbol] |
| 900029-3-B | single siRNA, 0.9 nmol | 351 | 2042 | EPHA3 | EPH receptor A3 |
| 900029-3-B | single siRNA, 0.9 nmol | 352 | 2064 | ERBB2 | v-erb-b2 erythroblastic leukemia [symbol] |
| 900029-3-B | single siRNA, 0.9 nmol | 353 | 2107 | ETF1 | eukaryotic translation termination [symbol] |
| 900029-3-B | single siRNA, 0.9 nmol | 354 | 2214 | FCGR3A | Fc fragment of IgG, low affinity III [symbol] |
| 900029-3-B | single siRNA, 0.9 nmol | 355 | 2263 | FGFR2 | fibroblast growth factor receptor 2 |
| 900029-3-B | single siRNA, 0.9 nmol | 356 | 2321 | FLT1 | fms-related tyrosine kinase 1 (va [symbol] |
| 900029-3-B | single siRNA, 0.9 nmol | 357 | 2521 | FUS | fusion (involved in t(12;16) in mal [symbol] |
| 900029-3-B | single siRNA, 0.9 nmol | 358 | 2683 | B4GALT1 | UDP-Gal:betaGlcNAc beta 1,4-g [symbol] |
| 900029-3-B | single siRNA, 0.9 nmol | 359 | 2770 | GNAI1 | guanine nucleotide binding protei [symbol] |
| 900029-3-B | single siRNA, 0.9 nmol | 360 | 2886 | GRB7 | growth factor receptor-bound prot [symbol] |
| 900029-4-A | single siRNA, 0.9 nmol | 361 | 2887 | GRB10 | growth factor receptor-bound prot [symbol] |
| 900029-4-A | single siRNA, 0.9 nmol | 362 | 3064 | HD | huntingtin (Huntington disease) |
| 900029-4-A | single siRNA, 0.9 nmol | 363 | 3164 | NR4A1 | nuclear receptor subfamily 4, gro [symbol] |
| 900029-4-A | single siRNA, 0.9 nmol | 364 | 3312 | HSPA8 | heat shock 70 kDa protein 8 |
| 900029-4-A | single siRNA, 0.9 nmol | 365 | 3398 | ID2 | inhibitor of DNA binding 2, domin [symbol] |
| 900029-4-A | single siRNA, 0.9 nmol | 366 | 3561 | IL2RG | interleukin 2 receptor, gamma (s [symbol] |
| 900029-4-A | single siRNA, 0.9 nmol | 367 | 3643 | INSR | insulin receptor |
| 900029-4-A | single siRNA, 0.9 nmol | 368 | 3716 | JAK1 | Janus kinase 1 (a protein tyrosin [symbol] |
| 900029-4-A | single siRNA, 0.9 nmol | 369 | 3732 | CD82 | CD82 molecule |
| 900029-4-A | single siRNA, 0.9 nmol | 370 | 3855 | KRT7 | keratin 7 |
| 900029-4-A | single siRNA, 0.9 nmol | 371 | 2888 | GRB14 | growth factor receptor-bound prot [symbol] |
| 900029-4-A | single siRNA, 0.9 nmol | 372 | 3065 | HDAC1 | histone deacetylase 1 |
| 900029-4-A | single siRNA, 0.9 nmol | 373 | 3178 | HNRPA1 | heterogeneous nuclear ribonucle [symbol] |
| 900029-4-A | single siRNA, 0.9 nmol | 374 | 3313 | HSPA9 | heat shock 70 kDa protein 9 (mor [symbol] |
| 900029-4-A | single siRNA, 0.9 nmol | 375 | 3480 | IGF1R | insulin-like growth factor 1 recept [symbol] |
| 900029-4-A | single siRNA, 0.9 nmol | 376 | 3566 | IL4R | interleukin 4 receptor |
| 900029-4-A | single siRNA, 0.9 nmol | 377 | 3645 | INSRR | insulin receptor-related receptor |
| 900029-4-A | single siRNA, 0.9 nmol | 378 | 3717 | JAK2 | Janus kinase 2 (a protein tyrosin [symbol] |
| 900029-4-A | single siRNA, 0.9 nmol | 379 | 3791 | KDR | kinase insert domain receptor (a [symbol] |
| 900029-4-A | single siRNA, 0.9 nmol | 380 | 3856 | KRT8 | keratin 8 |
| 900029-4-A | single siRNA, 0.9 nmol | 381 | 2889 | RAPGEF1 | Rap guanine nucleotide exchang [symbol] |
| 900029-4-A | single siRNA, 0.9 nmol | 382 | 3082 | HGF | hepatocyte growth factor (hepapo [symbol] |
| 900029-4-A | single siRNA, 0.9 nmol | 383 | 3190 | HNRPK | heterogeneous nuclear ribonucle [symbol] |
| 900029-4-A | single siRNA, 0.9 nmol | 384 | 3320 | HSP90AA1 | heat shock protein 90 kDa alpha ( [symbol] |
| 900029-4-A | single siRNA, 0.9 nmol | 385 | 3491 | CYR61 | cysteine-rich, angiogenic inducer, [symbol] |
| 900029-4-A | single siRNA, 0.9 nmol | 386 | 3572 | IL6ST | interleukin 6 signal transducer (gr [symbol] |
| 900029-4-A | single siRNA, 0.9 nmol | 387 | 3667 | IRS1 | insulin receptor substrate 1 |
| 900029-4-A | single siRNA, 0.9 nmol | 388 | 3725 | JUN | jun oncogene |
| 900029-4-A | single siRNA, 0.9 nmol | 389 | 3845 | KRAS | v-Ki-ras2 Kirsten rat sarcoma vira [symbol] |
| 900029-4-A | single siRNA, 0.9 nmol | 390 | 3872 | KRT17 | keratin 17 |
| 900029-4-A | single siRNA, 0.9 nmol | 391 | 2932 | GSK3B | glycogen synthase kinase 3 beta [symbol] |
| 900029-4-A | single siRNA, 0.9 nmol | 392 | 3084 | NRG1 | neuregulin 1 |
| 900029-4-A | single siRNA, 0.9 nmol | 393 | 3265 | HRAS | v-Ha-ras Harvey rat sarcoma vira [symbol] |
| 900029-4-A | single siRNA, 0.9 nmol | 394 | 3329 | HSPD1 | heat shock 60 kDa protein 1 (cha [symbol] |
| 900029-4-A | single siRNA, 0.9 nmol | 395 | 3551 | IKBKB | inhibitor of kappa light polypeptid [symbol] |
| 900029-4-A | single siRNA, 0.9 nmol | 396 | 3609 | ILF3 | interleukin enhancer binding fact [symbol] |
| 900029-4-A | single siRNA, 0.9 nmol | 397 | 3678 | ITGA5 | integrin, alpha 5 (fibronectin rece [symbol] |
| 900029-4-A | single siRNA, 0.9 nmol | 398 | 3726 | JUNB | jun B proto-oncogene |
| 900029-4-A | single siRNA, 0.9 nmol | 399 | 3852 | KRT5 | keratin 5 (epidermolysis bullosa s [symbol] |
| 900029-4-A | single siRNA, 0.9 nmol | 400 | 3875 | KRT18 | keratin 18 |
| 900029-4-A | single siRNA, 0.9 nmol | 401 | 2934 | GSN | gelsolin (amyloidosis, Finnish typ [symbol] |
| 900029-4-A | single siRNA, 0.9 nmol | 402 | 3092 | HIP1 | huntingtin interacting protein 1 |
| 900029-4-A | single siRNA, 0.9 nmol | 403 | 3280 | HES1 | hairy and enhancer of split 1, (Dr [symbol] |
| 900029-4-A | single siRNA, 0.9 nmol | 404 | 3371 | TNC | tenascin C (hexabrachion) |
| 900029-4-A | single siRNA, 0.9 nmol | 405 | 3558 | IL2 | interleukin 2 |
| 900029-4-A | single siRNA, 0.9 nmol | 406 | 3635 | INPP5D | inositol polyphosphate-5-phospha [symbol] |
| 900029-4-A | single siRNA, 0.9 nmol | 407 | 3690 | ITGB3 | integrin, beta 3 (platelet glycoprot [symbol] |
| 900029-4-A | single siRNA, 0.9 nmol | 408 | 3727 | JUND | jun D proto-oncogene |
| 900029-4-A | single siRNA, 0.9 nmol | 409 | 3853 | KRT6A | keratin 6A |
| 900029-4-A | single siRNA, 0.9 nmol | 410 | 3897 | L1CAM | L1 cell adhesion molecule |
| 900029-4-A | single siRNA, 0.9 nmol | 411 | 3055 | HCK | hemopoietic cell kinase |
| 900029-4-A | single siRNA, 0.9 nmol | 412 | 3105 | HLA-A | major histocompatibility complex, [symbol] |
| 900029-4-A | single siRNA, 0.9 nmol | 413 | 3309 | HSPA5 | heat shock 70 kDa protein 5 (gluc [symbol] |
| 900029-4-A | single siRNA, 0.9 nmol | 414 | 3397 | ID1 | inhibitor of DNA binding 1, domin [symbol] |
| 900029-4-A | single siRNA, 0.9 nmol | 415 | 3560 | IL2RB | interleukin 2 receptor, beta |
| 900029-4-A | single siRNA, 0.9 nmol | 416 | 3636 | INPPL1 | inositol polyphosphate phosphata [symbol] |
| 900029-4-A | single siRNA, 0.9 nmol | 417 | 3691 | ITGB4 | integrin, beta 4 |
| 900029-4-A | single siRNA, 0.9 nmol | 418 | 3728 | JUP | junction plakoglobin |
| 900029-4-A | single siRNA, 0.9 nmol | 419 | 3854 | KRT68 | keratin 6B |
| 900029-4-A | single siRNA, 0.9 nmol | 420 | 3932 | LCK | lymphocyte-specific protein tyrosi [symbol] |
| 900029-4-B | single siRNA, 0.9 nmol | 421 | 2887 | GRB10 | growth factor receptor-bound prot [symbol] |
| 900029-4-B | single siRNA, 0.9 nmol | 422 | 3064 | HD | huntingtin (Huntington disease) |
| 900029-4-B | single siRNA, 0.9 nmol | 423 | 3164 | NR4A1 | nuclear receptor subfamily 4, gro [symbol] |
| 900029-4-B | single siRNA, 0.9 nmol | 424 | 3312 | HSPA8 | heat shock 70 kDa protein 8 |
| 900029-4-B | single siRNA, 0.9 nmol | 425 | 3398 | ID2 | inhibitor of DNA binding 2, domin [symbol] |
| 900029-4-B | single siRNA, 0.9 nmol | 426 | 3561 | IL2RG | interleukin 2 receptor, gamma (s [symbol] |
| 900029-4-B | single siRNA, 0.9 nmol | 427 | 3643 | INSR | insulin receptor |
| 900029-4-B | single siRNA, 0.9 nmol | 428 | 3716 | JAK1 | Janus kinase 1 (a protein tyrosin [symbol] |
| 900029-4-B | single siRNA, 0.9 nmol | 429 | 3732 | CD82 | CD82 molecule |
| 900029-4-B | single siRNA, 0.9 nmol | 430 | 3855 | KRT7 | keratin 7 |
| 900029-4-B | single siRNA, 0.9 nmol | 431 | 2888 | GRB14 | growth factor receptor-bound prot [symbol] |
| 900029-4-B | single siRNA, 0.9 nmol | 432 | 3065 | HDAC1 | histone deacetylase 1 |
| 900029-4-B | single siRNA, 0.9 nmol | 433 | 3178 | HNRPA1 | heterogeneous nuclear ribonucle [symbol] |
| 900029-4-B | single siRNA, 0.9 nmol | 434 | 3313 | HSPA9 | heat shock 70 kDa protein 9 (mor [symbol] |
| 900029-4-B | single siRNA, 0.9 nmol | 435 | 3480 | IGF1R | insulin-like growth factor 1 recept [symbol] |
| 900029-4-B | single siRNA, 0.9 nmol | 436 | 3566 | IL4R | interleukin 4 receptor |
| 900029-4-B | single siRNA, 0.9 nmol | 437 | 3645 | INSRR | insulin receptor-related receptor |
| 900029-4-B | single siRNA, 0.9 nmol | 438 | 3717 | JAK2 | Janus kinase 2 (a protein tyrosin [symbol] |
| 900029-4-B | single siRNA, 0.9 nmol | 439 | 3791 | KDR | kinase insert domain receptor (a [symbol] |
| 900029-4-B | single siRNA, 0.9 nmol | 440 | 3856 | KRT8 | keratin 8 |
| 900029-4-B | single siRNA, 0.9 nmol | 441 | 2889 | RAPGEF1 | Rap guanine nucleotide exchang [symbol] |
| 900029-4-B | single siRNA, 0.9 nmol | 442 | 3082 | HGF | hepatocyte growth factor (hepapo [symbol] |
| 900029-4-B | single siRNA, 0.9 nmol | 443 | 3190 | HNRPK | heterogeneous nuclear ribonucle [symbol] |
| 900029-4-B | single siRNA, 0.9 nmol | 444 | 3320 | HSP90AA1 | heat shock protein 90 kDa alpha [symbol] |
| 900029-4-B | single siRNA, 0.9 nmol | 445 | 3491 | CYR61 | cysteine-rich, angiogenic inducer, [symbol] |
| 900029-4-B | single siRNA, 0.9 nmol | 446 | 3572 | IL6ST | interleukin 6 signal transducer (g [symbol] |
| 900029-4-B | single siRNA, 0.9 nmol | 447 | 3667 | IRS1 | insulin receptor substrate 1 |
| 900029-4-B | single siRNA, 0.9 nmol | 448 | 3725 | JUN | jun oncogene |
| 900029-4-B | single siRNA, 0.9 nmol | 449 | 3845 | KRAS | v-Ki-ras2 Kirsten rat sarcoma vira [symbol] |
| 900029-4-B | single siRNA, 0.9 nmol | 450 | 3872 | KRT17 | keratin 17 |
| 900029-4-B | single siRNA, 0.9 nmol | 451 | 2932 | GSK3B | glycogen synthase kinase 3 beta [symbol] |
| 900029-4-B | single siRNA, 0.9 nmol | 452 | 3084 | NRG1 | neuregulin 1 |
| 900029-4-B | single siRNA, 0.9 nmol | 453 | 3265 | HRAS | v-Ha-ras Harvey rat sarcoma vira [symbol] |
| 900029-4-B | single siRNA, 0.9 nmol | 454 | 3329 | HSPD1 | heat shock 60 kDa protein 1 (cha [symbol] |
| 900029-4-B | single siRNA, 0.9 nmol | 455 | 3551 | IKBKB | inhibitor of kappa light polypeptid [symbol] |
| 900029-4-B | single siRNA, 0.9 nmol | 456 | 3609 | ILF3 | interleukin enhancer binding fact [symbol] |
| 900029-4-B | single siRNA, 0.9 nmol | 457 | 3678 | ITGA5 | integrin, alpha 5 (fibronectin rece [symbol] |
| 900029-4-B | single siRNA, 0.9 nmol | 458 | 3726 | JUNB | jun B proto-oncogene |
| 900029-4-B | single siRNA, 0.9 nmol | 459 | 3852 | KRT5 | keratin 5 (epidermolysis bullosa s [symbol] |
| 900029-4-B | single siRNA, 0.9 nmol | 460 | 3875 | KRT18 | keratin 18 |
| 900029-4-B | single siRNA, 0.9 nmol | 461 | 2934 | GSN | gelsolin (amyloidosis, Finnish typ [symbol] |
| 900029-4-B | single siRNA, 0.9 nmol | 462 | 3092 | HIP1 | huntingtin interacting protein 1 |
| 900029-4-B | single siRNA, 0.9 nmol | 463 | 3280 | HES1 | hairy and enhancer of split 1, (Dr [symbol] |
| 900029-4-B | single siRNA, 0.9 nmol | 464 | 3371 | TNC | tenascin C (hexabrachion) |
| 900029-4-B | single siRNA, 0.9 nmol | 465 | 3558 | IL2 | interleukin 2 |
| 900029-4-B | single siRNA, 0.9 nmol | 466 | 3635 | INPP5D | inositol polyphosphate-5-phosph [symbol] |
| 900029-4-B | single siRNA, 0.9 nmol | 467 | 3690 | ITGB3 | integrin, beta 3 (platelet glycoprot [symbol] |
| 900029-4-B | single siRNA, 0.9 nmol | 468 | 3727 | JUND | jun D proto-oncogene |
| 900029-4-B | single siRNA, 0.9 nmol | 469 | 3853 | KRT6A | keratin 6A |
| 900029-4-B | single siRNA, 0.9 nmol | 470 | 3897 | L1CAM | L1 cell adhesion molecule |
| 900029-4-B | single siRNA, 0.9 nmol | 471 | 3055 | HCK | hemopoietic cell kinase |
| 900029-4-B | single siRNA, 0.9 nmol | 472 | 3105 | HLA-A | major histocompatibility complex, [symbol] |
| 900029-4-B | single siRNA, 0.9 nmol | 473 | 3309 | HSPA5 | heat shock 70 kDa protein 5 (gluc [symbol] |
| 900029-4-B | single siRNA, 0.9 nmol | 474 | 3397 | ID1 | inhibitor of DNA binding 1, domin [symbol] |
| 900029-4-B | single siRNA, 0.9 nmol | 475 | 3560 | IL2RB | interleukin 2 receptor, beta |
| 900029-4-B | single siRNA, 0.9 nmol | 476 | 3636 | INPPL1 | inositol polyphosphate phosphata [symbol] |
| 900029-4-B | single siRNA, 0.9 nmol | 477 | 3691 | ITGB4 | integrin, beta 4 |
| 900029-4-B | single siRNA, 0.9 nmol | 478 | 3728 | JUP | junction plakoglobin |
| 900029-4-B | single siRNA, 0.9 nmol | 479 | 3854 | KRT6B | keratin 6B |
| 900029-4-B | single siRNA, 0.9 nmol | 480 | 3932 | LCK | lymphocyte-specific protein tyrosi [symbol] |
| 900029-5-A | single siRNA, 0.9 nmol | 481 | 3937 | LCP2 | lymphocyte cytosolic protein 2 (S [symbol] |
| 900029-5-A | single siRNA, 0.9 nmol | 482 | 4086 | SMAD1 | SMAD family member 1 |
| 900029-5-A | single siRNA, 0.9 nmol | 483 | 4194 | MDM4 | Mdm4, transformed 3T3 cell dou [symbol] |
| 900029-5-A | single siRNA, 0.9 nmol | 484 | 4296 | MAP3K11 | mitogen-activated protein kinase [symbol] |
| 900029-5-A | single siRNA, 0.9 nmol | 485 | 4690 | NCK1 | NCK adaptor protein 1 |
| 900029-5-A | single siRNA, 0.9 nmol | 486 | 4893 | NRAS | neuroblastoma RAS viral (v-ras) [symbol] |
| 900029-5-A | single siRNA, 0.9 nmol | 487 | 5017 | OVOL1 | ovo-like 1 (Drosophila) |
| 900029-5-A | single siRNA, 0.9 nmol | 488 | 5063 | PAK3 | p21 (CDKN1A)-activated kinase [symbol] |
| 900029-5-A | single siRNA, 0.9 nmol | 489 | 5290 | PIK3CA | phosphoinositide-3-kinase, cataly [symbol] |
| 900029-5-A | single siRNA, 0.9 nmol | 490 | 5300 | PIN1 | protein (peptidylprolyl cis/trans is [symbol] |
| 900029-5-A | single siRNA, 0.9 nmol | 491 | 3958 | LGALS3 | lectin, galactoside-binding, solubl [symbol] |
| 900029-5-A | single siRNA, 0.9 nmol | 492 | 4087 | SMAD2 | SMAD family member 2 |
| 900029-5-A | single siRNA, 0.9 nmol | 493 | 4214 | MAP3K1 | mitogen-activated protein kinase |
| 900029-5-A | single siRNA, 0.9 nmol | 494 | 4311 | MME | membrane metallo-endopeptidas [symbol] |
| 900029-5-A | single siRNA, 0.9 nmol | 495 | 4739 | NEDD9 | neural precursor cell expressed, [symbol] |
| 900029-5-A | single siRNA, 0.9 nmol | 496 | 4914 | NTRK1 | neurotrophic tyrosine kinase, rec [symbol] |
| 900029-5-A | single siRNA, 0.9 nmol | 497 | 5037 | PEBP1 | phosphatidylethanolamine bindin [symbol] |
| 900029-5-A | single siRNA, 0.9 nmol | 498 | 5130 | PCYT1A | phosphate cytidytyltransferase 1, [symbol] |
| 900029-5-A | single siRNA, 0.9 nmol | 499 | 5291 | PIK3CB | phosphoinositide-3-kinase, cataly [symbol] |
| 900029-5-A | single siRNA, 0.9 nmol | 500 | 5305 | PIP5K2A | phosphatidylinositol-4-phosphate [symbol] |
| 900029-5-A | single siRNA, 0.9 nmol | 501 | 4000 | LMNA | lamin A/C |
| 900029-5-A | single siRNA, 0.9 nmol | 502 | 4088 | SMAD3 | SMAD family member 3 |
| 900029-5-A | single siRNA, 0.9 nmol | 503 | 4215 | MAP3K3 | mitogen-activated protein kinase [symbol] |
| 900029-5-A | single siRNA, 0.9 nmol | 504 | 4486 | MST1R | macrophage stimulating 1 recept [symbol] |
| 900029-5-A | single siRNA, 0.9 nmol | 505 | 4790 | NFKB1 | nuclear factor of kappa light poly [symbol] |
| 900029-5-A | single siRNA, 0.9 nmol | 506 | 4915 | NTRK2 | neurotrophic tyrosine kinase, rec [symbol] |
| 900029-5-A | single siRNA, 0.9 nmol | 507 | 5042 | PABPC3 | poly(A) binding protein, cytoplas [symbol] |
| 900029-5-A | single siRNA, 0.9 nmol | 508 | 5159 | PDGFRB | platelet-derived growth factor rec [symbol] |
| 900029-5-A | single siRNA, 0.9 nmol | 509 | 5293 | PIK3CD | phosphoinositide-3-kinase, cataly [symbol] |
| 900029-5-A | single siRNA, 0.9 nmol | 510 | 5306 | PITPNA | phosphatidylinositol transfer prote [symbol] |
| 900029-5-A | single siRNA, 0.9 nmol | 511 | 4035 | LRP1 | low density lipoprotein-related pr [symbol] |
| 900029-5-A | single siRNA, 0.9 nmol | 512 | 4093 | SMAD9 | SMAD family member 9 |
| 900029-5-A | single siRNA, 0.9 nmol | 513 | 4216 | MAP3K4 | mitogen-activated protein kinase [symbol] |
| 900029-5-A | single siRNA, 0.9 nmol | 514 | 4582 | MUC1 | mucin 1, cell surface associated [symbol] |
| 900029-5-A | single siRNA, 0.9 nmol | 515 | 4804 | NGFR | nerve growth factor receptor (TN [symbol] |
| 900029-5-A | single siRNA, 0.9 nmol | 516 | 4916 | NTRK3 | neurotrophic tyrosine kinase, rec [symbol] |
| 900029-5-A | single siRNA, 0.9 nmol | 517 | 5048 | PAFAH1B1 | platelet-activating factor acetylhy [symbol] |
| 900029-5-A | single siRNA, 0.9 nmol | 518 | 5170 | PDPK1 | 3-phosphoinositide dependent pr [symbol] |
| 900029-5-A | single siRNA, 0.9 nmol | 519 | 5294 | PIK3CG | phosphoinositide-3-kinase, cataly [symbol] |
| 900029-5-A | single siRNA, 0.9 nmol | 520 | 5310 | PKD1 | polycystic kidney disease 1 (auto [symbol] |
| 900029-5-A | single siRNA, 0.9 nmol | 521 | 4058 | LTK | leukocyte tyrosine kinase |
| 900029-5-A | single siRNA, 0.9 nmol | 522 | 4145 | MATK | megakaryocyte-associated tyrosi [symbol] |
| 900029-5-A | single siRNA, 0.9 nmol | 523 | 4217 | MAP3K5 | mitogen-activated protein kinase [symbol] |
| 900029-5-A | single siRNA, 0.9 nmol | 524 | 4609 | MYC | v-myc myelocytomatosis viral on [symbol] |
| 900029-5-A | single siRNA, 0.9 nmol | 525 | 4853 | NOTCH2 | Notch homolog 2 (Drosophila) |
| 900029-5-A | single siRNA, 0.9 nmol | 526 | 4921 | DDR2 | discoidin domain receptor family, [symbol] |
| 900029-5-A | single siRNA, 0.9 nmol | 527 | 5058 | PAK1 | p21/Cdc42/Rac1-activated kinas [symbol] |
| 900029-5-A | single siRNA, 0.9 nmol | 528 | 5174 | PDZK1 | PDZ domain containing 1 |
| 900029-5-A | single siRNA, 0.9 nmol | 529 | 5295 | PIK3R1 | phosphoinositide-3-kinase, regul [symbol] |
| 900029-5-A | single siRNA, 0.9 nmol | 530 | 5335 | PLCG1 | phospholipase C, gamma 1 |
| 900029-5-A | single siRNA, 0.9 nmol | 531 | 4067 | LYN | v-yes-1 Yamaguchi sarcoma viral [symbol] |
| 900029-5-A | single siRNA, 0.9 nmol | 532 | 4168 | MCF2 | MCF2 cell line derived transform [symbol] |
| 900029-5-A | single siRNA, 0.9 nmol | 533 | 4233 | MET | met proto-oncogene (hepatocyte [symbol] |
| 900029-5-A | single siRNA, 0.9 nmol | 534 | 4627 | MYH9 | myosin, heavy chain 9, non-musc [symbol] |
| 900029-5-A | single siRNA, 0.9 nmol | 535 | 4867 | NPHP1 | nephronophthisis 1 (juvenile) |
| 900029-5-A | single siRNA, 0.9 nmol | 536 | 4929 | NR4A2 | nuclear receptor subfamily 4, gro [symbol] |
| 900029-5-A | single siRNA, 0.9 nmol | 537 | 5062 | PAK2 | p21 (CDKN1A)-activated kinase [symbol] |
| 900029-5-A | single siRNA, 0.9 nmol | 538 | 5287 | PIK3C2B | phosphoinositide-3-kinase, class [symbol] |
| 900029-5-A | single siRNA, 0.9 nmol | 539 | 5296 | PIK3R2 | phosphoinositide-3-kinase, regul [symbol] |
| 900029-5-A | single siRNA, 0.9 nmol | 540 | 5336 | PLCG2 | phospholipase C, gamma 2 (pho [symbol] |
| 900029-5-B | single siRNA, 0.9 nmol | 541 | 3937 | LCP2 | lymphocyte cytosolic protein 2 (S [symbol] |
| 900029-5-B | single siRNA, 0.9 nmol | 542 | 4086 | SMAD1 | SMAD family member 1 |
| 900029-5-B | single siRNA, 0.9 nmol | 543 | 4194 | MDM4 | Mdm4, transformed 3T3 cell dou [symbol] |
| 900029-5-B | single siRNA, 0.9 nmol | 544 | 4296 | MAP3K11 | mitogen-activated protein kinase [symbol] |
| 900029-5-B | single siRNA, 0.9 nmol | 545 | 4690 | NCK1 | NCK adaptor protein 1 |
| 900029-5-B | single siRNA, 0.9 nmol | 546 | 4893 | NRAS | neuroblastoma RAS viral (v-ras) [symbol] |
| 900029-5-B | single siRNA, 0.9 nmol | 547 | 5017 | OVOL1 | ovo-like 1 (Drosophila) |
| 900029-5-B | single siRNA, 0.9 nmol | 548 | 5063 | PAK3 | p21 (CDKN1A)-activated kinase [symbol] |
| 900029-5-B | single siRNA, 0.9 nmol | 549 | 5290 | PIK3CA | phosphoinositide-3-kinase, cataly [symbol] |
| 900029-5-B | single siRNA, 0.9 nmol | 550 | 5300 | PIN1 | protein (peptidylprolyl cis/trans is [symbol] |
| 900029-5-B | single siRNA, 0.9 nmol | 551 | 3958 | LGALS3 | lectin, galactoside-binding, solubl [symbol] |
| 900029-5-B | single siRNA, 0.9 nmol | 552 | 4087 | SMAD2 | SMAD family member 2 |
| 900029-5-B | single siRNA, 0.9 nmol | 553 | 4214 | MAP3K1 | mitogen-activated protein kinase |
| 900029-5-B | single siRNA, 0.9 nmol | 554 | 4311 | MME | membrane metallo-endopeptidas [symbol] |
| 900029-5-B | single siRNA, 0.9 nmol | 555 | 4739 | NEDD9 | neural precursor cell expressed, [symbol] |
| 900029-5-B | single siRNA, 0.9 nmol | 556 | 4914 | NTRK1 | neurotrophic tyrosine kinase, rec [symbol] |
| 900029-5-B | single siRNA, 0.9 nmol | 557 | 5037 | PEBP1 | phosphatidylethanolamine binding [symbol] |
| 900029-5-B | single siRNA, 0.9 nmol | 558 | 5130 | PCYT1A | phosphate cytidylyltransferase 1, [symbol] |
| 900029-5-B | single siRNA, 0.9 nmol | 559 | 5291 | PIK3CB | phosphoinositide-3-kinase, cataly [symbol] |
| 900029-5-B | single siRNA, 0.9 nmol | 560 | 5305 | PIP5K2A | phosphatidylinositol-4-phosphate [symbol] |
| 900029-5-B | single siRNA, 0.9 nmol | 561 | 4000 | LMNA | lamin A/C |
| 900029-5-B | single siRNA, 0.9 nmol | 562 | 4088 | SMAD3 | SMAD family member 3 |
| 900029-5-B | single siRNA, 0.9 nmol | 563 | 4215 | MAP3K3 | mitogen-activated protein kinase [symbol] |
| 900029-5-B | single siRNA, 0.9 nmol | 564 | 4486 | MST1R | macrophage stimulating 1 recept [symbol] |
| 900029-5-B | single siRNA, 0.9 nmol | 565 | 4790 | NFKB1 | nuclear factor of kappa light polyp [symbol] |
| 900029-5-B | single siRNA, 0.9 nmol | 566 | 4915 | NTRK2 | neurotrophic tyrosine kinase, rece [symbol] |
| 900029-5-B | single siRNA, 0.9 nmol | 567 | 5042 | PABPC3 | poly(A) binding protein, cytoplas [symbol] |
| 900029-5-B | single siRNA, 0.9 nmol | 568 | 5159 | PDGFRB | platelet-derived growth factor rece [symbol] |
| 900029-5-B | single siRNA, 0.9 nmol | 569 | 5293 | PIK3CD | phosphoinositide-3-kinase, cataly [symbol] |
| 900029-5-B | single siRNA, 0.9 nmol | 570 | 5306 | PITPNA | phosphatidylinositol transfer prote [symbol] |
| 900029-5-B | single siRNA, 0.9 nmol | 571 | 4035 | LRP1 | low density lipoprotein-related pr [symbol] |
| 900029-5-B | single siRNA, 0.9 nmol | 572 | 4093 | SMAD9 | SMAD family member 9 |
| 900029-5-B | single siRNA, 0.9 nmol | 573 | 4216 | MAP3K4 | mitogen-activated protein kinase [symbol] |
| 900029-5-B | single siRNA, 0.9 nmol | 574 | 4582 | MUC1 | mucin 1, cell surface associated [symbol] |
| 900029-5-B | single siRNA, 0.9 nmol | 575 | 4804 | NGFR | nerve growth factor receptor (TN [symbol] |
| 900029-5-B | single siRNA, 0.9 nmol | 576 | 4916 | NTRK3 | neurotrophic tyrosine kinase, rec [symbol] |
| 900029-5-B | single siRNA, 0.9 nmol | 577 | 5048 | PAFAH1B1 | platelet-activating factor acetylhy [symbol] |
| 900029-5-B | single siRNA, 0.9 nmol | 578 | 5170 | PDPK1 | 3-phosphoinositide dependent pr [symbol] |
| 900029-5-B | single siRNA, 0.9 nmol | 579 | 5294 | PIK3CG | phosphoinositide-3-kinase, cataly [symbol] |
| 900029-5-B | single siRNA, 0.9 nmol | 580 | 5310 | PKD1 | polycystic kidney disease 1 (auto [symbol] |
| 900029-5-B | single siRNA, 0.9 nmol | 581 | 4058 | LTK | leukocyte tyrosine kinase |
| 900029-5-B | single siRNA, 0.9 nmol | 582 | 4145 | MATK | megakaryocyte-associated tyrosi [symbol] |
| 900029-5-B | single siRNA, 0.9 nmol | 583 | 4217 | MAP3K5 | mitogen-activated protein kinase [symbol] |
| 900029-5-B | single siRNA, 0.9 nmol | 584 | 4609 | MYC | v-myc myelocytomatosis viral on [symbol] |
| 900029-5-B | single siRNA, 0.9 nmol | 585 | 4853 | NOTCH2 | Notch homolog 2 (Drosophila) |
| 900029-5-B | single siRNA, 0.9 nmol | 586 | 4921 | DDR2 | discoidin domain receptor family, [symbol] |
| 900029-5-B | single siRNA, 0.9 nmol | 587 | 5058 | PAK1 | p21/Cdc42/Rac1-activated kinase [symbol] |
| 900029-5-B | single siRNA, 0.9 nmol | 588 | 5174 | PDZK1 | PDZ domain containing 1 |
| 900029-5-B | single siRNA, 0.9 nmol | 589 | 5295 | PIK3R1 | phosphoinositide-3-kinase, regul [symbol] |
| 900029-5-B | single siRNA, 0.9 nmol | 590 | 5335 | PLCG1 | phospholipase C, gamma 1 |
| 900029-5-B | single siRNA, 0.9 nmol | 591 | 4067 | LYN | v-yes-1 Yamaguchi sarcoma viral [symbol] |
| 900029-5-B | single siRNA, 0.9 nmol | 592 | 4168 | MCF2 | MCF.2 cell line derived transform [symbol] |
| 900029-5-B | single siRNA, 0.9 nmol | 593 | 4233 | MET | met proto-oncogene (hepatocyte [symbol] |
| 900029-5-B | single siRNA, 0.9 nmol | 594 | 4627 | MYH9 | myosin, heavy chain 9, non-mus [symbol] |
| 900029-5-B | single siRNA, 0.9 nmol | 595 | 4867 | NPHP1 | nephronophthisis 1 (juvenile) |
| 900029-5-B | single siRNA, 0.9 nmol | 596 | 4929 | NR4A2 | nuclear receptor subfamily 4, gro [symbol] |
| 900029-5-B | single siRNA, 0.9 nmol | 597 | 5062 | PAK2 | p21 (CDKN1A)-activated kinase [symbol] |
| 900029-5-B | single siRNA, 0.9 nmol | 598 | 5287 | PIK3C2B | phosphoinositide-3-kinase, class [symbol] |
| 900029-5-B | single siRNA, 0.9 nmol | 599 | 5296 | PIK3R2 | phosphoinositide-3-kinase, regul [symbol] |
| 900029-5-B | single siRNA, 0.9 nmol | 600 | 5336 | PLCG2 | phospholipase C, gamma 2 (pho [symbol] |
| 900029-6-A | single siRNA, 0.9 nmol | 601 | 5337 | PLD1 | phospholipase D1, phosphatidylc [symbol] |
| 900029-6-A | single siRNA, 0.9 nmol | 602 | 5455 | POU3F3 | POU domain, class 3, transcripti [symbol] |
| 900029-6-A | single siRNA, 0.9 nmol | 603 | 5566 | PRKACA | protein kinase, cAMP-dependent, [symbol] |
| 900029-6-A | single siRNA, 0.9 nmol | 604 | 5580 | PRKCD | protein kinase C, delta |
| 900029-6-A | single siRNA, 0.9 nmol | 605 | 5588 | PRKCQ | protein kinase C, theta |
| 900029-6-A | single siRNA, 0.9 nmol | 606 | 5601 | MAPK9 | mitogen-activated protein kinase [symbol] |
| 900029-6-A | single siRNA, 0.9 nmol | 607 | 5613 | PRKX | protein kinase, X-linked |
| 900029-6-A | single siRNA, 0.9 nmol | 608 | 5770 | PTPN1 | protein tyrosine phosphatase, no [symbol] |
| 900029-6-A | single siRNA, 0.9 nmol | 609 | 5794 | PTPRH | protein tyrosine phosphatase, rec [symbol] |
| 900029-6-A | single siRNA, 0.9 nmol | 610 | 5881 | RAC3 | ras-related C3 botulinum toxin su [symbol] |
| 900029-6-A | single siRNA, 0.9 nmol | 611 | 5338 | PLD2 | phospholipase D2 |
| 900029-6-A | single siRNA, 0.9 nmol | 612 | 5456 | POU3F4 | POU domain, class 3, transcripti [symbol] |
| 900029-6-A | single siRNA, 0.9 nmol | 613 | 5567 | PRKACB | protein kinase, cAMP-dependent, [symbol] |
| 900029-6-A | single siRNA, 0.9 nmol | 614 | 5581 | PRKCE | protein kinase C, epsilon |
| 900029-6-A | single siRNA, 0.9 nmol | 615 | 5590 | PRKCZ | protein kinase C, zeta |
| 900029-6-A | single siRNA, 0.9 nmol | 616 | 5602 | MAPK10 | mitogen-activated protein kinase [symbol] |
| 900029-6-A | single siRNA, 0.9 nmol | 617 | 5625 | PRODH | proline dehydrogenase (oxidase) [symbol] |
| 900029-6-A | single siRNA, 0.9 nmol | 618 | 5771 | PTPN2 | protein tyrosine phosphatase, no [symbol] |
| 900029-6-A | single siRNA, 0.9 nmol | 619 | 5795 | PTPRJ | protein tyrosine phosphatase, rec [symbol] |
| 900029-6-A | single siRNA, 0.9 nmol | 620 | 5894 | RAF1 | v-raf-1 murine leukemia viral onc [symbol] |
| 900029-6-A | single siRNA, 0.9 nmol | 621 | 5339 | PLEC1 | plectin 1, intermediate filament bi [symbol] |
| 900029-6-A | single siRNA, 0.9 nmol | 622 | 5478 | PPIA | peptidylprolyl isomerase A (cyclo [symbol] |
| 900029-6-A | single siRNA, 0.9 nmol | 623 | 5568 | PRKACG | protein kinase, cAMP-dependent, [symbol] |
| 900029-6-A | single siRNA, 0.9 nmol | 624 | 5582 | PRKCG | protein kinase C, gamma |
| 900029-6-A | single siRNA, 0.9 nmol | 625 | 5591 | PRKDC | protein kinase, DNA-activated, ca [symbol] |
| 900029-6-A | single siRNA, 0.9 nmol | 626 | 5604 | MAP2K1 | mitogen-activated protein kinase [symbol] |
| 900029-6-A | single siRNA, 0.9 nmol | 627 | 5728 | PTEN | phosphatase and tensin homolog [symbol] |
| 900029-6-A | single siRNA, 0.9 nmol | 628 | 5777 | PTPN6 | protein tyrosine phosphatase, no [symbol] |
| 900029-6-A | single siRNA, 0.9 nmol | 629 | 5829 | PXN | paxillin |
| 900029-6-A | single siRNA, 0.9 nmol | 630 | 5898 | RALA | v-ral simian leukemia viral oncog [symbol] |
| 900029-6-A | single siRNA, 0.9 nmol | 631 | 5359 | PLSCR1 | phospholipid scramblase 1 |
| 900029-6-A | single siRNA, 0.9 nmol | 632 | 5500 | PPP1CB | protein phosphatase 1, catalytic s [symbol] |
| 900029-6-A | single siRNA, 0.9 nmol | 633 | 5573 | PRKAR1A | protein kinase, cAMP-dependent, [symbol] |
| 900029-6-A | single siRNA, 0.9 nmol | 634 | 5584 | PRKCI | protein kinase C, iota |
| 900029-6-A | single siRNA, 0.9 nmol | 635 | 5594 | MAPK1 | mitogen-activated protein kinase [symbol] |
| 900029-6-A | single siRNA, 0.9 nmol | 636 | 5605 | MAP2K2 | mitogen-activated protein kinase [symbol] |
| 900029-6-A | single siRNA, 0.9 nmol | 637 | 5734 | PTGER4 | prostaglandin E receptor 4 (subty [symbol] |
| 900029-6-A | single siRNA, 0.9 nmol | 638 | 5781 | PTPN11 | protein tyrosine phosphatase, no [symbol] |
| 900029-6-A | single siRNA, 0.9 nmol | 639 | 5868 | RAB5A | RAB5A, member RAS oncogene [symbol] |
| 900029-6-A | single siRNA, 0.9 nmol | 640 | 5899 | RALB | v-ral simian leukemia viral oncog [symbol] |
| 900029-6-A | single siRNA, 0.9 nmol | 641 | 5453 | POU3F1 | POU domain, class 3, transcripti [symbol] |
| 900029-6-A | single siRNA, 0.9 nmol | 642 | 5514 | PPP1R10 | protein phosphatase 1, regulatory [symbol] |
| 900029-6-A | single siRNA, 0.9 nmol | 643 | 5578 | PRKCA | protein kinase C, alpha |
| 900029-6-A | single siRNA, 0.9 nmol | 644 | 5586 | PKN2 | protein kinase N2 |
| 900029-6-A | single siRNA, 0.9 nmol | 645 | 5595 | MAPK3 | mitogen-activated protein kinase [symbol] |
| 900029-6-A | single siRNA, 0.9 nmol | 646 | 5606 | MAP2K3 | mitogen-activated protein kinase [symbol] |
| 900029-6-A | single siRNA, 0.9 nmol | 647 | 5747 | PTK2 | PTK2 protein tyrosine kinase 2 |
| 900029-6-A | single siRNA, 0.9 nmol | 648 | 5782 | PTPN12 | protein tyrosine phosphatase, no [symbol] |
| 900029-6-A | single siRNA, 0.9 nmol | 649 | 5879 | RAC1 | ras-related C3 botulinum toxin su [symbol] |
| 900029-6-A | single siRNA, 0.9 nmol | 650 | 5906 | RAP1A | RAP1A, member of RAS oncoge [symbol] |
| 900029-6-A | single siRNA, 0.9 nmol | 651 | 5454 | POU3F2 | POU domain, class 3, transcripti [symbol] |
| 900029-6-A | single siRNA, 0.9 nmol | 652 | 5525 | PPP2R5A | protein phosphatase 2, regulatory [symbol] |
| 900029-6-A | single siRNA, 0.9 nmol | 653 | 5579 | PRKCB1 | protein kinase C, beta 1 |
| 900029-6-A | single siRNA, 0.9 nmol | 654 | 5587 | PRKD1 | protein kinase D1 |
| 900029-6-A | single siRNA, 0.9 nmol | 655 | 5599 | MAPK8 | mitogen-activated protein kinase [symbol] |
| 900029-6-A | single siRNA, 0.9 nmol | 656 | 5609 | MAP2K7 | mitogen-activated protein kinase [symbol] |
| 900029-6-A | single siRNA, 0.9 nmol | 657 | 5753 | PTK6 | PTK6 protein tyrosine kinase 6 |
| 900029-6-A | single siRNA, 0.9 nmol | 658 | 5792 | PTPRF | protein tyrosine phosphatase, re [symbol] |
| 900029-6-A | single siRNA, 0.9 nmol | 659 | 5880 | RAC2 | ras-related C3 botulinum toxin su [symbol] |
| 900029-6-A | single siRNA, 0.9 nmol | 660 | 5908 | RAP1B | RAP1B, member of RAS oncoge [symbol] |
| 900029-6-B | single siRNA, 0.9 nmol | 661 | 5337 | PLD1 | phospholipase D1, phosphatidylc [symbol] |
| 900029-6-B | single siRNA, 0.9 nmol | 662 | 5455 | POU3F3 | POU domain, class 3, transcripti [symbol] |
| 900029-6-B | single siRNA, 0.9 nmol | 663 | 5566 | PRKACA | protein kinase, cAMP-dependent, [symbol] |
| 900029-6-B | single siRNA, 0.9 nmol | 664 | 5580 | PRKCD | protein kinase C, delta |
| 900029-6-B | single siRNA, 0.9 nmol | 665 | 5588 | PRKCQ | protein kinase C, theta |
| 900029-6-B | single siRNA, 0.9 nmol | 666 | 5601 | MAPK9 | mitogen-activated protein kinase [symbol] |
| 900029-6-B | single siRNA, 0.9 nmol | 667 | 5613 | PRKX | protein kinase, X-linked |
| 900029-6-B | single siRNA, 0.9 nmol | 668 | 5770 | PTPN1 | protein tyrosine phosphatase, no [symbol] |
| 900029-6-B | single siRNA, 0.9 nmol | 669 | 5794 | PTPRH | protein tyrosine phosphatase, rec [symbol] |
| 900029-6-B | single siRNA, 0.9 nmol | 670 | 5881 | RAC3 | ras-related C3 botulinum toxin su [symbol] |
| 900029-6-B | single siRNA, 0.9 nmol | 671 | 5338 | PLD2 | phospholipase D2 |
| 900029-6-B | single siRNA, 0.9 nmol | 672 | 5456 | POU3F4 | POU domain, class 3, transcripti [symbol] |
| 900029-6-B | single siRNA, 0.9 nmol | 673 | 5567 | PRKACB | protein kinase, cAMP-dependent, [symbol] |
| 900029-6-B | single siRNA, 0.9 nmol | 674 | 5581 | PRKCE | protein kinase C, epsilon |
| 900029-6-B | single siRNA, 0.9 nmol | 675 | 5590 | PRKCZ | protein kinase C, zeta |
| 900029-6-B | single siRNA, 0.9 nmol | 676 | 5602 | MAPK10 | mitogen-activated protein kinase [symbol] |
| 900029-6-B | single siRNA, 0.9 nmol | 677 | 5625 | PRODH | proline dehydrogenase (oxidase) [symbol] |
| 900029-6-B | single siRNA, 0.9 nmol | 678 | 5771 | PTPN2 | protein tyrosine phosphatase, no [symbol] |
| 900029-6-B | single siRNA, 0.9 nmol | 679 | 5795 | PTPRJ | protein tyrosine phosphatase, rec [symbol] |
| 900029-6-B | single siRNA, 0.9 nmol | 680 | 5894 | RAF1 | v-raf-1 murine leukemia viral onc [symbol] |
| 900029-6-B | single siRNA, 0.9 nmol | 681 | 5339 | PLEC1 | plectin 1, intermediate filament bi [symbol] |
| 900029-6-B | single siRNA, 0.9 nmol | 682 | 5478 | PPIA | peptidylprolyl isomerase A (cyclo [symbol] |
| 900029-6-B | single siRNA, 0.9 nmol | 683 | 5568 | PRKACG | protein kinase, cAMP-dependent, [symbol] |
| 900029-6-B | single siRNA, 0.9 nmol | 684 | 5582 | PRKCG | protein kinase C, gamma |
| 900029-6-B | single siRNA, 0.9 nmol | 685 | 5591 | PRKDC | protein kinase, DNA-activated, ca [symbol] |
| 900029-6-B | single siRNA, 0.9 nmol | 686 | 5604 | MAP2K1 | mitogen-activated protein kinase [symbol] |
| 900029-6-B | single siRNA, 0.9 nmol | 687 | 5728 | PTEN | phosphatase and tensin homolog [symbol] |
| 900029-6-B | single siRNA, 0.9 nmol | 688 | 5777 | PTPN6 | protein tyrosine phosphatase, no [symbol] |
| 900029-6-B | single siRNA, 0.9 nmol | 689 | 5829 | PXN | paxillin |
| 900029-6-B | single siRNA, 0.9 nmol | 690 | 5898 | RALA | v-ral simian leukemia viral oncog [symbol] |
| 900029-6-B | single siRNA, 0.9 nmol | 691 | 5359 | PLSCR1 | phospholipid scramblase 1 |
| 900029-6-B | single siRNA, 0.9 nmol | 692 | 5500 | PPP1CB | protein phosphatase 1, catalytic s [symbol] |
| 900029-6-B | single siRNA, 0.9 nmol | 693 | 5573 | PRKAR1A | protein kinase, cAMP-dependent, [symbol] |
| 900029-6-B | single siRNA, 0.9 nmol | 694 | 5584 | PRKCI | protein kinase C, iota |
| 900029-6-B | single siRNA, 0.9 nmol | 695 | 5594 | MAPK1 | mitogen-activated protein kinase [symbol] |
| 900029-6-B | single siRNA, 0.9 nmol | 696 | 5605 | MAP2K2 | mitogen-activated protein kinase [symbol] |
| 900029-6-B | single siRNA, 0.9 nmol | 697 | 5734 | PTGER4 | prostaglandin E receptor 4 (subty [symbol] |
| 900029-6-B | single siRNA, 0.9 nmol | 698 | 5781 | PTPN11 | protein tyrosine phosphatase, no [symbol] |
| 900029-6-B | single siRNA, 0.9 nmol | 699 | 5868 | RAB5A | RAB5A, member RAS oncogene [symbol] |
| 900029-6-B | single siRNA, 0.9 nmol | 700 | 5899 | RALB | v-ral simian leukemia viral oncog [symbol] |
| 900029-6-B | single siRNA, 0.9 nmol | 701 | 5453 | POU3F1 | POU domain, class 3, transcripti [symbol] |
| 900029-6-B | single siRNA, 0.9 nmol | 702 | 5514 | PPP1R10 | protein phosphatase 1, regulatory [symbol] |
| 900029-6-B | single siRNA, 0.9 nmol | 703 | 5578 | PRKCA | protein kinase C, alpha |
| 900029-6-B | single siRNA, 0.9 nmol | 704 | 5586 | PKN2 | protein kinase N2 |
| 900029-6-B | single siRNA, 0.9 nmol | 705 | 5595 | MAPK3 | mitogen-activated protein kinase [symbol] |
| 900029-6-B | single siRNA, 0.9 nmol | 706 | 5606 | MAP2K3 | mitogen-activated protein kinase [symbol] |
| 900029-6-B | single siRNA, 0.9 nmol | 707 | 5747 | PTK2 | PTK2 protein tyrosine kinase 2 |
| 900029-6-B | single siRNA, 0.9 nmol | 708 | 5782 | PTPN12 | protein tyrosine phosphatase, no [symbol] |
| 900029-6-B | single siRNA, 0.9 nmol | 709 | 5879 | RAC1 | ras-related C3 botulinum toxin su [symbol] |
| 900029-6-B | single siRNA, 0.9 nmol | 710 | 5906 | RAP1A | RAP1A, member of RAS oncoge [symbol] |
| 900029-6-B | single siRNA, 0.9 nmol | 711 | 5454 | POU3F2 | POU domain, class 3, transcripti [symbol] |
| 900029-6-B | single siRNA, 0.9 nmol | 712 | 5525 | PPP2R5A | protein phosphatase 2, regulatory [symbol] |
| 900029-6-B | single siRNA, 0.9 nmol | 713 | 5579 | PRKCB1 | protein kinase C, beta 1 |
| 900029-6-B | single siRNA, 0.9 nmol | 714 | 5587 | PRKD1 | protein kinase D1 |
| 900029-6-B | single siRNA, 0.9 nmol | 715 | 5599 | MAPK8 | mitogen-activated protein kinase [symbol] |
| 900029-6-B | single siRNA, 0.9 nmol | 716 | 5609 | MAP2K7 | mitogen-activated protein kinase [symbol] |
| 900029-6-B | single siRNA, 0.9 nmol | 717 | 5753 | PTK6 | PTK6 protein tyrosine kinase 6 |
| 900029-6-B | single siRNA, 0.9 nmol | 718 | 5792 | PTPRF | protein tyrosine phosphatase, rec [symbol] |
| 900029-6-B | single siRNA, 0.9 nmol | 719 | 5880 | RAC2 | ras-related C3 botulinum toxin su [symbol] |
| 900029-6-B | single siRNA, 0.9 nmol | 720 | 5908 | RAP1B | RAP1B, member of RAS oncoge [symbol] |
| 900029-7-A | single siRNA, 0.9 nmol | 721 | 5911 | RAP2A | RAP2A, member of RAS oncoge [symbol] |
| 900029-7-A | single siRNA, 0.9 nmol | 722 | 5925 | RB1 | retinoblastoma 1 (including osteo [symbol] |
| 900029-7-A | single siRNA, 0.9 nmol | 723 | 6010 | RHO | rhodopsin (opsin 2, rod pigment) [symbol] |
| 900029-7-A | single siRNA, 0.9 nmol | 724 | 6197 | RPS6KA3 | ribosomal protein S6 kinase, 90k [symbol] |
| 900029-7-A | single siRNA, 0.9 nmol | 725 | 6416 | MAP2K4 | mitogen-activated protein kinase [symbol] |
| 900029-7-A | single siRNA, 0.9 nmol | 726 | 6622 | SNCA | synuclein, alpha (non A4 compon [symbol] |
| 900029-7-A | single siRNA, 0.9 nmol | 727 | 6655 | SOS2 | son of sevenless homolog 2 (Dro [symbol] |
| 900029-7-A | single siRNA, 0.9 nmol | 728 | 6722 | SRF | serum response factor (c-fos ser [symbol] |
| 900029-7-A | single siRNA, 0.9 nmol | 729 | 6776 | STAT5A | signal transducer and activator of [symbol] |
| 900029-7-A | single siRNA, 0.9 nmol | 730 | 6907 | TBL1X | transducin (beta)-like 1X-linked |
| 900029-7-A | single siRNA, 0.9 nmol | 731 | 5914 | RARA | retinoic acid receptor, alpha |
| 900029-7-A | single siRNA, 0.9 nmol | 732 | 5928 | RBBP4 | retinoblastoma binding protein 4 |
| 900029-7-A | single siRNA, 0.9 nmol | 733 | 6018 | RLF | rearranged L-myc fusion |
| 900029-7-A | single siRNA, 0.9 nmol | 734 | 6237 | RRAS | related RAS viral (r-ras) oncogen [symbol] |
| 900029-7-A | single siRNA, 0.9 nmol | 735 | 6457 | SH3GL3 | SH3-domain GRB2-like 3 |
| 900029-7-A | single siRNA, 0.9 nmol | 736 | 6624 | FSCN1 | fascin homolog 1, actin-bundling [symbol] |
| 900029-7-A | single siRNA, 0.9 nmol | 737 | 6667 | SP1 | Sp1 transcription factor |
| 900029-7-A | single siRNA, 0.9 nmol | 738 | 6747 | SSR3 | signal sequence receptor, gamm [symbol] |
| 900029-7-A | single siRNA, 0.9 nmol | 739 | 6777 | STAT5B | signal transducer and activator of [symbol] |
| 900029-7-A | single siRNA, 0.9 nmol | 740 | 6925 | TCF4 | transcription factor 4 |
| 900029-7-A | single siRNA, 0.9 nmol | 741 | 5915 | RARB | retinoic acid receptor, beta |
| 900029-7-A | single siRNA, 0.9 nmol | 742 | 5931 | RBBP7 | retinoblastoma binding protein 7 |
| 900029-7-A | single siRNA, 0.9 nmol | 743 | 6098 | ROS1 | v-ros UR2 sarcoma virus oncoge [symbol] |
| 900029-7-A | single siRNA, 0.9 nmol | 744 | 6239 | RREB1 | ras responsive element binding p [symbol] |
| 900029-7-A | single siRNA, 0.9 nmol | 745 | 6464 | SHC1 | SHC (Src homology 2 domain co [symbol] |
| 900029-7-A | single siRNA, 0.9 nmol | 746 | 6633 | SNRPD2 | small nuclear ribonucleoprotein D [symbol] |
| 900029-7-A | single siRNA, 0.9 nmol | 747 | 6687 | SPG7 | spastic paraplegia 7 (pure and co [symbol] |
| 900029-7-A | single siRNA, 0.9 nmol | 748 | 6772 | STAT1 | signal transducer and activator of [symbol] |
| 900029-7-A | single siRNA, 0.9 nmol | 749 | 6778 | STAT6 | signal transducer and activator of [symbol] |
| 900029-7-A | single siRNA, 0.9 nmol | 750 | 6929 | TCF3 | transcription factor 3 (E2A immun [symbol] |
| 900029-7-A | single siRNA, 0.9 nmol | 751 | 5916 | RARG | retinoic acid receptor, gamma |
| 900029-7-A | single siRNA, 0.9 nmol | 752 | 5979 | RET | ret proto-oncogene |
| 900029-7-A | single siRNA, 0.9 nmol | 753 | 6134 | RPL10 | ribosomal protein L10 |
| 900029-7-A | single siRNA, 0.9 nmol | 754 | 6261 | RYR1 | ryanodine receptor 1 (skeletal) |
| 900029-7-A | single siRNA, 0.9 nmol | 755 | 6498 | SKIL | SKI-like oncogene |
| 900029-7-A | single siRNA, 0.9 nmol | 756 | 6642 | SNX1 | sorting nexin 1 |
| 900029-7-A | single siRNA, 0.9 nmol | 757 | 6709 | SPTAN1 | spectrin, alpha, non-erythrocytic 1 [symbol] |
| 900029-7-A | single siRNA, 0.9 nmol | 758 | 6773 | STAT2 | signal transducer and activator of [symbol] |
| 900029-7-A | single siRNA, 0.9 nmol | 759 | 6788 | STK3 | serine/threonine kinase 3 (STE2C [symbol] |
| 900029-7-A | single siRNA, 0.9 nmol | 760 | 6938 | TCF12 | transcription factor 12 (HTF4, heli [symbol] |
| 900029-7-A | single siRNA, 0.9 nmol | 761 | 5921 | RASA1 | RAS p21 protein activator (GTPa [symbol] |
| 900029-7-A | single siRNA, 0.9 nmol | 762 | 5999 | RGS4 | regulator of G-protein signalling 4 [symbol] |
| 900029-7-A | single siRNA, 0.9 nmol | 763 | 6195 | RPS6KA1 | ribosomal protein S6 kinase, 90 k [symbol] |
| 900029-7-A | single siRNA, 0.9 nmol | 764 | 6307 | SC4MOL | sterol-C4-methyl oxidase-like |
| 900029-7-A | single siRNA, 0.9 nmol | 765 | 6590 | SLPI | secretory leukocyte peptidase inh [symbol] |
| 900029-7-A | single siRNA, 0.9 nmol | 766 | 6643 | SNX2 | sorting nexin 2 |
| 900029-7-A | single siRNA, 0.9 nmol | 767 | 6713 | SQLE | squalene epoxidase |
| 900029-7-A | single siRNA, 0.9 nmol | 768 | 6774 | STAT3 | signal transducer and activator of [symbol] |
| 900029-7-A | single siRNA, 0.9 nmol | 769 | 6789 | STK4 | serine/threonine kinase 4 |
| 900029-7-A | single siRNA, 0.9 nmol | 770 | 7029 | TFDP2 | transcription factor Dp-2 (E2F di [symbol] |
| 900029-7-A | single siRNA, 0.9 nmol | 771 | 5922 | RASA2 | RAS p21 protein activator 2 |
| 900029-7-A | single siRNA, 0.9 nmol | 772 | 6004 | RGS16 | regulator of G-protein signalling 1 [symbol] |
| 900029-7-A | single siRNA, 0.9 nmol | 773 | 6196 | RPS6KA2 | ribosomal protein S6 kinase, 90 k [symbol] |
| 900029-7-A | single siRNA, 0.9 nmol | 774 | 6387 | CXCL12 | chemokine (C—X—C motif) ligand 1 [symbol] |
| 900029-7-A | single siRNA, 0.9 nmol | 775 | 6598 | SMARCB1 | SWI/SNF related, matrix associat [symbol] |
| 900029-7-A | single siRNA, 0.9 nmol | 776 | 6654 | SOS1 | son of sevenless homolog 1 (Dro [symbol] |
| 900029-7-A | single siRNA, 0.9 nmol | 777 | 6714 | SRC | v-src sarcoma (Schmidt-Ruppin A [symbol] |
| 900029-7-A | single siRNA, 0.9 nmol | 778 | 6775 | STAT4 | signal transducer and activator of [symbol] |
| 900029-7-A | single siRNA, 0.9 nmol | 779 | 6790 | AURKA | aurora kinase A |
| 900029-7-A | single siRNA, 0.9 nmol | 780 | 7039 | TGFA | transforming growth factor, alpha [symbol] |
| 900029-7-B | single siRNA, 0.9 nmol | 781 | 5911 | RAP2A | RAP2A, member of RAS oncoge [symbol] |
| 900029-7-B | single siRNA, 0.9 nmol | 782 | 5925 | RB1 | retinoblastoma 1 (including osteo [symbol] |
| 900029-7-B | single siRNA, 0.9 nmol | 783 | 6010 | RHO | rhodopsin (opsin 2, rod pigment) [symbol] |
| 900029-7-B | single siRNA, 0.9 nmol | 784 | 6197 | RPS6KA3 | ribosomal protein S6 kinase, 90 k [symbol] |
| 900029-7-B | single siRNA, 0.9 nmol | 785 | 6416 | MAP2K4 | mitogen-activated protein kinase [symbol] |
| 900029-7-B | single siRNA, 0.9 nmol | 786 | 6622 | SNCA | synuclein, alpha (non A4 compon [symbol] |
| 900029-7-B | single siRNA, 0.9 nmol | 787 | 6655 | SOS2 | son of sevenless homolog 2 (Dro [symbol] |
| 900029-7-B | single siRNA, 0.9 nmol | 788 | 6722 | SRF | serum response factor (c-fos sen [symbol] |
| 900029-7-B | single siRNA, 0.9 nmol | 789 | 6776 | STAT5A | signal transducer and activator of [symbol] |
| 900029-7-B | single siRNA, 0.9 nmol | 790 | 6907 | TBL1X | transducin (beta)-like 1X-linked [symbol] |
| 900029-7-B | single siRNA, 0.9 nmol | 791 | 5914 | RARA | retinoic acid receptor, alpha |
| 900029-7-B | single siRNA, 0.9 nmol | 792 | 5928 | RBBP4 | retinoblastoma binding protein 4 |
| 900029-7-B | single siRNA, 0.9 nmol | 793 | 6018 | RLF | rearranged L-myc fusion |
| 900029-7-B | single siRNA, 0.9 nmol | 794 | 6237 | RRAS | related RAS viral (r-ras) oncogen [symbol] |
| 900029-7-B | single siRNA, 0.9 nmol | 795 | 6457 | SH3GL3 | SH3-domain GRB2-like 3 |
| 900029-7-B | single siRNA, 0.9 nmol | 796 | 6624 | FSCN1 | fascin homolog 1, actin-bundling [symbol] |
| 900029-7-B | single siRNA, 0.9 nmol | 797 | 6667 | SP1 | Sp1 transcription factor |
| 900029-7-B | single siRNA, 0.9 nmol | 798 | 6747 | SSR3 | signal sequence receptor, gamm [symbol] |
| 900029-7-B | single siRNA, 0.9 nmol | 799 | 6777 | STAT5B | signal transducer and activator of |
| 900029-7-B | single siRNA, 0.9 nmol | 800 | 6925 | TCF4 | transcription factor 4 |
| 900029-7-B | single siRNA, 0.9 nmol | 801 | 5915 | RARB | retinoic acid receptor, beta |
| 900029-7-B | single siRNA, 0.9 nmol | 802 | 5931 | RBBP7 | retinoblastoma binding protein 7 |
| 900029-7-B | single siRNA, 0.9 nmol | 803 | 6098 | ROS1 | v-ros UR2 sarcoma virus oncoge [symbol] |
| 900029-7-B | single siRNA, 0.9 nmol | 804 | 6239 | RREB1 | ras responsive element binding p [symbol] |
| 900029-7-B | single siRNA, 0.9 nmol | 805 | 6464 | SHC1 | SHC (Src homology 2 domain co [symbol] |
| 900029-7-B | single siRNA, 0.9 nmol | 806 | 6633 | SNRPD2 | small nuclear ribonucleoprotein D [symbol] |
| 900029-7-B | single siRNA, 0.9 nmol | 807 | 6687 | SPG7 | spastic paraplegia 7 (pure and co [symbol] |
| 900029-7-B | single siRNA, 0.9 nmol | 808 | 6772 | STAT1 | signal transducer and activator of [symbol] |
| 900029-7-B | single siRNA, 0.9 nmol | 809 | 6778 | STAT6 | signal transducer and activator of [symbol] |
| 900029-7-B | single siRNA, 0.9 nmol | 810 | 6929 | TCF3 | transcription factor 3 (E2A immun [symbol] |
| 900029-7-B | single siRNA, 0.9 nmol | 811 | 5916 | RARG | retinoic acid receptor, gamma |
| 900029-7-B | single siRNA, 0.9 nmol | 812 | 5979 | RET | ret proto-oncogene |
| 900029-7-B | single siRNA, 0.9 nmol | 813 | 6134 | RPL10 | ribosomal protein L10 |
| 900029-7-B | single siRNA, 0.9 nmol | 814 | 6261 | RYR1 | ryanodine receptor 1 (skeletal) |
| 900029-7-B | single siRNA, 0.9 nmol | 815 | 6498 | SKIL | SKI-like oncogene |
| 900029-7-B | single siRNA, 0.9 nmol | 816 | 6642 | SNX1 | sorting nexin 1 |
| 900029-7-B | single siRNA, 0.9 nmol | 817 | 6709 | SPTAN1 | spectrin, alpha, non-erythrocytic 1 [symbol] |
| 900029-7-B | single siRNA, 0.9 nmol | 818 | 6773 | STAT2 | signal transducer and activator of [symbol] |
| 900029-7-B | single siRNA, 0.9 nmol | 819 | 6788 | STK3 | serine/threonine kinase 3 (STE2C [symbol] |
| 900029-7-B | single siRNA, 0.9 nmol | 820 | 6938 | TCF12 | transcription factor 12 (HTF4, heli [symbol] |
| 900029-7-B | single siRNA, 0.9 nmol | 821 | 5921 | RASA1 | RAS p21 protein activator (GTPa [symbol] |
| 900029-7-B | single siRNA, 0.9 nmol | 822 | 5999 | RGS4 | regulator of G-protein signalling 4 [symbol] |
| 900029-7-B | single siRNA, 0.9 nmol | 823 | 6195 | RPS6KA1 | ribosomal protein S6 kinase, 90 k [symbol] |
| 900029-7-B | single siRNA, 0.9 nmol | 824 | 6307 | SC4MOL | sterol-C4-methyl oxidase-like |
| 900029-7-B | single siRNA, 0.9 nmol | 825 | 6590 | SLPI | secretory leukocyte peptidase inh [symbol] |
| 900029-7-B | single siRNA, 0.9 nmol | 826 | 6643 | SNX2 | sorting nexin 2 |
| 900029-7-B | single siRNA, 0.9 nmol | 827 | 6713 | SQLE | squalene epoxidase |
| 900029-7-B | single siRNA, 0.9 nmol | 828 | 6774 | STAT3 | signal transducer and activator of [symbol] |
| 900029-7-B | single siRNA, 0.9 nmol | 829 | 6789 | STK4 | serine/threonine kinase 4 |
| 900029-7-B | single siRNA, 0.9 nmol | 830 | 7029 | TFDP2 | transcription factor Dp-2 (E2F di [symbol] |
| 900029-7-B | single siRNA, 0.9 nmol | 831 | 5922 | RASA2 | RAS p21 protein activator 2 |
| 900029-7-B | single siRNA, 0.9 nmol | 832 | 6004 | RGS16 | regulator of G-protein signalling 1 [symbol] |
| 900029-7-B | single siRNA, 0.9 nmol | 833 | 6196 | RPS6KA2 | ribosomal protein S6 kinase, 90 k [symbol] |
| 900029-7-B | single siRNA, 0.9 nmol | 834 | 6387 | CXCL12 | chemokine (C—X—C motif) ligand 1 [symbol] |
| 900029-7-B | single siRNA, 0.9 nmol | 835 | 6598 | SMARCB1 | SWI/SNF related, matrix associat [symbol] |
| 900029-7-B | single siRNA, 0.9 nmol | 836 | 6654 | SOS1 | son of sevenless homolog 1 (Dro [symbol] |
| 900029-7-B | single siRNA, 0.9 nmol | 837 | 6714 | SRC | v-src sarcoma (Schmidt-Ruppin A [symbol] |
| 900029-7-B | single siRNA, 0.9 nmol | 838 | 6775 | STAT4 | signal transducer and activator of [symbol] |
| 900029-7-B | single siRNA, 0.9 nmol | 839 | 6790 | AURKA | aurora kinase A |
| 900029-7-B | single siRNA, 0.9 nmol | 840 | 7039 | TGFA | transforming growth factor, alpha [symbol] |
| 900029-8-A | single siRNA, 0.9 nmol | 841 | 7046 | TGFBR1 | transforming growth factor, beta [symbol] |
| 900029-8-A | single siRNA, 0.9 nmol | 842 | 7112 | TMPO | thymopoietin |
| 900029-8-A | single siRNA, 0.9 nmol | 843 | 7278 | TUBA3C | tubulin, alpha 3c |
| 900029-8-A | single siRNA, 0.9 nmol | 844 | 7520 | XRCC5 | X-ray repair complementing defe [symbol] |
| 900029-8-A | single siRNA, 0.9 nmol | 845 | 7620 | ZNF69 | zinc finger protein 69 |
| 900029-8-A | single siRNA, 0.9 nmol | 846 | 8396 | PIP5K2B | phosphatidylinositol-4-phosphate [symbol] |
| 900029-8-A | single siRNA, 0.9 nmol | 847 | 8560 | DEGS1 | degenerative spermatocyte homo [symbol] |
| 900029-8-A | single siRNA, 0.9 nmol | 848 | 8737 | RIPK1 | receptor (TNFRSF)-interacting se [symbol] |
| 900029-8-A | single siRNA, 0.9 nmol | 849 | 8915 | BCL10 | B-cell CLL/lymphoma 10 |
| 900029-8-A | single siRNA, 0.9 nmol | 850 | 9093 | DNAJA3 | DnaJ (Hsp40) homolog, subfamil [symbol] |
| 900029-8-A | single siRNA, 0.9 nmol | 851 | 7071 | KLF10 | Kruppel-like factor 10 |
| 900029-8-A | single siRNA, 0.9 nmol | 852 | 7157 | TP53 | tumor protein p53 (Li-Fraumeni s [symbol] |
| 900029-8-A | single siRNA, 0.9 nmol | 853 | 7332 | UBE2L3 | ubiquitin-conjugating enzyme E2 [symbol] |
| 900029-8-A | single siRNA, 0.9 nmol | 854 | 7525 | YES1 | v-yes-1 Yamaguchi sarcoma viral [symbol] |
| 900029-8-A | single siRNA, 0.9 nmol | 855 | 7791 | ZYX | zyxin |
| 900029-8-A | single siRNA, 0.9 nmol | 856 | 8412 | BCAR3 | breast cancer anti-estrogen resist [symbol] |
| 900029-8-A | single siRNA, 0.9 nmol | 857 | 8651 | SOCS1 | suppressor of cytokine signaling [symbol] |
| 900029-8-A | single siRNA, 0.9 nmol | 858 | 8761 | PABPC4 | poly(A) binding protein, cytoplasm [symbol] |
| 900029-8-A | single siRNA, 0.9 nmol | 859 | 8945 | BTRC | beta-transducin repeat containing [symbol] |
| 900029-8-A | single siRNA, 0.9 nmol | 860 | 9134 | CCNE2 | cyclin E2 |
| 900029-8-A | single siRNA, 0.9 nmol | 861 | 7082 | TJP1 | tight junction protein 1 (zona occ [symbol] |
| 900029-8-A | single siRNA, 0.9 nmol | 862 | 7171 | TPM4 | tropomyosin 4 |
| 900029-8-A | single siRNA, 0.9 nmol | 863 | 7386 | UQCRFS1 | ubiquinol-cytochrome c reductase [symbol] |
| 900029-8-A | single siRNA, 0.9 nmol | 864 | 7529 | YWHAB | tyrosine 3-monooxygenase/trypto [symbol] |
| 900029-8-A | single siRNA, 0.9 nmol | 865 | 7846 | TUBA1A | tubulin, alpha 1a |
| 900029-8-A | single siRNA, 0.9 nmol | 866 | 8440 | NCK2 | NCK adaptor protein 2 |
| 900029-8-A | single siRNA, 0.9 nmol | 867 | 8655 | DYNLL1 | dynein, light chain, LC8-type 1 |
| 900029-8-A | single siRNA, 0.9 nmol | 868 | 8805 | TRIM24 | tripartite motif-containing 24 |
| 900029-8-A | single siRNA, 0.9 nmol | 869 | 8976 | WASL | Wiskott-Aldrich syndrome-like |
| 900029-8-A | single siRNA, 0.9 nmol | 870 | 9231 | DLG5 | discs, large homolog 5 (Drosophil [symbol] |
| 900029-8-A | single siRNA, 0.9 nmol | 871 | 7090 | TLE3 | transducin-like enhancer of split 3 [symbol] |
| 900029-8-A | single siRNA, 0.9 nmol | 872 | 7175 | TPR | translocated promoter region (to [symbol] |
| 900029-8-A | single siRNA, 0.9 nmol | 873 | 7409 | VAV1 | vav 1 oncogene |
| 900029-8-A | single siRNA, 0.9 nmol | 874 | 7531 | YWHAE | tyrosine 3-monooxygenase/trypto [symbol] |
| 900029-8-A | single siRNA, 0.9 nmol | 875 | 8379 | MAD1L1 | MAD1 mitotic arrest deficient-like [symbol] |
| 900029-8-A | single siRNA, 0.9 nmol | 876 | 8492 | PRSS12 | protease, serine, 12 (neurotrypsi [symbol] |
| 900029-8-A | single siRNA, 0.9 nmol | 877 | 8660 | IRS2 | insulin receptor substrate 2 |
| 900029-8-A | single siRNA, 0.9 nmol | 878 | 8825 | LIN7A | lin-7 homolog A (C. elegans) |
| 900029-8-A | single siRNA, 0.9 nmol | 879 | 9020 | MAP3K14 | mitogen-activated protein kinase [symbol] |
| 900029-8-A | single siRNA, 0.9 nmol | 880 | 9252 | RPS6KA5 | ribosomal protein S6 kinase, 90 k [symbol] |
| 900029-8-A | single siRNA, 0.9 nmol | 881 | 7094 | TLN1 | talin 1 |
| 900029-8-A | single siRNA, 0.9 nmol | 882 | 7184 | HSP90B1 | heat shock protein 90 kDa beta ( [symbol] |
| 900029-8-A | single siRNA, 0.9 nmol | 883 | 7410 | VAV2 | vav 2 oncogene |
| 900029-8-A | single siRNA, 0.9 nmol | 884 | 7534 | YWHAZ | tyrosine 3-monooxygenase/trypto [symbol] |
| 900029-8-A | single siRNA, 0.9 nmol | 885 | 8394 | PIP5K1A | phosphatidylinositol-4-phosphate [symbol] |
| 900029-8-A | single siRNA, 0.9 nmol | 886 | 8503 | PIK3R3 | phosphoinositide-3-kinase, regul [symbol] |
| 900029-8-A | single siRNA, 0.9 nmol | 887 | 8662 | EIF3S9 | eukaryotic translation initiation fa [symbol] |
| 900029-8-A | single siRNA, 0.9 nmol | 888 | 8826 | IQGAP1 | IQ motif containing GTPase activ [symbol] |
| 900029-8-A | single siRNA, 0.9 nmol | 889 | 9021 | SOCS3 | suppressor of cytokine signaling [symbol] |
| 900029-8-A | single siRNA, 0.9 nmol | 890 | 9260 | PDLIM7 | PDZ and LIM domain 7 (enigma) [symbol] |
| 900029-8-A | single siRNA, 0.9 nmol | 891 | 7109 | TMEM1 | transmembrane protein 1 |
| 900029-8-A | single siRNA, 0.9 nmol | 892 | 7205 | TRIP6 | thyroid hormone receptor interact [symbol] |
| 900029-8-A | single siRNA, 0.9 nmol | 893 | 7424 | VEGFC | vascular endothelial growth factor [symbol] |
| 900029-8-A | single siRNA, 0.9 nmol | 894 | 7535 | ZAP70 | zeta-chain (TCR) associated prot [symbol] |
| 900029-8-A | single siRNA, 0.9 nmol | 895 | 8395 | PIP5K1B | phosphatidylinositol-4-phosphate [symbol] |
| 900029-8-A | single siRNA, 0.9 nmol | 896 | 8517 | IKBKG | inhibitor of kappa light polypeptide [symbol] |
| 900029-8-A | single siRNA, 0.9 nmol | 897 | 8723 | SNX4 | sorting nexin 4 |
| 900029-8-A | single siRNA, 0.9 nmol | 898 | 8882 | ZNF259 | zinc finger protein 259 |
| 900029-8-A | single siRNA, 0.9 nmol | 899 | 9046 | DOK2 | docking protein 2, 56 kDa |
| 900029-8-A | single siRNA, 0.9 nmol | 900 | 9261 | MAPKAPK2 | mitogen-activated protein kinase- [symbol] |
| 900029-8-B | single siRNA, 0.9 nmol | 901 | 7046 | TGFBR1 | transforming growth factor, beta [symbol] |
| 900029-8-B | single siRNA, 0.9 nmol | 902 | 7112 | TMPO | thymopoietin |
| 900029-8-B | single siRNA, 0.9 nmol | 903 | 7278 | TUBA3C | tubulin, alpha 3c |
| 900029-8-B | single siRNA, 0.9 nmol | 904 | 7520 | XRCC5 | X-ray repair complementing defec [symbol] |
| 900029-8-B | single siRNA, 0.9 nmol | 905 | 7620 | ZNF69 | zinc finger protein 69 |
| 900029-8-B | single siRNA, 0.9 nmol | 906 | 8396 | PIP5K2B | phosphatidylinositol-4-phosphate [symbol] |
| 900029-8-B | single siRNA, 0.9 nmol | 907 | 8560 | DEGS1 | degenerative spermatocyte homo [symbol] |
| 900029-8-B | single siRNA, 0.9 nmol | 908 | 8737 | RIPK1 | receptor (TNFRSF)-interacting se [symbol] |
| 900029-8-B | single siRNA, 0.9 nmol | 909 | 8915 | BCL10 | B-cell CLL/lymphoma 10 |
| 900029-8-B | single siRNA, 0.9 nmol | 910 | 9093 | DNAJA3 | DnaJ (Hsp40) homolog, subfamily [symbol] |
| 900029-8-B | single siRNA, 0.9 nmol | 911 | 7071 | KLF10 | Kruppel-like factor 10 |
| 900029-8-B | single siRNA, 0.9 nmol | 912 | 7157 | TP53 | tumor protein p53 (Li-Fraumeni s [symbol] |
| 900029-8-B | single siRNA, 0.9 nmol | 913 | 7332 | UBE2L3 | ubiquitin-conjugating enzyme E2 [symbol] |
| 900029-8-B | single siRNA, 0.9 nmol | 914 | 7525 | YES1 | v-yes-1 Yamaguchi sarcoma viral [symbol] |
| 900029-8-B | single siRNA, 0.9 nmol | 915 | 7791 | ZYX | zyxin |
| 900029-8-B | single siRNA, 0.9 nmol | 916 | 8412 | BCAR3 | breast cancer anti-estrogen resist [symbol] |
| 900029-8-B | single siRNA, 0.9 nmol | 917 | 8651 | SOCS1 | suppressor of cytokine signaling [symbol] |
| 900029-8-B | single siRNA, 0.9 nmol | 918 | 8761 | PABPC4 | poly(A) binding protein, cytoplasm [symbol] |
| 900029-8-B | single siRNA, 0.9 nmol | 919 | 8945 | BTRC | beta-transducin repeat containing [symbol] |
| 900029-8-B | single siRNA, 0.9 nmol | 920 | 9134 | CCNE2 | cyclin E2 |
| 900029-8-B | single siRNA, 0.9 nmol | 921 | 7082 | TJP1 | tight junction protein 1 (zona occl [symbol] |
| 900029-8-B | single siRNA, 0.9 nmol | 922 | 7171 | TPM4 | tropomyosin 4 |
| 900029-8-B | single siRNA, 0.9 nmol | 923 | 7386 | UQCRFS1 | ubiquinol-cytochrome c reductase [symbol] |
| 900029-8-B | single siRNA, 0.9 nmol | 924 | 7529 | YWHAB | tyrosine 3-monooxygenase/trypto [symbol] |
| 900029-8-B | single siRNA, 0.9 nmol | 925 | 7846 | TUBA1A | tubulin, alpha 1a |
| 900029-8-B | single siRNA, 0.9 nmol | 926 | 8440 | NCK2 | NCK adaptor protein 2 |
| 900029-8-B | single siRNA, 0.9 nmol | 927 | 8655 | DYNLL1 | dynein, light chain, LC8-type 1 |
| 900029-8-B | single siRNA, 0.9 nmol | 928 | 8805 | TRIM24 | tripartite motif-containing 24 |
| 900029-8-B | single siRNA, 0.9 nmol | 929 | 8976 | WASL | Wiskott-Aldrich syndrome-like |
| 900029-8-B | single siRNA, 0.9 nmol | 930 | 9231 | DLG5 | discs, large homolog 5 (Drosophil [symbol] |
| 900029-8-B | single siRNA, 0.9 nmol | 931 | 7090 | TLE3 | transducin-like enhancer of split 3 [symbol] |
| 900029-8-B | single siRNA, 0.9 nmol | 932 | 7175 | TPR | translocated promoter region (to [symbol] |
| 900029-8-B | single siRNA, 0.9 nmol | 933 | 7409 | VAV1 | vav 1 oncogene |
| 900029-8-B | single siRNA, 0.9 nmol | 934 | 7531 | YWHAE | tyrosine 3-monooxygenase/trypto [symbol] |
| 900029-8-B | single siRNA, 0.9 nmol | 935 | 8379 | MAD1L1 | MAD1 mitotic arrest deficient-like [symbol] |
| 900029-8-B | single siRNA, 0.9 nmol | 936 | 8492 | PRSS12 | protease, serine, 12 (neurotrypsin [symbol] |
| 900029-8-B | single siRNA, 0.9 nmol | 937 | 8660 | IRS2 | insulin receptor substrate 2 |
| 900029-8-B | single siRNA, 0.9 nmol | 938 | 8825 | LIN7A | lin-7 homolog A (C. elegans) |
| 900029-8-B | single siRNA, 0.9 nmol | 939 | 9020 | MAP3K14 | mitogen-activated protein kinase [symbol] |
| 900029-8-B | single siRNA, 0.9 nmol | 940 | 9252 | RPS6KA5 | ribosomal protein S6 kinase, 90 k [symbol] |
| 900029-8-B | single siRNA, 0.9 nmol | 941 | 7094 | TLN1 | talin 1 |
| 900029-8-B | single siRNA, 0.9 nmol | 942 | 7184 | HSP90B1 | heat shock protein 90 kDa beta ( [symbol] |
| 900029-8-B | single siRNA, 0.9 nmol | 943 | 7410 | VAV2 | vav 2 oncogene |
| 900029-8-B | single siRNA, 0.9 nmol | 944 | 7534 | YWHAZ | tyrosine 3-monooxygenase/trypto [symbol] |
| 900029-8-B | single siRNA, 0.9 nmol | 945 | 8394 | PIP5K1A | phosphatidylinositol-4-phosphate [symbol] |
| 900029-8-B | single siRNA, 0.9 nmol | 946 | 8503 | PIK3R3 | phosphoinositide-3-kinase, regula [symbol] |
| 900029-8-B | single siRNA, 0.9 nmol | 947 | 8662 | EIF3S9 | eukaryotic translation initiation fa [symbol] |
| 900029-8-B | single siRNA, 0.9 nmol | 948 | 8826 | IQGAP1 | IQ motif containing GTPase activ [symbol] |
| 900029-8-B | single siRNA, 0.9 nmol | 949 | 9021 | SOCS3 | suppressor of cytokine signaling [symbol] |
| 900029-8-B | single siRNA, 0.9 nmol | 950 | 9260 | PDLIM7 | PDZ and LIM domain 7 (enigma) |
| 900029-8-B | single siRNA, 0.9 nmol | 951 | 7109 | TMEM1 | transmembrane protein 1 |
| 900029-8-B | single siRNA, 0.9 nmol | 952 | 7205 | TRIP6 | thyroid hormone receptor interact [symbol] |
| 900029-8-B | single siRNA, 0.9 nmol | 953 | 7424 | VEGFC | vascular endothelial growth factor [symbol] |
| 900029-8-B | single siRNA, 0.9 nmol | 954 | 7535 | ZAP70 | zeta-chain (TCR) associated prot [symbol] |
| 900029-8-B | single siRNA, 0.9 nmol | 955 | 8395 | PIP5K1B | phosphatidylinositol-4-phosphate [symbol] |
| 900029-8-B | single siRNA, 0.9 nmol | 956 | 8517 | IKBKG | inhibitor of kappa light polypeptid [symbol] |
| 900029-8-B | single siRNA, 0.9 nmol | 957 | 8723 | SNX4 | sorting nexin 4 |
| 900029-8-B | single siRNA, 0.9 nmol | 958 | 8882 | ZNF259 | zinc finger protein 259 |
| 900029-8-B | single siRNA, 0.9 nmol | 959 | 9046 | DOK2 | docking protein 2, 56 kDa |
| 900029-8-B | single siRNA, 0.9 nmol | 960 | 9261 | MAPKAPK2 | mitogen-activated protein kinase- [symbol] |
| 900029-9-A | single siRNA, 0.9 nmol | 961 | 9320 | TRIP12 | thyroid hormone receptor interact [symbol] |
| 900029-9-A | single siRNA, 0.9 nmol | 962 | 9564 | BCAR1 | breast cancer anti-estrogen resist [symbol] |
| 900029-9-A | single siRNA, 0.9 nmol | 963 | 9815 | GIT2 | G protein-coupled receptor kinas [symbol] |
| 900029-9-A | single siRNA, 0.9 nmol | 964 | 10044 | SH2D3C | SH2 domain containing 3C |
| 900029-9-A | single siRNA, 0.9 nmol | 965 | 10188 | TNK2 | tyrosine kinase, non-receptor, 2 |
| 900029-9-A | single siRNA, 0.9 nmol | 966 | 10278 | EFS | embryonal Fyn-associated substr [symbol] |
| 900029-9-A | single siRNA, 0.9 nmol | 967 | 10627 | MRCL3 | myosin regulatory light chain MR [symbol] |
| 900029-9-A | single siRNA, 0.9 nmol | 968 | 10971 | YWHAQ | tyrosine 3-monooxygenase/trypto [symbol] |
| 900029-9-A | single siRNA, 0.9 nmol | 969 | 11083 | DIDO1 | death inducer-obliterator 1 |
| 900029-9-A | single siRNA, 0.9 nmol | 970 | 23198 | PSME4 | proteasome (prosome, macropai [symbol] |
| 900029-9-A | single siRNA, 0.9 nmol | 971 | 9349 | RPL23 | ribosomal protein L23 |
| 900029-9-A | single siRNA, 0.9 nmol | 972 | 9592 | IER2 | immediate early response 2 |
| 900029-9-A | single siRNA, 0.9 nmol | 973 | 9846 | GAB2 | GRB2-associated binding protein [symbol] |
| 900029-9-A | single siRNA, 0.9 nmol | 974 | 10045 | SH2D3A | SH2 domain containing 3A |
| 900029-9-A | single siRNA, 0.9 nmol | 975 | 10241 | CALCOCO2 | calcium binding and coiled-coil d [symbol] |
| 900029-9-A | single siRNA, 0.9 nmol | 976 | 10376 | TUBA1B | tubulin, alpha 1b |
| 900029-9-A | single siRNA, 0.9 nmol | 977 | 10628 | TXNIP | thioredoxin interacting protein |
| 900029-9-A | single siRNA, 0.9 nmol | 978 | 10987 | COPS5 | COP9 constitutive photomorphog [symbol] |
| 900029-9-A | single siRNA, 0.9 nmol | 979 | 11184 | MAP4K1 | mitogen-activated protein kinase [symbol] |
| 900029-9-A | single siRNA, 0.9 nmol | 980 | 23236 | PLCB1 | phospholipase C, beta 1 (phosph [symbol] |
| 900029-9-A | single siRNA, 0.9 nmol | 981 | 9402 | GRAP2 | GRB2-related adaptor protein 2 |
| 900029-9-A | single siRNA, 0.9 nmol | 982 | 9618 | TRAF4 | TNF receptor-associated factor 4 |
| 900029-9-A | single siRNA, 0.9 nmol | 983 | 9948 | WDR1 | WD repeat domain 1 |
| 900029-9-A | single siRNA, 0.9 nmol | 984 | 10096 | ACTR3 | ARP3 actin-related protein 3 hom [symbol] |
| 900029-9-A | single siRNA, 0.9 nmol | 985 | 10251 | SPRY3 | sprouty homolog 3 (Drosophila) |
| 900029-9-A | single siRNA, 0.9 nmol | 986 | 10398 | MYL9 | myosin, light chain 9, regulatory [symbol] |
| 900029-9-A | single siRNA, 0.9 nmol | 987 | 10677 | AVIL | advillin |
| 900029-9-A | single siRNA, 0.9 nmol | 988 | 11052 | CPSF6 | cleavage and polyadenylation sp [symbol] |
| 900029-9-A | single siRNA, 0.9 nmol | 989 | 22800 | RRAS2 | related RAS viral (r-ras) oncogen [symbol] |
| 900029-9-A | single siRNA, 0.9 nmol | 990 | 23443 | SLC35A3 | solute carrier family 35 (UDP-N-a [symbol] |
| 900029-9-A | single siRNA, 0.9 nmol | 991 | 9463 | PICK1 | protein interacting with PRKCA 1 |
| 900029-9-A | single siRNA, 0.9 nmol | 992 | 9667 | SAFB2 | scaffold attachment factor B2 |
| 900029-9-A | single siRNA, 0.9 nmol | 993 | 10000 | AKT3 | v-akt murine thymoma viral onco [symbol] |
| 900029-9-A | single siRNA, 0.9 nmol | 994 | 10140 | TOB1 | transducer of ERBB2, 1 |
| 900029-9-A | single siRNA, 0.9 nmol | 995 | 10252 | SPRY1 | sprouty homolog 1, antagonist of [symbol] |
| 900029-9-A | single siRNA, 0.9 nmol | 996 | 10399 | GNB2L1 | guanine nucleotide binding protei [symbol] |
| 900029-9-A | single siRNA, 0.9 nmol | 997 | 10750 | GRAP | GRB2-related adaptor protein |
| 900029-9-A | single siRNA, 0.9 nmol | 998 | 11057 | ABHD2 | abhydrolase domain containing 2 |
| 900029-9-A | single siRNA, 0.9 nmol | 999 | 22821 | RASA3 | RAS p21 protein activator 3 |
| 900029-9-A | single siRNA, 0.9 nmol | 1000 | 23476 | BRD4 | bromodomain containing 4 |
| 900029-9-A | single siRNA, 0.9 nmol | 1001 | 9468 | PCYT1B | phosphate cytidylyltransferase 1 [symbol] |
| 900029-9-A | single siRNA, 0.9 nmol | 1002 | 9743 | RICS | Rho GTPase-activating protein [symbol] |
| 900029-9-A | single siRNA, 0.9 nmol | 1003 | 10006 | ABI1 | abi-interactor 1 |
| 900029-9-A | single siRNA, 0.9 nmol | 1004 | 10153 | CEBPZ | CCAAT/enhancer binding protein [symbol] |
| 900029-9-A | single siRNA, 0.9 nmol | 1005 | 10253 | SPRY2 | sprouty homolog 2 (Drosophila) |
| 900029-9-A | single siRNA, 0.9 nmol | 1006 | 10419 | PRMT5 | protein arginine methyltransferas [symbol] |
| 900029-9-A | single siRNA, 0.9 nmol | 1007 | 10752 | CHL1 | cell adhesion molecule with hom [symbol] |
| 900029-9-A | single siRNA, 0.9 nmol | 1008 | 11059 | WWP1 | WW domain containing E3 ubiqui [symbol] |
| 900029-9-A | single siRNA, 0.9 nmol | 1009 | 23013 | SPEN | spen homolog, transcriptional reg [symbol] |
| 900029-9-A | single siRNA, 0.9 nmol | 1010 | 23607 | CD2AP | CD2-associated protein |
| 900029-9-A | single siRNA, 0.9 nmol | 1011 | 9542 | NRG2 | neuregulin 2 |
| 900029-9-A | single siRNA, 0.9 nmol | 1012 | 9775 | EIF4A3 | eukaryotic translation initiation fa [symbol] |
| 900029-9-A | single siRNA, 0.9 nmol | 1013 | 10013 | HDAC6 | histone deacetylase 6 |
| 900029-9-A | single siRNA, 0.9 nmol | 1014 | 10174 | SORBS3 | sorbin and SH3 domain containin [symbol] |
| 900029-9-A | single siRNA, 0.9 nmol | 1015 | 10273 | STUB1 | STIP1 homology and U-box cont [symbol] |
| 900029-9-A | single siRNA, 0.9 nmol | 1016 | 10451 | VAV3 | vav 3 oncogene |
| 900029-9-A | single siRNA, 0.9 nmol | 1017 | 10856 | RUVBL2 | RuvB-like 2 (E. coli) |
| 900029-9-A | single siRNA, 0.9 nmol | 1018 | 11060 | WWP2 | WW domain containing E3 ubiqui [symbol] |
| 900029-9-A | single siRNA, 0.9 nmol | 1019 | 23136 | EPB41L3 | erythrocyte membrane protein ba [symbol] |
| 900029-9-A | single siRNA, 0.9 nmol | 1020 | 23624 | CBLC | Cas-Br-M (murine) ecotropic retr [symbol] |
| 900029-9-B | single siRNA, 0.9 nmol | 1021 | 9320 | TRIP12 | thyroid hormone receptor interact [symbol] |
| 900029-9-B | single siRNA, 0.9 nmol | 1022 | 9564 | BCAR1 | breast cancer anti-estrogen resist [symbol] |
| 900029-9-B | single siRNA, 0.9 nmol | 1023 | 9815 | GIT2 | G protein-coupled receptor kinas [symbol] |
| 900029-9-B | single siRNA, 0.9 nmol | 1024 | 10044 | SH2D3C | SH2 domain containing 3C |
| 900029-9-B | single siRNA, 0.9 nmol | 1025 | 10188 | TNK2 | tyrosine kinase, non-receptor, 2 |
| 900029-9-B | single siRNA, 0.9 nmol | 1026 | 10278 | EFS | embryonal Fyn-associated subst [symbol] |
| 900029-9-B | single siRNA, 0.9 nmol | 1027 | 10627 | MRCL3 | myosin regulatory light chain MR [symbol] |
| 900029-9-B | single siRNA, 0.9 nmol | 1028 | 10971 | YWHAQ | tyrosine 3-monooxygenase/trypto [symbol] |
| 900029-9-B | single siRNA, 0.9 nmol | 1029 | 11083 | DIDO1 | death inducer-obliterator 1 |
| 900029-9-B | single siRNA, 0.9 nmol | 1030 | 23198 | PSME4 | proteasome (prosome, macropai [symbol] |
| 900029-9-B | single siRNA, 0.9 nmol | 1031 | 9349 | RPL23 | ribosomal protein L23 |
| 900029-9-B | single siRNA, 0.9 nmol | 1032 | 9592 | IER2 | immediate early response 2 |
| 900029-9-B | single siRNA, 0.9 nmol | 1033 | 9846 | GAB2 | GRB2-associated binding protein [symbol] |
| 900029-9-B | single siRNA, 0.9 nmol | 1034 | 10045 | SH2D3A | SH2 domain containing 3A |
| 900029-9-B | single siRNA, 0.9 nmol | 1035 | 10241 | CALCOCO2 | calcium binding and coiled-coil d [symbol] |
| 900029-9-B | single siRNA, 0.9 nmol | 1036 | 10376 | TUBA1B | tubulin, alpha 1b |
| 900029-9-B | single siRNA, 0.9 nmol | 1037 | 10628 | TXNIP | thioredoxin interacting protein |
| 900029-9-B | single siRNA, 0.9 nmol | 1038 | 10987 | COPS5 | COP9 constitutive photomorphog [symbol] |
| 900029-9-B | single siRNA, 0.9 nmol | 1039 | 11184 | MAP4K1 | mitogen-activated protein kinase [symbol] |
| 900029-9-B | single siRNA, 0.9 nmol | 1040 | 23236 | PLCB1 | phospholipase C, beta 1 (phosph [symbol] |
| 900029-9-B | single siRNA, 0.9 nmol | 1041 | 9402 | GRAP2 | GRB2-related adaptor protein 2 |
| 900029-9-B | single siRNA, 0.9 nmol | 1042 | 9618 | TRAF4 | TNF receptor-associated factor 4 |
| 900029-9-B | single siRNA, 0.9 nmol | 1043 | 9948 | WDR1 | WD repeat domain 1 |
| 900029-9-B | single siRNA, 0.9 nmol | 1044 | 10096 | ACTR3 | ARP3 actin-related protein 3 hom [symbol] |
| 900029-9-B | single siRNA, 0.9 nmol | 1045 | 10251 | SPRY3 | sprouty homolog 3 (Drosophila) |
| 900029-9-B | single siRNA, 0.9 nmol | 1046 | 10398 | MYL9 | myosin, light chain 9, regulatory [symbol] |
| 900029-9-B | single siRNA, 0.9 nmol | 1047 | 10677 | AVIL | advillin |
| 900029-9-B | single siRNA, 0.9 nmol | 1048 | 11052 | CPSF6 | cleavage and polyadenylation sp [symbol] |
| 900029-9-B | single siRNA, 0.9 nmol | 1049 | 22800 | RRAS2 | related RAS viral (r-ras) oncogen [symbol] |
| 900029-9-B | single siRNA, 0.9 nmol | 1050 | 23443 | SLC35A3 | solute carrier family 35 (UDP-N-a [symbol] |
| 900029-9-B | single siRNA, 0.9 nmol | 1051 | 9463 | PICK1 | protein interacting with PRKCA 1 |
| 900029-9-B | single siRNA, 0.9 nmol | 1052 | 9667 | SAFB2 | scaffold attachment factor B2 |
| 900029-9-B | single siRNA, 0.9 nmol | 1053 | 10000 | AKT3 | v-akt murine thymoma viral onco [symbol] |
| 900029-9-B | single siRNA, 0.9 nmol | 1054 | 10140 | TOB1 | transducer of ERBB2, 1 |
| 900029-9-B | single siRNA, 0.9 nmol | 1055 | 10252 | SPRY1 | sprouty homolog 1, antagonist of [symbol] |
| 900029-9-B | single siRNA, 0.9 nmol | 1056 | 10399 | GNB2L1 | guanine nucleotide binding protei [symbol] |
| 900029-9-B | single siRNA, 0.9 nmol | 1057 | 10750 | GRAP | GRB2-related adaptor protein |
| 900029-9-B | single siRNA, 0.9 nmol | 1058 | 11057 | ABHD2 | abhydrolase domain containing 2 |
| 900029-9-B | single siRNA, 0.9 nmol | 1059 | 22821 | RASA3 | RAS p21 protein activator 3 |
| 900029-9-B | single siRNA, 0.9 nmol | 1060 | 23476 | BRD4 | bromodomain containing 4 |
| 900029-9-B | single siRNA, 0.9 nmol | 1061 | 9468 | PCYT1B | phosphate cytidylyltransferase 1, [symbol] |
| 900029-9-B | single siRNA, 0.9 nmol | 1062 | 9743 | RICS | Rho GTPase-activating protein |
| 900029-9-B | single siRNA, 0.9 nmol | 1063 | 10006 | ABI1 | abi-interactor 1 |
| 900029-9-B | single siRNA, 0.9 nmol | 1064 | 10153 | CEBPZ | CCAAT/enhancer binding protein [symbol] |
| 900029-9-B | single siRNA, 0.9 nmol | 1065 | 10253 | SPRY2 | sprouty homolog 2 (Drosophila) |
| 900029-9-B | single siRNA, 0.9 nmol | 1066 | 10419 | PRMT5 | protein arginine methyltransferas [symbol] |
| 900029-9-B | single siRNA, 0.9 nmol | 1067 | 10752 | CHL1 | cell adhesion molecule with hom [symbol] |
| 900029-9-B | single siRNA, 0.9 nmol | 1068 | 11059 | WWP1 | WW domain containing E3 ubiqui [symbol] |
| 900029-9-B | single siRNA, 0.9 nmol | 1069 | 23013 | SPEN | spen homolog, transcriptional reg [symbol] |
| 900029-9-B | single siRNA, 0.9 nmol | 1070 | 23607 | CD2AP | CD2-associated protein |
| 900029-9-B | single siRNA, 0.9 nmol | 1071 | 9542 | NRG2 | neuregulin 2 |
| 900029-9-B | single siRNA, 0.9 nmol | 1072 | 9775 | EIF4A3 | eukaryotic translation initiation fa [symbol] |
| 900029-9-B | single siRNA, 0.9 nmol | 1073 | 10013 | HDAC6 | histone deacetylase 6 |
| 900029-9-B | single siRNA, 0.9 nmol | 1074 | 10174 | SORBS3 | sorbin and SH3 domain containin [symbol] |
| 900029-9-B | single siRNA, 0.9 nmol | 1075 | 10273 | STUB1 | STIP1 homology and U-box cont [symbol] |
| 900029-9-B | single siRNA, 0.9 nmol | 1076 | 10451 | VAV3 | vav 3 oncogene |
| 900029-9-B | single siRNA, 0.9 nmol | 1077 | 10856 | RUVBL2 | RuvB-like 2 (E. coli) |
| 900029-9-B | single siRNA, 0.9 nmol | 1078 | 11060 | WWP2 | WW domain containing E3 ubiqui [symbol] |
| 900029-9-B | single siRNA, 0.9 nmol | 1079 | 23136 | EPB41L3 | erythrocyte membrane protein ba [symbol] |
| 900029-9-B | single siRNA, 0.9 nmol | 1080 | 23624 | CBLC | Cas-Br-M (murine) ecotropic retr [symbol] |
| 900029-10-A | single siRNA, 0.9 nmol | 1081 | 23760 | PITPNB | phosphatidylinositol transfer prot [symbol] |
| 900029-10-A | single siRNA, 0.9 nmol | 1082 | 27020 | NPTN | neuroplastin |
| 900029-10-A | single siRNA, 0.9 nmol | 1083 | 30011 | SH3KBP1 | SH3-domain kinase binding prote [symbol] |
| 900029-10-A | single siRNA, 0.9 nmol | 1084 | 51513 | ETV7 | ets variant gene 7 (TEL2 oncoge [symbol] |
| 900029-10-A | single siRNA, 0.9 nmol | 1085 | 54567 | DLL4 | delta-like 4 (Drosophila) |
| 900029-10-A | single siRNA, 0.9 nmol | 1086 | 55914 | ERBB2IP | erbb2 interacting protein [symbol] |
| 900029-10-A | single siRNA, 0.9 nmol | 1087 | 57561 | ARRDC3 | arrestin domain containing 3 |
| 900029-10-A | single siRNA, 0.9 nmol | 1088 | 64130 | LIN7B | lin-7 homolog B (C. elegans) |
| 900029-10-A | single siRNA, 0.9 nmol | 1089 | 79718 | TBL1XR1 | transducin (beta)-like 1X-linked r [symbol] |
| 900029-10-A | single siRNA, 0.9 nmol | 1090 | 80821 | DDHD1 | DDHD domain containing 1 |
| 900029-10-A | single siRNA, 0.9 nmol | 1091 | 25759 | SHC2 | SHC (Src homology 2 domain co [symbol] |
| 900029-10-A | single siRNA, 0.9 nmol | 1092 | 28514 | DLL1 | delta-like 1 (Drosophila) |
| 900029-10-A | single siRNA, 0.9 nmol | 1093 | 50807 | DDEF1 | development and differentiation e [symbol] |
| 900029-10-A | single siRNA, 0.9 nmol | 1094 | 51582 | AZIN1 | antizyme inhibitor 1 |
| 900029-10-A | single siRNA, 0.9 nmol | 1095 | 54822 | TRPM7 | transient receptor potential cation [symbol] |
| 900029-10-A | single siRNA, 0.9 nmol | 1096 | 56288 | PARD3 | par-3 partitioning defective 3 hom [symbol] |
| 900029-10-A | single siRNA, 0.9 nmol | 1097 | 58513 | EPS15L1 | epidermal growth factor receptor [symbol] |
| 900029-10-A | single siRNA, 0.9 nmol | 1098 | 64319 | FBRS | fibrosin |
| 900029-10-A | single siRNA, 0.9 nmol | 1099 | 79801 | SHCBP1 | SHC SH2-domain binding protein [symbol] |
| 900029-10-A | single siRNA, 0.9 nmol | 1100 | 81848 | SPRY4 | sprouty homolog 4 (Drosophila) |
| 900029-10-A | single siRNA, 0.9 nmol | 1101 | 25800 | SLC39A6 | solute carrier family 39 (zinc tran [symbol] |
| 900029-10-A | single siRNA, 0.9 nmol | 1102 | 28964 | GIT1 | G protein-coupled receptor kinas [symbol] |
| 900029-10-A | single siRNA, 0.9 nmol | 1103 | 50855 | PARD6A | par-6 partitioning defective 6 hom [symbol] |
| 900029-10-A | single siRNA, 0.9 nmol | 1104 | 51616 | TAF9B | TAF9B RNA polymerase II, TAT [symbol] |
| 900029-10-A | single siRNA, 0.9 nmol | 1105 | 54876 | C4orf30 | chromosome 4 open reading fra [symbol] |
| 900029-10-A | single siRNA, 0.9 nmol | 1106 | 56751 | BARHL1 | BarH-like 1 (Drosophila) |
| 900029-10-A | single siRNA, 0.9 nmol | 1107 | 58533 | SNX6 | sorting nexin 6 |
| 900029-10-A | single siRNA, 0.9 nmol | 1108 | 64780 | MICAL1 | microtubule associated monoxyg [symbol] |
| 900029-10-A | single siRNA, 0.9 nmol | 1109 | 80005 | DOCK5 | dedicator of cytokinesis 5 |
| 900029-10-A | single siRNA, 0.9 nmol | 1110 | 83481 | EPPK1 | epiplakin 1 |
| 900029-10-A | single siRNA, 0.9 nmol | 1111 | 25983 | NGDN | neuroguidin, EIF4E binding protei [symbol] |
| 900029-10-A | single siRNA, 0.9 nmol | 1112 | 29123 | ANKRD11 | ankyrin repeat domain 11 |
| 900029-10-A | single siRNA, 0.9 nmol | 1113 | 51079 | NDUFA13 | NADH dehydrogenase (ubiquinor [symbol] |
| 900029-10-A | single siRNA, 0.9 nmol | 1114 | 51655 | RASD1 | RAS, dexamethasone-induced 1 |
| 900029-10-A | single siRNA, 0.9 nmol | 1115 | 55236 | UBE1L2 | ubiquitin-activating enzyme E1-lik [symbol] |
| 900029-10-A | single siRNA, 0.9 nmol | 1116 | 56776 | FMN2 | formin 2 |
| 900029-10-A | single siRNA, 0.9 nmol | 1117 | 60412 | EXOC4 | exocyst complex component 4 |
| 900029-10-A | single siRNA, 0.9 nmol | 1118 | 64866 | CDCP1 | CUB domain containing protein 1 |
| 900029-10-A | single siRNA, 0.9 nmol | 1119 | 80218 | NAT13 | N-acetyltransferase 13 |
| 900029-10-A | single siRNA, 0.9 nmol | 1120 | 83737 | ITCH | itchy homolog E3 ubiquitin protei [symbol] |
| 900029-10-A | single siRNA, 0.9 nmol | 1121 | 26960 | NBEA | neurobeachin |
| 900029-10-A | single siRNA, 0.9 nmol | 1122 | 29127 | RACGAP1 | Rac GTPase activating protein 1 |
| 900029-10-A | single siRNA, 0.9 nmol | 1123 | 51343 | FZR1 | fizzy/cell division cycle 20 related [symbol] |
| 900029-10-A | single siRNA, 0.9 nmol | 1124 | 53358 | SHC3 | SHC (Src homology 2 domain co [symbol] |
| 900029-10-A | single siRNA, 0.9 nmol | 1125 | 55327 | LIN7C | lin-7 homolog C (C. elegans) |
| 900029-10-A | single siRNA, 0.9 nmol | 1126 | 56907 | SPIRE1 | spire homolog 1 (Drosophila) |
| 900029-10-A | single siRNA, 0.9 nmol | 1127 | 63920 | LOC63920 | transposon-derived Buster3 trans [symbol] |
| 900029-10-A | single siRNA, 0.9 nmol | 1128 | 79009 | DDX50 | DEAD (Asp-Glu-Ala-Asp) box po [symbol] |
| 900029-10-A | single siRNA, 0.9 nmol | 1129 | 80336 | C20orf119 | chromosome 20 open reading fra |
| 900029-10-A | single siRNA, 0.9 nmol | 1130 | 84790 | TUBA1C | tubulin, alpha 1c |
| 900029-10-A | single siRNA, 0.9 nmol | 1131 | 26986 | PABPC1 | poly(A) binding protein, cytoplas [symbol] |
| 900029-10-A | single siRNA, 0.9 nmol | 1132 | 29924 | EPN1 | epsin 1 |
| 900029-10-A | single siRNA, 0.9 nmol | 1133 | 51473 | DCDC2 | doublecortin domain containing 2 |
| 900029-10-A | single siRNA, 0.9 nmol | 1134 | 54206 | ERRFI1 | ERBB receptor feedback inhibitor [symbol] |
| 900029-10-A | single siRNA, 0.9 nmol | 1135 | 55824 | PAG1 | phosphoprotein associated with g [symbol] |
| 900029-10-A | single siRNA, 0.9 nmol | 1136 | 57403 | RAB22A | RAB22A, member RAS oncogen [symbol] |
| 900029-10-A | single siRNA, 0.9 nmol | 1137 | 63932 | CXorf56 | chromosome X open reading fra [symbol] |
| 900029-10-A | single siRNA, 0.9 nmol | 1138 | 79090 | TRAPPC6A | trafficking protein particle comple [symbol] |
| 900029-10-A | single siRNA, 0.9 nmol | 1139 | 80725 | SNIP | SNAP25-interacting protein |
| 900029-10-A | single siRNA, 0.9 nmol | 1140 | 84918 | LRP11 | low density lipoprotein receptor-r [symbol] |
| 900029-10-B | single siRNA, 0.9 nmol | 1141 | 23760 | PITPNB | phosphatidylinositol transfer prot [symbol] |
| 900029-10-B | single siRNA, 0.9 nmol | 1142 | 27020 | NPTN | neuroplastin |
| 900029-10-B | single siRNA, 0.9 nmol | 1143 | 30011 | SH3KBP1 | SH3-domain kinase binding prote [symbol] |
| 900029-10-B | single siRNA, 0.9 nmol | 1144 | 51513 | ETV7 | ets variant gene 7 (TEL2 oncoge [symbol] |
| 900029-10-B | single siRNA, 0.9 nmol | 1145 | 54567 | DLL4 | delta-like 4 (Drosophila) |
| 900029-10-B | single siRNA, 0.9 nmol | 1146 | 55914 | ERBB2IP | erbb2 interacting protein |
| 900029-10-B | single siRNA, 0.9 nmol | 1147 | 57561 | ARRDC3 | arrestin domain containing 3 |
| 900029-10-B | single siRNA, 0.9 nmol | 1148 | 64130 | LIN7B | lin-7 homolog B (C. elegans) |
| 900029-10-B | single siRNA, 0.9 nmol | 1149 | 79718 | TBL1XR1 | transducin (beta)-like 1X-linked re [symbol] |
| 900029-10-B | single siRNA, 0.9 nmol | 1150 | 80821 | DDHD1 | DDHD domain containing 1 |
| 900029-10-B | single siRNA, 0.9 nmol | 1151 | 25759 | SHC2 | SHC (Src homology 2 domain co [symbol] |
| 900029-10-B | single siRNA, 0.9 nmol | 1152 | 28514 | DLL1 | delta-like 1 (Drosophila) |
| 900029-10-B | single siRNA, 0.9 nmol | 1153 | 50807 | DDEF1 | development and differentiation e [symbol] |
| 900029-10-B | single siRNA, 0.9 nmol | 1154 | 51582 | AZIN1 | antizyme inhibitor 1 |
| 900029-10-B | single siRNA, 0.9 nmol | 1155 | 54822 | TRPM7 | transient receptor potential cation [symbol] |
| 900029-10-B | single siRNA, 0.9 nmol | 1156 | 56288 | PARD3 | par-3 partitioning defective 3 hom [symbol] |
| 900029-10-B | single siRNA, 0.9 nmol | 1157 | 58513 | EPS15L1 | epidermal growth factor receptor [symbol] |
| 900029-10-B | single siRNA, 0.9 nmol | 1158 | 64319 | FBRS | fibrosin |
| 900029-10-B | single siRNA, 0.9 nmol | 1159 | 79801 | SHCBP1 | SHC SH2-domain binding protein [symbol] |
| 900029-10-B | single siRNA, 0.9 nmol | 1160 | 81848 | SPRY4 | sprouty homolog 4 (Drosophila) |
| 900029-10-B | single siRNA, 0.9 nmol | 1161 | 25800 | SLC39A6 | solute carrier family 39 (zinc tran [symbol] |
| 900029-10-B | single siRNA, 0.9 nmol | 1162 | 28964 | GIT1 | G protein-coupled receptor kinas [symbol] |
| 900029-10-B | single siRNA, 0.9 nmol | 1163 | 50855 | PARD6A | par-6 partitioning defective 6 hom [symbol] |
| 900029-10-B | single siRNA, 0.9 nmol | 1164 | 51616 | TAF9B | TAF9B RNA polymerase II, TAT [symbol] |
| 900029-10-B | single siRNA, 0.9 nmol | 1165 | 54876 | C4orf30 | chromosome 4 open reading fra [symbol] |
| 900029-10-B | single siRNA, 0.9 nmol | 1166 | 56751 | BARHL1 | BarH-like 1 (Drosophila) |
| 900029-10-B | single siRNA, 0.9 nmol | 1167 | 58533 | SNX6 | sorting nexin 6 |
| 900029-10-B | single siRNA, 0.9 nmol | 1168 | 64780 | MICAL1 | microtubule associated monoxyg [symbol] |
| 900029-10-B | single siRNA, 0.9 nmol | 1169 | 80005 | DOCK5 | dedicator of cytokinesis 5 |
| 900029-10-B | single siRNA, 0.9 nmol | 1170 | 83481 | EPPK1 | epiplakin 1 |
| 900029-10-B | single siRNA, 0.9 nmol | 1171 | 25983 | NGDN | neuroguidin, EIF4E binding prote [symbol] |
| 900029-10-B | single siRNA, 0.9 nmol | 1172 | 29123 | ANKRD11 | ankyrin repeat domain 11 |
| 900029-10-B | single siRNA, 0.9 nmol | 1173 | 51079 | NDUFA13 | NADH dehydrogenase (ubiquino [symbol] |
| 900029-10-B | single siRNA, 0.9 nmol | 1174 | 51655 | RASD1 | RAS, dexamethasone-induced 1 |
| 900029-10-B | single siRNA, 0.9 nmol | 1175 | 55236 | UBE1L2 | ubiquitin-activating enzyme E1-lik [symbol] |
| 900029-10-B | single siRNA, 0.9 nmol | 1176 | 56776 | FMN2 | formin 2 |
| 900029-10-B | single siRNA, 0.9 nmol | 1177 | 60412 | EXOC4 | exocyst complex component 4 |
| 900029-10-B | single siRNA, 0.9 nmol | 1178 | 64866 | CDCP1 | CUB domain containing protein 1 |
| 900029-10-B | single siRNA, 0.9 nmol | 1179 | 80218 | NAT13 | N-acetyltransferase 13 |
| 900029-10-B | single siRNA, 0.9 nmol | 1180 | 83737 | ITCH | itchy homolog E3 ubiquitin protei [symbol] |
| 900029-10-B | single siRNA, 0.9 nmol | 1181 | 26960 | NBEA | neurobeachin |
| 900029-10-B | single siRNA, 0.9 nmol | 1182 | 29127 | RACGAP1 | Rac GTPase activating protein 1 |
| 900029-10-B | single siRNA, 0.9 nmol | 1183 | 51343 | FZR1 | fizzy/cell division cycle 20 related [symbol] |
| 900029-10-B | single siRNA, 0.9 nmol | 1184 | 53358 | SHC3 | SHC (Src homology 2 domain co [symbol] |
| 900029-10-B | single siRNA, 0.9 nmol | 1185 | 55327 | LIN7C | lin-7 homolog C (C. elegans) |
| 900029-10-B | single siRNA, 0.9 nmol | 1186 | 56907 | SPIRE1 | spire homolog 1 (Drosophila) |
| 900029-10-B | single siRNA, 0.9 nmol | 1187 | 63920 | LOC63920 | transposon-derived Buster3 trans [symbol] |
| 900029-10-B | single siRNA, 0.9 nmol | 1188 | 79009 | DDX50 | DEAD (Asp-Glu-Ala-Asp) box po [symbol] |
| 900029-10-B | single siRNA, 0.9 nmol | 1189 | 80336 | C20orf119 | chromosome 20 open reading fra |
| 900029-10-B | single siRNA, 0.9 nmol | 1190 | 84790 | TUBA1C | tubulin, alpha 1c |
| 900029-10-B | single siRNA, 0.9 nmol | 1191 | 26986 | PABPC1 | poly(A) binding protein, cytoplas [symbol] |
| 900029-10-B | single siRNA, 0.9 nmol | 1192 | 29924 | EPN1 | epsin 1 |
| 900029-10-B | single siRNA, 0.9 nmol | 1193 | 51473 | DCDC2 | doublecortin domain containing 2 |
| 900029-10-B | single siRNA, 0.9 nmol | 1194 | 54206 | ERRFI1 | ERBB receptor feedback inhibitor [symbol] |
| 900029-10-B | single siRNA, 0.9 nmol | 1195 | 55824 | PAG1 | phosphoprotein associated with g [symbol] |
| 900029-10-B | single siRNA, 0.9 nmol | 1196 | 57403 | RAB22A | RAB22A, member RAS oncogen [symbol] |
| 900029-10-B | single siRNA, 0.9 nmol | 1197 | 63932 | CXorf56 | chromosome X open reading fra [symbol] |
| 900029-10-B | single siRNA, 0.9 nmol | 1198 | 79090 | TRAPPC6A | trafficking protein particle comple [symbol] |
| 900029-10-B | single siRNA, 0.9 nmol | 1199 | 80725 | SNIP | SNAP25-interacting protein |
| 900029-10-B | single siRNA, 0.9 nmol | 1200 | 84918 | LRP11 | low density lipoprotein receptor-r [symbol] |
| 900029-11-A | single siRNA, 0.9 nmol | 1201 | 84962 | JUB | jub, ajuba homolog (Xenopus lae [symbol] |
| 900029-11-A | single siRNA, 0.9 nmol | 1202 | 94030 | LRRC4B | leucine rich repeat containing 4B [symbol] |
| 900029-11-A | single siRNA, 0.9 nmol | 1203 | 144983 | RP11-78J21.1 | heterogeneous nuclear ribonucle [symbol] |
| 900029-11-A | single siRNA, 0.9 nmol | 1204 | 284393 | LOC284393 | similar to ribosomal protein L10 |
| 900029-11-A | single siRNA, 0.9 nmol | 1205 | 642045 | LOC642045 | similar to myosin regulatory light [symbol] |
| 900029-11-A | single siRNA, 0.9 nmol | 1206 | 648695 | LOC648695 | similar to retinoblastoma binding [symbol] |
| 900029-11-A | single siRNA, 0.9 nmol | 1207 | 731292 | LOC731292 | similar to Phosphatidylinositol-3,4 |
| 900029-11-A | single siRNA, 0.9 nmol | 1208 | 84981 | MGC14376 | hypothetical protein MGC14376 |
| 900029-11-A | single siRNA, 0.9 nmol | 1209 | 103910 | MRLC2 | myosin regulatory light chain MR [symbol] |
| 900029-11-A | single siRNA, 0.9 nmol | 1210 | 150094 | SNF1LK | SNF1-like kinase |
| 900029-11-A | single siRNA, 0.9 nmol | 1211 | 286887 | KRT6C | keratin 6C |
| 900029-11-A | single siRNA, 0.9 nmol | 1212 | 642954 | LOC642954 | similar to retinoblastoma binding [symbol] |
| 900029-11-A | single siRNA, 0.9 nmol | 1213 | 643997 | LOC643997 | similar to peptidylprolyl isomeras [symbol] |
| 900029-11-A | single siRNA, 0.9 nmol | 1214 | 731751 | LOC731751 | similar to protein kinase, DNA-ac [symbol] |
| 900029-11-A | single siRNA, 0.9 nmol | 1215 | 85021 | REPS1 | RALBP1 associated Eps domain [symbol] |
| 900029-11-A | single siRNA, 0.9 nmol | 1216 | 121536 | AEBP2 | AE binding protein 2 |
| 900029-11-A | single siRNA, 0.9 nmol | 1217 | 151987 | PPP4R2 | protein phosphatase 4, regulatory [symbol] |
| 900029-11-A | single siRNA, 0.9 nmol | 1218 | 343472 | BARHL2 | BarH-like 2 (Drosophila) |
| 900029-11-A | single siRNA, 0.9 nmol | 1219 | 643751 | LOC643751 | similar to cell division cycle 42 |
| 900029-11-A | single siRNA, 0.9 nmol | 1220 | 727897 | MUC5B | mucin 5B, oligomeric mucus/gel-f [symbol] |
| 900029-11-A | single siRNA, 0.9 nmol | 1221 | 85458 | DIXDC1 | DIX domain containing 1 |
| 900029-11-A | single siRNA, 0.9 nmol | 1227 | 139322 | APOOL | apolipoprotein O-like |
| 900029-11-A | single siRNA, 0.9 nmol | 1223 | 253738 | EBF3 | early B-cell factor 3 |
| 900029-11-A | single siRNA, 0.9 nmol | 1224 | 387927 | LOC387927 | similar to NIMA (never in mitosis [symbol] |
| 900029-11-A | single siRNA, 0.9 nmol | 1225 | 643752 | hCG_1757335 | hCG1757335 |
| 900029-11-A | single siRNA, 0.9 nmol | 1226 | 728153 | LOC728153 | hypothetical protein LOC728153 |
| 900029-11-A | single siRNA, 0.9 nmol | 1227 | 90665 | TBL1Y | transducin (beta)-like 1Y-linked |
| 900029-11-A | single siRNA, 0.9 nmol | 1228 | 140735 | DYNLL2 | dynein, light chain, LC8-type 2 |
| 900029-11-A | single siRNA, 0.9 nmol | 1229 | 255324 | EPGN | epithelial mitogen homolog (mou [symbol] |
| 900029-11-A | single siRNA, 0.9 nmol | 1230 | 389342 | LOC389342 | similar to ribosomal protein L10 |
| 900029-11-A | single siRNA, 0.9 nmol | 1231 | 643997 | LOC643997 | similar to peptidylprolyl isomeras [symbol] |
| 900029-11-A | single siRNA, 0.9 nmol | 1232 | 728198 | LOC728198 | similar to transcription associated |
| 900029-11-A | single siRNA, 0.9 nmol | 1233 | 92521 | SPECC1 | sperm antigen with calponin hom [symbol] |
| 900029-11-A | single siRNA, 0.9 nmol | 1234 | 140801 | RPL10L | ribosomal protein L10-like |
| 900029-11-A | single siRNA, 0.9 nmol | 1235 | 283455 | KSR2 | kinase suppressor of ras 2 |
| 900029-11-A | single siRNA, 0.9 nmol | 1236 | 390006 | LOC390006 | similar to peptidylprolyl isomerase |
| 900029-11-A | single siRNA, 0.9 nmol | 1237 | 645691 | LOC645691 | similar to Heterogeneous nuclear [symbol] |
| 900029-11-A | single siRNA, 0.9 nmol | 1238 | 728024 | hCG_1640171 | hCG1640171 |
| 900029-11-B | single siRNA, 0.9 nmol | 1239 | 84962 | JUB | jub, ajuba homolog (Xenopus lae [symbol] |
| 900029-11-B | single siRNA, 0.9 nmol | 1240 | 94030 | LRRC4B | leucine rich repeat containing 4B |
| 900029-11-B | single siRNA, 0.9 nmol | 1241 | 144983 | RP11-78J21.1 | heterogeneous nuclear ribonucle [symbol] |
| 900029-11-B | single siRNA, 0.9 nmol | 1242 | 284393 | LOC284393 | similar to ribosomal protein L10 |
| 900029-11-B | single siRNA, 0.9 nmol | 1243 | 642045 | LOC642045 | similar to myosin regulatory light [symbol] |
| 900029-11-B | single siRNA, 0.9 nmol | 1244 | 648695 | LOC648695 | similar to retinoblastoma binding [symbol] |
| 900029-11-B | single siRNA, 0.9 nmol | 1245 | 731292 | LOC731292 | similar to Phosphatidylinositol-3,4 |
| 900029-11-B | single siRNA, 0.9 nmol | 1246 | 84981 | MGC14376 | hypothetical protein MGC14376 |
| 900029-11-B | single siRNA, 0.9 nmol | 1247 | 103910 | MRLC2 | myosin regulatory light chain MR [symbol] |
| 900029-11-B | single siRNA, 0.9 nmol | 1248 | 150094 | SNF1LK | SNF1-like kinase |
| 900029-11-B | single siRNA, 0.9 nmol | 1249 | 286887 | KRT6C | keratin 6C |
| 900029-11-B | single siRNA, 0.9 nmol | 1250 | 642954 | LOC642954 | similar to retinoblastoma binding [symbol] |
| 900029-11-B | single siRNA, 0.9 nmol | 1251 | 643997 | LOC643997 | similar to peptidylprolyl isomeras [symbol] |
| 900029-11-B | single siRNA, 0.9 nmol | 1252 | 731751 | LOC731751 | similar to protein kinase, DNA-ac [symbol] |
| 900029-11-B | single siRNA, 0.9 nmol | 1253 | 85021 | REPS1 | RALBP1 associated Eps domain [symbol] |
| 900029-11-B | single siRNA, 0.9 nmol | 1254 | 121536 | AEBP2 | AE binding protein 2 |
| 900029-11-8 | single siRNA, 0.9 nmol | 1255 | 151987 | PPP4R2 | protein phosphatase 4, regulatory [symbol] |
| 900029-11-B | single siRNA, 0.9 nmol | 1256 | 343472 | BARHL2 | BarH-like 2 (Drosophila) |
| 900029-11-B | single siRNA, 0.9 nmol | 1257 | 643751 | LOC643751 | similar to cell division cycle 42 |
| 900029-11-B | single siRNA, 0.9 nmol | 1258 | 727897 | MUC5B | mucin 5B, oligomeric mucus/gel- [symbol] |
| 900029-11-B | single siRNA, 0.9 nmol | 1259 | 85458 | DIXDC1 | DIX domain containing 1 |
| 900029-11-B | single siRNA, 0.9 nmol | 1260 | 139322 | APOOL | apolipoprotein O-like |
| 900029-11-B | single siRNA, 0.9 nmol | 1261 | 253738 | EBF3 | early B-cell factor 3 |
| 900029-11-B | single siRNA, 0.9 nmol | 1262 | 387927 | LOC387927 | similar to NIMA (never in mitosis [symbol] |
| 900029-11-B | single siRNA, 0.9 nmol | 1263 | 643752 | hCG_1757335 | hCG1757335 |
| 900029-11-B | single siRNA, 0.9 nmol | 1264 | 728153 | LOC728153 | hypothetical protein LOC728153 |
| 900029-11-B | single siRNA, 0.9 nmol | 1265 | 90665 | TBL1Y | transducin (beta)-like 1Y-linked |
| 900029-11-B | single siRNA, 0.9 nmol | 1266 | 140735 | DYNLL2 | dynein, light chain, LC8-type 2 |
| 900029-11-B | single siRNA, 0.9 nmol | 1267 | 255324 | EPGN | epithelial mitogen homolog (mou [symbol] |
| 900029-11-B | single siRNA, 0.9 nmol | 1268 | 389342 | LOC389342 | similar to ribosomal protein L10 |
| 900029-11-B | single siRNA, 0.9 nmol | 1269 | 643997 | LOC643997 | similar to peptidylprolyl isomeras [symbol] |
| 900029-11-B | single siRNA, 0.9 nmol | 1270 | 728198 | LOC728198 | similar to transcription associated [symbol] |
| 900029-11-B | single siRNA, 0.9 nmol | 1271 | 92521 | SPECC1 | sperm antigen with calponin hom [symbol] |
| 900029-11-B | single siRNA, 0.9 nmol | 1272 | 140801 | RPL10L | ribosomal protein L10-like |
| 900029-11-B | single siRNA, 0.9 nmol | 1273 | 283455 | KSR2 | kinase suppressor of ras 2 |
| 900029-11-B | single siRNA, 0.9 nmol | 1274 | 390006 | LOC390006 | similar to peptidylprolyl isomeras [symbol] |
| 900029-11-B | single siRNA, 0.9 nmol | 1275 | 645691 | LOC645691 | similar to Heterogeneous nuclear [symbol] |
| 900029-11-B | single siRNA, 0.9 nmol | 1276 | 728024 | hCG_1640171 | hCG1640171 |

| | mRNA | | | |
|---|---|---|---|---|
| Plate Id | Accessions | siRNA Target Sequence | Product Id | Product Name |
| 900029-1-A | NM_001085 | AAGGTTCTACTTGAGCAAGAA | SI00715540 | Hs_SERPINA3_4 |
| 900029-1-A | NM_001014431 N [symbol] | CACCATGAGCGACGTGGCTAT | SI02758406 | Hs_AKT1_11 |
| 900029-1-A | NM_001167 | AAGTGCTTTCACTGTGGAGGA | SI00299446 | Hs_BIRC4_5 |
| 900029-1-A | NM_001660 XM_ [symbol] | ACCAAGGACATGTTTGATAAA | SI00300076 | Hs_ARF4_4 |
| 900029-1-A | NM_001042678 N [symbol] | CCCTACTGTCTTTGAGAACTA | SI02663913 | Hs_RHOC_6 |
| 900029-1-A | NM_053056 | CCCTACTGTCTTTGAGAACTA | SI02654547 | Hs_CCND1_6 |
| 900029-1-A | NM_001202 NM_ [symbol] | GCGAGCCATGCTAGTTTGATA | SI03105193 | Hs_BMP4_7 |
| 900029-1-A | NM_004342 NM_ [symbol] | CGCCAAGAAAGATACGAGATA | SI03193498 | Hs_CALD1_6 |
| 900029-1-A | NM_001223 NM_ [symbol] | TACCTCTTCCCAGGACATTAA | SI02662443 | Hs_CASP1_15 |
| 900029-1-A | NM_170662 | CAGGTGTTGCAGCATCATTGA | SI03072398 | Hs_CBLB_5 |
| 900029-1-A | NM_005157 NM_ [symbol] | AACGGCTGATGTGGACTGTCT | SI00299110 | Hs_ABL1_9 |
| 900029-1-A | NM_001626 | AAGGTACTTCGATGATGAATT | SI00299173 | Hs_AKT2_6 |
| 900029-1-A | NM_000484 NM_ [symbol] | CTGGTCTTCAATTACCAAGAA | SI02780288 | Hs_APP_10 |
| 900029-1-A | NM_001662 | TTCGCGGATCTTCGGGAAGAA | SI03242351 | Hs_ARF5_5 |
| 900029-1-A | NM_001665 | CACGCTGTGCGCTACCTCGAA | SI00702884 | Hs_RHOG_4 |
| 900029-1-A | NM_005178 | CAACGTGAACGCGCAAATGTA | SI02654554 | Hs_BCL3_5 |
| 900029-1-A | NM_004329 | GCGGCAGACATTAAAGGTACA | SI02659629 | Hs_BMPR1A_6 |
| 900029-1-A | NM_006888 | CTGGTTGTATCTTATTAGCAA | SI02224222 | Hs_CALM1_6 |
| 900029-1-A | NM_001753 | AAGCAAGTGTACGACGCGCAC | SI00299642 | Hs_CAV1_10 |
| 900029-1-A | NM_001759 | CAGGGCCGTGCGGGACCGCAA | SI03071369 | Hs_CCND2_5 |
| 900029-1-A | NM_005159 | CTGATCGTATGCAGAAGGAAA | SI00291389 | Hs_ACTC_3 |
| 900029-1-A | NM_004304 | CTGGGCCTGTATACCGGATAA | SI02632854 | Hs_ALK_6 |
| 900029-1-A | NM_001654 | CCGGGATGGCATGAGTGTCTA | SI00287693 | Hs_ARAF_6 |
| 900029-1-A | NM_000045 | AGCGCCAAGTCCAGAACCATA | SI03043859 | Hs_ARG1_5 |
| 900029-1-A | NM_004316 | CCAGTTGTACTTCAGCACCAA | SI03075793 | Hs_ASCL1_5 |
| 900029-1-A | NM_004327 NM_ [symbol] | AAGGTCAACGACAAAGAGGTG | SI00299425 | Hs_BCR_5 |
| 900029-1-A | NM_001203 | ACGGATATTGTTTCACGATGA | SI00604989 | Hs_BMPR1B_6 |
| 900029-1-A | NM_001743 | AAGCCCTTCTGCACATCTAAA | SI02758420 | Hs_CALM2_9 |
| 900029-1-A | NM_001233 NM_ [symbol] | ACGCTAATAAGTGACAAATAA | SI02664389 | Hs_CAV2_10 |
| 900029-1-A | NM_001760 | CATGCGGAAGATGCTGGCTTA | SI03073924 | Hs_CCND3_5 |
| 900029-1-A | NM_004924 | CCCGCAAATCATCAACTCCAA | SI02779980 | Hs_ACTN4_6 |
| 900029-1-A | NM_000479 | CCAATAAAGACCAGCAAGCAA | SI02623572 | Hs_AMH_4 |
| 900029-1-A | NM_001657 | ATGATTGACAGTAGTTTATCA | SI03049683 | Hs_AREG_5 |
| 900029-1-A | NM_001172 | AACCTTGTATCTCTTCTGCAA | SI00301210 | Hs_ARG2_4 |
| 900029-1-A | NM_005170 | CTCGACTTCTCCAGCTGGTTA | SI03091018 | Hs_ASCL2_6 |
| 900029-1-A | NM_001024912 N [symbol] | CTCCATCCGTTGGTTCTTCAA | SI03089632 | Hs_CEACAM1_7 |
| 900029-1-A | NM_004333 | AACATATAGAGGCCCTATTGG | SI00299488 | Hs_BRAF_1 |
| 900029-1-A | NM_005184 | CCGCAGAGCTGCGTCACGTAA | SI02659685 | Hs_CALM3_7 |
| 900029-1-A | NM_001234 NM_ [symbol] | CAGCTTTGACGGCGTGTGGAA | SI03068730 | Hs_CAV3_9 |
| 900029-1-A | NM_001238 NM_ [symbol] | AAGGCAAACGTGACCGTTTTT | SI00299691 | Hs_CCNE1_5 |
| 900029-1-A | NM_014203 NM_ [symbol] | CACCGTCATCAATGCCCTCAA | SI02661610 | Hs_AP2A1_6 |
| 900029-1-A | NM_001002857 N [symbol] | CACGGCCTGAGCGTCCAGAAA | SI03060855 | Hs_ANXA2_10 |
| 900029-1-A | NM_001024226 N [symbol] | AGGGAAGACCACGATCCTCTA | SI02757279 | Hs_ARF1_11 |
| 900029-1-A | NM_001664 | TACCTTATAGTTACTGTGTAA | SI02776907 | Hs_RHOA_8 |
| 900029-1-A | NM_001679 XM_ [symbol] | AAGGATAGATTGTGTTTCAAA | SI00306614 | Hs_ATP1B3_4 |
| 900029-1-A | NM_001715 | CTGGTAAGCGACTGTCATCAA | SI02626785 | Hs_BLK_5 |
| 900029-1-A | NM_001729 | ATCCATGAGATAGCTATTATA | SI02626820 | Hs_BTC_6 |
| 900029-1-A | NM_001222 NM_ [symbol] | CTCGGATATGTCGACTTCTGA | SI02758427 | Hs_CAMK2G_7 |
| 900029-1-A | NM_001001890 N [symbol] | CAGGATACAAGGCAGATCCAA | SI03069766 | Hs_RUNX1_6 |
| 900029-1-A | NM_000733 | CTCAGTATCCTGGATCTGAAA | SI03088848 | Hs_CD3E_7 |
| 900029-1-A | NM_012305 | AGGCTCTTGATGGCTATAGTA | SI03146633 | Hs_AP2A2_5 |
| 900029-1-A | NM_001155 NM_ [symbol] | CCGACAAACTTTACAAATCCA | SI00297024 | Hs_ANXA6_4 |
| 900029-1-A | NM_001659 | CACCTATATGACCAATCCCTA | SI02654477 | Hs_ARF3_5 |
| 900029-1-A | NM_004040 | CCCGGACTCGCTGGAGAACAT | SI03078257 | Hs_RHOB_6 |
| 900029-1-A | NM_001699 NM_ [symbol] | TCCAAGATTCTAGATGATTAA | SI00605311 | Hs_AXL_10 |
| 900029-1-A | NM_001200 | CACCGAATTAATATTTATGAA | SI00023373 | Hs_BMP2_4 |
| 900029-1-A | NM_001954 NM_ [symbol] | CAGGAATGATTTCCTGAAAGA | SI00605444 | Hs_DDR1_10 |
| 900029-1-A | NM_001745 | AGGGCTGAGTTTGTATTATTA | SI02777110 | Hs_CAMLG_8 |
| 900029-1-A | NM_005188 | CCGTACTATCTTGTCAAGATA | SI02757321 | Hs_CBL_8 |
| 900029-1-A | NM_000734 NM_ [symbol] | AACGAGCTCAATCTAGGACGA | SI03029397 | Hs_CD247_1 |
| 900029-1-B | NM_001085 | CCCAAGATACTCATCAGTCAA | SI00715533 | Hs_SERPINA3_3 |
| 900029-1-B | NM_001014431 N [symbol] | CACGCTTGGTCCCGAGGCCAA | SI02757244 | Hs_AKT1_10 |
| 900029-1-B | NM_001167 | GGCCGGAATCTTAATATTCGA | SI03105739 | Hs_BIRC4_8 |
| 900029-1-B | NM_001660 | CCCATTTGGAATTATTCCTAA | SI00300069 | Hs_ARF4_3 |
| 900029-1-B | NM_001042678 N [symbol] | CACCATGGCTGCAATCCGAAA | SI02663906 | Hs_RHOC_5 |
| 900029-1-B | NM_053056 | AACACCAGCTCCTGTGCTGCG | SI02654540 | Hs_CCND1_5 |
| 900029-1-B | NM_001202 NM_ [symbol] | CAGCAGCATCCCTGAGAACGA | SI03065643 | Hs_BMP4_6 |
| 900029-1-B | NM_004342 NM_ [symbol] | ATGGATCAAAGTTTGAATTAA | SI02731232 | Hs_CALD1_5 |
| 900029-1-B | NM_001223 NM_ [symbol] | CTCATTGAACATATGCAAGAA | SI02661932 | Hs_CASP1_14 |
| 900029-1-B | NM_170662 | ACGGGCAATAAGACTCTTTAA | SI00156779 | Hs_CBLB_3 |
| 900029-1-B | NM_005157 NM_ [symbol] | AACCAAGCCTTTGAAACAATG | SI00299103 | Hs_ABL1_8 |
| 900029-1-B | NM_001626 | AACAACTTCTCCGTAGCAGAA | SI00299166 | Hs_AKT2_5 |
| 900029-1-B | NM_000484 NM_ [symbol] | ACCCAATTAAGTCCTACTTTA | SI02780281 | Hs_APP_9 |
| 900029-1-B | NM_001662 | CATCTTTGTGGTGGACAGTAA | SI00300321 | Hs_ARF5_4 |
| 900029-1-B | NM_001665 | GAGAAGGTGAATGTACCCTAA | SI00702877 | Hs_RHOG_3 |
| 900029-1-B | NM_005178 | CCGGCCGGAGGCGCTTTACTA | SI03082156 | Hs_BCL3_6 |
| 900029-1-B | NM_004329 | CAGCTACGCCGGACAATAGAA | SI02659622 | Hs_BMPR1A_5 |
| 900029-1-B | NM_006888 | CGGCAACTTACACACATTGAA | SI02224215 | Hs_CALM1_5 |
| 900029-1-B | NM_001753 | CACCTTCACTGTGACGAAATA | SI00299614 | Hs_CAV1_6 |
| 900029-1-B | NM_001759 | AAGAAATAGACTTGCACCTTA | SI00027853 | Hs_CCND2_4 |
| 900029-1-B | NM_005159 | TCCTAGCACCATGAAGATTAA | SI00291382 | Hs_ACTC_2 |
| 900029-1-B | NM_004304 | CACCTACGTATTTAAGATGAA | SI02632847 | Hs_ALK_5 |
| 900029-1-B | NM_001654 | CCGACTCATCAAGGGACGAAA | SI00287686 | Hs_ARAF_5 |
| 900029-1-B | NM_000045 | AAGCATAGAGTTATCCTTCTA | SI00000707 | Hs_ARG1_4 |
| 900029-1-B | NM_004316 | ACGCGTTATAGTAACTCCCAT | SI00062587 | Hs_ASCL1_3 |
| 900029-1-B | NM_004327 NM_ [symbol] | CAGCATTCCGCTGACCATCAA | SI00288141 | Hs_BCR_8 |
| 900029-1-B | NM_001203 | AACGAATGTAATAAAGACCTA | SI00604982 | Hs_BMPR1B_5 |
| 900029-1-B | NM_001743 | GACCTTGTACAGAATGTGTTA | SI02758413 | Hs_CALM2_8 |
| 900029-1-B | NM_001233 NM_ [symbol] | CCGGCTCAACTCGCATCTCAA | SI00299663 | Hs_CAV2_7 |
| 900029-1-B | NM_001760 | TGGGACAGAATTGGATACATA | SI00027881 | Hs_CCND3_4 |
| 900029-1-B | NM_004924 | ACGCAGCATCGTGGACTACAA | SI02779973 | Hs_ACTN4_5 |
| 900029-1-B | NM_000479 | CAGGCCATCCGCGGAACTCGA | SI02623565 | Hs_AMH_3 |
| 900029-1-B | NM_001657 | ATGGATTTGAGGTTACCTCAA | SI00299936 | Hs_AREG_3 |
| 900029-1-B | NM_001172 | TTGGATAACCTTCCTTCTAAA | SI00301203 | Hs_ARG2_3 |
| 900029-1-B | NM_005170 | CCGCGTGAAGCTGGTGAACTT | SI03081218 | Hs_ASCL2_5 |
| 900029-1-B | NM_001024912 N [symbol] | CAAGACGATCATAGTCACTGA | SI03053694 | Hs_CEACAM1_6 |
| 900029-1-B | NM_004333 | TTGCTTATATGTTAAATTGAA | SI02632966 | Hs_BRAF_5 |
| 900029-1-B | NM_005184 | CACCAATTGATTGACTGAGAA | SI02622060 | Hs_CALM3_5 |
| 900029-1-B | NM_001234 NM_ [symbol] | TTGCGTTCACTTGTACTGTAA | SI02625665 | Hs_CAV3_7 |
| 900029-1-B | NM_001238 NM_ [symbol] | CAAGATTTCTTTGACCGGTAT | SI03054037 | Hs_CCNE1_8 |
| 900029-1-B | NM_014203 NM_ [symbol] | TTGGATGGCTACAGTAAGAAA | SI02661603 | Hs_AP2A1_5 |
| 900029-1-B | NM_001002857 N [symbol] | CGGCAAGTCCCTGTACTATTA | SI02632385 | Hs_ANXA2_8 |
| 900029-1-B | NM_001024226 N [symbol] | CACGATCCTCTACAAGCTTAA | SI02757272 | Hs_ARF1_10 |
| 900029-1-B | NM_001664 | CAAGCTAGACGTGGGAAGAAA | SI02654267 | Hs_RHOA_7 |
| 900029-1-B | NM_001679 XM_ [symbol] | CAGGATGATCGTGACAAGTTT | SI00306607 | Hs_ATP1B3_3 |
| 900029-1-B | NM_001715 | CAACATGAAGGTGGCCATTAA | SI00027055 | Hs_BLK_3 |
| 900029-1-B | NM_001729 | CACCAGAAGTCCTGAAACTAA | SI02626813 | Hs_BTC_5 |
| 900029-1-B | NM_001222 NM_ [symbol] | CCGATGAGAAACCTCGTGTTA | SI00157402 | Hs_CAMK2G_3 |
| 900029-1-B | NM_001001890 N [symbol] | CAGGTCGTTCTTATCTAGAGA | SI00027769 | Hs_RUNX1_4 |
| 900029-1-B | NM_000733 | CACAATTGTCATAGTGGACAT | SI03055598 | Hs_CD3E_6 |
| 900029-1-B | NM_012305 | TCGGATATCCGCAACTGTAAA | SI00297444 | Hs_AP2A2_4 |
| 900029-1-B | NM_001155 NM_ [symbol] | AAGGCTCTTCAAGGCTATGAA | SI00297017 | Hs_ANXA6_3 |
| 900029-1-B | NM_001659 | TTCCAATTTACTGGATTTAAA | SI00300020 | Hs_ARF3_4 |
| 900029-1-B | NM_004040 | ACAAGGCATTCTCTAAAGCTA | SI03037531 | Hs_RHOB_5 |
| 900029-1-B | NM_001699 NM_ [symbol] | CCGGTGTTCTAAGATGTGATA | SI00605304 | Hs_AXL_9 |
| 900029-1-B | NM_001200 | CTCAGCATGTTCGGCCTGAAA | SI00023366 | Hs_BMP2_3 |
| 900029-1-B | NM_001954 NM_ [symbol] | ACGGTGTGAATCACACATCCA | SI00605437 | Hs_DDR1_9 |
| 900029-1-B | NM_001745 | ATCGATCAATGGATACCTATA | SI02777103 | Hs_CAMLG_7 |
| 900029-1-B | NM_005188 | CCCGCCGAACTCTCTCAGATA | SI02662471 | Hs_CBL_7 |
| 900029-1-B | NM_000734 NM_ [symbol] | CAGGAAGGCCTGTACAATGAA | SI00014462 | Hs_CD3Z_4 |
| 900029-2-A | NM_001771 | AAGCAGAATACATTCACGCTA | SI03032820 | Hs_CD22_7 |
| 900029-2-A | NM_004360 | TCGGCCTGAAGTGACTCGTAA | SI02654029 | Hs_CDH1_13 |
| 900029-2-A | NM_020549 NM_ [symbol] | CACGGAGATGTTCTGCTGCTA | SI00127022 | Hs_CHAT_4 |
| 900029-2-A | NM_001008490 N [symbol] | CAGGAAGATCTGTGGACCAAA | SI03068968 | Hs_KLF6_5 |
| 900029-2-A | NM_000760 NM_ [symbol] | CCAGGCGATCTGCATACTTTA | SI00156723 | Hs_CSF3R_5 |
| 900029-2-A | NM_001904 XM_ [symbol] | CTCGGGATGTTCACAACCGAA | SI02662478 | Hs_CTNNB1_5 |
| 900029-2-A | NM_001365 | CAGGATATGAGTTGCAGGTGA | SI02626106 | Hs_DLG4_6 |
| 900029-2-A | NM_001394 NM_ [symbol] | AAGGACTCCGAATACATAATA | SI03132227 | Hs_DUSP4_5 |
| 900029-2-A | NM_001955 | CAGGTCGGAGACCATGAGAAA | SI02627380 | Hs_EDN1_6 |
| 900029-2-A | NM_000399 | GAGCGAGGAGCAATTGATTAA | SI00008792 | Hs_EGR2_4 |
| 900029-2-A | NM_000610 NM_ [symbol] | AACTCCATCTGTGCAGCAAAC | SI00299705 | Hs_CD44_5 |
| 900029-2-A | NM_001793 | AAGCCTCTTACCTGCCGTAAA | SI02663941 | Hs_CDH3_6 |
| 900029-2-A | NM_005198 NM_ [symbol] | GACCATGGAGCGGTACCTAAA | SI03101511 | Hs_CHKB_5 |
| 900029-2-A | NM_004379 NM_ [symbol] | AAGCCCAGCCACAGATTGCCA | SI00299908 | Hs_CREB1_7 |
| 900029-2-A | NM_004383 | AAGTACAACTTCCACGGCACT | SI00299943 | Hs_CSK_1 |
| 900029-2-A | NM_001331 XM_ [symbol] | CTGGTGTTGATCAACAAATCA | SI02626001 | Hs_CTNND1_5 |
| 900029-2-A | NM_001005336 N [symbol] | CAGGTCATGCTTCTCATCGAT | SI03071894 | Hs_DNM1_5 |
| 900029-2-A | NM_001946 NM_ [symbol] | GTCGGAAATGGCGATCAGCAA | SI03106404 | Hs_DUSP6_6 |
| 900029-2-A | NM_001402 | CAGAATAGGAACAAGGTTCTA | SI03063235 | Hs_EEF1A1_8 |
| 900029-2-A | NM_004430 XM_ [symbol] | CGGCAACAAGACCGTGACCTA | SI03195766 | Hs_EGR3_6 |
| 900029-2-A | NM_000611 NM_ [symbol] | TAGGTGTGACTTGAACTAGAT | SI03112200 | Hs_CD59_6 |
| 900029-2-A | NM_001795 | CAAGGACATAACACCACGAAA | SI00028497 | Hs_CDH5_4 |
| 900029-2-A | NM_001278 | AAGCAGAAGATTATTGATCTA | SI02654659 | Hs_CHUK_8 |
| 900029-2-A | NM_005206 NM_ [symbol] | AATCCGGGACAAGCCTGAAGA | SI00299929 | Hs_CRK_5 |
| 900029-2-A | NM_001895 NM_ [symbol] | CTGGTCGCTTACATCACTTTA | SI02660504 | Hs_CSNK2A1_10 |
| 900029-2-A | NM_001913 NM_ [symbol] | AACAGAATTATTTGACCTGAA | SI02660994 | Hs_CUTL1_6 |
| 900029-2-A | NM_001380 | AACGAGGTCCAGCGATTTGAA | SI00372526 | Hs_DOCK1_4 |
| 900029-2-A | NM_001947 | CAAGGTGGTTTCAACAAGTTT | SI02658663 | Hs_DUSP7_7 |
| 900029-2-A | NM_001958 | CTGGAAGTTCGAGACCACCAA | SI02662492 | Hs_EEF1A2_7 |
| 900029-2-A | NM_004431 | TCGGACAGACATATAGGATAT | SI02223515 | Hs_EPHA2_8 |
| 900029-2-A | NM_006726 | CCGGCGACGATTTGTGCGTAA | SI03190880 | Hs_LRBA_5 |
| 900029-2-A | NM_001798 NM_ [symbol] | AAGATGGACGGAGCTTGTTAT | SI02654638 | Hs_CDK2_12 |
| 900029-2-A | NM_001295 | GACGGAAGAGTTGAGACCTAA | SI02625889 | Hs_CCR1_6 |
| 900029-2-A | NM_005207 | TAGTTTATCATTAACCACTTA | SI02634688 | Hs_CRKL_6 |
| 900029-2-A | NM_001897 | TACGCTGGGAATATTCTGTAT | SI00355446 | Hs_CSPG4_4 |
| 900029-2-A | NM_001343 | TAGAGCATGAACATCCAGTAA | SI02780386 | Hs_DAB2_6 |
| 900029-2-A | NM_004946 | CCCGGTTACAGCTGAGAATGA | SI00070350 | Hs_DOCK2_4 |
| 900029-2-A | NM_001395 | CTGAATAAACAGTTTATTTAA | SI02665572 | Hs_DUSP9_5 |
| 900029-2-A | NM_001963 | GCGGTTGTTCCTCACCCGATA | SI03105613 | Hs_EGF_5 |
| 900029-2-A | NM_001416 | ACGAGACGTGATTATGAGGGA | SI02655849 | Hs_EIF4A1_6 |
| 900029-2-A | NM_001789 NM_ [symbol] | AAGGGTTATCTCTTTCATACA | SI02653273 | Hs_CDC25A_9 |
| 900029-2-A | NM_004364 | CCGGACTTGGTGCGTCTAAGA | SI03081806 | Hs_CEBPA_6 |
| 900029-2-A | NM_000647 NM_ [symbol] | CTGGTCGTCCTCATCTTAATA | SI03099677 | Hs_CCR2_6 |
| 900029-2-A | NM_001315 NM_ [symbol] | CTCAGTGATACGTACAGCCAA | SI00605164 | Hs_MAPK14_7 |
| 900029-2-A | NM_001901 | AAAGATTCCCACCCAATTCAA | SI00029694 | Hs_CTGF_4 |
| 900029-2-A | NM_004393 | CAAGAAGATTGCCTTGGTAAA | SI02633176 | Hs_DAG1_7 |
| 900029-2-A | NM_001945 | TCCCATAATTGCTTTGCCAAA | SI03114195 | Hs_HBEGF_7 |
| 900029-2-A | NM_001951 | GACGGCGTTCTGGATCTCAAA | SI03102141 | Hs_E2F5_7 |
| 900029-2-A | NM_005228 NM_ [symbol] | CAGGAACTGGATATTCTGAAA | SI02663983 | Hs_EGFR_12 |
| 900029-2-A | NM_001967 | TGCCATATTGCACATGTCTTA | SI03236051 | Hs_EIF4A2_6 |
| 900029-2-A | NM_001039802 N [symbol] | CATCAGATTTGAAATATTTAA | SI02757328 | Hs_CDC42_7 |
| 900029-2-A | NM_005194 | CGGGCCCTGAGTAATCGCTTA | SI02777292 | Hs_CEBPB_5 |
| 900029-2-A | NM_000579 | AAGATGGATTATCAAGTGTCA | SI00012180 | Hs_CCR5_2 |
| 900029-2-A | NM_000395 | AAGGACAGCCCTGTGGCTATA | SI03034381 | Hs_CSF2RB_6 |
| 900029-2-A | NM_001903 | CACCCTGATGTCGCAGCCTAT | SI02757377 | Hs_CTNNA1_9 |
| 900029-2-A | NM_001920 NM_ [symbol] | AAGCTAGATACTGGAAACCTA | SI02647960 | Hs_DCN_9 |
| 900029-2-A | NM_004417 | CTGGTTCAACGAGGCCATTGA | SI03100048 | Hs_DUSP1_5 |
| 900029-2-A | NM_024007 | TACAAGGGACACTATAAATAA | SI00375746 | Hs_EBF_4 |
| 900029-2-A | NM_001964 | CCCGTCGGTGGCCACCACGTA | SI03078950 | Hs_EGR1_7 |
| 900029-2-A | NM_004433 | TCAGATGTACATAGAGATCTA | SI00378462 | Hs_ELF3_4 |
| 900029-2-B | NM_001771 | CACGAATATTATGCCCAGTTT | SI02627016 | Hs_CD22_6 |
| 900029-2-B | NM_004360 | CTAGGTATTGTCTACTCTGAA | SI02653546 | Hs_CDH1_12 |
| 900029-2-B | NM_020549 NM_ [symbol] | CCCGAGATGTTCATGGATGAA | SI00127015 | Hs_CHAT_3 |
| 900029-2-B | NM_001008490 N [symbol] | CACGGCCAAGTTTACCTCCGA | SI00025095 | Hs_KLF6_4 |
| 900029-2-B | NM_000760 NM_ [symbol] | TCCTATAACTTCAGTATTGTA | SI00015022 | Hs_CSF3R_4 |
| 900029-2-B | NM_001904 | TAAGAATTGAGTAATGGTGTA | SI00029750 | Hs_CTNNB1_4 |
| 900029-2-B | NM_001365 | GACGAGAGTGGTCAAGGTTAA | SI02626099 | Hs_DLG4_5 |
| 900029-2-B | NM_001394 NM_ [symbol] | TACCCAGAATTCTGTTCTAAA | SI00374934 | Hs_DUSP4_4 |
| 900029-2-B | NM_001955 | ACCGAGCACATTGGTGACAGA | SI02627373 | Hs_EDN1_5 |
| 900029-2-B | NM_000399 | TCCACTCTCTACAATCCGTAA | SI00008785 | Hs_EGR2_3 |
| 900029-2-B | NM_000610 NM_ [symbol] | CTGGCGCAGATCGATTTGAAT | SI03098123 | Hs_CD44_10 |
| 900029-2-B | NM_001793 | CAGATGAAATCGGCAACTTTA | SI02663934 | Hs_CDH3_5 |
| 900029-2-B | NM_005198 NM_ [symbol] | CTGGTAGAAGTCAGTCGGTAT | SI02634639 | Hs_CHKB_4 |
| 900029-2-B | NM_004379 NM_ [symbol] | AACTGATTCCCAAAAGCGAAG | SI00299901 | Hs_CREB1_6 |
| 900029-2-B | NM_004383 | TACGCGCCTCATTAAACCAAA | SI00288169 | Hs_CSK_3 |
| 900029-2-B | NM_001331 XM_ [symbol] | AAGGGTTAAGATCTATGGGAA | SI00025396 | Hs_CTNND1_4 |
| 900029-2-B | NM_001005336 N [symbol] | GAGGAATGTCTACAAGGATTA | SI00063392 | Hs_DNM1_3 |
| 900029-2-B | NM_001946 NM_ [symbol] | TACGGACACTATTATCACTAA | SI02627338 | Hs_DUSP6_5 |
| 900029-2-B | NM_001402 | CACCGAGACATTTAGGTGAAA | SI03058230 | Hs_EEF1A1_7 |
| 900029-2-B | NM_004430 XM_ [symbol] | CAGCGACTCGGTAGTCCATTA | SI03171623 | Hs_EGR3_5 |
| 900029-2-B | NM_000611 NM_ [symbol] | CAAAGCTGGGTTACAAGTGTA | SI03052616 | Hs_CD59_5 |
| 900029-2-B | NM_001795 | ACCACGAAACGTGAAGTTCAA | SI00028490 | Hs_CDH5_3 |
| 900029-2-B | NM_001278 | TTCCATAAGCTTGGTGACAAA | SI00605122 | Hs_CHUK_6 |
| 900029-2-B | NM_005206 NM_ [symbol] | CAGGATGTACCGAGCACTTTA | SI00073780 | Hs_CRK_1 |
| 900029-2-B | NM_001895 NM_ [symbol] | TCCATTGAAGCTGAAATGGTA | SI02660497 | Hs_CSNK2A1_9 |
| 900029-2-B | NM_001913 NM_ [symbol] | CAGCGCCTGCACGATATTGAA | SI00357070 | Hs_CUTL1_4 |
| 900029-2-B | NM_001380 | CCGAGGTTACACGTTACGAAA | SI00372519 | Hs_DOCK1_3 |
| 900029-2-B | NM_001947 | TACGACTTTGTCAAGAGGAAA | SI02658656 | Hs_DUSP7_6 |
| 900029-2-B | NM_001958 | CAAGGAGAAGACCCACATCAA | SI02661967 | Hs_EEF1A2_6 |
| 900029-2-B | NM_004431 | CAGCGCCAAGTAAACAGGGTA | SI02223508 | Hs_EPHA2_7 |
| 900029-2-B | NM_006726 | CAGGCCATTCTTAATTACCTA | SI00623224 | Hs_LRBA_4 |
| 900029-2-B | NM_001798 NM_ [symbol] | AACTTGCCTTAAACACTCACC | SI02654631 | Hs_CDK2_11 |
| 900029-2-B | NM_001295 | CTGGATCGACTACAAGTTGAA | SI02625882 | Hs_CCR1_5 |
| 900029-2-B | NM_005207 | CAGGTGTTTATAATTAATCCT | SI00073829 | Hs_CRKL_4 |
| 900029-2-B | NM_001897 | CACGGTGAGGGATGTAAATGA | SI00355439 | Hs_CSPG4_3 |
| 900029-2-B | NM_001343 | AAGGTTGGCCTTAGTAGTCAA | SI02780316 | Hs_DAB2_5 |
| 900029-2-B | NM_004946 | CAGGTCTATGCTGCGCTCATA | SI00070343 | Hs_DOCK2_3 |
| 900029-2-B | NM_001395 | CCGGGATTCCGCCAATTTGGA | SI03191349 | Hs_DUSP9_6 |
| 900029-2-B | NM_001963 | TACGACTAATCACCTACTCAA | SI00030681 | Hs_EGF_4 |
| 900029-2-B | NM_001416 | AGGGATGGACATCTTGTCATT | SI02655842 | Hs_EIF4A1_5 |
| 900029-2-B | NM_001789 NM_ [symbol] | CTGGCCAAATAGCAAAGACAA | SI02225545 | Hs_CDC25A_6 |
| 900029-2-B | NM_004364 | CCCGGCAACTCTAGTATTTAG | SI03078425 | Hs_CEBPA_5 |
| 900029-2-B | NM_000647 NM_ [symbol] | AGGGCTGTATCACATCGGTTA | SI03046036 | Hs_CCR2_5 |
| 900029-2-B | NM_001315 NM_ [symbol] | CAGAGAACTGCGGTTACTTAA | SI00605157 | Hs_MAPK14_6 |
| 900029-2-B | NM_001901 | CTGATCGTTCAAAGCATGAAA | SI00029687 | Hs_CTGF_3 |
| 900029-2-B | NM_004393 | AAGGGTGTGCATTACATTTCA | SI02633169 | Hs_DAG1_6 |
| 900029-2-B | NM_001945 | GTGCTGGATTTGATGAGTTAA | SI03106726 | Hs_HBEGF_6 |
| 900029-2-B | NM_001951 | CTGGAGGTACCCATTCCAGAA | SI03097276 | Hs_E2F5_6 |
| 900029-2-B | NM_005228 NM_ [symbol] | ATAGGTATTGGTGAATTTAAA | SI02660147 | Hs_EGFR_11 |
| 900029-2-B | NM_001967 | AAAGATATAAGTGCTGTATAA | SI02655877 | Hs_EIF4A2_5 |
| 900029-2-B | NM_044472 | TACAATTGTGGTTACCTTCAA | SI03107965 | Hs_CDC42_14 |
| 900029-2-B | NM_005194 | CACCCTGCGGAACTTGTTCAA | SI03058062 | Hs_CEBPB_7 |
| 900029-2-B | NM_000579 | AAGGGACATATTCATTTGGAA | SI00012194 | Hs_CCR5_4 |
| 900029-2-B | NM_000395 | AAGCATGTCTGTGATCCACCA | SI03033079 | Hs_CSF2RB_5 |
| 900029-2-B | NM_001903 | GCGAATTGTGGCAGAGTGTAA | SI02663962 | Hs_CTNNA1_8 |
| 900029-2-B | NM_001920 NM_ [symbol] | TACGTGCGCTCTGCCATTCAA | SI00150290 | Hs_DCN_8 |
| 900029-2-B | NM_004417 | CAGTTGTATGTTTGCTGATTA | SI00374822 | Hs_DUSP1_4 |
| 900029-2-B | NM_024007 | AGGGTGTTGGTTAAAGTTGTA | SI00375739 | Hs_EBF_3 |
| 900029-2-B | NM_001964 | CAGGACAATTGAAATTTGCTA | SI03069108 | Hs_EGR1_6 |
| 900029-2-B | NM_004433 | CAGACTCTGACAATCATTAAA | SI00378455 | Hs_ELF3_3 |
| 900029-3-A | NM_005229 | AGGGAGTCATCTCTTCCTATA | SI02662506 | Hs_ELK1_7 |
| 900029-3-A | NM_004439 NM_ [symbol] | ACCAGTTGGATCTCCAATGAA | SI02223536 | Hs_EPHA5_5 |
| 900029-3-A | NM_001982 | CCCAGTGAGAAGGCTAACAAA | SI02660252 | Hs_ERBB3_7 |
| 900029-3-A | NM_001987 | TAGCATTAAGCAGGAACGAAT | SI03111038 | Hs_ETV6_5 |
| 900029-3-A | NM_004462 | CTGGCGGTTCATGGAGAGCAA | SI03098221 | Hs_FDFT1_8 |
| 900029-3-A | NM_002011 NM_ [symbol] | CAGGCTCTTCCGGCAAGTCAA | SI02665306 | Hs_FGFR4_6 |
| 900029-3-A | NM_004119 | TACGTTGATTTCAGAGAATAT | SI02659615 | Hs_FLT3_7 |
| 900029-3-A | NM_002037 | AAGAAGCAGGATGCTGATCTA | SI02659545 | Hs_FYN_8 |
| 900029-3-A | NM_000163 | CAGGTGAGCGACATTACACCA | SI03072104 | Hs_GHR_6 |
| 900029-3-A | NM_002070 | CCGGGCGGTTGTCTACAGCAA | SI02780981 | Hs_GNAI2_6 |
| 900029-3-A | NM_005231 NM_ [symbol] | ATGCAACTTATTGTATCTGAA | SI02662485 | Hs_CTTN_6 |
| 900029-3-A | NM_004440 | CAGGCTGCGAAGGAAGTACTA | SI02223550 | Hs_EPHA7_6 |
| 900029-3-A | NM_001042599 N [symbol] | TCGGGATTTGGCAGCCCGTAA | SI02223900 | Hs_ERBB4_6 |
| 900029-3-A | NM_000503 NM_ [symbol] | CTCACCGTATCCAGCACATTA | SI03088407 | Hs_EYA1_10 |
| 900029-3-A | NM_005246 | CAGAACAACTTAGTAGGATAA | SI02622067 | Hs_FER_6 |
| 900029-3-A | NM_001042729 N [symbol] | CACCACACGGGTTCAGTTCAA | SI03057047 | Hs_FGR_6 |
| 900029-3-A | NM_002020 NM_ [symbol] | CACGCTCTTGGTCAACAGGAA | SI02225454 | Hs_FLT4_9 |
| 900029-3-A | NM_001469 | ACCGAGGGCGATGAAGAAGCA | SI03039855 | Hs_XRCC6_4 |
| 900029-3-A | NM_000165 | ATGCTTAGAGTGGACTATTAA | SI02780491 | Hs_GJA1_5 |
| 900029-3-A | NM_006496 | CAGATGATGCCCGGCAATTAT | SI03064803 | Hs_GNAI3_5 |
| 900029-3-A | NM_004437 NM_ [symbol] | GAAGGAGTAGATATCATCCTA | SI03101084 | Hs_EPB41_6 |
| 900029-3-A | NM_000121 | CAGATGATCAGGGATCCAATA | SI02780400 | Hs_EPOR_6 |
| 900029-3-A | NM_001432 | CAGGAATATTAAGTTCTATAA | SI03019387 | Hs_EREG_7 |
| 900029-3-A | NM_005244 NM_ [symbol] | TGGGTGCTTTGTGATGGATAA | SI00382214 | Hs_EYA2_4 |
| 900029-3-A | NM_002005 | CAGCCTGAGGCTGAGTACCAA | SI02659496 | Hs_FES_6 |
| 900029-3-A | NM_004469 | TTCTATGACATTGAAACACTA | SI02633358 | Hs_FIGF_6 |
| 900029-3-A | NM_005252 | TGGGTTCATTATTGGAATTAA | SI02781464 | Hs_FOS_6 |
| 900029-3-A | NM_002039 NM_ [symbol] | TAGATGCTGGATTGACATTTA | SI02654736 | Hs_GAB1_6 |
| 900029-3-A | NM_005270 NM_ [symbol] | TACCCGGGCTACAGTCCGCAA | SI03108847 | Hs_GLI2_9 |
| 900029-3-A | NM_020988 NM_ [symbol] | CCGGGAGTATCAACTCAACGA | SI03082506 | Hs_GNAO1_8 |
| 900029-3-A | NM_012156 NM_ [symbol] | CACGTTACAGTTAGCAGACGA | SI02639014 | Hs_EPB41L1_7 |
| 900029-3-A | NM_004447 | TACCTTTGTCCTGGATCGGAA | SI03109302 | Hs_EPS8_5 |
| 900029-3-A | NM_004100 NM_ [symbol] | CAGCTTTAGCAAAGAACTCTT | SI00059717 | Hs_EYA4_4 |
| 900029-3-A | NM_001990 NM_ [symbol] | CAGACTCAATACCAGACACTA | SI03167612 | Hs_EYA3_5 |
| 900029-3-A | NM_000604 NM_ [symbol] | CCGGCCTCTATGCTTGCGTAA | SI02224684 | Hs_FGFR1_7 |
| 900029-3-A | NM_002015 | CCCGAGTTTAGTAACAGTGCA | SI02781450 | Hs_FOXO1A_7 |
| 900029-3-A | NM_006732 | TTCCTGGTTTCCGAAAGGCAA | SI00091378 | Hs_FOSB_4 |
| 900029-3-A | NM_002046 | AAGGTCGGAGTCAACGGATTT | SI03571113 | Hs_GAPDH_3 |
| 900029-3-A | NM_000168 | CTGACCGATGGAGGTAGTATA | SI00003591 | Hs_GLI3_4 |
| 900029-3-A | NM_006142 | CCGGGAGAAGGTGGAGACTGA | SI02653679 | Hs_SFN_6 |
| 900029-3-A | NM_001431 | TCCCATGCATTTAATATATTA | SI00380254 | Hs_EPB41L2_4 |
| 900029-3-A | NM_001981 | TACATCGGTAGAAACGTTGAA | SI03108350 | Hs_EPS15_8 |
| 900029-3-A | NM_000125 | GAGACTTGAATTAATAAGTGA | SI02781401 | Hs_ESR1_8 |
| 900029-3-A | NM_004103 NM_ [symbol] | CAGGAGAACTTAAAGCCCAAA | SI02225328 | Hs_PTK2B_14 |
| 900029-3-A | NM_000142 NM_ [symbol] | CCGATGTTATTAGATGTTACA | SI00604779 | Hs_FGFR3_6 |
| 900029-3-A | NM_001456 | GTGGAAGAAGATCCAGCAGAA | SI02654722 | Hs_FLNA_5 |
| 900029-3-A | NM_002031 | CTGGGAGTACCTAGAACCCTA | SI00287427 | Hs_FRK_6 |
| 900029-3-A | NM_005264 NM_ [symbol] | TCGGTGCTTCCAGCCACATAA | SI03117205 | Hs_GFRA1_7 |
| 900029-3-A | NM_007353 | CGGCCGCGAGTTCGACCAGAA | SI03086195 | Hs_GNA12_5 |
| 900029-3-A | NM_002086 NM_ [symbol] | CAAGAACTACATAGAAATGAA | SI02654750 | Hs_GRB2_8 |
| 900029-3-A | NM_005233 | TTGGATAGTTTCCTACGTAAA | SI00287553 | Hs_EPHA3_6 |
| 900029-3-A | NM_001005862 N [symbol] | CACGTTTGAGTCCATGCCCAA | SI02223578 | Hs_ERBB2_15 |
| 900029-3-A | NM_004730 | TTGGAACTGCATCTAACATTA | SI03244416 | Hs_ETF1_5 |
| 900029-3-A | NM_000569 | CTGGAAGGACCATAAATTTAA | SI03209913 | Hs_FCGR3A_5 |
| 900029-3-A | NM_000141 NM_ [symbol] | CAGCATATGTGTAAAGATTTA | SI02665299 | Hs_FGFR2_7 |
| 900029-3-A | NM_002019 | CACTACAGTATTAGCAAGCAA | SI02627576 | Hs_FLT1_6 |
| 900029-3-A | NM_001010850 N [symbol] | AAGATCAATCCTCCATGAGTA | SI03032225 | Hs_FUS_5 |
| 900029-3-A | NM_001497 | GGCGGGAGACACTATATTCAA | SI03105907 | Hs_B4GALT1_6 |
| 900029-3-A | NM_002069 | TACGACCTGGTTCTAGCTGAA | SI03109414 | Hs_GNAI1_6 |
| 900029-3-A | NM_001030002 N [symbol] | CTGGATCTGTCTCCACCTCAT | SI03097598 | Hs_GRB7_7 |
| 900029-3-B | NM_005229 | CTGCTGAGAGAGCAAGGCAAT | SI00300146 | Hs_ELK1_5 |
| 900029-3-B | NM_004439 NM_ [symbol] | CCCGGCAGTATGTGTCTGTAA | SI00287511 | Hs_EPHA5_6 |
| 900029-3-B | NM_001005915 N [symbol] | CTTCGTCATGTTGAACTATAA | SI02660245 | Hs_ERBB3_6 |
| 900029-3-B | NM_001987 | GCGCCACTACTACAAACTAAA | SI00030996 | Hs_ETV6_3 |
| 900029-3-B | NM_004462 | TTGAATGTTCGTAATAGTAGA | SI02633330 | Hs_FDFT1_6 |
| 900029-3-B | NM_002011 NM_ [symbol] | CCGCCTGACCTTCGGACCCTA | SI02659979 | Hs_FGFR4_5 |
| 900029-3-B | NM_004119 | ACCAATTCAAGTGAAGATTAT | SI00059878 | Hs_FLT3_3 |
| 900029-3-B | NM_002037 NM_ [symbol] | GTGGCCCTTTATGACTATGAA | SI02654729 | Hs_FYN_7 |
| 900029-3-B | NM_000163 | AAGTGTTAATCCAGGCCTAAA | SI03037090 | Hs_GHR_5 |
| 900029-3-B | NM_002070 | CAGCCCAAGTCCAAATGTTTA | SI02780505 | Hs_GNAI2_5 |
| 900029-3-B | NM_005231 NM_ [symbol] | CACCAGGAGCATATCAACATA | SI02661960 | Hs_CTTN_5 |
| 900029-3-B | NM_004440 | ACCAGTCATGATAGTAATAGA | SI02223543 | Hs_EPHA7_5 |
| 900029-3-B | NM_001042599 N [symbol] | CTACGTGTTAGTGGCTCTTAA | SI02223893 | Hs_ERBB4_5 |
| 900029-3-B | NM_000503 NM_ [symbol] | AGCCGACGGGTCTTTAAACAA | SI03043334 | Hs_EYA1_9 |
| 900029-3-B | NM_005246 | CAGATAGATCCTAGTACAGAA | SI00287756 | Hs_FER_5 |
| 900029-3-B | NM_001042729 N [symbol] | CACGTGGAACGGCAGCACTAA | SI02634807 | Hs_FGR_5 |
| 900029-3-B | NM_002020 NM_ [symbol] | CACCGTGTGGGCTGAGTTTAA | SI02225447 | Hs_FLT4_8 |
| 900029-3-B | NM_001469 | AAGCTCTATCGGGAAACAAAT | SI03033884 | Hs_XRCC6_3 |
| 900029-3-B | NM_000165 | CAGGGAATCAAGCCATGCTTA | SI00003507 | Hs_GJA1_4 |
| 900029-3-B | NM_006496 | AAAGTGTGATTCGATCGTCAA | SI00088956 | Hs_GNAI3_4 |
| 900029-3-B | NM_004437 NM_ [symbol] | TTCGAGCGTACAGCAAGTAAA | SI03022698 | Hs_EPB41_5 |
| 900029-3-B | NM_000121 | CCAGTGCAGACTCAAGACTTA | SI02780351 | Hs_EPOR_5 |
| 900029-3-B | NM_001432 | CACCATGGTATCATATATTAA | SI02655863 | Hs_EREG_5 |
| 900029-3-B | NM_005244 NM_ [symbol] | AAGGCAGTTCAAGCTGTTGAA | SI00382207 | Hs_EYA2_3 |
| 900029-3-B | NM_002005 | AAGGCCAAGTTTCTACAGGAA | SI02659489 | Hs_FES_5 |
| 900029-3-B | NM_004469 | CAGGTTGTAAGTGCTTGCCAA | SI02633351 | Hs_FIGF_5 |
| 900029-3-B | NM_005252 | GACCAATATTATACTAAGAAA | SI02781429 | Hs_FOS_5 |
| 900029-3-B | NM_002039 NM_ [symbol] | CCCGACCAGATTCAGTGCATA | SI03077403 | Hs_GAB1_7 |
| 900029-3-B | NM_005270 | CTCGCTAGTGGCCTACATCAA | SI03091445 | Hs_GLI2_8 |
| 900029-3-B | NM_020988 NM_ [symbol] | ATGGACACTTTGGGCATCGAA | SI03050523 | Hs_GNAO1_7 |
| 900029-3-B | NM_012156 NM_ [symbol] | CAGAGTCTCCGCTATGGATAA | SI00160489 | Hs_EPB41L1_5 |
| 900029-3-B | NM_004447 | CAGGTGGATGTTAGAAGTCGA | SI00380751 | Hs_EPS8_3 |
| 900029-3-B | NM_004100 NM_ [symbol] | CTGGGTCAGTAATTACAAGTA | SI00059710 | Hs_EYA4_3 |
| 900029-3-B | NM_001990 NM_ [symbol] | CAGTCACATAAGTATAATAAA | SI00382242 | Hs_EYA3_4 |
| 900029-3-B | NM_000604 NM_ [symbol] | CAGAGATTTACCCATCGGGTA | SI02224677 | Hs_FGFR1_6 |
| 900029-3-B | NM_002015 | AACCAAGTAGCCTGTTATCAA | SI02781415 | Hs_FOXO1A_6 |
| 900029-3-B | NM_006732 | AAGGGTGCGCCGGGAACGAAA | SI00091371 | Hs_FOSB_3 |
| 900029-3-B | NM_002046 | CCGAGCCACATCGCTCAGACA | SI02653266 | Hs_GAPD_5 |
| 900029-3-B | NM_000168 | CCGCCTTATCTAGTAGCCCTA | SI00003584 | Hs_GLI3_3 |
| 900029-3-B | NM_006142 | ACCATGTTTCCTCTCAATAAA | SI02653637 | Hs_SFN_5 |
| 900029-3-B | NM_001431 | CAGGCTAAGGGTGATGCTGAA | SI00380247 | Hs_EPB41L2_3 |
| 900029-3-B | NM_001981 | TAGCCTATAAATAAATTCCAA | SI02627457 | Hs_EPS15_7 |
| 900029-3-B | NM_000125 | TCCGAGTATGATCCTACCAGA | SI03114979 | Hs_ESR1_11 |
| 900029-3-B | NM_004103 NM_ [symbol] | AAGCTGATCGGCATCATTGAA | SI02225321 | Hs_PTK2B_13 |
| 900029-3-B | NM_000142 NM_ [symbol] | ACCCTACGTTACCGTGCTCAA | SI00604772 | Hs_FGFR3_5 |
| 900029-3-B | NM_001456 | CACCCATGGAGTAGTGAACAA | SI02655912 | Hs_FLNA_7 |
| 900029-3-B | NM_002031 | CAGGACAGTCAAGGTGATATA | SI00287420 | Hs_FRK_5 |
| 900029-3-B | NM_005264 NM_ [symbol] | AAGGCAGTGCGTGGCGGGCAA | SI03035256 | Hs_GFRA1_6 |
| 900029-3-B | NM_007353 | GTCCGTTTAACTCGATAGAAA | SI00096572 | Hs_GNA12_4 |
| 900029-3-B | NM_002086 | AAGTTTGGAAACGATGTGCAG | SI00300328 | Hs_GRB2_5 |
| 900029-3-B | NM_005233 | TCGGATATGATTGTTTCTCAA | SI00287546 | Hs_EPHA3_5 |
| 900029-3-B | NM_001005862 N [symbol] | AACAAAGAAATCTTAGACGAA | SI02223571 | Hs_ERBB2_14 |
| 900029-3-B | NM_004730 | AGGGAGTATGTCTTAAATGTA | SI00381346 | Hs_ETF1_4 |
| 900029-3-B | NM_000569 | CAAGTTGCTAAGTGAACAGAA | SI00386099 | Hs_FCGR3A_3 |
| 900029-3-B | NM_000141 NM_ [symbol] | TTAGTTGAGGATACCACATTA | SI02623047 | Hs_FGFR2_6 |
| 900029-3-B | NM_002019 | ACCGCATATGGTATCCCTCAA | SI02627569 | Hs_FLT1_5 |
| 900029-3-B | NM_001010850 N [symbol] | ACAGGATAATTCAGACAACAA | SI00070518 | Hs_FUS_4 |
| 900029-3-B | NM_001497 | CAGAGGTTTGACCGAATTGCA | SI03064250 | Hs_B4GALT1_5 |
| 900029-3-B | NM_002069 | CGGGCGGATGATGCACGCCAA | SI03087140 | Hs_GNAI1_5 |
| 900029-3-B | NM_001030002 N [symbol] | CCTGCAGAAAGTGAAGCATTA | SI03083381 | Hs_GRB7_6 |
| 900029-4-A | NM_001001549 N [symbol] | CTAGATGACGGGAACACCAAA | SI02780526 | Hs_GRB10_6 |
| 900029-4-A | NM_002111 | CCCGCTGACATTTCCGTTGTA | SI02662555 | Hs_HD_7 |
| 900029-4-A | NM_002135 NM_ [symbol] | CTCCAGTGGCTCTGACTACTA | SI03089576 | Hs_NR4A1_6 |
| 900029-4-A | NM_006597 NM_ [symbol] | AGGAAATAACATTGCACTTTA | SI03145317 | Hs_HSPA8_9 |
| 900029-4-A | NM_002166 | TTGGACTGTGATATTCGTTAT | SI03244605 | Hs_ID2_6 |
| 900029-4-A | NM_000206 | AAGAACTCGGATAATGATAAA | SI00004466 | Hs_IL2RG_4 |
| 900029-4-A | NM_000208 NM_ [symbol] | TCCGGGTACCGCGAAGGGCAA | SI03115434 | Hs_INSR_6 |
| 900029-4-A | NM_002227 | CACGGATAACATCAGCTTCAT | SI00605521 | Hs_JAK1_6 |
| 900029-4-A | NM_001024844 N [symbol] | CGGCTGGGTCAGCTTCTACAA | SI03086650 | Hs_CD82_4 |
| 900029-4-A | NM_005556 | CCGCGAGGTCACCATTAACCA | SI00465073 | Hs_KRT7_4 |
| 900029-4-A | NM_004490 XM_ [symbol] | CAGACCTATTTCCCAAAGCAA | SI00430717 | Hs_GRB14_4 |
| 900029-4-A | NM_004964 | CACCCGGAGGAAAGTCTGTTA | SI02663472 | Hs_HDAC1_6 |
| 900029-4-A | NM_002136 NM_ [symbol] | AATCATGACTGACCGAGGCAG | SI00300419 | Hs_HNRPA1_1 |
| 900029-4-A | NM_004134 | AATTGTATTCTCCGAGTCAGA | SI02654813 | Hs_HSPA9B_5 |
| 900029-4-A | NM_000875 | CTGGACTCAGTACGCCGTTTA | SI03096926 | Hs_IGF1R_8 |
| 900029-4-A | NM_000418 NM_ [symbol] | CACGTGTATCCCTGAGAACAA | SI03061765 | Hs_IL4R_6 |
| 900029-4-A | NM_014215 | CAAGATCTACTTCGCCTTCAA | SI00103642 | Hs_INSRR_4 |
| 900029-4-A | NM_004972 | CTGCCTTACGATGACAGAAAT | SI02659664 | Hs_JAK2_8 |
| 900029-4-A | NM_002253 | AAGGCTAATACAACTCTTCAA | SI00605535 | Hs_KDR_6 |
| 900029-4-A | NM_002273 | CCTGAGAGCTCTCCTCACCAA | SI03083304 | Hs_KRT8_8 |
| 900029-4-A | NM_005312 NM_ [symbol] | CTGGGTCCGGTCCATAATCAT | SI03099194 | Hs_RAPGEF1_7 |
| 900029-4-A | NM_000601 NM_ [symbol] | TAGCATGTCAAGTGGAGTGAA | SI03111031 | Hs_HGF_9 |
| 900029-4-A | NM_002140 NM_ [symbol] | AATCTGATGCTGTGGAATGCT | SI00300468 | Hs_HNRPK_1 |
| 900029-4-A | NM_001017963 N [symbol] | TGCACTGTAAGACGTATGTAA | SI03117814 | Hs_HSP90AA1_2 |
| 900029-4-A | NM_001554 | CAAGAACGTCATGATGATCCA | SI03053477 | Hs_CYR61_8 |
| 900029-4-A | NM_002184 NM_ [symbol] | ATGGACCAACCCAAGTATTAA | SI03050544 | Hs_IL6ST_5 |
| 900029-4-A | NM_005544 | ACAATAGGCCATGTTAATTAA | SI00078652 | Hs_IRS1_4 |
| 900029-4-A | NM_002228 | AAGAACGTGACAGATGAGCAG | SI00300580 | Hs_JUN_5 |
| 900029-4-A | NM_004985 NM_ [symbol] | AAGGAGAATTTAATAAAGATA | SI02662051 | Hs_KRAS2_8 |
| 900029-4-A | NM_000422 | TCCTTGCCTGATGACAATAAA | SI03233307 | Hs_KRT17_5 |
| 900029-4-A | NM_002093 | CTGCATTTATCGTTAACCTAA | SI00605479 | Hs_GSK3B_8 |
| 900029-4-A | NM_004495 NM_ [symbol] | TGGCTGATTCTGGAGAGTATA | SI03120530 | Hs_NRG1_11 |
| 900029-4-A | NM_005343 NM_ [symbol] | AGGAGCGATGACGGAATATAA | SI02662576 | Hs_HRAS_8 |
| 900029-4-A | NM_002156 NM_ [symbol] | CAGGGTTTGGTGACAATAGAA | SI02654162 | Hs_HSPD1_8 |
| 900029-4-A | NM_001556 | CTGGAGAAGTACAGCGAGCAA | SI02777376 | Hs_IKBKB_9 |
| 900029-4-A | NM_004516 NM_ [symbol] | CTCGCCCTTGATGCCAACAAA | SI03091361 | Hs_ILF3_12 |
| 900029-4-A | NM_002205 | AATCCTTAATGGCTCAGACAT | SI02654841 | Hs_ITGA5_5 |
| 900029-4-A | NM_002229 | CCCGACGACCACCATCAGCTA | SI03077445 | Hs_JUNB_5 |
| 900029-4-A | NM_000424 | ACGCATGTCTCTGACACCTCA | SI03140788 | Hs_KRT5_6 |
| 900029-4-A | NM_000224 NM_ [symbol] | CCGCCGGATAGTGGATGGCAA | SI02653658 | Hs_KRT18_3 |
| 900029-4-A | NM_000177 NM_ [symbol] | CAGCTACATCATTCTGTACAA | SI02664046 | Hs_GSN_6 |
| 900029-4-A | NM_005338 | CAGGTTAGGGTGCTAGAGCTA | SI00075859 | Hs_HIP1_4 |
| 900029-4-A | NM_005524 | CCAGATCAATGCCATGACCTA | SI03075016 | Hs_HES1_5 |
| 900029-4-A | NM_002160 | CACGCCTTATAGAGTCTCCAT | SI03060232 | Hs_TNC_6 |
| 900029-4-A | NM_000586 | GTGCACCTACTTCAAGTTCTA | SI03106600 | Hs_IL2_5 |
| 900029-4-A | NM_001017915 N [symbol] | AACCTCCTTAGGGTTCGTCAA | SI03029005 | Hs_INPP5D_5 |
| 900029-4-A | NM_000212 | CACGTGTGGCCTGTTCTTCTA | SI02623159 | Hs_ITGB3_5 |
| 900029-4-A | NM_005354 | CGCGGCCGGCAGCATGATGAA | SI03085201 | Hs_JUND_6 |
| 900029-4-A | NM_058242 | TTCACTCTTTGCAATTGCTAA | SI03240776 | Hs_KRT6C_7 |
| 900029-4-A | NM_000425 NM_ [symbol] | CACCCTCAAGCTGTCGCCCTA | SI00009296 | Hs_L1CAM_4 |
| 900029-4-A | NM_002110 | CCGGGATAGCGAGACCACTAA | SI02665327 | Hs_HCK_8 |
| 900029-4-A | NM_002116 | TGCCGTGATGTGGAGGAGGAA | SI03236338 | Hs_HLA-A_5 |
| 900029-4-A | NM_005347 | TGGGATAAGGAAACACTTCTA | SI02781016 | Hs_HSPA5_7 |
| 900029-4-A | NM_002165 NM_ [symbol] | CCAGTCGCCAAGAATCATGAA | SI03075653 | Hs_ID1_7 |
| 900029-4-A | NM_000878 | CTGCCAGGTGTACTTTACTTA | SI03094714 | Hs_IL2RB_6 |
| 900029-4-A | NM_001567 | ACCCAAGAACAGCTTCAATAA | SI00447825 | Hs_INPPL1_4 |
| 900029-4-A | NM_000213 NM_ [symbol] | AACGATGACAACCGACCTATT | SI02664109 | Hs_ITGB4_6 |
| 900029-4-A | NM_002230 NM_ [symbol] | CCGCATCTCCGAGGACAAGAA | SI00034741 | Hs_JUP_4 |
| 900029-4-A | NM_005555 | CTCTCTTTAATTGCTAACCAT | SI00464933 | Hs_KRT6B_4 |
| 900029-4-A | NM_001042771 N [symbol] | AGGCATCAAGTTGACCATCAA | SI02635066 | Hs_LCK_5 |
| 900029-4-B | NM_001001549 N [symbol] | CCGCCGCAAAGCAGGATGTTA | SI03080868 | Hs_GRB10_7 |
| 900029-4-B | NM_002111 | CACACGCTAACTACAAGGTCA | SI03055696 | Hs_HD_9 |
| 900029-4-B | NM_002135 NM_ [symbol] | CACAGGAGAGTTTGACACCTT | SI03056221 | Hs_NR4A1_5 |
| 900029-4-B | NM_006597 NM_ [symbol] | AACACTGTATTGTAAGTGGAA | SI03123218 | Hs_HSPA8_8 |
| 900029-4-B | NM_002166 | AAAGTTTAGTTGTAAACTTAA | SI03122518 | Hs_ID2_5 |
| 900029-4-B | NM_000206 | CACGACAATTCTGACGCCCAA | SI00004459 | Hs_IL2RG_3 |
| 900029-4-B | NM_000208 NM_ [symbol] | TTGGACGGTGGTAGACATTGA | SI03024581 | Hs_INSR_5 |
| 900029-4-B | NM_002227 | ACCGGATGAGGTTCTATTTCA | SI00605514 | Hs_JAK1_5 |
| 900029-4-B | NM_001024844 N [symbol] | CGCAGGTGGGCTGGACTTCTA | SI03084123 | Hs_CD82_3 |
| 900029-4-B | NM_005556 | TGCCCTGAATGATGAGATCAA | SI00465066 | Hs_KRT7_3 |
| 900029-4-B | NM_004490 XM_ [symbol] | AAGAATGTATTTCACCTGCAA | SI00430710 | Hs_GRB14_3 |
| 900029-4-B | NM_004964 | CCCGTTCTTAACTTTGAACCA | SI02634149 | Hs_HDAC1_5 |
| 900029-4-B | NM_002136 NM_ [symbol] | CTCTTAAGCCATCTTGGTAAA | SI03205657 | Hs_HNRPA1_10 |
| 900029-4-B | NM_004134 | AAGAGACTTCCTGAGCAGAAA | SI03128496 | Hs_HSPA9B_6 |
| 900029-4-B | NM_000875 | TCGAAGAATCGCATCATCATA | SI02624552 | Hs_IGF1R_7 |
| 900029-4-B | NM_000418 NM_ [symbol] | AAGCCCAGCGAGCATGTGAAA | SI03033177 | Hs_IL4R_5 |
| 900029-4-B | NM_014215 | CAGCTCGGATTTCGAGATCCA | SI00103635 | Hs_INSRR_3 |
| 900029-4-B | NM_004972 | AGCCATCATACGAGATCTTAA | SI02659657 | Hs_JAK2_7 |
| 900029-4-B | NM_002253 | AACGCTGACATGTACGGTCTA | SI00605528 | Hs_KDR_5 |
| 900029-4-B | NM_002273 | CACCGCAGTTACGGTCAACCA | SI03058356 | Hs_KRT8_7 |
| 900029-4-B | NM_005312 NM_ [symbol] | CAGGAAAGATTTGGTGTTGTA | SI02634989 | Hs_RAPGEF1_5 |
| 900029-4-B | NM_000601 NM_ [symbol] | CTGGAGTTCCATGATACCACA | SI03097395 | Hs_HGF_8 |
| 900029-4-B | NM_002140 NM_ [symbol] | TTCCATTGTATGCAAATTGAA | SI02650844 | Hs_HNRPK_5 |
| 900029-4-B | NM_001017963 N [symbol] | AACCCTGACCATTCCATTATT | SI03028606 | Hs_HSP90AA1_1 |
| 900029-4-B | NM_001554 | AACCCTTTACAAGGCCAGAAA | SI03028655 | Hs_CYR61_7 |
| 900029-4-B | NM_002184 NM_ [symbol] | CACATTGTACATGGTACGAAT | SI00033740 | Hs_IL6ST_4 |
| 900029-4-B | NM_005544 | CCGGTATCGTTTCGCATGGAA | SI00078645 | Hs_IRS1_3 |
| 900029-4-B | NM_002228 | CCCGAGCTGGAGCGCCTGATA | SI03077599 | Hs_JUN_7 |
| 900029-4-B | NM_004985 NM_ [symbol] | GTGGACGAATATGATCCAACA | SI03106824 | Hs_KRAS_2 |
| 900029-4-B | NM_000422 | CACCGTGGACAATGCCAACAT | SI00464513 | Hs_KRT17_4 |
| 900029-4-B | NM_002093 | AACACTGGTCACGTTTGGAAA | SI00605472 | Hs_GSK3B_7 |
| 900029-4-B | NM_004495 NM_ [symbol] | TCGGCTGCAGGTTCCAAACTA | SI03116974 | Hs_NRG1_10 |
| 900029-4-B | NM_005343 NM_ [symbol] | CACAGATGGGATCACAGTAAA | SI02662030 | Hs_HRAS_7 |
| 900029-4-B | NM_002156 NM_ [symbol] | CACCACCAGATGAGAAGTTAA | SI02653007 | Hs_HSPD1_7 |
| 900029-4-B | NM_001556 | AAACCGAGTTTGGGATCACAT | SI00300545 | Hs_IKKB2_1 |
| 900029-4-B | NM_004516 NM_ [symbol] | CACAACCGCCCTCCTGGACAA | SI03055297 | Hs_ILF3_11 |
| 900029-4-B | NM_002205 | CAGGGTCTACGTCTACCTGCA | SI03071572 | Hs_ITGA5_7 |
| 900029-4-B | NM_002229 | AAACACGCACTTAGTCTCTAA | SI00034713 | Hs_JUNB_4 |
| 900029-4-B | NM_000424 | AAGCCGAGTCCTGGTATCAGA | SI03130414 | Hs_KRT5_5 |
| 900029-4-B | NM_000224 NM_{—} [symbol] | TCGCTCCACCTTCTCCACCAA | SI03234091 | Hs_KRT18_10 |
| 900029-4-B | NM_000177 NM_ [symbol] | AACGATGCCTTTGTTCTGAAA | SI02664039 | Hs_GSN_5 |
| 900029-4-B | NM_005338 | CACAACAATGGGTATCCTTAA | SI00075852 | Hs_HIP1_3 |
| 900029-4-B | NM_005524 | AAAGACGAAGAGCAAGAATAA | SI00078344 | Hs_HES1_4 |
| 900029-4-B | NM_002160 | CACGATGGCTTTGCAGGCGAT | SI03059987 | Hs_TNC_5 |
| 900029-4-B | NM_000586 | CTGGAGGAAGTGCTAAATTTA | SI00012271 | Hs_IL2_3 |
| 900029-4-B | NM_001017915 N [symbol] | CCCGGGACTGTTGACAGCCAA | SI00078589 | Hs_INPP5D_3 |
| 900029-4-B | NM_000212 | CAAGCTGAACCTAATAGCCAT | SI00004606 | Hs_ITGB3_4 |
| 900029-4-B | NM_005354 | CCCGCCGTTGTCGCCCATCGA | SI03077956 | Hs_JUND_5 |
| 900029-4-B | NM_005554 NM_ [symbol] | CTGGAGCTTCACTGTTACTAA | SI03210725 | Hs_KRT6C_6 |
| 900029-4-B | NM_000425 NM_ [symbol] | CCGCGGATACAATGTGACGTA | SI00009289 | Hs_L1CAM_3 |
| 900029-4-B | NM_002110 | CGGCAGGGAGATACCGTGAAA | SI02665320 | Hs_HCK_7 |
| 900029-4-B | NM_002116 | TAGAACCTTAGTATAAATTTA | SI00438641 | Hs_HLA-A_4 |
| 900029-4-B | NM_005347 | TAGGGTGTGTGTTCACCTTCA | SI02780554 | Hs_HSPA5_6 |
| 900029-4-B | NM_002165 NM_ [symbol] | CAGTTGGAGCTGAACTCGGAA | SI03073525 | Hs_ID1_6 |
| 900029-4-B | NM_000878 | ACAGACGGCGGTGGAACCAAA | SI03037664 | Hs_IL2RB_5 |
| 900029-4-B | NM_001567 | CAAGTTCTTCATCGAGTTCTA | SI00447818 | Hs_INPPL1_3 |
| 900029-4-B | NM_000213 NM_ [symbol] | GTGGATGAGTTCCGGAATAAA | SI02664102 | Hs_ITGB4_5 |
| 900029-4-B | NM_002230 NM_ [symbol] | AACCATCGGCTTGATCAGGAA | SI00034734 | Hs_JUP_3 |
| 900029-4-B | NM_005555 | TCAGGAGTTCTCATCTGACAA | SI00464926 | Hs_KRT6B_3 |
| 900029-4-B | NM_001042771 N [symbol] | CTACGGGACATTCACCATCAA | SI00076013 | Hs_LCK_4 |
| 900029-5-A | NM_005565 | CCGGGTGCCGATTCTCAGTAA | SI02656185 | Hs_LCP2_8 |
| 900029-5-A | NM_001003688 N [symbol] | CTGGTGCTCTATTGTCTACTA | SI03099824 | Hs_SMAD1_9 |
| 900029-5-A | NM_002393 | GACCACGAGACGGGAACATTA | SI03101476 | Hs_MDM4_5 |
| 900029-5-A | NM_002419 | CCCGACGTCTGGAGGACTCAA | SI02659552 | Hs_MAP3K11_6 |
| 900029-5-A | NM_006153 | ACCGTTATGCAGAATAATCCA | SI02654918 | Hs_NCK1_5 |
| 900029-5-A | NM_002524 | CTGAGATACGTCTGTGACTTA | SI02662632 | Hs_NRAS_6 |
| 900029-5-A | NM_004561 XM_ [symbol] | CAAGGCTGCCTTCAATTAGAA | SI00065261 | Hs_OVOL1_4 |
| 900029-5-A | NM_002578 | TTCCAGTACTTTGTACAGGAA | SI00287434 | Hs_PAK3_5 |
| 900029-5-A | NM_006218 | CTCCGTGAGGCTACATTAATA | SI02665369 | Hs_PIK3CA_8 |
| 900029-5-A | NM_006221 | CAGTATTTATTGTTCCCACAA | SI02662667 | Hs_PIN1_6 |
| 900029-5-A | NM_002306 NM_ [symbol] | CAATACAAAGCTGGATAATAA | SI02731183 | Hs_LGALS3_6 |
| 900029-5-A | NM_001003652 N [symbol] | CAGGTAATGTATCATGATCCA | SI02757496 | Hs_SMAD2_6 |
| 900029-5-A | XM_001128827 X [symbol] | CTCCGGGTGTTTCAACTAGAA | SI02659965 | Hs_MAP3K1_11 |
| 900029-5-A | NM_000902 NM_ [symbol] | AGGTGTGGTGTGGAACCTATA | SI03046302 | Hs_MME_5 |
| 900029-5-A | NM_006403 NM_ [symbol] | CCTGACCGTCATAGAGCAGAA | SI03192406 | Hs_NEDD9_6 |
| 900029-5-A | NM_001007204 N [symbol] | CTGGGAGTGGTTAGCCGGAAT | SI03098697 | Hs_NTRK1_6 |
| 900029-5-A | NM_002567 | CAGGTCTACAGTGATAGAGCA | SI03246376 | Hs_PEBP1_2 |
| 900029-5-A | NM_005017 | AAGCGCCACCTCAGAAGATAA | SI00681016 | Hs_PCYT1A_4 |
| 900029-5-A | NM_006219 | TCGGGAAGCTACCATTTCTTA | SI02622221 | Hs_PIK3CB_6 |
| 900029-5-A | NM_005028 | CTGCCCGATGGTCTTCCGTAA | SI02223830 | Hs_PIP5K2A_6 |
| 900029-5-A | NM_005572 NM_ [symbol] | CCAGGAGCTTCTGGACATCAA | SI02662597 | Hs_LMNA_14 |
| 900029-5-A | NM_005902 | ATGGTGCGAGAAGGCGGTCAA | SI03052126 | Hs_SMAD3_5 |
| 900029-5-A | NM_002401 NM_ [symbol] | CCACGTGTCTGTGCACCACAA | SI00605619 | Hs_MAP3K3_6 |
| 900029-5-A | NM_002447 | CAGGTCTGCGTAGATGGTGAA | SI02758539 | Hs_MST1R_7 |
| 900029-5-A | NM_003998 | GACGCCATCTATGACAGTAAA | SI02662618 | Hs_NFKB1_10 |
| 900029-5-A | NM_001007097 N [symbol] | GGGCTCCTTAAGGATAACTAA | SI03106173 | Hs_NTRK2_7 |
| 900029-5-A | NM_030979 | CACGGTTCCACGGTATAAATA | SI03164812 | Hs_PABPC3_5 |
| 900029-5-A | NM_002609 | CTGCCGAGCAACTTTGATCAA | SI00605745 | Hs_PDGFRB_6 |
| 900029-5-A | NM_005026 | CGCCGTGATCGAGAAAGCCAA | SI02223816 | Hs_PIK3CD_6 |
| 900029-5-A | NM_006224 | CTGCACAATATGAGCATGCAA | SI03019436 | Hs_PITPNA_5 |
| 900029-5-A | NM_002332 | TACGACCGCATCGAGACGATA | SI03109400 | Hs_LRP1_5 |
| 900029-5-A | NM_005905 | CTGGTGCTCGGTCGCCTACTA | SI03213882 | Hs_SMAD9_5 |
| 900029-5-A | NM_005922 NM_ [symbol] | CTCAAGCATCGCATAGTTTAA | SI02224012 | Hs_MAP3K4_6 |
| 900029-5-A | NM_001018016 N [symbol] | CAGCAGCCTCTCTTACACAAA | SI02780673 | Hs_MUC1_7 |
| 900029-5-A | NM_002507 | CCGAGCACATAGACTCCTTTA | SI03080119 | Hs_NGFR_7 |
| 900029-5-A | NM_001007155 N [symbol] | CTGGTTGGAGCGAATCTGCTA | SI00605689 | Hs_NTRK3_10 |
| 900029-5-A | NM_000430 | ATGCGCATGAACACTTTGTTA | SI03152576 | Hs_PAFAH1B1_5 |
| 900029-5-A | NM_002613 NM_ [symbol] | CACGCCTAACAGGACGTATTA | SI00605787 | Hs_PDPK1_9 |
| 900029-5-A | NM_002649 | CACCTTTACTCTATAACTCAA | SI00605843 | Hs_PIK3CG_6 |
| 900029-5-A | NM_000296 NM_ [symbol] | TCCGTCTTTGGCAAGACATTA | SI03115560 | Hs_PKD1_6 |
| 900029-5-A | NM_002344 NM_ [symbol] | CCTGCAGATGCTTCTAATAAA | SI00605563 | Hs_LTK_6 |
| 900029-5-A | NM_002378 NM_ [symbol] | GACGGATTCTAAGGACTCTAA | SI00605605 | Hs_MATK_10 |
| 900029-5-A | NM_005923 | AACGAAGCGAGCCAAGGGCAA | SI02654890 | Hs_MAP3K5_7 |
| 900029-5-A | NM_002467 | CTCGGTGCAGCCGTATTTCTA | SI02662611 | Hs_MYC_7 |
| 900029-5-A | NM_024408 XM_ [symbol] | CAGCGGTGTACCATTGACATT | SI03067526 | Hs_NOTCH2_5 |
| 900029-5-A | NM_001014796 N [symbol] | CACCCACAACCTATGATCCAA | SI03649499 | Hs_DDR2_6 |
| 900029-5-A | NM_002576 | TTGAAGAGAACTGCAACTGAA | SI00605703 | Hs_PAK1_9 |
| 900029-5-A | NM_002614 | AAGGCCTATGATTATTTCCAA | SI00681800 | Hs_PDZK1_4 |
| 900029-5-A | NM_181504 NM_ [symbol] | TAGCTGGTTATGAACTAGTAA | SI02225412 | Hs_PIK3R1_6 |
| 900029-5-A | NM_002660 NM_ [symbol] | CAAGTTCTTCTTGACAGACAA | SI00041195 | Hs_PLCG1_4 |
| 900029-5-A | NM_002350 | CGGGAAATATGGGATGTATAA | SI00605577 | Hs_LYN_13 |
| 900029-5-A | NM_005369 | GAGGTGCAATGAGCTACGTTA | SI03104227 | Hs_MCF2_6 |
| 900029-5-A | NM_000245 | CAACACCCATCCAGAATGTCA | SI02654897 | Hs_MET_10 |
| 900029-5-A | NM_002473 | CAGGAGCAGCTCCAGGCAGAA | SI02654911 | Hs_MYH9_6 |
| 900029-5-A | NM_000272 NM_ [symbol] | CCAGTTAAAGCAGGCAATAGA | SI03075772 | Hs_NPHP1_7 |
| 900029-5-A | NM_006186 NM_ [symbol] | TACGACACTGTCCACCTTTAA | SI00085330 | Hs_NR4A2_4 |
| 900029-5-A | NM_002577 XM_ [symbol] | CCGGATCATACGAAATCAATT | SI00605717 | Hs_PAK2_9 |
| 900029-5-A | NM_002646 | CACTGTAGACTTGCTTATCTA | SI02660070 | Hs_PIK3C2B_6 |
| 900029-5-A | NM_005027 | TTGGTACGTGGGCAAGATCAA | SI00287539 | Hs_PIK3R2_6 |
| 900029-5-A | NM_002661 | GACGACGGTTGTGAATGATAA | SI02662681 | Hs_PLCG2_5 |
| 900029-5-B | NM_005565 | AAGCTGCTCTTAGAAAGATAA | SI02656178 | Hs_LCP2_7 |
| 900029-5-B | NM_001003688 N [symbol] | CTCCCAATAGCAGTTACCCAA | SI03089716 | Hs_SMAD1_8 |
| 900029-5-B | NM_002393 | GACCTAAAGATGCGTATATAA | SI00037163 | Hs_MDM4_4 |
| 900029-5-B | NM_002419 | CCGGAGGAGAAACGTCTTCGA | SI00605626 | Hs_MAP3K11_5 |
| 900029-5-B | NM_006153 | AGCAGTCGTCAATAACCTAAA | SI03042767 | Hs_NCK1_6 |
| 900029-5-B | NM_002524 | AACCTGTTTGTTGGACATACT | SI00300993 | Hs_NRAS_5 |
| 900029-5-B | NM_004561 | CCGCGCGTTCCTGGTGAAGAA | SI00065254 | Hs_OVOL1_3 |
| 900029-5-B | NM_002578 | CAAGAAGGAATTAATTATTAA | SI02628983 | Hs_PAK3_6 |
| 900029-5-B | NM_006218 | CTGAGTCAGTATAAGTATATA | SI02622207 | Hs_PIK3CA_5 |
| 900029-5-B | NM_006221 | GACCGCCAGATTCTCCCTTAA | SI02662128 | Hs_PIN1_5 |
| 900029-5-B | NM_002306 NM_ [symbol] | ATGCGTTATCTGGGTCTGGAA | SI02707292 | Hs_LGALS3_5 |
| 900029-5-B | NM_001003652 N [symbol] | AAGCCGTCTATCAGCTAACTA | SI03033275 | Hs_SMAD2_8 |
| 900029-5-B | XM_001128827 X [symbol] | CACGCATGTCAAATTCTCATA | SI02659958 | Hs_MAP3K1_10 |
| 900029-5-B | NM_000902 NM_ [symbol] | CACCGTTACAAGTATACTTAT | SI00018130 | Hs_MME_4 |
| 900029-5-B | NM_006403 NM_ [symbol] | CAGAAGCTCTATCAAGTGCCA | SI03166898 | Hs_NEDD9_5 |
| 900029-5-B | NM_001007204 N [symbol] | CACATCATCGAGAACCCACAA | SI02628836 | Hs_NTRK1_5 |
| 900029-5-B | NM_002567 | CCGCTATGTCTGGCTGGTTTA | SI03189739 | Hs_PEBP1_1 |
| 900029-5-B | NM_005017 | CAGCTTTATCAACGAGAAGAA | SI00681009 | Hs_PCYT1A_3 |
| 900029-5-B | NM_006219 | CCCTTCGATAAGATTATTGAA | SI02622214 | Hs_PIK3CB_5 |
| 900029-5-B | NM_005028 | ATGGAATTAAGTGCCATGAAA | SI02223823 | Hs_PIP5K2A_5 |
| 900029-5-B | NM_005572 NM_ [symbol] | AACTGGACTTCCAGAAGAACA | SI02654862 | Hs_LMNA_13 |
| 900029-5-B | NM_005902 | AAGGAGCACCTTGACAGACTT | SI00082502 | Hs_SMAD3_4 |
| 900029-5-B | NM_002401 NM_ [symbol] | CAGGAATACTCAGATCGGGAA | SI00605612 | Hs_MAP3K3_5 |
| 900029-5-B | NM_002447 | TCCCGGTGACACAGACACAAA | SI02758532 | Hs_MST1R_6 |
| 900029-5-B | NM_003998 | CTGGGTATACTTCATGTGACA | SI02662093 | Hs_NFKB1_9 |
| 900029-5-B | NM_001007097 N [symbol] | CAGCGCTTCAGTGGTTCTATA | SI03067246 | Hs_NTRK2_6 |
| 900029-5-B | NM_030979 | CTCCAACTACGCGTATGTGAA | SI00676697 | Hs_PABPC3_3 |
| 900029-5-B | NM_002609 | CCGAGCAACTTTGATCAACGA | SI00605738 | Hs_PDGFRB_5 |
| 900029-5-B | NM_005026 | CCGGTCACGCATGAAGGCAAA | SI02223809 | Hs_PIK3CD_5 |
| 900029-5-B | NM_006224 | AAGGATGGAAGAAGAGACGAA | SI00685188 | Hs_PITPNA_4 |
| 900029-5-B | NM_002332 | CTGGCTATTGACTTCCCTGAA | SI00036204 | Hs_LRP1_3 |
| 900029-5-B | NM_005905 | CAGATGAAACAAAGCAGTGAA | SI00726572 | Hs_SMAD9_4 |
| 900029-5-B | NM_005922 NM_ [symbol] | CACCAATCCCTGAAAGATTAA | SI02224005 | Hs_MAP3K4_5 |
| 900029-5-B | NM_001018016 N [symbol] | CTGGCCATTGTCTATCTCATT | SI03097927 | Hs_MUC1_8 |
| 900029-5-B | NM_002507 | CACAGCGGTGAGTGCTGCAAA | SI03056151 | Hs_NGFR_6 |
| 900029-5-B | NM_001007155 N [symbol] | CACGGATAACTTTATCTTGTT | SI00605682 | Hs_NTRK3_9 |
| 900029-5-B | NM_000430 | CAGAATCTTCTGAACCATCTA | SI00677012 | Hs_PAFAH1B1_4 |
| 900029-5-B | NM_002613 NM_ [symbol] | AAGCGGTTAGGCTGTGAGGAA | SI00605780 | Hs_PDPK1_8 |
| 900029-5-B | NM_002649 | ATCGAAGTTTGCAGAGACAAA | SI00605836 | Hs_PIK3CG_5 |
| 900029-5-B | NM_000296 NM_ [symbol] | TCCGGAGGTCACCCACGCTTA | SI03115287 | Hs_PKD1_5 |
| 900029-5-B | NM_002344 NM_ [symbol] | ACAGATCTTTGGAGTGCCTAA | SI00605556 | Hs_LTK_5 |
| 900029-5-B | NM_002378 NM_ [symbol] | ACGGATTCTAAGGACTCTAAA | SI00605598 | Hs_MATK_9 |
| 900029-5-B | NM_005923 | CCGGGAATCTATACTCAATGA | SI02224026 | Hs_MAP3K5_6 |
| 900029-5-B | NM_002467 | GATCCCGGAGTTGGAAAACAA | SI00300902 | Hs_MYC_5 |
| 900029-5-B | NM_024408 XM_ [symbol] | TTAGATGATAATGGACAACTA | SI00136206 | Hs_NOTCH2_3 |
| 900029-5-B | NM_001014796 N [symbol] | AAAGAAGCGTTTCACAACAAA | SI03649492 | Hs_DDR2_5 |
| 900029-5-B | NM_002576 | TCCACTGATTGCTGCAGCTAA | SI00605696 | Hs_PAK1_8 |
| 900029-5-B | NM_002614 XM_ [symbol] | ATGACTGATATTACACCTCAA | SI00681793 | Hs_PDZK1_3 |
| 900029-5-B | NM_181504 NM_ [symbol] | CAGCATTAAACCAGACCTTAT | SI02225405 | Hs_PIK3R1_5 |
| 900029-5-B | NM_002660 NM_ [symbol] | CACGCTCTCTTTCTGGCGGAA | SI00041188 | Hs_PLCG1_3 |
| 900029-5-B | NM_002350 | CCCGGACGACTTGTCTTTCAA | SI00605570 | Hs_LYN_12 |
| 900029-5-B | NM_005369 | CAGCGTTTGGATAGGGCACAA | SI03067862 | Hs_MCF2_5 |
| 900029-5-B | NM_000245 | CGCGCCGTGATGAATATCGAA | SI00604821 | Hs_MET_9 |
| 900029-5-B | NM_002473 | AACCGGGACGAAGCCATCAAA | SI00038360 | Hs_MYH9_3 |
| 900029-5-B | NM_000272 NM_ [symbol] | CAGGGCCTTGCTGTGACAATA | SI03071397 | Hs_NPHP1_6 |
| 900029-5-B | NM_006186 NM_ [symbol] | CTGGATTTAGAACATGGACTA | SI00085323 | Hs_NR4A2_3 |
| 900029-5-B | NM_002577 XM_ [symbol] | CCGCGACCGGATCATACGAAA | SI00605710 | Hs_PAK2_8 |
| 900029-5-B | NM_002646 | GAGGGAGGAGCTAAACGGTTA | SI02660063 | Hs_PIK3C2B_5 |
| 900029-5-B | NM_005027 | CCGCGAGTATGACCAGCTTTA | SI00287532 | Hs_PIK3R2_5 |
| 900029-5-B | NM_002661 | GGGCGGGACCCTGAAATACTA | SI03106138 | Hs_PLCG2_7 |
| 900029-6-A | NM_002662 | TCCGAATTGATAATCTTTCAA | SI03232152 | Hs_PLD1_5 |
| 900029-6-A | NM_006236 | CACGCCAGAGCTGGCCGAGCA | SI00690284 | Hs_POU3F3_4 |
| 900029-6-A | NM_002730 NM_ [symbol] | CAGAAGGTGGTGAAACTGAAA | SI00605864 | Hs_PRKACA_6 |
| 900029-6-A | NM_006254 NM_ [symbol] | CAGCAGCAAGTGCAACATCAA | SI02660539 | Hs_PRKCD_11 |
| 900029-6-A | NM_006257 | CACAAGAAGTGTATTGATAAA | SI03571148 | Hs_PRKCQ_12 |
| 900029-6-A | NM_002752 NM_ [symbol] | ATCGTGAACTTGTCCTCTTAA | SI02222920 | Hs_MAPK9_7 |
| 900029-6-A | NM_005044 | TTGGAATACTCTAAGAGAATA | SI02223858 | Hs_PRKX_6 |
| 900029-6-A | NM_002827 | ACGGACGTTGGTTCTGCACTA | SI00043827 | Hs_PTPN1_4 |
| 900029-6-A | NM_002842 | CACGACCATCTGGGACGGAAT | SI03059588 | Hs_PTPRH_6 |
| 900029-6-A | NM_005052 | GACATGCTTGCTGATCAGCTA | SI02634310 | Hs_RAC3_5 |
| 900029-6-A | NM_002663 | CAGCAAGGTGCTCATCGCAGA | SI03065335 | Hs_PLD2_6 |
| 900029-6-A | NM_000307 | CCCGACCAGCATTGACAAGAT | SI03077410 | Hs_POU3F4_7 |
| 900029-6-A | NM_002731 NM_ [symbol] | CTGACCAATCAAGTACACTAA | SI02225468 | Hs_PRKACB_10 |
| 900029-6-A | NM_005400 | CACGGAAACACCCGTACCTTA | SI02622088 | Hs_PRKCE_6 |
| 900029-6-A | NM_001033581 N [symbol] | GACCAAATTTACGCCATGAAA | SI00605976 | Hs_PRKCZ_6 |
| 900029-6-A | NM_002753 NM_ [symbol] | TCCGAGCACAATAAACTCAAA | SI02222934 | Hs_MAPK10_6 |
| 900029-6-A | NM_016335 | CCGCACCTACTTCTACGCCAA | SI03080518 | Hs_PRODH_5 |
| 900029-6-A | NM_002828 NM_ [symbol] | CACAAAGGAGTTACATCTTAA | SI02759239 | Hs_PTPN2_10 |
| 900029-6-A | NM_002843 | TCCGAGTATGTCTACCATTTA | SI02658817 | Hs_PTPRJ_6 |
| 900029-6-A | NM_002880 | TGGGAAATAGAAGCCAGTGAA | SI02223039 | Hs_RAF1_7 |
| 900029-6-A | NM_000445 NM_ [symbol] | CCAGACTAATATATTAATATA | SI02662142 | Hs_PLEC1_9 |
| 900029-6-A | NM_021130 NM_ [symbol] | TTCAAGATGACTAATGTCAAA | SI00690921 | Hs_PPIA_3 |
| 900029-6-A | NM_002732 | CTGGATCGCCATCTATGAGAA | SI00605878 | Hs_PRKACG_6 |
| 900029-6-A | NM_002739 | CTACGCCATCAAGATCTTGAA | SI03087763 | Hs_PRKCG_6 |
| 900029-6-A | NM_001081640 N [symbol] | GACCCTGTTGACAGTACTTTA | SI02663633 | Hs_PRKDC_8 |
| 900029-6-A | NM_002755 | CTGGATCAAGTCCTGAAGAAA | SI02222962 | Hs_MAP2K1_8 |
| 900029-6-A | NM_000314 | AAGGCGTATACAGGAACAATA | SI00301504 | Hs_PTEN_6 |
| 900029-6-A | NM_002831 NM_ [symbol] | TAGGCCCTGATGAGAACGCTA | SI02658733 | Hs_PTPN6_6 |
| 900029-6-A | NM_002859 | TCCGACTTTGATAGATTTCTA | SI02757601 | Hs_PXN_7 |
| 900029-6-A | NM_005402 | CGCGGTGCAGATTCTTCTTAA | SI02662835 | Hs_RALA_7 |
| 900029-6-A | NM_021105 | CCAGTGTATAATCAGCCAGTA | SI03075751 | Hs_PLSCR1_6 |
| 900029-6-A | NM_002709 NM_ [symbol] | CACTATTGGATGTGATTCTAA | SI02759204 | Hs_PPP1CB_6 |
| 900029-6-A | NM_002734 NM_ [symbol] | CTGGACCGACCTAGATTTGAA | SI00605906 | Hs_PRKAR1A_8 |
| 900029-6-A | NM_002740 | GTGCCGGTACATTTACTTAAA | SI02660105 | Hs_PRKCI_11 |
| 900029-6-A | NM_002745 | ATCATGGTAGTCACTAACATA | SI00605990 | Hs_MAPK1_13 |
| 900029-6-A | NM_030662 | CCGGCCTGCCATGGCCATCTT | SI02225097 | Hs_MAP2K2_6 |
| 900029-6-A | NM_000958 | AGGCTCTATTCCAATAAACTA | SI02624699 | Hs_PTGER4_6 |
| 900029-6-A | NM_002834 | CCGCTCATGACTATACGCTAA | SI02225909 | Hs_PTPN11_7 |
| 900029-6-A | NM_004162 | AACCCAAACTGTATGTCTTGA | SI02655037 | Hs_RAB5A_8 |
| 900029-6-A | NM_002881 | TGACGAGTTTGTAGAAGACTA | SI03117492 | Hs_RALB_7 |
| 900029-6-A | NM_002699 | CCGCCCGCGCCCTTAATTTAA | SI00690228 | Hs_POU3F1_4 |
| 900029-6-A | NM_002714 | CTCAAACGTCAGAGCAACGTA | SI03088141 | Hs_PPP1R10_6 |
| 900029-6-A | NM_002737 | TACAAGTTGCTTAACCAAGAA | SI00605934 | Hs_PRKCA_7 |
| 900029-6-A | NM_006256 | CACGTCAAAGTATGATATCTA | SI02224096 | Hs_PKN2_6 |
| 900029-6-A | NM_001040056 N [symbol] | CTCCCTGACCCGTCTAATATA | SI00606004 | Hs_MAPK3_7 |
| 900029-6-A | NM_002756 NM_ [symbol] | CCGGGCCACCGTGAACTCACA | SI02222976 | Hs_MAP2K3_6 |
| 900029-6-A | NM_005607 NM_ [symbol] | CCGGTCGAATGATAAGGTGTA | SI02622130 | Hs_PTK2_10 |
| 900029-6-A | NM_002835 | AAGCTTAATGAGGAAATATCA | SI02658768 | Hs_PTPN12_8 |
| 900029-6-A | NM_006908 NM_ [symbol] | ATGCATTTCCTGGAGAATATA | SI02655051 | Hs_RAC1_6 |
| 900029-6-A | NM_001010935 N [symbol] | CAGGGCCAGAATTTAGCAAGA | SI02662296 | Hs_RAP1A_6 |
| 900029-6-A | NM_005604 | TACCCGCTTTATCGAAGGCAA | SI03224683 | Hs_POU3F2_6 |
| 900029-6-A | NM_006243 | CAGCGTATTCTGATATAGTAA | SI02225846 | Hs_PPP2R5A_6 |
| 900029-6-A | NM_002738 NM_ [symbol] | CCGGATGAAACTGACCGATTT | SI00605948 | Hs_PRKCB1_6 |
| 900029-6-A | NM_002742 | AAGCGGCACATTCCCATTTAA | SI00301350 | Hs_PRKCM_2 |
| 900029-6-A | NM_002750 NM_ [symbol] | ATGATGTGTCTTCAATGTCAA | SI02758651 | Hs_MAPK8_15 |
| 900029-6-A | NM_145185 | CAGGAAGAGACCAAAGTATAA | SI02660588 | Hs_MAP2K7_9 |
| 900029-6-A | NM_005975 | CTCCGCGACTCTGATGAGAAA | SI03090024 | Hs_PTK6_5 |
| 900029-6-A | NM_002840 NM_ [symbol] | CATCGTGTTTGCAAAGGTTAA | SI02658796 | Hs_PTPRF_6 |
| 900029-6-A | NM_002872 | AACTATTCAGCCAATGTGATG | SI02655058 | Hs_RAC2_5 |
| 900029-6-A | NM_001010942 N [symbol] | CAGTATAATGTCTTAGATTAA | SI02662849 | Hs_RAP1B_7 |
| 900029-6-B | NM_002662 | ATGGGATATTTGATTACGTAA | SI00686357 | Hs_PLD1_3 |
| 900029-6-B | NM_006236 | CGGCTCTATTGTGCACTCGGA | SI00690277 | Hs_POU3F3_3 |
| 900029-6-B | NM_002730 NM_ [symbol] | CAAGGACAACTCAAACTTATA | SI00605857 | Hs_PRKACA_5 |
| 900029-6-B | NM_006254 NM_ [symbol] | AACTCTACCGTGCCACGTTTT | SI00301329 | Hs_PRKCD_7 |
| 900029-6-B | NM_006257 | AACCATAGTTATTTACTTGAA | SI02758623 | Hs_PRKCQ_8 |
| 900029-6-B | NM_002752 NM_ [symbol] | AAAGTCGATTTATGTGTATTA | SI02222913 | Hs_MAPK9_6 |
| 900029-6-B | NM_005044 | CGGATGGGATTCACTTAAGAA | SI02223851 | Hs_PRKX_5 |
| 900029-6-B | NM_002827 | CAGGAATAGGCATTTGCCTAA | SI00043820 | Hs_PTPN1_3 |
| 900029-6-B | NM_002842 | ATCACCGTGGATAGACTTGAA | SI03046904 | Hs_PTPRH_5 |
| 900029-6-B | NM_005052 | CCGGGAGATTGGCTCTGTGAA | SI00071890 | Hs_RAC3_4 |
| 900029-6-B | NM_002663 | TGGGCGGACGGTTCTGAACAA | SI03020857 | Hs_PLD2_5 |
| 900029-6-B | NM_000307 | CAGAAACTTCTCCAAAGTGAT | SI03062675 | Hs_POU3F4_6 |
| 900029-6-B | NM_002731 NM_ [symbol] | CAGCCTGTGTAGTGTGACAAA | SI02225461 | Hs_PRKACB_9 |
| 900029-6-B | NM_005400 | CCCGACCATGGTAGTGTTCAA | SI00287784 | Hs_PRKCE_5 |
| 900029-6-B | NM_001033581 N [symbol] | CGGAAGCATGACAGCATTAAA | SI00605969 | Hs_PRKCZ_5 |
| 900029-6-B | NM_002753 NM_ [symbol] | CCGCATGTGTCTGTATTCATA | SI02222927 | Hs_MAPK10_5 |
| 900029-6-B | NM_016335 | CTGGCATTTGTCAGGAATATA | SI00115451 | Hs_PRODH_3 |
| 900029-6-B | NM_002828 NM_ [symbol] | CCGCTGTACTTGGAAATTCGA | SI02225895 | Hs_PTPN2_9 |
| 900029-6-B | NM_002843 | TCGGGTAGAAATAACCACCAA | SI02658810 | Hs_PTPRJ_5 |
| 900029-6-B | NM_002880 | CAGATCTTAGTAAGCTATATA | SI02223032 | Hs_RAF1_6 |
| 900029-6-B | NM_000445 NM_ [symbol] | CCGCCAGGTGAAGCTGGTGAA | SI02654988 | Hs_PLEC1_8 |
| 900029-6-B | NM_021130 NM_ [symbol] | CAGTAATGGGTTACTTCTGAA | SI00690914 | Hs_PPIA_2 |
| 900029-6-B | NM_002732 | AACCAGCTGGATCGCCATCTA | SI00605871 | Hs_PRKACG_5 |
| 900029-6-B | NM_002739 | CCCGGAGATCATTGCCTACCA | SI03078306 | Hs_PRKCG_5 |
| 900029-6-B | NM_001081640 N [symbol] | TTCGGCTAACTCGCCAGTTTA | SI02224236 | Hs_PRKDC_6 |
| 900029-6-B | NM_002755 | CTGGAAGAATTCCTGAACAAA | SI02222955 | Hs_MAP2K1_7 |
| 900029-6-B | NM_000314 | TCGACTTAGACTTGACCTATA | SI03116092 | Hs_PTEN_9 |
| 900029-6-B | NM_002831 NM_ [symbol] | CCGGAACAAATGCGTCCCATA | SI02658726 | Hs_PTPN6_5 |
| 900029-6-B | NM_002859 | CCGACTGAAACTGGAACCCTT | SI02757594 | Hs_PXN_6 |
| 900029-6-B | NM_005402 | TAGGAACTCACTCTTTAGATA | SI00076608 | Hs_RALA_3 |
| 900029-6-B | NM_021105 | CAGCGCCACAGCCTCCATTAA | SI03067043 | Hs_PLSCR1_5 |
| 900029-6-B | NM_002709 NM_ [symbol] | TACGAGGATGTCGTCCAGGAA | SI02225762 | Hs_PPP1CB_5 |
| 900029-6-B | NM_002734 NM_ [symbol] | CAGCTAGTGCCAAATAATTGA | SI00605899 | Hs_PRKAR1A_7 |
| 900029-6-B | NM_002740 | CAGATTGTTCTTTGTTATAGA | SI02660098 | Hs_PRKCI_10 |
| 900029-6-B | NM_002745 | AACACTTGTCAAGAAGCGTTA | SI00605983 | Hs_MAPK1_12 |
| 900029-6-B | NM_030662 | CAGCATTTGCATGGAACACAT | SI02225090 | Hs_MAP2K2_5 |
| 900029-6-B | NM_000958 | ACCCATAATTGAAGTGTATAA | SI02624692 | Hs_PTGER4_5 |
| 900029-6-B | NM_002834 | CAGAAGCACAGTACCGATTTA | SI02225902 | Hs_PTPN11_6 |
| 900029-6-B | NM_004162 | ATTCATGGAGACATCCGCTAA | SI00301588 | Hs_RAB5A_5 |
| 900029-6-B | NM_002881 | CAAGGTGTTCTTTGACCTAAT | SI03054793 | Hs_RALB_6 |
| 900029-6-B | NM_002699 | CCCGCCCATGGACGATGTATA | SI00690221 | Hs_POU3F1_3 |
| 900029-6-B | NM_002714 | AAGCAATAGTCAGGAGCGATA | SI03032666 | Hs_PPP1R10_5 |
| 900029-6-B | NM_002737 | CGCAGTGGAATGAGTCCTTTA | SI00605927 | Hs_PRKCA_6 |
| 900029-6-B | NM_006256 | AAAGTATGATATCTACGCAAA | SI02224089 | Hs_PKN2_5 |
| 900029-6-B | NM_001040056 N [symbol] | CCCGTCTAATATATAAATATA | SI00605997 | Hs_MAPK3_6 |
| 900029-6-B | NM_002756 NM_ [symbol] | ACGGATATCCTGCATGTCCAA | SI02222969 | Hs_MAP2K3_5 |
| 900029-6-B | NM_005607 NM_ [symbol] | AATCACACACCAAATTCGAGT | SI00301532 | Hs_PTK2_9 |
| 900029-6-B | NM_002835 | TTGCAGGTTATCAGAGATCAA | SI02658761 | Hs_PTPN12_7 |
| 900029-6-B | NM_006908 NM_ [symbol] | CAGCACGTGTTCCCGACATAA | SI03065531 | Hs_RAC1_10 |
| 900029-6-B | NM_001010935 N [symbol] | CCCAACGATAGAAGATTCCTA | SI03075961 | Hs_RAP1A_8 |
| 900029-6-B | NM_005604 | CCGCAGCGTCTAACCACTACA | SI03188626 | Hs_POU3F2_5 |
| 900029-6-B | NM_006243 | CTGTATCATGGCCATAGTATA | SI02225839 | Hs_PPP2R5A_5 |
| 900029-6-B | NM_002738 | CAAGAGCTAAGTAGATGTGTA | SI00605941 | Hs_PRKCB1_5 |
| 900029-6-B | NM_002742 | TCGATTATTTCCAGTGTTCTA | SI00042378 | Hs_PRKD1_3 |
| 900029-6-B | NM_002750 NM_ [symbol] | ATGAAATGTGTTAATCACAAA | SI02758644 | Hs_MAPK8_14 |
| 900029-6-B | NM_145185 | AAAGATGACAGTGGCGATTGT | SI00300720 | Hs_MAP2K7_1 |
| 900029-6-B | NM_005975 | ACGCGTGTGCTCCTCTCCTTA | SI00083314 | Hs_PTK6_3 |
| 900029-6-B | NM_002840 NM_ [symbol] | CAGCGCTATCTAGATAGGTAA | SI02658789 | Hs_PTPRF_5 |
| 900029-6-B | NM_002872 | CAATGTGATGGTGGACAGCAA | SI00044947 | Hs_RAC2_4 |
| 900029-6-B | NM_001010942 N [symbol] | GACGAGTACTGTGGATGTGAA | SI02662303 | Hs_RAP1B_6 |
| 900029-7-A | NM_021033 | CCAGTGTATTCCGTCAAGTAA | SI02663087 | Hs_RAP2A_6 |
| 900029-7-A | NM_000321 | CAGGGTTGTGTCGAAATTGGA | SI02653931 | Hs_RB1_8 |
| 900029-7-A | NM_000539 | CAGCTTAGGGATAAGTGTCTA | SI03068555 | Hs_RHO_5 |
| 900029-7-A | NM_004586 | TCCAAACATTATCACTCTAAA | SI00288197 | Hs_RPS6KA3_6 |
| 900029-7-A | NM_003010 | TTGGACGAGGAGCTTATGGTT | SI02655079 | Hs_MAP2K4_10 |
| 900029-7-A | NM_000345 NM_ [symbol] | ATGGATGTATTCATGAAAGGA | SI03051209 | Hs_SNCA_6 |
| 900029-7-A | NM_006939 | AAGCATTTGATCCTTATGAAA | SI00729344 | Hs_SOS2_4 |
| 900029-7-A | NM_003131 | CAAGATGGAGTTCATCGACAA | SI02757622 | Hs_SRF_5 |
| 900029-7-A | NM_003152 | AGGCACGTGGAGGAACTCTTA | SI03045014 | Hs_STAT5A_5 |
| 900029-7-A | NM_005647 | CCCGCATGTCACTTAGTCTAA | SI00740432 | Hs_TBL1X_4 |
| 900029-7-A | NM_000964 NM_ [symbol] | CAGGAAATGTTGGAGAACTCA | SI03068821 | Hs_RARA_6 |
| 900029-7-A | NM_005610 | CAGCTATCCCTCTATATAATA | SI02653420 | Hs_RBBP4_8 |
| 900029-7-A | NM_012421 | CACCTTAGGATTCATTATAAA | SI00703752 | Hs_RLF_4 |
| 900029-7-A | NM_006270 | CTCGGCCAAACTGCGTCTCAA | SI02663115 | Hs_RRAS_6 |
| 900029-7-A | NM_003027 | CAAGGAGAATTAGGATTTAAA | SI00717388 | Hs_SH3GL3_4 |
| 900029-7-A | NM_003088 | CTGAGCCTTATTTCTCTGGAA | SI00421813 | Hs_FSCN1_4 |
| 900029-7-A | NM_138473 | CAGGTCAGTTGGCAGACTCTA | SI03071887 | Hs_SP1_8 |
| 900029-7-A | NM_007107 | CTCCGCGCTCTTCTTCGGAAA | SI03090031 | Hs_SSR3_10 |
| 900029-7-A | NM_012448 | ATGGGACTCAGTAGATCTTGA | SI03051678 | Hs_STAT5B_5 |
| 900029-7-A | NM_003199 | GACGACAAGAAGGATATCAAA | SI03101805 | Hs_TCF4_5 |
| 900029-7-A | NM_000965 NM_ [symbol] | GAGCGTGTAATTACCTTGAAA | SI00019411 | Hs_RARB_4 |
| 900029-7-A | NM_002893 XM_ [symbol] | CCGACGCAAGATGGCGAGTAA | SI02664466 | Hs_RBBP7_6 |
| 900029-7-A | NM_002944 | ACCGAGAAGGGTTAAACTATA | SI02223053 | Hs_ROS1_6 |
| 900029-7-A | NM_001003698 N [symbol] | CGGAGGTGCATCAGCGAGCAA | SI03195605 | Hs_RREB1_6 |
| 900029-7-A | NM_003029 NM_ [symbol] | CTGAAATTTGCTGGAATGCCA | SI02655100 | Hs_SHC1_11 |
| 900029-7-A | NM_004597 NM_ [symbol] | TGCCATTGGTGTTGAGAATAA | SI03236156 | Hs_SNRPD2_5 |
| 900029-7-A | NM_003119 NM_ [symbol] | AAGGTTGAAGCAGAAGAATAA | SI03019695 | Hs_SPG7_5 |
| 900029-7-A | NM_007315 NM_ [symbol] | CCAGATGTCTATGATCATTTA | SI02662884 | Hs_STAT1_7 |
| 900029-7-A | NM_003153 | ACGGATAGGCAGGAACATACA | SI02662905 | Hs_STAT6_5 |
| 900029-7-A | NM_003200 | GAGCGGAACCTGAATCCCAAA | SI03103086 | Hs_TCF3_5 |
| 900029-7-A | NM_001042728 X | TCCTGTTTCGCCGGACTTGAA | SI04025616 | Hs_RARG_9 |
| 900029-7-A | NM_000323 NM_ [symbol] | TAGGCTGGTTCTCAACCGGAA | SI02224992 | Hs_RET_10 |
| 900029-7-A | NM_006013 | CAGCAAAGCCTTGCAATCCCA | SI02636473 | Hs_RPL10_5 |
| 900029-7-A | NM_000540 NM_ [symbol] | CCGCTATGGCCTCCTCATAAA | SI00011494 | Hs_RYR1_4 |
| 900029-7-A | NM_005414 | TTGGTTCAGGGCTCAACTAAA | SI00076776 | Hs_SKIL_4 |
| 900029-7-A | NM_003099 NM_ [symbol] | GAGGGCCGCTTTAGAAAGGTA | SI03104003 | Hs_SNX1_7 |
| 900029-7-A | NM_003127 | AACGCTTCCTTGCTGACTTCC | SI00301861 | Hs_SPTAN1_5 |
| 900029-7-A | NM_005419 | AACGTTCAGGTGGTTCAGGAA | SI02662891 | Hs_STAT2_7 |
| 900029-7-A | NM_006281 | CGGCGCCTAAGAGTAAACTAA | SI02622263 | Hs_STK3_6 |
| 900029-7-A | NM_003205 NM_ [symbol] | ATGGTTGGAACTCATCGGGAA | SI03052231 | Hs_TCF12_6 |
| 900029-7-A | NM_002890 NM_ [symbol] | CAGACCTAATAGGTTATTACA | SI00045367 | Hs_RASA1_4 |
| 900029-7-A | NM_005613 | CTGGTCCCTCAGTGTGCCTAA | SI03099600 | Hs_RGS4_11 |
| 900029-7-A | NM_001006665 N [symbol] | TGCCACGTACTCCGCACTCAA | SI02223067 | Hs_RPS6KA1_10 |
| 900029-7-A | NM_001017369 N [symbol] | TTGAAGATACTTGGCACTATT | SI03243149 | Hs_SC4MOL_8 |
| 900029-7-A | NM_003064 | AAGGCTCTGGAAAGTCCTTCA | SI00726404 | Hs_SLPI_4 |
| 900029-7-A | NM_003100 | AAGTGCTGCCATGTTAGGTAA | SI03135454 | Hs_SNX2_5 |
| 900029-7-A | NM_003129 | TGGGAGACGCATATAATATGA | SI03120894 | Hs_SQLE_6 |
| 900029-7-A | NM_003150 NM_ [symbol] | CAGGCTGGTAATTTATATAAT | SI02662898 | Hs_STAT3_8 |
| 900029-7-A | NM_006282 | CACCATTTGCTGTGCGAATTA | SI02622277 | Hs_STK4_6 |
| 900029-7-A | NM_006286 | CATGATGACATAGAAGTACTA | SI03073868 | Hs_TFDP2_6 |
| 900029-7-A | NM_006506 | CCGCAGTTTAATGAAATCTTT | SI00698740 | Hs_RASA2_4 |
| 900029-7-A | NM_002928 | CAGGACCATGGCACCCTTAGA | SI03069178 | Hs_RGS16_7 |
| 900029-7-A | NM_001006932 N [symbol] | CCGGAGGTCCTGAAGCGTCAA | SI02225006 | Hs_RPS6KA2_10 |
| 900029-7-A | NM_000609 NM_ [symbol] | GTGCATTGACCCGAAGCTAAA | SI03649149 | Hs_CXCL12_11 |
| 900029-7-A | NM_001007468 N [symbol] | CTCCACAACCATCAACAGGAA | SI00726824 | Hs_SMARCB1_4 |
| 900029-7-A | NM_005633 | AAGGAGGTCCTAGGTTATAAA | SI02655121 | Hs_SOS1_5 |
| 900029-7-A | NM_005417 NM_ [symbol] | CTCCATGTGCGTCCATATTTA | SI02664151 | Hs_SRC_10 |
| 900029-7-A | NM_003151 | TCAGAGGCCGTTGGTACTTAA | SI00048412 | Hs_STAT4_4 |
| 900029-7-A | NM_003600 NM_ [symbol] | CACCTTCGGCATCCTAATATT | SI02223305 | Hs_STK6_5 |
| 900029-7-A | NM_003236 | CTGGCTGTCCTTATCATCACA | SI03098403 | Hs_TGFA_5 |
| 900029-7-B | NM_021033 | ACCGGCACCTTCATCGAGAAA | SI03040198 | Hs_RAP2A_7 |
| 900029-7-B | NM_000321 | CGCGTGTAAATTCTACTGCAA | SI02653819 | Hs_RB1_7 |
| 900029-7-B | NM_000539 | CCACATTTAATTAACAGCTGA | SI00011466 | Hs_RHO_4 |
| 900029-7-B | NM_004586 | AGCGCTGAGAATGGACAGCAA | SI00288190 | Hs_RPS6KA3_5 |
| 900029-7-B | NM_003010 | AGGGTGTATAGTGTTCACAAA | SI02223102 | Hs_MAP2K4_8 |
| 900029-7-B | NM_000345 NM_ [symbol] | AAAGAGGGTGTTCTCTATGTA | SI03026478 | Hs_SNCA_5 |
| 900029-7-B | NM_006939 | CAGCATAATATTTGCTGCTAA | SI00729337 | Hs_SOS2_3 |
| 900029-7-B | NM_003131 | AAGGAGCGGCCTCGCCATAAA | SI03034731 | Hs_SRF_7 |
| 900029-7-B | NM_003152 | TTCGAAGTTAGGAGGACTCAA | SI00048440 | Hs_STAT5A_4 |
| 900029-7-B | NM_005647 | TAGAGTCATCCCTGTAATCAA | SI00740425 | Hs_TBL1X_3 |
| 900029-7-B | NM_000964 NM_ [symbol] | CACTGAGATCTACTGGATAAA | SI03062318 | Hs_RARA_5 |
| 900029-7-B | NM_005610 | AAGACTCCTTCCAGTGATGTT | SI00301658 | Hs_RBBP4_5 |
| 900029-7-B | NM_012421 | AAGGATGAATGTAGTTCTGAA | SI00703745 | Hs_RLF_3 |
| 900029-7-B | NM_006270 | CCGGGTCACTGCTGTATATAA | SI02663108 | Hs_RRAS_5 |
| 900029-7-B | NM_003027 | CGCCTGGATTACGATTATAAA | SI00717381 | Hs_SH3GL3_3 |
| 900029-7-B | NM_003088 | CAGCTGCTACTTTGACATCGA | SI00421806 | Hs_FSCN1_3 |
| 900029-7-B | NM_138473 | ATGCCTAATATTCAGTATCAA | SI03050054 | Hs_SP1_7 |
| 900029-7-B | NM_007107 | CAGCCGCAATCTCTCGGCCAA | SI03066371 | Hs_SSR3_9 |
| 900029-7-B | NM 012448 | GCCACCCTAATTTGACATCAA | SI00100415 | Hs_STAT5B_4 |
| 900029-7-B | NM_003199 | AGCCGAATTGAAGATCGTTTA | SI00048965 | Hs_TCF4_4 |
| 900029-7-B | NM_000965 NM_ [symbol] | CTGCCAGTTCAGTTAATCAAA | SI00019404 | Hs_RARB_3 |
| 900029-7-B | NM_002893 XM_ [symbol] | GCGGATAAGACCGTAGCTTTA | SI02664459 | Hs_RBBP7_5 |
| 900029-7-B | NM_002944 | AAGGTAATTGCTCTAACTTTA | SI02223046 | Hs_ROS1_5 |
| 900029-7-B | NM_001003698 N [symbol] | AACGCGCTTGTCCACAAACAA | SI03125843 | Hs_RREB1_5 |
| 900029-7-B | NM_003029 NM_ [symbol] | AAGAGCCACCTGACCATCAGT | SI00301791 | Hs_SHC1_9 |
| 900029-7-B | NM_004597 NM_ [symbol] | CCGCAACAATAAGAAACTCCT | SI00728385 | Hs_SNRPD2_3 |
| 900029-7-B | NM_003119 NM_ [symbol] | GAGCGACGTGGTGGAAGTCTA | SI03217753 | Hs_SPG7_7 |
| 900029-7-B | NM_007315 NM_ [symbol] | CAGAAAGAGCTTGACAGTAAA | SI02662324 | Hs_STAT1_6 |
| 900029-7-B | NM_003153 | CAGCGGCTCTATGTCGACTTT | SI03067414 | Hs_STAT6_6 |
| 900029-7-B | NM_003200 | CTGGATGATTGGGACTTTAAA | SI00048993 | Hs_TCF3_4 |
| 900029-7-B | NM_001042728 X | TACGACTGTATGGAAACGTTT | SI04025609 | Hs_RARG_8 |
| 900029-7-B | NM_000323 NM_ [symbol] | CCGCTGGTGGACTGTAATAAT | SI02224985 | Hs_RET_9 |
| 900029-7-B | NM_006013 | CAGAAACAGGTTGACAACTCA | SI00083685 | Hs_RPL10_2 |
| 900029-7-B | NM_000540 NM_ [symbol] | CTGGGAATGGACGATAGAGAA | SI00011487 | Hs_RYR1_3 |
| 900029-7-B | NM_005414 | AGGCAAGTAAGTCCATATCAA | SI00076769 | Hs_SKIL_3 |
| 900029-7-B | NM_003099 NM_ [symbol] | CTGGGTCTTTATGAGAAGCTT | SI02630250 | Hs_SNX1_6 |
| 900029-7-B | NM_003127 | CCGGCTGCGTACGTGAAGAAA | SI00048069 | Hs_SPTAN1_4 |
| 900029-7-B | NM_005419 | TAGGCCGATTAACTACCCTAA | SI02662331 | Hs_STAT2_6 |
| 900029-7-B | NM_006281 | CCGGCGCCTAAGAGTAAACTA | SI02622256 | Hs_STK3_5 |
| 900029-7-B | NM_003205 NM_ [symbol] | AACCGTGAATCTCCTAGTTAT | SI03028893 | Hs_TCF12_5 |
| 900029-7-B | NM_002890 NM_ [symbol] | ACGGACCTGTCCCGTGATTTA | SI00045360 | Hs_RASA1_3 |
| 900029-7-B | NM_005613 | CTGGATTCTTGCACCAGGGAA | SI03097766 | Hs_RGS4_10 |
| 900029-7-B | NM_001006665 N [symbol] | CCCAACATCATCACTCTGAAA | SI02223060 | Hs_RPS6KA1_9 |
| 900029-7-B | NM_001017369 N [symbol] | CTCTCAGTATAATGCCTATAA | SI03205398 | Hs_SC4MOL_7 |
| 900029-7-B | NM_003064 | CAGTGCCTTAGATACAAGAAA | SI00726397 | Hs_SLPI_3 |
| 900029-7-B | NM_003100 | CTGGATCAGCAACTTAGGAAA | SI00728840 | Hs_SNX2_4 |
| 900029-7-B | NM_003129 | CTCGAGTACTTGTTGACATTA | SI03091123 | Hs_SQLE_5 |
| 900029-7-B | NM_003150 NM_ [symbol] | CAGCCTCTCTGCAGAATTCAA | SI02662338 | Hs_STAT3_7 |
| 900029-7-B | NM_006282 | AGGATTCATAGCATCACTATA | SI02622270 | Hs_STK4_5 |
| 900029-7-B | NM_006286 | CAGAGGCGGATAGAACGGATA | SI00086954 | Hs_TFDP2_4 |
| 900029-7-B | NM_006506 | CTACCTGAAAGTAACATTAAA | SI00698733 | Hs_RASA2_3 |
| 900029-7-B | NM_002928 | CAGACTGTTCTGGGCACGGAA | SI03063760 | Hs_RGS16_6 |
| 900029-7-B | NM_001006932 N [symbol] | CCGAGTGAGATCGAAGATGGA | SI02224999 | Hs_RPS6KA2_9 |
| 900029-7-B | NM_000609 NM_ [symbol] | CTGAAGAACAACAACAGACAA | SI03649100 | Hs_CXCL12_10 |
| 900029-7-B | NM_001007468 N [symbol] | ACCAGAGAAGTTTGCCCTGAA | SI00726817 | Hs_SMARCB1_3 |
| 900029-7-B | NM_005633 | TGGGTTGAATCCATCACTAAA | SI00079807 | Hs_SOS1_3 |
| 900029-7-B | NM_005417 NM_ [symbol] | CGGCTTGTGGGTGATGTTTGA | SI02223928 | Hs_SRC_7 |
| 900029-7-B | NM_003151 | TGGAATCAAGTCCAACAGTTA | SI00048405 | lis_STAT4_3 |
| 900029-7-B | NM_003600 NM_ [symbol] | TCCCAGCGCATTCCTTTGCAA | SI03114111 | Hs_AURKA_1 |
| 900029-7-B | NM_003236 | AAGCCGGTAAATGCCTCAATA | SI00049448 | Hs_TGFA_4 |
| 900029-8-A | NM_004612 | CTGCCTTATTATGATCTTGTA | SI02664158 | Hs_TGFBR1_9 |
| 900029-8-A | NM_001032283 N [symbol] | CCCAGACAAGAAGATAAAGAT | SI03183061 | Hs_TMPO_6 |
| 900029-8-A | NM_006001 NM_ [symbol] | TGGATTTAAGGTGGGCATTAA | SI03119858 | Hs_TUBA2_8 |
| 900029-8-A | NM_021141 | AAGCGAGTAACCAGCTCATAA | SI02663773 | Hs_XRCC5_7 |
| 900029-8-A | NM_021915 | TTGGCCATGTTTATGATTTGA | SI03245186 | Hs_ZNF69_8 |
| 900029-8-A | NM_003559 NM_ [symbol] | CACGATCAATGAGCTGAGCAA | SI02660133 | Hs_PIP5K2B_9 |
| 900029-8-A | NM_003676 NM_ [symbol] | TACGTATCTGGAAGTTATCAA | SI03109946 | Hs_DEGS1_8 |
| 900029-8-A | NM_003804 | CCGACATTTCCTGGCATTGAA | SI02621983 | Hs_RIPK1_6 |
| 900029-8-A | NM_003921 | CTTGTCGAACATCAAGTAGAA | SI03100958 | Hs_BCL10_8 |
| 900029-8-A | NM_005147 | ATGATTCTGTATTAATGTAAA | SI00370678 | Hs_DNAJA3_4 |
| 900029-8-A | NM_001032282 N [symbol] | CCGGCGGTTCATGAGGAGTGA | SI03082268 | Hs_KLF10_9 |
| 900029-8-A | NM_000546 | AAGGAAATTTGCGTGTGGAGT | SI02655170 | Hs_TP53_9 |
| 900029-8-A | NM_003347 NM_ [symbol] | CCGCACTAGTAGAGAATCCAT | SI03188563 | Hs_UBE2L3_5 |
| 900029-8-A | NM_005433 | GAGGCTCCTGCTTATTTATAA | SI02223942 | Hs_YES1_7 |
| 900029-8-A | NM_001010972 N [symbol] | AAGGTGAGCAGTATTGATTTG | SI00302225 | Hs_ZYX_1 |
| 900029-8-A | NM_003567 | CCGGAACTCTGGCGTCAACTA | SI03081603 | Hs_BCAR3_6 |
| 900029-8-A | NM_003745 | TACCCAGTATCTTTGCACAAA | SI03108812 | Hs_SOCS1_6 |
| 900029-8-A | NM_003819 | AACTTTGATGTGATTAAGGGA | SI00301035 | Hs_PABPC4_1 |
| 900029-8-A | NM_003939 NM_ [symbol] | CAGGATGAGCAACAACAGTAA | SI02632322 | Hs_BTRC_10 |
| 900029-8-A | NM_004702 NM_ [symbol] | CTCCAAGTTGATGCTCTTAAA | SI02653238 | Hs_CCNE2_8 |
| 900029-8-A | NM_003257 NM_ [symbol] | CCAGTATCTGATAATGAAGAA | SI02655149 | Hs_TJP1_7 |
| 900029-8-A | NM_003290 | CTGGCCCAACTTCATTTCCAT | SI03211551 | Hs_TPM4_5 |
| 900029-8-A | NM_006003 | TAGATAGTACGAAGTCTTCAA | SI03227154 | Hs_UQCRFS1_6 |
| 900029-8-A | NM_003404 NM_ [symbol] | AAAGAGTACCGTGAGAAGATA | SI03026499 | Hs_YWHAB_6 |
| 900029-8-A | NM_006009 | AAGTTGTGGTCTGATCAGTTA | SI03037251 | Hs_TUBA3_5 |
| 900029-8-A | NM_001004720 N [symbol] | GACGGGCTATGTACCGTCCAA | SI03102190 | Hs_NCK2_11 |
| 900029-8-A | NM_001037494 N [symbol] | CAGAATAGCCTACATTTGTAT | SI03167052 | Hs_DYNLL1_3 |
| 900029-8-A | NM_003852 NM_ [symbol] | CAGAACGGTCCAGTCACCAAA | SI03062899 | Hs_TRIM24_1 |
| 900029-8-A | NM_003941 | AACCTTAATGTAATTTACTTA | SI02757692 | Hs_WASL_7 |
| 900029-8-A | NM_004747 | CTGTGGCATATTTGTCACTAA | SI00067879 | Hs_DLG5_4 |
| 900029-8-A | NM_005078 | CACCATGAACTCGATCACAGA | SI00745696 | Hs_TLE3_4 |
| 900029-8-A | NM_003292 | CAGCGTGATATGTACCGTATT | SI03172260 | Hs_TPR_5 |
| 900029-8-A | NM_005428 | CAAGGAGAGGTTCCTCGTCTA | SI03054401 | Hs_VAV1_5 |
| 900029-8-A | NM_006761 | ACCATTAGCAAATGGAAATTA | SI03038868 | Hs_YWHAE_8 |
| 900029-8-A | NM_001013836 N [symbol] | CACGCTCAGGTTGAAGGTCGA | SI03060365 | Hs_MAD1L1_7 |
| 900029-8-A | NM_003619 | CTCAGTGTAGTGGAAGTGATA | SI00053830 | Hs_PRSS12_4 |
| 900029-8-A | NM_003749 | CAGTGTATTGACGCATATTTA | SI02662583 | Hs_IRS2_6 |
| 900029-8-A | NM_004664 | CCCATTTATATCTCTCGCATA | SI00066822 | Hs_LIN7A_3 |
| 900029-8-A | NM_003954 | TACCTAGTGCATGCTCTGCAA | SI02632343 | Hs_MAP3K14_6 |
| 900029-8-A | NM_182398 | CTGGATCTCTTACGTAATTCA | SI02225440 | Hs_RPS6KA5_9 |
| 900029-8-A | NM_006289 | AACAGAGACCCCTGAAGATCC | SI00301931 | Hs_TLN1_5 |
| 900029-8-A | NM_003299 | TCGCCTCAGTTTGAACATTGA | SI02663738 | Hs_TRA1_9 |
| 900029-8-A | NM_003371 | CTGAAAGTCTGCCACGATAAA | SI02662947 | Hs_VAV2_6 |
| 900029-8-A | NM_003406 NM_ [symbol] | CAATCCAAGCATAATTGTTAA | SI03158295 | Hs_YWHAZ_5 |
| 900029-8-A | NM_003557 | CCCTGCCTTGATAATATGTTA | SI02223263 | Hs_PIP5K1A_6 |
| 900029-8-A | NM_003629 | ATGAATTATGATAAATTGAAA | SI02777446 | Hs_PIK3R3_9 |
| 900029-8-A | NM_001037283 N [symbol] | CCGCACTTCCATATTCTGGAA | SI00377846 | Hs_EIF3S9_4 |
| 900029-8-A | NM_003870 | AAGGAGACGTCAGAACGTGGC | SI02655268 | Hs_IQGAP1_5 |
| 900029-8-A | NM_003955 | TCGGGAGTTCCTGGACCAGTA | SI00058345 | Hs_SOCS3_1 |
| 900029-8-A | NM_005451 NM_ [symbol] | CTGAGCATCGATGGCGAGAAT | SI03093405 | Hs_PDLIM7_10 |
| 900029-8-A | NM_001001723 N [symbol] | TCCGAAGATGATTCACCTAGA | SI03114860 | Hs_TMEM1_8 |
| 900029-8-A | NM_003302 | CAGGAGGAGACTGTGAGAATT | SI03069654 | Hs_TRIP6_5 |
| 900029-8-A | NM_005429 | TTGCTGCAGCACATTATAATA | SI02664221 | Hs_VEGFC_6 |
| 900029-8-A | NM_001079 NM_ [symbol] | CACGGAAGAGATGATGCGCGA | SI03060540 | Hs_ZAP70_6 |
| 900029-8-A | NM_001031687 N [symbol] | AAGGGTTACCTTCCAGTTCAA | SI03571155 | Hs_PIP5K1B_7 |
| 900029-8-A | NM_003639 | TTCGGAAATGCCTCACATATA | SI02223340 | Hs_IKBKG_6 |
| 900029-8-A | NM_003794 | TGGCGGCGATATAGTGAATTT | SI03120376 | Hs_SNX4_10 |
| 900029-8-A | NM_003904 | TAGGAGGAAACCCAGAAATGA | SI03228267 | Hs_ZNF259_8 |
| 900029-8-A | NM_003974 NM_ [symbol] | GAGTATGACAATGTTGTACTA | SI03104346 | Hs_DOK2_6 |
| 900029-8-A | NM_004759 NM_ [symbol] | CTACGAGCAGATCAAGATAAA | SI02223697 | Hs_MAPKAPK2_5 |
| 900029-8-B | NM_004612 | TGGGATTGTACTATACCAGTA | SI02223634 | Hs_TGFBR1_7 |
| 900029-8-B | NM_001032283 N [symbol] | TAGATGTAACAGAGCTCACTA | SI03227217 | Hs_TMPO_9 |
| 900029-8-B | NM_006001 NM_ [symbol] | CAGCTGATCACCGGGAAGGAA | SI03068296 | Hs_TUBA2_7 |
| 900029-8-B | NM_021141 | AAGCATAACTATGAGTGTTTA | SI02663766 | Hs_XRCC5_6 |
| 900029-8-B | NM_021915 | TACCAACAGTTATCTCATGTA | SI03224417 | Hs_ZNF69_7 |
| 900029-8-B | NM_003559 NM_ [symbol] | GCGGAGATGCACAACATCTTA | SI02660126 | Hs_PIP5K2B_8 |
| 900029-8-B | NM_003676 NM_ [symbol] | ATGCGTTTGGCAGTTGCATTA | SI03050201 | Hs_DEGS1_7 |
| 900029-8-B | NM_003804 | TACCACTAGTCTGACGGATAA | SI00288092 | Hs_RIPK1_5 |
| 900029-8-B | NM_003921 | CAGAAGGAGAATCCAGCACGA | SI03063144 | Hs_BCL10_7 |
| 900029-8-B | NM_005147 | TCCGACCTCTTTATTTCTATA | SI00370671 | Hs_DNAJA3_3 |
| 900029-8-B | NM_001032282 N [symbol] | AGCCCGTTGTGCAGAGTTCAA | S103043236 | Hs_KLF10_8 |
| 900029-8-B | NM_000546 | CAGAGTGCATTGTGAGGGTTA | SI00011655 | Hs_TP53_3 |
| 900029-8-B | NM_003347 NM_ [symbol] | CACACTCCAGTTTGTAATAAA | SI00754649 | Hs_UBE2L3_3 |
| 900029-8-B | NM_005433 | CCAGCCTACATTCACTTCTAA | SI02223935 | Hs_YES1_6 |
| 900029-8-B | NM_001010972 N [symbol] | ACCAAGAATGATCCTTTCAAA | SI02651334 | Hs_ZYX_5 |
| 900029-8-B | NM_003567 | CCGAGCGGCCACTCTGAGTAA | SI03080196 | Hs_BCAR3_5 |
| 900029-8-B | NM_003745 | TAAAGTCAGTTTAGGTAATAA | SI03107342 | Hs_SOCS1_5 |
| 900029-8-B | NM_003819 | ACGGAAATTTGAACAGTTGAA | SI02651474 | Hs_PABPC4_7 |
| 900029-8-B | NM_003939 NM_ [symbol] | CACGTTGATTCACCATTGTGA | SI03061807 | Hs_BTRC_11 |
| 900029-8-B | NM_004702 NM_ [symbol] | AAGAAGAGTATTAAATATATA | SI02653035 | Hs_CCNE2_7 |
| 900029-8-B | NM_003257 NM_ [symbol] | AAGGATCCATATCCCGAGGAA | SI03034983 | Hs_TJP1_8 |
| 900029-8-B | NM_003290 | TTGCATATTTCCTTCATTCTA | SI00750148 | Hs_TPM4_4 |
| 900029-8-B | NM_006003 | ATGCTCAGTCATACACGCGAA | SI03152765 | Hs_UQCRFS1_5 |
| 900029-8-B | NM_003404 NM_ [symbol] | CAGCAATATGTTCACTATGTT | SI02631104 | Hs_YWHAB_5 |
| 900029-8-B | NM_006009 | TCCAACCTATACTAACCTGAA | SI00753305 | Hs_TUBA3_3 |
| 900029-8-B | NM_001004720 N [symbol] | GACACTAATACAGATGATTAA | SI02624993 | Hs_NCK2_9 |
| 900029-8-B | NM_001037494 N [symbol] | CACACCCAGTGATCCATCCAA | SI03158939 | Hs_DYNLL1_2 |
| 900029-8-B | NM_003852 NM_ [symbol] | CCGAGACTTATCTAAACCAGA | SI00056805 | Hs_TIF1_4 |
| 900029-8-B | NM_003941 | CAGATACGACAGGGTATCCAA | SI02664263 | Hs_WASL_6 |
| 900029-8-B | NM_004747 | TTGCCTGTTTGTCGACTATAA | SI00067872 | Hs_DLG5_3 |
| 900029-8-B | NM_005078 | CCCGGGCAGCCGGGATTTAAA | SI00745689 | Hs_TLE3_3 |
| 900029-8-B | NM_003292 | GAGGGTGAAGATAGTAATGAA | SI00750232 | Hs_TPR_4 |
| 900029-8-B | NM_005428 | GTCGAGGTCAAGCACATTAAA | SI00077021 | Hs_VAV1_4 |
| 900029-8-B | NM_006761 | AAGCATCTAAGAGAGAGGTTA | SI03033023 | Hs_YWHAE_7 |
| 900029-8-B | NM_001013836 N [symbol] | CACGAGCAGCAGATTAAGGAT | SI03059791 | Hs_MAD1L1_6 |
| 900029-8-B | NM_003619 | AAGCGGATCATTGGTGGGAAA | SI00053823 | Hs_PRSS12_3 |
| 900029-8-B | NM_003749 | TCGCTATAGAATAATGCATTA | SI02662037 | Hs_IRS2_5 |
| 900029-8-B | NM_004664 | TTCGAGAGGTGTATCAATATA | SI00066815 | Hs_LIN7A_2 |
| 900029-8-B | NM_003954 | CACATGCATGTGACTCCTCAA | SI02632336 | Hs_MAP3K14_5 |
| 900029-8-B | NM_004755 NM_ [symbol] | AAGCCAGTCATTCGAGATGAA | SI02225433 | Hs_RPS6KA5_8 |
| 900029-8-B | NM_006289 | TGGGAAAGCTTTGGACTACTA | SI00086982 | Hs_TLN1_4 |
| 900029-8-B | NM_003299 | AAGTTGATGTGGATGGTACAT | SI02655177 | Hs_TRA1_8 |
| 900029-8-B | NM_003371 | TTGGCGATGTACATCAATGAA | SI02662380 | Hs_VAV2_5 |
| 900029-8-B | NM_003406 NM_ [symbol] | CAGGTTTATGTTACTTCTATT | SI00764813 | Hs_YWHAZ_3 |
| 900029-8-B | NM_003557 | CAGCCTCTTGATGTCAATCCA | SI02223256 | Hs_PIP5K1A_5 |
| 900029-8-B | NM_003629 | CAGGGCTGTAGTATTCAGTAA | SI02777439 | Hs_PIK3R3_8 |
| 900029-8-B | NM_001037283 N [symbol] | CAGGTTGAACATGGAAATAAA | SI00377839 | Hs_EIF3S9_3 |
| 900029-8-B | NM_003870 | TCCTATGGTTGTGGTCCGAAA | SI03115770 | Hs_IQGAP1_6 |
| 900029-8-B | NM_003955 | CAGAAGAGCCTATTACATCTA | SI03062997 | Hs_SOCS3_7 |
| 900029-8-B | NM_005451 NM_ [symbol] | AGGCACCGAGTTCATGCAAGA | SI03044979 | Hs_PDLIM7_9 |
| 900029-8-B | NM_001001723 N [symbol] | CTGGTTAATAGTGATAGTTGA | SI03099999 | Hs_TMEM1_7 |
| 900029-8-B | NM_003302 | TGGGCTGCTTTGTATGTTCTA | SI02630866 | Hs_TRIP6_4 |
| 900029-8-B | NM_005429 | CACGGCTTATGCAAGCAAAGA | SI03060939 | Hs_VEGFC_7 |
| 900029-8-B | NM_001079 NM_ [symbol] | CCGCAACGTCCTGCTGGTTAA | SI00021672 | Hs_ZAP70_3 |
| 900029-8-B | NM_003558 | TACACTCTATTCAAACAGCAA | SI02660273 | Hs_PIP5K1B_5 |
| 900029-8-B | NM_003639 | CTCCTCTAGTTCAGAGACATA | SI02223333 | Hs_IKBKG_5 |
| 900029-8-B | NM_003794 | AGGCGACGGATTGGTTTAGAA | SI03045168 | Hs_SNX4_9 |
| 900029-8-B | NM_003904 | CAGCTTGTGATGCAAGTGTGA | SI03173331 | Hs_ZNF259_7 |
| 900029-8-B | NM_003974 NM_ [symbol] | TTGGGCTGCTTGGCTATGCAA | SI03025344 | Hs_DOK2_5 |
| 900029-8-B | NM_004759 NM_ [symbol] | CGCCATCATCGATGACTACAA | SI00288246 | Hs_MAPKAPK2_6 |
| 900029-9-A | NM_004238 | CAGATGTTTCATCATTTGAAA | SI04023145 | Hs_TRIP12_8 |
| 900029-9-A | NM_014567 | AAGCAGTTTGAACGACTGGAA | SI02757741 | Hs_BCAR1_6 |
| 900029-9-A | NM_014776 NM_ [symbol] | CCCGTTGATTATGCAAGGCAA | SI02660420 | Hs_GIT2_6 |
| 900029-9-A | NM_005489 NM_ [symbol] | TAGGATACTGGGCGTTACCAA | SI03111717 | Hs_SH2D3C_6 |
| 900029-9-A | NM_001010938 N [symbol] | CGGCAGTCAGATCCTGCATAA | SI02622151 | Hs_TNK2_6 |
| 900029-9-A | NM_005864 NM_ [symbol] | CAGCACTGCACTGTTAGCTGA | SI03169929 | Hs_EFS_6 |
| 900029-9-A | NM_006471 | CGGGACTTTCTATTAATATCA | SI00647976 | Hs_MRCL3_4 |
| 900029-9-A | NM_006826 | TCGGGAGAAAGTGGAGTCCGA | SI03117030 | Hs_YWHAQ_8 |
| 900029-9-A | NM_022105 NM_ [symbol] | TAGCGAAGACCAAGGGATAAA | SI03111185 | Hs_DIDO1_2 |
| 900029-9-A | NM_014614 | TAGGATTAAATTGGTTTCCTA | SI00695128 | Hs_PSME4_4 |
| 900029-9-A | NM_000978 | TCCCAGTGTCCTTTGAATCGA | SI03231389 | Hs_RPL23_7 |
| 900029-9-A | NM_004907 | CGCCGTCGAGTCGCCATGGAA | SI03194079 | Hs_IER2_6 |
| 900029-9-A | NM_012296 NM_ [symbol] | CACTTGGCTACCCATCAACAA | SI03062570 | Hs_GAB2_8 |
| 900029-9-A | NM_005490 | GCCGTGATTTCTAGCAGTTGA | SI03105074 | Hs_SH2D3A_7 |
| 900029-9-A | NM_005831 | CAGGGACTGAACAAATGGAAA | SI00656040 | Hs_NDP52_4 |
| 900029-9-A | NM_006082 | CAGCTTAACTGACAGACGTTA | SI02636634 | Hs_K-ALPHA-1_6 |
| 900029-9-A | NM_006472 | AAGAGCCAATTTAACAAACTA | SI03648827 | Hs_TXNIP_8 |
| 900029-9-A | NM_006837 | CTGGACTAAGGATCACCATTA | SI03096905 | Hs_COPS5_7 |
| 900029-9-A | NM_001042600 N [symbol] | TACGTGGGTGTACTCCATCAA | SI02224257 | Hs_MAP4K1_6 |
| 900029-9-A | NM_015192 NM_ [symbol] | CAGAGATGATCCGGTCATATA | SI02781184 | Hs_PLCB1_6 |
| 900029-9-A | NM_004810 | CACAGTTAATGGATCTGTAAA | SI00068705 | Hs_GRAP2_4 |
| 900029-9-A | NM_004295 NM_ [symbol] | CCGGAGCTGGAAGTACAAGTA | SI02665159 | Hs_TRAF4_8 |
| 900029-9-A | NM_005112 NM_ [symbol] | AAAGTGCGTCATCCTAAGGAA | SI03122448 | Hs_WDR1_5 |
| 900029-9-A | NM_005721 | ATCGATGTTGGTTATGAGAGA | SI02664305 | Hs_ACTR3_8 |
| 900029-9-A | NM_005840 | CTGAAGGGAGAAGCTGAGCAA | SI03206063 | Hs_SPRY3_5 |
| 900029-9-A | NM_006097 NM_ [symbol] | CACATCCAATGTCTTCGCAAT | SI03159926 | Hs_MYL9_5 |
| 900029-9-A | NM_006576 | CAGGAAATAATTTATCCACCA | SI00308490 | Hs_AVIL_4 |
| 900029-9-A | NM_007007 | ACCGTCATGACGATTATTACA | SI03139563 | Hs_CPSF6_5 |
| 900029-9-A | NM_012250 | AACAGTTAGCACGGCAGCTTA | SI02662870 | Hs_RRAS2_6 |
| 900029-9-A | NM_012243 | TCCCACTAAAGCATAGTTGTA | SI03231270 | Hs_SLC35A3_5 |
| 900029-9-A | NM_001039583 N [symbol] | CACCTCGGCGGCCTTTATTTA | SI02660350 | Hs_PRKCABP_7 |
| 900029-9-A | NM_014649 | GCGGACGGTCGTGATGGATAA | SI03220441 | Hs_SAFB2_5 |
| 900029-9-A | NM_005465 NM_ [symbol] | ACCAGAGGTGTTAGAAGATAA | SI02757762 | Hs_AKT3_12 |
| 900029-9-A | NM_005749 | CCAGCCTGTTATGGCTAACTA | SI02663248 | Hs_TOB1_7 |
| 900029-9-A | NM_005841 NM_ [symbol] | AAGCATGAAAGGACTCATGAA | SI03033030 | Hs_SPRY1_8 |
| 900029-9-A | NM_006098 | ACCAGGGATGAGACCAACTAT | SI03038651 | Hs_GNB2L1_7 |
| 900029-9-A | NM_006613 XM_ [symbol] | TGGCTTCTTCCCACGGAGTTA | SI03238564 | Hs_GRAP_8 |
| 900029-9-A | NM_007011 NM_ [symbol] | TTCGTTGACTACGCCCAGAAA | SI03242806 | Hs_ABHD2_6 |
| 900029-9-A | NM_007368 | AACCTGAAGTTTGGAGATGAA | SI00698768 | Hs_RASA3_4 |
| 900029-9-A | NM_014299 NM_ [symbol] | TGGCGTTTCCACGGTACCAAA | SI03238361 | Hs_BRD4_7 |
| 900029-9-A | NM_004845 | CTCAACTCGTTTATAAATAAA | SI00681044 | Hs_PCYT1B_4 |
| 900029-9-A | NM_014715 | CCTGATGAATCTACTGCTAAA | SI00107702 | Hs_RICS_4 |
| 900029-9-A | NM_001012750 N [symbol] | CTAGTGTTGCTTATCAAATAA | SI02655338 | Hs_ABI1_5 |
| 900029-9-A | NM_005760 | TGGATCGATTTGTATACCGAA | SI03237710 | Hs_CEBPZ_7 |
| 900029-9-A | NM_005842 | CAGGTCCATTCTTCTGCACGA | SI03071943 | Hs_SPRY2_7 |
| 900029-9-A | NM_001039619 N [symbol] | AAGAGGGAGTTCATTCAGGAA | SI02657067 | Hs_SKB1_6 |
| 900029-9-A | NM_006614 | ACCCAAGGAAATGATTATAAA | SI03138058 | Hs_CHL1_5 |
| 900029-9-A | NM_007013 | CAGTTCTATTTAACTGAATTA | SI03179001 | Hs_WWP1_5 |
| 900029-9-A | NM_015001 | CCCGATCACGCCGCAAGCGAA | SI03077697 | Hs_SPEN_8 |
| 900029-9-A | NM_012120 | TAGCAAGTCTTGTACAACGAA | SI03110884 | Hs_CD2AP_8 |
| 900029-9-A | NM_004883 NM_ [symbol] | CAGCCTCAAGTCAGTGCAGGA | SI03066483 | Hs_NRG2_6 |
| 900029-9-A | NM_014740 | CCGCATCTTGGTGAAACGTGA | SI02663794 | Hs_DDX48_5 |
| 900029-9-A | NM_006044 | CACTTCGAAGCGAAATATTAA | SI02757769 | Hs_HDAC6_6 |
| 900029-9-A | NM_001018003 N [symbol] | TACGTGCAGGTGTCTCGTGAA | SI03225971 | Hs_SORBS3_4 |
| 900029-9-A | NM_005861 | TACGCGAACGCCCACCCTTAA | SI03109659 | Hs_STUB1_5 |
| 900029-9-A | NM_006113 | CACGACTTTCTCGAACACCTA | SI02781233 | Hs_VAV3_5 |
| 900029-9-A | NM_006666 | CCAGGACGCCTTCCTCTTCAA | SI00709268 | Hs_RUVBL2_4 |
| 900029-9-A | NM_007014 NM_ [symbol] | TACCCGCACCACCACCTTTAA | SI03108826 | Hs_WWP2_9 |
| 900029-9-A | NM_012307 | CTGCACGTTCATAACCAACAA | SI03207491 | Hs_EPB41L3_5 |
| 900029-9-A | NM_012116 | CTCACCTATGATGAGGTCCAA | SI03088414 | Hs_CBLC_6 |
| 900029-9-B | NM_004238 | CAGTGCTAGTCCAGACTACAA | SI03073182 | Hs_TRIP12_7 |
| 900029-9-B | NM_014567 | CTGGATGGAGGACTATGACTA | SI02757734 | Hs_BCAR1_5 |
| 900029-9-B | NM_014776 NM_ [symbol] | CAGCGTTGAGAGTCAAGACAA | SI02660413 | Hs_GIT2_5 |
| 900029-9-B | NM_005489 NM_ [symbol] | CCGGCGCTATGAGAAGTTCGA | SI03082198 | Hs_SH2D3C_5 |
| 900029-9-B | NM_001010938 N [symbol] | ACGCAAGTCGTGGATGAGTAA | SI02622144 | Hs_TNK2_5 |
| 900029-9-B | NM_005864 NM_ [symbol] | ACGAGCGTCAGCCTTACTCAA | SI03140431 | Hs_EFS_5 |
| 900029-9-B | NM_006471 | TTGGGCATATGTATCTTTATA | SI00647969 | Hs_MRCL3_3 |
| 900029-9-B | NM_006826 | CTGCATGAAGGCAGTGACCGA | SI03094581 | Hs_YWHAQ_7 |
| 900029-9-B | NM_022105 NM_ [symbol] | CCGACTCGGTGTACTGCAGTA | SI03080042 | Hs_DIDO1_1 |
| 900029-9-B | NM_014614 | CACGTGTAGTTCTTTATTATA | SI00695121 | Hs_PSME4_3 |
| 900029-9-B | NM_000978 | GGCGTTAAAGTTCATATCCCA | SI03221211 | Hs_RPL23_6 |
| 900029-9-B | NM_004907 | AGCAAGAAAGCCCGTCTGGAA | SI03143000 | Hs_IER2_5 |
| 900029-9-B | NM_012296 NM_ [symbol] | CACCAATTCTGAAGACAACTA | SI02639301 | Hs_GAB2_6 |
| 900029-9-B | NM_005490 | CAGGTGCCACAGGATGGAGAA | SI03072167 | Hs_SH2D3A_6 |
| 900029-9-B | NM_005831 | CAGCAGGAAGTCCAATTCAAA | SI00656033 | Hs_NDP52_3 |
| 900029-9-B | NM_006082 | CCCGCCCTAGTGCGTTACTTA | SI02636627 | Hs_K-ALPHA-1_5 |
| 900029-9-B | NM_006472 | TGCAACATCCTTCGAGTTGAA | SI03117723 | Hs_TXNIP_7 |
| 900029-9-B | NM_006837 | ATGCAATCGGGTGGTATCATA | SI03049774 | Hs_COPS5_6 |
| 900029-9-B | NM_001042600 N [symbol] | CTGACTAAGAGTCCCAAGAAA | SI02224250 | Hs_MAP4K1_5 |
| 900029-9-B | NM_015192 NM_ [symbol] | TCGAGATTACATGGATGTTAA | SI02780939 | Hs_PLCB1_5 |
| 900029-9-B | NM_004810 | CTGGTAATTAGTTGATGCAAA | SI00068698 | Hs_GRAP2_3 |
| 900029-9-B | NM_004295 NM_ [symbol] | CTGCAGGAGTTCCTCAGTGAA | SI02665152 | Hs_TRAF4_7 |
| 900029-9-B | NM_005112 NM_ [symbol] | CAGTAACAGTTTGCACATGAA | SI00761712 | Hs_WDR1_4 |
| 900029-9-B | NM_005721 | CCGGCTGAAATTAAGTGAGGA | SI02664298 | Hs_ACTR3_7 |
| 900029-9-B | NM_005840 | CAGGGTAGTATGAGTAAGGAA | SI00732620 | Hs_SPRY3_4 |
| 900029-9-B | NM_006097 NM_ [symbol] | CGCCAAGGATAAAGACGACTA | SI00653072 | Hs_MYL9_4 |
| 900029-9-B | NM_006576 | CTGGACCAAAGTGGAACCAAA | SI00308483 | Hs_AVIL_3 |
| 900029-9-B | NM_007007 | TAGATGTAGTGTTGTAATAAA | SI00353227 | Hs_CPSF6_3 |
| 900029-9-B | NM_012250 | TAGTCCTTTCTGTGAAGCTAA | SI03112340 | Hs_RRAS2_9 |
| 900029-9-B | NM_012243 | GAGAATAAATATCAAATCTAA | SI00723464 | Hs_SLC35A3_4 |
| 900029-9-B | NM_001039583 N [symbol] | TCGCCTCACCATCAAGAAGTA | SI00287574 | Hs_PRKCABP_5 |
| 900029-9-B | NM_014649 | CCGGACCTACAAATCGGTCTA | SI00710080 | Hs_SAFB2_4 |
| 900029-9-B | NM_181690 | CAGCAGGCACGTTAACTCGAA | SI02622494 | Hs_AKT3_8 |
| 900029-9-B | NM_005749 | ACCAAGTTCGGCTCTACCAAA | SI03038322 | Hs_TOB1_8 |
| 900029-9-B | NM_005841 NM_ [symbol] | TTGGATAGCCGTCAGAGATTA | SI03024805 | Hs_SPRY1_7 |
| 900029-9-B | NM_006098 | TTGGCACACGCTAGAAGTTTA | SI02636662 | Hs_GNB2L1_5 |
| 900029-9-B | NM_006613 XM_ [symbol] | CCGCCGTGGCGACATCATTGA | SI03189088 | Hs_GRAP_7 |
| 900029-9-B | NM_007011 NM_ [symbol] | ACGATCCGTTGGTGCATGAAA | SI03140557 | Hs_ABHD2_5 |
| 900029-9-B | NM_007368 | TAGGAACTTTCTTGTCACAAA | SI00698761 | Hs_RASA3_3 |
| 900029-9-B | NM_014299 NM_ [symbol] | CCGGCCTGTGAAACCTCCAAA | SI03190845 | Hs_BRD4_6 |
| 900029-9-B | NM_004845 | CCGGGTGTATCCCATGTGCAA | SI00681037 | Hs_PCYT1B_3 |
| 900029-9-B | NM_014715 | TAGGCAGTTCTGTGAGTCAAA | SI00107695 | Hs_RICS_3 |
| 900029-9-B | NM_001012750 N [symbol] | GAGGGCGCTGATCGAGAGTTA | SI03218754 | Hs_ABI1_6 |
| 900029-9-B | NM_005760 | CAGTACCACACCGAAAGTAAA | SI03178007 | Hs_CEBPZ_6 |
| 900029-9-B | NM_005842 | AACCAACATAGCATCATTAAT | SI02636151 | Hs_SPRY2_6 |
| 900029-9-B | NM_001039619 N [symbol] | TGGGCAAGGGATTATAATTAA | SI02657060 | Hs_SKB1_5 |
| 900029-9-B | NM_006614 | CCCAGAGATGGTCATATTTAA | SI00345954 | Hs_CHL1_4 |
| 900029-9-B | NM_007013 | AAGGATTCATTGAGCAGCATA | SI00763784 | Hs_WWP1_4 |
| 900029-9-B | NM_015001 | CAGAACTGCCTTTGCACTAAA | SI02641128 | Hs_SPEN_7 |
| 900029-9-B | NM_012120 | AAGCGTCAGTGTAAAGTTCTT | SI03033681 | Hs_CD2AP_7 |
| 900029-9-B | NM_004883 NM_ [symbol] | CAGAAAGAACTCACGACTACA | SI03062682 | Hs_NRG2_5 |
| 900029-9-B | NM_014740 | ATGATTCGTCGCAGAAGCCTA | SI03049676 | Hs_DDX48_6 |
| 900029-9-B | NM_006044 | CACCGTCAACGTGGCATGGAA | SI02663808 | Hs_HDAC6_5 |
| 900029-9-B | NM_001018003 N [symbol] | CGGAACGTTCCCTGGAAATTA | SI03195248 | Hs_SORBS3_3 |
| 900029-9-B | NM_005861 | CCCGAGCGCGCAGGAGCTCAA | SI00081977 | Hs_STUB1_3 |
| 900029-9-B | NM_001079874 N [symbol] | ATGGACTGCGAAGAACTCCTA | SI03050733 | Hs_VAV3_6 |
| 900029-9-B | NM_006666 | AACCGTTACAGCCACAACCAA | SI00709261 | Hs_RUVBL2_3 |
| 900029-9-B | NM_007014 NM_ [symbol] | CTGCGCTACTTTGACGAGAAA | SI03095344 | Hs_WWP2_8 |
| 900029-9-B | NM_012307 | TAACCTTATATTTACAATAAA | SI00380282 | Hs_EPB41L3_4 |
| 900029-9-B | NM_012116 | ATGGCCAACACTCCTCAAGAA | SI03051349 | Hs_CBLC_5 |
| 900029-10-A | NM_012399 | CACGTCGGCTGCTGATGTCTA | SI03165050 | Hs_PITPNB_8 |
| 900029-10-A | NM_012428 NM_ [symbol] | CAGACTGGATATGGCGCAAGA | SI03063739 | Hs_NPTN_1 |
| 900029-10-A | NM_001024666 N [symbol] | TAGCTTATATATGACGGTATA | SI00287903 | Hs_SH3KBP1_6 |
| 900029-10-A | NM_016135 | CAGCTGCTCCTTGATACCCGA | SI03068387 | Hs_ETV7_6 |
| 900029-10-A | NM_019074 | CCGGGTCATCTGCAGTGACAA | SI03082653 | Hs_DLL4_6 |
| 900029-10-A | NM_001006600 N [symbol] | CAAGATGTTATCAATTGGTTA | SI02778195 | Hs_ERBB2IP_11 |
| 900029-10-A | NM_020801 | CAGAATTACACTGAAGAAGTA | SI03167220 | Hs_ARRDC3_5 |
| 900029-10-A | NM_022165 | CTCGAGGTTGAAACCACAGAT | SI03091116 | Hs_LIN7B_7 |
| 900029-10-A | NM_024665 | AAGGAGCTATCTGTTTATGTA | SI00136808 | Hs_TBL1XR1_1 |
| 900029-10-A | NM_030637 | CTGACAAAGTACGAGGTTTAA | SI03206238 | Hs_DDH D1_5 |
| 900029-10-A | XM_001129272 X [symbol] | TTCGGATCGGTTTGTAATTAA | SI02825263 | Hs_SHC2_7 |
| 900029-10-A | NM_005618 | AAGGATGAGTGCGTCATAGCA | SI03132836 | Hs_DLL1_5 |
| 900029-10-A | NM_018482 | CACCTTGGATTCTTTGTTAAA | SI00360598 | Hs_DDEF1_4 |
| 900029-10-A | NM_015878 NM_ [symbol] | ACACTCGCAGTTAATATCATA | SI03037622 | Hs_AZIN1_1 |
| 900029-10-A | NM_017672 | CTGCTAGCGTATATTCATAAA | SI03095806 | Hs_TRPM7_8 |
| 900029-10-A | NM_019619 | TAGGTATAGCTGACGAGACTA | SI03228932 | Hs_PARD3_6 |
| 900029-10-A | NM_021235 | TACCTCGGATCCATTCACGAA | SI03109176 | Hs_EPS15L1_7 |
| 900029-10-A | NM_022452 | CCAAGGAATCTGTGCGGGTAA | SI03179960 | Hs_FBS1_5 |
| 900029-10-A | NM_024745 | CAAGATATCCATGGTGAATAA | SI00717864 | Hs_SHCBP1_4 |
| 900029-10-A | NM_030964 | CACGAACAGCGTCATCTGCAA | SI00732648 | Hs_SPRY4_4 |
| 900029-10-A | NM_012319 | CACGAGCATCACTCTGACCAT | SI03059805 | Hs_SLC39A6_8 |
| 900029-10-A | NM_014030 | GCCGCTGAGGATGTCCCGAAA | SI02622340 | Hs_GIT1_6 |
| 900029-10-A | NM_001037281 N [symbol] | CCAGGTTTCCTCAGTCATAGA | SI02664340 | Hs_PARD6A_5 |
| 900029-10-A | NM_015975 | CAGAGTATGAACCAAGGGTTA | SI03168480 | Hs_TAF9B_1 |
| 900029-10-A | NM_017741 | AAGGAAACAACTCGTACTGAA | SI00397250 | Hs_FLJ20280_4 |
| 900029-10-A | NM_020064 | AAGTGTATATGAAGTTATTTA | SI00310338 | Hs_BARHL1_4 |
| 900029-10-A | NM_021249 NM_ [symbol] | CCGCGGACTTAAAGCAATAAA | SI02778538 | Hs_SNX6_9 |
| 900029-10-A | NM_022765 | CTACAGCTCGTTGACAAGAAA | SI00658644 | Hs_NICAL_4 |
| 900029-10-A | NM_024940 | AACATCCTAGACCCTGACGAA | SI03123645 | Hs_DOCK5_5 |
| 900029-10-A | NM_031308 | AGGCTTCATCATCGACCCAAA | SI00380730 | Hs_EPPK1_4 |
| 900029-10-A | NM_001042635 N [symbol] | CGGGAGAAGAAGCGTCTAGAA | SI00318598 | Hs_C14orf120_4 |
| 900029-10-A | NM_013275 | CCCTCGGAGCACGAATATATA | SI03186925 | Hs_ANKRD11_6 |
| 900029-10-A | NM_015965 | CGCGGTCGGATAGTTACACTA | SI00430941 | Hs_GRIM19_4 |
| 900029-10-A | NM_016084 | GAGGGTGGATTTATCTTCTCA | SI03104115 | Hs_RASD1_6 |
| 900029-10-A | NM_018227 | AAGGGAAACATCAGTAAGAAA | SI00389942 | Hs_FLJ10808_4 |
| 900029-10-A | NM_020066 XM_ [symbol] | CTGGACCAGGATTCAACTACA | SI03210186 | Hs_FMN2_8 |
| 900029-10-A | NM_001037126 N [symbol] | TAGCCGAGTTGTGCAGCGTAA | SI03227595 | Hs_EXOC4_1 |
| 900029-10-A | NM_022842 NM_ [symbol] | AACCCTGATGTCTGCCAACTA | SI00341446 | Hs_CDCP1_4 |
| 900029-10-A | NM_025146 | CGGCGTTGATATCGGTGGTAA | SI03196508 | Hs_NAT13_1 |
| 900029-10-A | NM_031483 | TGCCGCCGACAAATACAAATA | SI03118437 | Hs_ITCH_6 |
| 900029-10-A | NM_015678 | AAGAAATAAATTCACCAACAA | SI00655480 | Hs_NBEA_4 |
| 900029-10-A | NM_013277 | CTGGTAGATAGAAGAGCTAAA | SI02639840 | Hs_RACGAP1_5 |
| 900029-10-A | NM_016263 | CGGGTCGATCTTCCACATTCA | SI00114905 | Hs_FZR1_1 |
| 900029-10-A | NM_016848 | CTCCAAACAGATCATAGCGAA | SI03199805 | Hs_SHC3_5 |
| 900029-10-A | NM_018362 | TCAACCGTACATCAAATTATA | SI02778048 | Hs_LIN7C_6 |
| 900029-10-A | NM_020148 | TACGTGGGCTATACTGTATTA | SI03226027 | Hs_SPIRE1_8 |
| 900029-10-A | NM_022090 | AAAGAGATACCTCATATCGTA | SI03121783 | Hs_LOC63920_5 |
| 900029-10-A | NM_024045 | AAGAAATCAAGAAACAATTAA | SI00361662 | Hs_DDX50_4 |
| 900029-10-A | XM_001130728 X [symbol] | TCCCAATTAGTCTGTATCTAT | SI03020038 | Hs_C20orf119_4 |
| 900029-10-A | NM_032704 | CTGTAAATGTCTATTGCCGTA | SI02654071 | Hs_TUBA6_3 |
| 900029-10-A | NM_002568 | TTAAAGCATTCCTTTCTTTAA | SI03240111 | Hs_PABPC1_6 |
| 900029-10-A | NM_013333 | CCGGAAGACGCCGGAGTCATT | SI00380618 | Hs_EPN1_4 |
| 900029-10-A | NM_016356 | AAAGTCATATAGAAATATTAA | SI00360066 | Hs_DCDC2_4 |
| 900029-10-A | NM_018948 | AACCAATTAGTTACTATCGTA | SI00645792 | Hs_MIG-6_4 |
| 900029-10-A | NM_018440 | CTGCTTGTATGAAACTGTGAA | SI02643305 | Hs_PAG_8 |
| 900029-10-A | NM_020673 | AAGGCCTATCAGCCAATTAAA | SI02662737 | Hs_RAB22A_6 |
| 900029-10-A | NM_022101 | TAGACTAGAGGCAAGCATTTA | SI03226818 | Hs_CXorf56_2 |
| 900029-10-A | NM_024108 | TGGCGGTGTTCCAGAAGCAGA | SI03238319 | Hs_TRAPPC6A_2 |
| 900029-10-A | NM_025248 | TCGCATCATCGCAGAGCTAGA | SI03233727 | Hs_SNIP_5 |
| 900029-10-A | NM_032832 | AAACATGATTCCTTTAATAAA | SI00623644 | Hs_LRP11_4 |
| 900029-10-B | NM_012399 | AAGGGACAGTATACGCACAAA | SI03133788 | Hs_PITPNB_7 |
| 900029-10-B | NM_012428 NM_ [symbol] | CTGGTACATAAAGATGAGTAA | SI02639672 | Hs_SDFR1_9 |
| 900029-10-B | NM_001024666 N [symbol] | CTCTGAGATCTCAATGCGAAA | SI00287896 | Hs_SH3KBP1_5 |
| 900029-10-B | NM_016135 | CACGTGCAAGCCAGATGTGAA | SI03061590 | Hs_ETV7_5 |
| 900029-10-B | NM_019074 | TTCGGGCTGTCATGAACAGAA | SI03023076 | Hs_DLL4_5 |
| 900029-10-B | NM_001006600 N [symbol] | CCCGAAGAGCCAAATATAATA | SI02778188 | Hs_ERBB2IP_10 |
| 900029-10-B | NM_020801 | CACGAAAGAGATGATGATAAT | SI00304598 | Hs_ARRDC3_4 |
| 900029-10-B | NM_022165 | CTCCGCTATCCGAGAGGTGTA | SI03090066 | Hs_LIN7B_6 |
| 900029-10-B | NM_024665 | TAGCACCTTAGGGCAGCATAA | SI03110905 | Hs_TBL1XR1_11 |
| 900029-10-B | NM_030637 | CCAGGAAATACTGGAAGTCAA | SI00360682 | Hs_DDHD1_4 |
| 900029-10-B | XM_001129272 X [symbol] | ACGGAACCATTTCGCCTTTAA | SI02825256 | Hs_SHC2_6 |
| 900029-10-B | NM_005618 | CCGCGTGTGCCTCAAGCACTA | SI00369719 | Hs_DLL1_3 |
| 900029-10-B | NM_018482 | CACCTTCAAGTCAATTCATAA | SI00360591 | Hs_DDEF1_3 |
| 900029-10-B | NM_015878 NM_ [symbol] | CGGATTTGCTTGTTCCAGTAA | SI00112322 | Hs_OAZIN_4 |
| 900029-10-B | NM_017672 | CCTGTAAGATCTATCGTTCAA | SI03083549 | Hs_TRPM7_7 |
| 900029-10-B | NM_019619 | CAGCTTAAGAAAGGTACAGAA | SI03173121 | Hs_PARD3_5 |
| 900029-10-B | NM_021235 | CACCGCCTAAATTTCACGACA | SI03058398 | Hs_EPS15L1_6 |
| 900029-10-B | NM_022452 | CAGGCTAAGGGTGGCCAGAGA | SI00385210 | Hs_FBS1_4 |
| 900029-10-B | NM_024745 | TTGCATAATACTTGTCTTAAA | SI00717857 | Hs_SHCBP1_3 |
| 900029-10-B | NM_030964 | CTGAATGTACTGATTTAGAAA | SI00732641 | Hs_SPRY4_3 |
| 900029-10-B | NM_012319 | CTAGTTAAGGTTTAAATGCTA | SI02639371 | Hs_SLC39A6_6 |
| 900029-10-B | NM_014030 | CAGCCTTGACTTATCCGAATT | SI02224467 | Hs_GIT1_5 |
| 900029-10-B | NM_001037281 N [symbol] | ATCGTCGAGGTGAAGAGCAAA | SI03048619 | Hs_PARD6A_6 |
| 900029-10-B | NM_015975 | CAGAAGTTTCATCTTAGGATA | SI00739081 | Hs_TAF9L_3 |
| 900029-10-B | NM_017741 | CAGCAAGAGTCCTAACATCTA | SI00397243 | Hs_FLJ20280_3 |
| 900029-10-B | NM_020064 | CAGGACTAAATGGAAGCGACA | SI00310331 | Hs_BARHL1_3 |
| 900029-10-B | NM_021249 NM_ [symbol] | CAGGCCGAAACTTCCCAACAA | SI03070424 | Hs_SNX6_11 |
| 900029-10-B | NM_022765 | CCAGCGGTTGTCCTCCCTTAA | SI00658637 | Hs_NICAL_3 |
| 900029-10-B | NM_024940 | CACATTCAGCTTATAATGGAA | SI00372666 | Hs_DOCK5_4 |
| 900029-10-B | NM_031308 | AAGCAGGAAACCAGCAACAAA | SI00380723 | Hs_EPPK1_3 |
| 900029-10-B | NM_001042635 N [symbol] | CTCTGTCATTCGTGAACTTAA | SI00318591 | Hs_C14orf120_3 |
| 900029-10-B | NM_013275 | CACGAGAGCCTTCTAATGCCA | SI03163370 | Hs_ANKRD11_5 |
| 900029-10-B | NM_015965 | AGGGATTGGAACCCTGATCTA | SI00430934 | Hs_GRIM19_3 |
| 900029-10-B | NM_016084 | CCGCAAGGTCTCGGTGCAGTA | SI03080483 | Hs_RASD1_5 |
| 900029-10-B | NM_018227 | ATGGATCTATACAACAAATAA | SI00389935 | Hs_FLJ10808_3 |
| 900029-10-B | NM_020066 XM_ [symbol] | CTGATACTATCTCAAAGACGA | SI03206952 | Hs_FMN2_7 |
| 900029-10-B | NM_001037126 N [symbol] | CAGCAAGAAGATGAACCTTCA | SI00714301 | Hs_SEC8L1_3 |
| 900029-10-B | NM_022842 NM_ [symbol] | AAGATGCAAGAAGGAGTGAAA | SI00341439 | Hs_CDCP1_3 |
| 900029-10-B | NM_025146 | TTGGTGCAGTTAAGAATTAAA | SI00627200 | Hs_MAK3_4 |
| 900029-10-B | NM_031483 | ATGGGTAGCCTCACCATGAAA | SI03051839 | Hs_ITCH_5 |
| 900029-10-B | NM_015678 | CGGGATGTGGATGATAGCAAA | SI00655473 | Hs_NBEA_3 |
| 900029-10-B | NM_013277 | CAGGTGGATGTAGAGATCAAA | SI00101178 | Hs_RACGAP1_3 |
| 900029-10-B | NM_016263 | TGCAGTGACGCTGTCATTAAA | SI00114912 | Hs_FZR1_2 |
| 900029-10-B | NM_016848 | CAGCAAACACCTTTAAGGCAA | SI00717836 | Hs_SHC3_4 |
| 900029-10-B | NM_018362 | TGGCATATTGACCCTATATAA | SI02778041 | Hs_LIN7C_5 |
| 900029-10-B | NM_020148 | TACGAGAATCAGTCTAACAGA | SI03225355 | Hs_SPIRE1_7 |
| 900029-10-B | NM_022090 | AAGCACTAGAATATCCAGCTA | SI00620284 | Hs_LOC63920_4 |
| 900029-10-B | NM_024045 | CACAAGAATGGAACAAATCTA | SI00361655 | Hs_DDX50_3 |
| 900029-10-B | XM_001130728 X [symbol] | TCCATTGACAACAAGGCTTTA | SI03020031 | Hs_C20orf119_3 |
| 900029-10-B | NM_032704 | ATTGCCGTAAATTGTTAATAA | SI02653777 | Hs_TUBA6_2 |
| 900029-10-B | NM_002568 | ATGCCAGGTCTAGCAAACATA | SI03152352 | Hs_PABPC1_5 |
| 900029-10-B | NM_013333 | CCCGACGAGTTCTCTGACTTT | SI00380611 | Hs_EPN1_3 |
| 900029-10-B | NM_016356 | AAGCACATAGTTATTGCTGAA | SI00360059 | Hs_DCDC2_3 |
| 900029-10-B | NM_018948 | TAGACTGTATTAATAAACATA | SI00645785 | Hs_MIG-6_3 |
| 900029-10-B | NM_018440 | AACAGAGAGCCTTAATCTTTA | SI02643298 | Hs_PAG_7 |
| 900029-10-B | NM_020673 | CAGGTTTAATTTGATGGTCTA | SI02662184 | Hs_RAB22A_5 |
| 900029-10-B | NM_022101 | CGGATTCCTATTTATGCCCTA | SI03195731 | Hs_CXorf56_1 |
| 900029-10-B | NM_024108 | CAGGAAGTGGGTATCAAATTG | SI03173653 | Hs_TRAPPC6A_1 |
| 900029-10-B | NM_025248 | CAGGCCACTAAACCATCTAAA | SI00728161 | Hs_SNIP_3 |
| 900029-10-B | NM_032832 | CAGCAACTCATTTATATATAT | SI00623637 | Hs_LRP11_3 |
| 900029-11-A | NM_032876 NM_ [symbol] | CCCAAAGATCATGATATTCCA | SI00450093 | Hs_JUB_4 |
| 900029-11-A | NM_001080457 X [symbol] | GAGCAAGAACCTGGTGCGCAA | SI03102869 | Hs_LRRC4B_5 |
| 900029-11-A | NM_001011724 N [symbol] | TAGGACATGCTCCAAAGAAGA | SI03228127 | Hs_RP11-78J21.1_4 |
| 900029-11-A | XM_209178 XM_ [symbol] | CCGCACCAAGCTGCAGAACAA | SI02779532 | Hs_LOC284393_5 |
| 900029-11-A | XM_936114 | GAGATGGTCTATCCTAACCAA | SI02795142 | Hs_LOC642045_4 |
| 900029-11-A | XM_944239 XM_9 | TTCAACAAAGCTACAGAGTTA | SI03240524 | Hs_LOC648695_4 |
| 900029-11-A | XM_001130021 | CACGATGGGAGGACAAGTTCA | SI03533467 | Hs_LOC731292_4 |
| 900029-11-A | NM_001001870 N [symbol] | CTGGATGACAGTTGGGTGATA | SI03211068 | Hs_MGC14376_6 |
| 900029-11-A | NM_033546 | CAGGGCCAATCAATTTCACCA | SI03176495 | Hs_MRLC2_5 |
| 900029-11-A | NM_173354 | TCCACACATCATAAAGCTTTA | SI02660490 | Hs_SNF1LK_6 |
| 900029-11-A | NM_173086 | CAGGCTGAATGGCGAAGGCAT | SI03175949 | Hs_KRT6E_7 |
| 900029-11-A | XM_931847 XM_ [symbol] | TTCAACAAAGCTACAGAGTTA | SI03240517 | Hs_LOC642954_4 |
| 900029-11-A | XM_939418 | CCAAGTCTGAGTGGTTGGATA | SI03180002 | Hs_LOC650332_4 |
| 900029-11-A | XM_001129414 | CAGGATACTGTTTCCTTACTA | SI03528063 | Hs_LOC731751_4 |
| 900029-11-A | NM_031922 | AACGCTCTTCAAGCTCACAAA | SI03125871 | Hs_REPS1_5 |
| 900029-11-A | NM_153207 | CTGGATGCGATAAGACATCGA | SI03211145 | Hs_AEBP2_5 |
| 900029-11-A | NM_174907 | CAAGGAGAAACTATACAGGAA | SI00691628 | Hs_PPP4R2_4 |
| 900029-11-A | NM_020063 | CTCCCTGTTCGGAGATTGATA | SI03200540 | Hs_BARHL2_5 |
| 900029-11-A | XM_928854 XM_ [symbol] | TGGAGTGTTCTGCACTTACAA | SI03237640 | Hs_LOC643751_4 |
| 900029-11-A | XM_001126093 X [symbol] | CCCGGCCTTCAACGAGTTCTA | SI03528147 | Hs_LOC649768_4 |
| 900029-11-A | NM_001037954 N [symbol] | AACTACCGAGATTATTTGATA | SI03126746 | Hs_DIXDC1_5 |
| 900029-11-A | NM_198450 | TAGTTAAATTAGAATGGTGTA | SI03229394 | Hs_FAM121A_2 |
| 900029-11-A | NM_001005463 | AAAGTTGTAGCTAAATATTTA | SI03122511 | Hs_EBF3_1 |
| 900029-11-A | XM_370726 XM_ [symbol] | ATGAAGCTCCAGGAACTTTAA | SI00510405 | Hs_LOC387927_4 |
| 900029-11-A | XM_001129413 X [symbol] | CTAGAGTATGCAGCTGGTAAA | SI03198174 | Hs_LOC643752_4 |
| 900029-11-A | XM_001128002 X [symbol] | CCCGGCAGGGTTGTTTCTTAA | SI03515610 | Hs_LOC728153_4 |
| 900029-11-A | NM_033284 NM_ [symbol] | TTCGCTCTGAAATGGAACAAA | SI03242407 | Hs_TBL1Y_5 |
| 900029-11-A | NM_080677 | CTGCATGGACTGTATACTCGA | SI00369390 | Hs_Dlc2_4 |
| 900029-11-A | NM_001013442 X | TTGGATTACTATTAAGTGGTT | SI03245004 | Hs_UNQ3072_4 |
| 900029-11-A | XM_371781 XM_ [symbol] | CCGCCTGTTGTTACCGGTATT | SI03189249 | Hs_LOC389342_7 |
| 900029-11-A | XM_292963 | CCAAGTCTGAGTGGTTGGATA | SI02792762 | Hs_LOC643997_4 |
| 900029-11-A | XM_001126120 | CAGAGTATGAACCAAGGGTTA | SI03499167 | Hs_LOC728198_4 |
| 900029-11-A | NM_001033553 N [symbol] | CCAAATGTCGATGGAACATCA | SI03179883 | Hs_SPECC1_1 |
| 900029-11-A | NM_080746 | CCAGAAGATTCATATCTCCAA | SI00705572 | Hs_RPL10L_4 |
| 900029-11-A | NM_173598 | AAGGAAATCCATTACTTCAAA | SI02665551 | Hs_KSR2_6 |
| 900029-11-A | XM_001130327 X [symbol] | CAAGATGAAAGAAGGCATAAA | SI00529053 | Hs_LOC390006_4 |
| 900029-11-A | XM_928701 XM_ [symbol] | CATGGAATTATTGGTTATAAA | SI03179596 | Hs_LOC645691_4 |
| 900029-11-A | XM_001129715 | CACAGAAGACGAAGAGACTAT | SI03530506 | Hs_LOC731173_4 |
| 900029-11-B | NM_032876 NM_ [symbol] | CTGCTAGGCAACCAAATTTAA | SI00450086 | Hs_JUB_3 |
| 900029-11-B | NM_001080457 X | CACCTCCATGACCTCCGTCAA | SI00624288 | Hs_LRRC4B_4 |
| 900029-11-B | NM_001011724 N [symbol] | ATGAAGCAGCTTCATATCTAA | SI03151204 | Hs_RP11-78J21.1_3 |
| 900029-11-B | XM_209178 XM_ [symbol] | TCCCTTCAGTGTGCCACTGAA | SI03114790 | Hs_LOC284393_8 |
| 900029-11-B | XM_936114 | CAGAGAAGCGCCTATTAACAA | SI02795135 | Hs_LOC642045_3 |
| 900029-11-B | XM_939603 XM_ [symbol] | GACCGAATGTCTAGGATTTAA | SI03216171 | Hs_LOC648695_3 |
| 900029-11-B | XM_001130021 | ACGAACTGGTATAATGATTTA | SI03533460 | Hs_LOC731292_3 |
| 900029-11-B | NM_001001870 N [symbol] | AGGAGTAGAAGGCTCAAACAA | SI03145779 | Hs_MGC14376_5 |
| 900029-11-B | NM_033546 | ACTGAAAGAACTTTAGCTAAA | SI00648025 | Hs_MRLC2_3 |
| 900029-11-B | NM_173354 | TACCTCGGACGTCTACGGAAA | SI03109162 | Hs_SNF1LK_9 |
| 900029-11-B | NM_173086 | CAGCCCTCTCATCTCCTGGAA | SI03171028 | Hs_KRT6E_6 |
| 900029-11-B | XM_927104 XM_ [symbol] | GACCGAATGTCTAGGATTTAA | SI03216164 | Hs_LOC642954_3 |
| 900029-11-B | XM_939418 | CAGGTCCTGGCATATTGTCCA | SI03177258 | Hs_LOC650332_3 |
| 900029-11-B | XM_001129414 | CAACGTGTCAAGCTACTTAAA | SI03528056 | Hs_LOC731751_3 |
| 900029-11-B | NM_031922 | ATGGCTATGATTTACCAGAAA | SI00701288 | Hs_REPS1_4 |
| 900029-11-B | NM_153207 | AAGCACCTTCTTTAACAATAA | SI00292880 | Hs_AEBP2_4 |
| 900029-11-8 | NM_174907 | AAGAGGCAAATTTGCAGCAAA | SI00691621 | Hs_PPP4R2_3 |
| 900029-11-B | NM_020063 | TCCGACCACCAGCTCAATCAA | SI00310366 | Hs_BARHL2_4 |
| 900029-11-B | XM_928854 XM_ [symbol] | GGCGATGGTGCTGTTAGTAAA | SI03221078 | Hs_LOC643751_3 |
| 900029-11-B | XM_001126093 X [symbol] | CCCGATGGGTTTCCTAAATTT | SI03528140 | Hs_LOC649768_3 |
| 900029-11-B | NM_001037954 N [symbol] | CTGCTGAAATGTAAACAAGAA | SI00364259 | Hs_DIXDC1_3 |
| 900029-11-B | NM_198450 | AAGAGTCACTCCCTAAACCTA | SI03128951 | Hs_FAM121A_1 |
| 900029-11-B | NM_001005463 | TACACTACAATTGTTAATAAA | SI00368039 | Hs_DKFZp667B021 [symbol] |
| 900029-11-B | XM_370726 XM_9 | TTGGAGAATATTTCTACTACA | SI00510398 | Hs_LOC387927_3 |
| 900029-11-B | XM_001129413 X [symbol] | CGCCCAGATTCAGGCGTGTAA | SI03193708 | Hs_LOC643752_3 |
| 900029-11-B | XM_001128002 X [symbol] | AAAGGTAAATAAGCTCCCTAA | SI03515603 | Hs_LOC728153_3 |
| 900029-11-B | NM_033284 NW_ [symbol] | CAGGAGTGTATATGTTTCATA | SI00740460 | Hs_TBL1Y_4 |
| 900029-11-B | NM_080677 | TTGACAAGAAATATAACCCTA | SI00369383 | Hs_Dlc2_3 |
| 900029-11-B | NM_001013442 | CTGCTATATAAGAAAGAGGTA | SI03209052 | Hs_UNQ3072_3 |
| 900029-11-B | XM_371781 XM_ [symbol] | CACCAATAAATTCTACTAACT | SI03160570 | Hs_LOC389342_5 |
| 900029-11-B | XM_292963 | CAGGTCCTGGCATATTGTCCA | SI02792755 | Hs_LOC643997_3 |
| 900029-11-B | XM_001126120 | CAGAAGTTTCATCTTAGGATA | SI03499160 | Hs_LOC728198_3 |
| 900029-11-B | NM_001033553 N [symbol] | CAGCAACAAATGGTACAGGAA | SI00434777 | Hs_HCMOGT-1_4 |
| 900029-11-B | NM_080746 | CCGTATTTGTGCCAACAAATA | SI00705565 | Hs_RPL10L_3 |
| 900029-11-B | NM_173598 | CAGGCTTACCGTGGACGCCTA | SI02665544 | Hs_KSR2_5 |
| 900029-11-B | XM_001130327 X [symbol] | TTCTTCAACATTGCCATCAAT | SI00529046 | Hs_LOC390006_3 |
| 900029-11-B | XM_928701 XM_9 | ATCCTATGCAATATATCTAAA | SI03149713 | Hs_LOC645691_3 |
| 900029-11-B | XM_001129715 | TTGAACTACAGTACAGAAAGA | SI03283343 | Hs_LOC731173_3 |

[symbol]

indicates data missing or illegible when filed

While certain preferred embodiments of the present invention have been described and specifically exemplified above, it is not intended that the invention be limited to such embodiments. Various modifications may be made to the invention without departing from the scope and spirit thereof as set forth in the following claims.

What is claimed is:

1. A method for identifying compounds which modulate sensitivity to EGFR/MEK-1 targeting agents, comprising;
a) providing an EGFR/NEDD9/TGF-β interactome, comprising genes which are involved in cellular proliferation and EGFR/MEK-1 signalling;
b) providing at least one compound which targets EGFR/NEDD9/TGF-β interactome genes;
c) contacting a cancer cell with at least one EGFR-MEK-1 targeting agent in the presence and absence of at least compound from step b);
d) determining cell viability in the presence of said agent alone and in the presence and absence of said at least one compound, compounds which increase or decrease sensitivity modulating cell sensitivity to said agent.

2. The method of claim 1, wherein said compound is an siRNA which modulates expression of at least one EGFR/NEDD9/TGF-β interactome gene.

3. The method of claim 2, wherein said at least one siRNA increases sensitivity.

4. The method of claim 2, wherein said at least one siRNA decreases sensitivity.

5. The method of claim 2, wherein said at least one siRNA is selected from the list of siRNAs provided in Table 3.

6. The method of claim 2, wherein said at least one siRNA inhibits expression of a gene target selected from the targets provided in Table 2.

7. The method of claim 1, wherein prior to performing steps a), b), c) and d) said cancer cells are incubated in the presence and absence of said at least one compound to determine whether said compound alters cellular viability in the absence of said agent.

8. The method of claim 7, wherein said compound is an siRNA.

9. The method of claim 1, wherein said cells are further examined for the presence of at least one parameter selected from the group consisting of morphological alterations, altered migratory properties, altered levels of apoptosis, altered angiogenic properties, and altered chromosomal or DNA integrity.

10. The method of claim 1, wherein said EGFR-MEK-1 targeting agent is selected from the group consisting of cetuximab, panitumumab, lapatinab, erlotinib, gefitinib, and U0126.

11. The method of claim 10, wherein said agent is panitumumab and said sensitizing molecule targets an EGFR/NEDD9/TGF-β interactome gene selected from the group consisting of ALK, ANXA6, EPHA5, RAPGEF1, PLSCR1, PRKACB, PRKCE, SPECC1, RHOA, POU3F2, LOC390006, and LOC284393.

12. The method of claim 11, wherein said at least one siRNA molecule targets an EGFR/NEDD9/TGF-β interactome gene selected from the group consisting of EPHA5 and PRKACB.

13. The method of claim 10, wherein said agent is erlotinib and said at least one siRNA targets a gene selected from the group consisting of ALK, ANXA6, ASCL2, BCL3, CAV2, CD22, CD59, CDC42, CTNNA1, DCN, EPB41L1, EPHA5, ERBB3, FGFR2, RAPGEF1, PLCG2, PLSCR1, PRKACB, PRKCE, SC4MOL, CXCL12, SLP1, SOS2, STAT3, TLN1, PIP5K1B, BCAR3, TRIP12, BCAR1, TOB1, VAV3, SPECC1, ARF4, ARF5, RHOA, CALD1, CDC25A, CDH3, DAG1, DLG4, DOCK2, DUSP4, DUSP7, EGR3, ERBB2, FCGR3A, FER, FES, FGR, FLNA, FUS, GAPDH, GNAI2, GRB7. GRB14, NRG1, HIP1, HES1, INPPL1, LTK, MATK, MAP3K1, MYC, POU3F2, PKN2, MAP2K1, RET, RPL10, RPS6KA3, SHC1, SRF, KLF10, TMEM1, RPS6KA5, RPL23, NRG2, SH2D3C, ANKRD11, DLL4, PARD3, LOC63920, DIXDC1, and TBL1Y.

14. The method of claim 13, wherein said targets an EGFR/NEDD9/TGF-β interactome gene selected from the group consisting of ANXA6, EPG41L1, TOB1, FLNA, and MAP2K1.

15. The method of claim 10, wherein said agent is U0126 and said sensitizing siRNA molecule targets an EGFR/NEDD9/TGF-β interactome gene selected from the group consisting of ANXA6, ERBB3, PLCG2, CXCL12, STAT3, SPECC1, GNAI2, LTK, LYN, and LOC390006.

16. The method of claim 1, further comprising contacting said cells with cpt-11.

17. The method of claim 1 wherein said cancer cell is selected from a cancer cell line selected from the group consisting of a breast cancer cell line, a colorectal cancer cell line, a lung cancer cell line, a kidney cancer cell line, an ovarian cancer cell line, melanoma cell line, bladder cancer cell line, a glioma cancer cell line, a prostate cancer cell line and brain cancer cell line.

18. A method for the treatment of cancer in a patient in need thereof comprising administration of an effective amount of an EGFR-MEK-1 targeting agent selected from the group consisting of cetuximab, erlotinib, lapatinib, panitumumab, and a compound which inhibits the activity of at least one gene selected from the group consisting of ANXA6, ARF4, ARF5, ASCL2, BCAR1, DLG4, CD59, DIXDC1, DUSP4, DUSP6, DUSP7, FER, MATK, NEDD9, PRIAP19/SLP1, INPPL1, SHC1, STAT3, AURKA, PRKACB, RAPGEF1, RPS6KA5, FLNA, PRKCE, SC4MOL and SH2DC3 in a pharmaceutically acceptable carrier.

19. The method of claim 18, wherein said compound is an siRNA and said carrier comprises a liposome.

20. The method of claim 19, wherein said cancer is selected from the group consisting of colorectal cancer, HNSCC, lung cancer, pancreatic cancer, glioma, breast cancer and ovarian cancer.

21. A pharmaceutical composition comprising an effective amount of an EGFR-MEK-1 targeting agent selected from the group consisting of cetuximab, erlotinib, panitumumab, gefitinib, U0126, and lapantinib and an effective amount of at least one siRNA provided in Table 2, in a pharmaceutically acceptable carrier.

22. The method of claim 1, wherein said cancer cell is isolated from a patient.

23. An EGFR/NEDD9/TGF-β interactome as claimed in claim 1, comprising a plurality of biomarkers associated with chemoresistance, said markers being listed in table 2 and immobilized on a solid support.

24. A method for determining whether a patient will respond to EGFR/MEK-1 targeting therapy, comprising assessing a cancer cell from said patient for expression levels of the biomarkers of claim 23.

25. The method of claim 1, wherein said interactome comprises the 638 gene targets provided in Table 1.

26. The method of claim 18, wherein said agent is erlotinib and said compound is stattic which down modulates STAT3.

27. The method of claim 18, wherein said agent is erlotinib and said compound down modulates PKRC and is enzastaurin or Ro-318220.

28. The method of claim 18, wherein said agent is erlotinib and said compound downmodulates expression of AURKA.

29. The method of claim 18, wherein said compound down modulates at least two gene targets selected from the group consisting of SH2DC3, BCAR1 and NEDD9.

30. The method of claim 28, wherein said agent is erlotinb.
